(19) United States
(12) Patent Application Publication
(10) Pub. No.: US 2024/0237512 A1
(43) Pub. Date: Jul. 11, 2024

(54) HETEROCYCLIC COMPOUND AND ORGANIC LIGHT EMITTING DEVICE COMPRISING SAME

(71) Applicant: LT MATERIALS CO., LTD., Yongin-si, Gyeonggi-do (KR)

(72) Inventors: Ji-Un KIM, Yongin-si (KR); Seung-Gyu YANG, Yongin-si (KR); Won-Jang JEONG, Yongin-si (KR); Dong-Jun KIM, Yongin-si (KR)

(73) Assignee: LT MATERIALS CO., LTD., Yongin-si, Gyeonggi-do (KR)

(21) Appl. No.: 18/285,447

(22) PCT Filed: Jan. 20, 2022
(86) PCT No.: PCT/KR2022/001043

§ 371 Date: Oct. 3, 2023

(30) Foreign Application Priority Data
Jun. 8, 2021 (KR) 10-2021-0074342

(51) Int. Cl. H10K 85/60; C07D 307/77; C07D 307/91; C07D 409/12; C07D 409/14; C09K 11/06; H10K 50/15; H10K 50/18

(52) U.S. Cl. CPC H10K 85/636; C07D 307/77; C07D 307/91; C07D 409/12; C07D 409/14; C09K 11/06; H10K 85/633; C09K 2211/1018; H10K 50/15; H10K 50/181; H10K 85/615; H10K 85/626; H10K 85/6574; H10K 85/6576

(57) ABSTRACT

The present application provides a heterocyclic compound and an organic light emitting device in which the heterocyclic compound is contained in an organic material layer.

### TECHNICAL FIELD

This application claims priority to and the benefit of Korean Patent Application No. 10-2021-0074342 filed in the Korean Intellectual Property Office on Jun. 8, 2021, the entire contents of which are incorporated herein by reference.

The present specification relates to a heterocyclic compound and an organic light emitting device including the same.

### BACKGROUND ART

An electroluminescence device is a kind of self-emitting type display device, and has an advantage in that the viewing angle is wide, the contrast is excellent, and the response speed is fast.

An organic light emitting device has a structure in which an organic thin film is disposed between two electrodes. When a voltage is applied to an organic light emitting device having the structure, electrons and holes injected from the two electrodes combine with each other in an organic thin film to make a pair, and then, emit light while being extinguished. The organic thin film may be composed of a single layer or multi layers, if necessary.

A material for the organic thin film may have a light emitting function, if necessary. For example, as the material for the organic thin film, it is also possible to use a compound, which may itself constitute a light emitting layer alone, or it is also possible to use a compound, which may serve as a host or a dopant of a host-dopant-based light emitting layer. In addition, as a material for the organic thin film, it is also possible to use a compound, which may perform a function such as hole injection, hole transport, electron blocking, hole blocking, electron transport or electron injection.

In order to improve the performance, life time, or efficiency of the organic light emitting device, there is a continuous need for developing a material for an organic thin film.

### RELATED ART DOCUMENT

### Patent Document

U.S. Pat. No. 4,356,429

### DISCLOSURE

### Technical Problem

The present specification has been made in an effort to provide a heterocyclic compound and an organic light emitting device including the same.

### Technical Solution

An exemplary embodiment of the present application provides a heterocyclic compound represented by the following Formula 1.

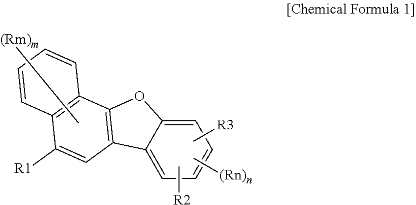

In Chemical Formula 1,
R1 to R3 are the same as or different from each other, and are each independently a substituted or unsubstituted aryl group having 6 to 60 carbon atoms; a substituted or unsubstituted heteroaryl group having 2 to 60 carbon atoms; or -(L1)ₐNR4R5, and one of R1 to R3 is -(L1)ₐNR4R5,
L1 is a direct bond; a substituted or unsubstituted arylene group having 6 to 60 carbon atoms; or a substituted or unsubstituted heteroarylene group having 2 to 60 carbon atoms, and a is an integer from 0 to 3, and when a is 2 or higher, L1's in the parenthesis are the same as or different from each other,
R4 and R5 are the same as or different from each other, and are each independently a substituted or unsubstituted aryl group having 6 to 60 carbon atoms; or a substituted or unsubstituted heteroaryl group having 2 to 60 carbon atoms,
Rm and Rn are the same as or different from each other, and are each independently hydrogen; deuterium; a halogen; a cyano group; a substituted or unsubstituted aryl group having 6 to 60 carbon atoms; or a substituted or unsubstituted alkyl group having 1 to 60 carbon atoms, m is an integer from 0 to 5, and when m is 2 or more, Rm's in the parenthesis are the same as or different from each other, and
n is an integer from 0 to 2, and when n is 2, Rn's in the parenthesis are the same as or different from each other.

Further, another exemplary embodiment of the present application provides an organic light emitting device including a first electrode, a second electrode and an organic material layer having one or more layers provided between the first electrode and the second electrode, in which one or more layers of the organic material layer include the heterocyclic compound represented by Chemical Formula 1.

### Advantageous Effects

A heterocyclic compound according to an exemplary embodiment of the present application can be used as a material for an organic material layer of an organic light emitting device. The heterocyclic compound can be used as a material for a hole injection layer, a hole transport layer, a light emitting layer, an electron transport layer, an electron injection layer, a charge generation layer, and the like in an organic light emitting device. In particular, the heterocyclic compound represented by Chemical Formula 1 can be used as a material for the light emitting layer of the organic light emitting device. In addition, when the heterocyclic compound represented by Chemical Formula 1 is used for an organic light emitting device, the driving voltage of the device can be lowered, the light efficiency of the device can be improved, and the life time characteristics of the device can be improved due to the thermal stability of the compound.

### DESCRIPTION OF DRAWINGS

FIGS. 1 to 3 each are views schematically illustrating a stacking structure of an organic light emitting device according to an exemplary embodiment of the present application.

### MODE FOR INVENTION

Hereinafter, the present application will be described in detail.

An exemplary embodiment of the present application provides a heterocyclic compound represented by the following Formula 1.

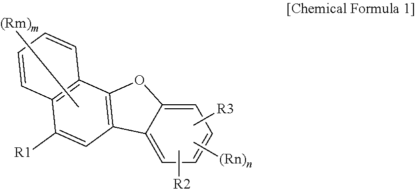

In Chemical Formula 1,
R1 to R3 are the same as or different from each other, and are each independently a substituted or unsubstituted aryl group having 6 to 60 carbon atoms; a substituted or unsubstituted heteroaryl group having 2 to 60 carbon atoms; or -(L1)ₐNR4R5, and one of R1 to R3 is -(L1)ₐNR4R5,
L1 is a direct bond; a substituted or unsubstituted arylene group having 6 to 60 carbon atoms; or a substituted or unsubstituted heteroarylene group having 2 to 60 carbon atoms, and a is an integer from 0 to 3, and when a is 2 or higher, L1's in the parenthesis are the same as or different from each other,
R4 and R5 are the same as or different from each other, and are each independently a substituted or unsubstituted aryl group having 6 to 60 carbon atoms; or a substituted or unsubstituted heteroaryl group having 2 to 60 carbon atoms,
Rm and Rn are the same as or different from each other, and are each independently hydrogen; deuterium; a halogen; a cyano group; a substituted or unsubstituted aryl group having 6 to 60 carbon atoms; or a substituted or unsubstituted alkyl group having 1 to 60 carbon atoms, m is an integer from 0 to 5, and when m is 2 or more, Pm's in the parenthesis are the same as or different from each other, and
n is an integer from 0 to 2, and when n is 2, Rn's in the parenthesis are the same as or different from each other.

Chemical Formula 1 is a dibenzofuran-based compound and has three substituents of R1 to R3, and at least one of the substituents has an amine group, so that electrons are more abundant, and accordingly, when the compound represented by Chemical Formula 1 is used for a device, the current flow is improved, so that there is an effect of lowering the driving voltage. Further, since Chemical Formula 1 has an excellent hole transporting ability by having an amine group having hole characteristics as a substituent, there is an effect of lowering the driving voltage when the compound represented by Chemical Formula 1 is used for a device.

In addition, Chemical Formula 1 has thermal superiority to a dibenzofuran-based compound having two or less substituents, such that there is an effect that the life time is increased.

In the present specification, the term “substitution” means that a hydrogen atom bonded to a carbon atom of a compound is changed into another substituent, and a position to be substituted is not limited as long as the position is a position at which the hydrogen atom is substituted, that is, a position at which the substituent may be substituted, and when two or more substituents are substituted, the two or more substituents may be the same as or different from each other.

In the present specification, “substituted or unsubstituted” means being unsubstituted or substituted with one or more substituents selected from the group consisting of a straight-chained or branched alkyl having 1 to 60 carbon atoms; a straight-chained or branched alkenyl having 2 to 60 carbon atoms; a straight-chained or branched alkynyl having 2 to 60 carbon atoms; a monocyclic or polycyclic cycloalkyl having 3 to 60 carbon atoms; a monocyclic or polycyclic heterocycloalkyl having 2 to 60 carbon atoms; a monocyclic or polycyclic aryl having 6 to 60 carbon atoms; a monocyclic or polycyclic heteroaryl having 2 to 60 carbon atoms; —SiRR′R″; —P(═O)RR′; an alkylamine having 1 to 20 carbon atoms; a monocyclic or polycyclic arylamine having 6 to 60 carbon atoms; and a monocyclic or polycyclic heteroarylamine having 2 to 60 carbon atoms, or being unsubstituted or substituted with a substituent to which two or more substituents selected from the above exemplified substituents are linked, and means that R, R′ and R″ are the same as or different from each other, and are each independently hydrogen; deuterium; a halogen; a substituted or unsubstituted alkyl having 1 to 60 carbon atoms; a substituted or unsubstituted aryl having 6 to 60 carbon atoms; or a substituted or unsubstituted heteroaryl having 2 to 60 carbon atoms.

In the present specification, “when a substituent is not indicated in the structure of a chemical formula or compound” means that a hydrogen atom is bonded to a carbon atom. However, since deuterium (²H) is an isotope of hydrogen, some hydrogen atoms may be deuterium.

In an exemplary embodiment of the present application, “when a substituent is not indicated in the structure of a chemical formula or compound” may mean that all the positions that may be reached by the substituent are hydrogen or deuterium. That is, deuterium is an isotope of hydrogen, and some hydrogen atoms may be deuterium which is an isotope, and in this case, the content of deuterium may be 0% to 100%.

In an exemplary embodiment of the present application, in “the case where a substituent is not indicated in the structure of a chemical formula or compound”, when the content of deuterium is 0%, the content of hydrogen is 100%, and all the substituents do not explicitly exclude deuterium such as hydrogen, hydrogen and deuterium may be mixed and used in the compound.

In an exemplary embodiment of the present application, deuterium is one of the isotopes of hydrogen, is an element that has a deuteron composed of one proton and one neutron as a nucleus, and may be represented by hydrogen-2, and the element symbol may also be expressed as D or 2H.

In an exemplary embodiment of the present application, the isotope means an atom with the same atomic number (Z), but different mass numbers (A), and the isotope may be interpreted as an element which has the same number of protons, but different number of neutrons.

In an exemplary embodiment of the present application, when the total number of substituents of a basic compound is defined as T1 and the number of specific substituents among the substituents is defined as T2, the meaning of the content T % of the specific substituent may be defined as T2/T1×100=T %.

That is, in an example, the deuterium content of 20% in a phenyl group represented by

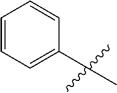 [SMILES: *c1ccccc1]

means that the total number of substituents that the phenyl group can have is 5 (T1 in the formula), and may be represented by 20% when the number of deuteriums among the substituents is 1 (T2 in the formula). That is, a deuterium content of 20, in the phenyl group may be represented by the following structural formula.

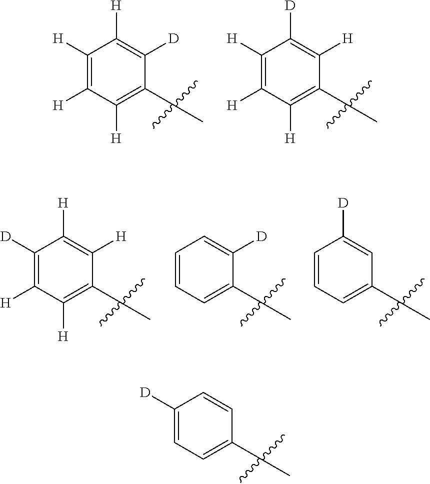 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

Further, in an exemplary embodiment of the present application, “a phenyl group having a deuterium content of 0%” may mean a phenyl group that does not include a deuterium atom, that is, has five hydrogen atoms.

In the present specification, the halogen may be fluorine, chlorine, bromine or iodine.

In the present specification, the alkyl group includes a straight-chain or branched-chain having 1 to 60 carbon atoms, and may be additionally substituted with another substituent. The number of carbon atoms of the alkyl group may be 1 to 60, specifically 1 to 40, and more specifically 1 to 20. Specific examples thereof include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group, and the like, but are not limited thereto.

In the present specification, the alkenyl group includes a straight-chain or branched-chain having 2 to 60 carbon atoms, and may be additionally substituted with another substituent. The number of carbon atoms of the alkenyl group may be 2 to 60, specifically 2 to 40, and more specifically 2 to 20. Specific examples thereof include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group, and the like, but are not limited thereto. In the present specification, the alkynyl group includes a straight-chain or branched-chain having 2 to 60 carbon atoms, and may be additionally substituted with another substituent. The number of carbon atoms of the alkynyl group may be 2 to 60, specifically 2 to 40, and more specifically 2 to 20.

In the present specification, an alkoxy group may be straight-chained, branched, or cyclic. The number of carbon atoms of the alkoxy group is not particularly limited, but is preferably 1 to 20. Specific examples thereof include methoxy, ethoxy, n-propoxy, isopropoxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, p-methylbenzyloxy, and the like, but are not limited thereto.

In the present specification, the cycloalkyl group includes a monocycle or polycycle having 3 to 60 carbon atoms, and may be additionally substituted with another substituent. Here, the polycycle means a group in which a cycloalkyl group is directly linked to or fused with another cyclic group. Here, another cyclic group may also be a cycloalkyl group, but may also be another kind of cyclic group, for example, a heterocycloalkyl group, an aryl group, a heteroaryl group, and the like. The number of carbon atoms of the cycloalkyl group may be 3 to 60, specifically 3 to 40, and more specifically 5 to 20.

Specific examples thereof include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group, and the like, but are not limited thereto.

In the present specification, the heterocycloalkyl group includes 0, S, Se, N, or Si as a heteroatom, includes a monocycle or polycycle having 2 to 60 carbon atoms, and may be additionally substituted with another substituent. Here, the polycycle means a group in which a heterocycloalkyl group is directly linked to or fused with another cyclic group. Here, another cyclic group may also be a heterocycloalkyl group, but may also be another kind of cyclic group, for example, a cycloalkyl group, an aryl group, a heteroaryl group, and the like. The number of carbon atoms of the heterocycloalkyl group may be 2 to 60, specifically 2 to 40, and more specifically 3 to 20.

In the present specification, the aryl group includes a monocycle or polycycle having 6 to 60 carbon atoms, and may be additionally substituted with another substituent. Here, the polycycle means a group in which an aryl group is directly linked to or fused with another cyclic group. Here, another cyclic group may also be an aryl group, but may also be another kind of cyclic group, for example, a cycloalkyl group, a heterocycloalkyl group, a heteroaryl group, and the like. The aryl group includes a spiro group. The number of carbon atoms of the aryl group may be 6 to 60, specifically 6 to 40, and more specifically 6 to 25. Specific examples of the aryl group include a phenyl group, a biphenyl group, a terphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused cyclic group thereof, and the like, but are not limited thereto.

In the present specification, a silyl group includes Si and is a substituent to which the Si atom is directly linked as a radical, and is represented by —SiR101R102R103, and R101 to R103 are the same as or different from each other, and may be each independently a substituent composed of at least one of hydrogen; deuterium; a halogen group; an alkyl group; an alkenyl group; an alkoxy group; a cycloalkyl group; an aryl group; and a heterocyclic group. Specific examples of the silyl group include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and the like, but are not limited thereto.

In the present specification, a phosphine oxide group is represented by —P(═O)R104R105, and R104 and R105 are the same as or different from each other, and may be each independently a substituent composed of at least one of hydrogen; deuterium; a halogen group; an alkyl group; an alkenyl group; an alkoxy group; a cycloalkyl group; an aryl group; and a heterocyclic group. Specific examples of the phosphine oxide group include a diphenylphosphine oxide group, dinaphthylphosphine oxide, and the like, but are not limited thereto.

In the present specification, the phosphine oxide group may be substituted, and adjacent substituents may be bonded to each other to form a ring.

In the present specification, the spiro group is a group including a spiro structure, and may have 15 to 60 carbon atoms. For example, the spiro group may include a structure in which a 2,3-dihydro-1H-indene group or a cyclohexane group is spiro-bonded to a fluorenyl group. Specifically, the spiro group may include any one of the groups of the following structural formulae.

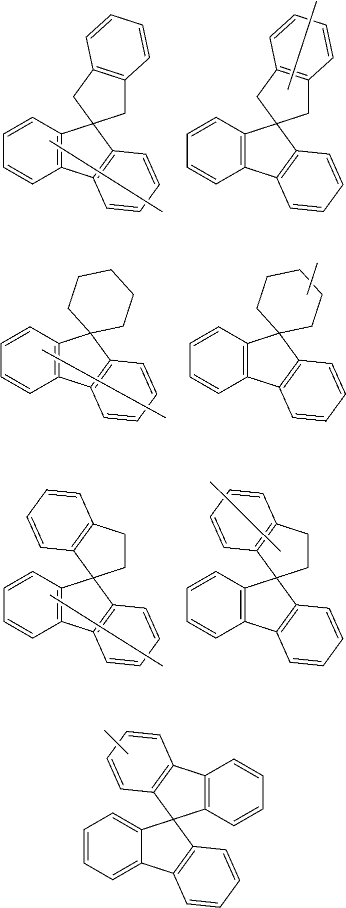

In the present specification, the heteroaryl group includes S, O, Se, N, or Si as a heteroatom, includes a monocycle or polycycle having 2 to 60 carbon atoms, and may be additionally substituted with another substituent. Here, the polycycle means a group in which a heteroaryl group is directly linked to or fused with another cyclic group. Here, another cyclic group may also be a heteroaryl group, but may also be another kind of cyclic group, for example, a cycloalkyl group, a heterocycloalkyl group, an aryl group, and the like. The number of carbon atoms of the heteroaryl group may be 2 to 60, specifically 2 to 40, and more specifically 3 to 25. Specific examples of the heteroaryl group include a pyridyl group, a pyrrolyl group, a pyrimidyl group, a pyridazinyl group, a furanyl group, a thiophene group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a quinozolilyl group, a naphthyridyl group, an acridinyl group, a phenanthridinyl group, an imidazopyridinyl group, a diaza naphthalenyl group, a triazaindene group, an indolyl group, an indolizinyl group, a benzothiazolyl group, a benzoxazolyl group, a benzimidazolyl group, a benzothiophene group, a benzofuran group, a dibenzothiophene group, a dibenzofuran group, a carbazolyl group, a benzocarbazolyl group, a dibenzocarbazolyl group, a phenazinyl group, a dibenzosilole group, spirobi (dibenzosilole), a dihydrophenazinyl group, a phenoxazinyl group, a phenanthridyl group, an imidazopyridinyl group, a thienyl group, an indolo[2,3-a]carbazolyl group, an indolo[2,3-b]carbazolyl group, an indolinyl group, a 10,11-dihydro-dibenzo[b,f]azepin group, a 9,10-dihydroacridinyl group, a phenanthrazinyl group, a phenothiazinyl group, a phthalazinyl group, a naphthylidinyl group, a phenanthrolinyl group, a benzo[c][1,2,5]thiadiazolyl group, a 5,10-dihydrodibenzo[b,e][1,4]azasilinyl, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group, and the like, but are not limited thereto.

In the present specification, the amine group may be selected from the group consisting of a monoalkylamine group; a monoarylamine group; a monoheteroarylamine group; —NH₂; a dialkylamine group; a diarylamine group; a diheteroarylamine group; an alkylarylamine group; an alkylheteroarylamine group; and an arylheteroarylamine group, and the number of carbon atoms thereof is not particularly limited, but is preferably 1 to 30. Specific examples of the amine group include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group, and the like, but are not limited thereto.

In the present specification, an arylene group means that there are two bonding positions in an aryl group, that is, a divalent group. The above-described description on the aryl group may be applied to the arylene group, except that the arylene groups are each a divalent group. Further, a heteroarylene group means that there are two bonding positions in a heteroaryl group, that is, a divalent group. The above-described description on the heteroaryl group may be applied to the heteroarylene group, except for a divalent heteroarylene group.

In the present specification, the “adjacent” group may mean a substituent substituted with an atom directly linked to an atom in which the corresponding substituent is substituted, a substituent disposed to be sterically closest to the corresponding substituent, or another substituent substituted with an atom in which the corresponding substituent is substituted. For example, two substituents substituted at the ortho position in a benzene ring and two substituents substituted with the same carbon in an aliphatic ring may be interpreted as groups which are “adjacent” to each other.

A heterocyclic compound according to an exemplary embodiment of the present application is represented by Chemical Formula 1. More specifically, the heterocyclic compound represented by Chemical Formula 1 may be used as a material for an organic material layer of an organic light emitting device by the structural characteristics of the core structure and the substituent as described above.

In an exemplary embodiment of the present application, the deuterium content of the heterocyclic compound represented by Chemical Formula 1 may be each independently 0-% to 100%.

In an exemplary embodiment of the present application, the deuterium content of the heterocyclic compound represented by Chemical Formula 1 may be each independently 10% to 100%.

In an exemplary embodiment of the present application, the deuterium content of the heterocyclic compound represented by Chemical Formula 1 may be each independently 20% to 100%.

In an exemplary embodiment of the present application, R1 to R3 of Chemical Formula 1 are the same as or different from each other, and may be each independently a substituted or unsubstituted aryl group having 6 to 60 carbon atoms; a substituted or unsubstituted heteroaryl group having 2 to 60 carbon atoms; or -(L1)ₐNR4R5, and one of R1 to R3 may be -(L1)ₐNR4R5.

In an exemplary embodiment of the present application, R1 to R3 of Chemical Formula 1 are the same as or different from each other, and may be each independently a substituted or unsubstituted aryl group having 6 to 40 carbon atoms; a substituted or unsubstituted heteroaryl group having 2 to 40 carbon atoms; or -(L1)ₐNR4R5, and one of R1 to R3 may be -(L1)ₐNR4R5.

In an exemplary embodiment of the present application, R1 to R3 of Chemical Formula 1 are the same as or different from each other, and may be each independently a substituted or unsubstituted aryl group having 6 to 20 carbon atoms; a substituted or unsubstituted heteroaryl group having 2 to 20 carbon atoms; or -(L1)ₐNR4R5, and one of R1 to R3 may be -(L1)ₐNR4R5.

In an exemplary embodiment of the present application, R1 to R3 of Chemical Formula 1 are the same as or different from each other, and may be each independently a substituted or unsubstituted aryl group having 6 to 60 carbon atoms; or -(L1)ₐNR4R5, and one of R1 to R3 may be -(L1)ₐNR4R5.

In an exemplary embodiment of the present application, R1 to R3 of Chemical Formula 1 are the same as or different from each other, and may be each independently a substituted or unsubstituted aryl group having 6 to 40 carbon atoms; or -(L1)ₐNR4R5, and one of R1 to R3 may be -(L1)ₐNR4R5.

In an exemplary embodiment of the present application, R1 to R3 of Chemical Formula 1 are the same as or different from each other, and may be each independently a substituted or unsubstituted aryl group having 6 to 20 carbon atoms; or -(L1)ₐNR4R5, and one of R1 to R3 may be -(L1)ₐNR4R5.

In an exemplary embodiment of the present application, R1 to R3 of Chemical Formula 1 are the same as or different from each other, and may be each independently a substituted or unsubstituted heteroaryl group having 2 to 60 carbon atoms; or -(L1)ₐNR4R5, and one of R1 to R3 may be -(L1)ₐNR4R5.

In an exemplary embodiment of the present application, R1 to R3 of Chemical Formula 1 are the same as or different from each other, and may be each independently a substituted or unsubstituted heteroaryl group having 2 to 40 carbon atoms; or -(L1)ₐNR4R5, and one of R1 to R3 may be -(L1)ₐNR4R5.

In an exemplary embodiment of the present application, R1 to R3 of Chemical Formula 1 are the same as or different from each other, and may be each independently a substituted or unsubstituted heteroaryl group having 2 to 20 carbon atoms; or -(L1)ₐNR4R5, and one of R1 to R3 may be -(L1)ₐNR4R5.

When one or more of R1 to R3 of Chemical Formula 1 are a heteroaryl group, the triplet energy level (T1) value is increased, so when the compound is used for a device, it is possible to enhance the light emitting characteristics of the device, and thus there is an advantage in that a device having high efficiency can be manufactured.

In an exemplary embodiment of the present application, R1 to R3 of Chemical Formula 1 are the same as or different from each other, and are each independently a substituted or unsubstituted phenyl group; a substituted or unsubstituted biphenyl group; a substituted or unsubstituted terphenyl group; a substituted or unsubstituted naphthyl group; a substituted or unsubstituted fluorenyl group; a substituted or unsubstituted dibenzofuran group; or a substituted or unsubstituted dibenzothiophene group; or -(L1)ₐNR4R5, and one of R1 to R3 may be -(L1)ₐNR4R5.

In an exemplary embodiment of the present application, R1 is -(L1)ₐNR4R5, and R2 and R3 are the same as or different from each other, and may be each independently a substituted or unsubstituted aryl group having 6 to 60 carbon atoms; a substituted or unsubstituted heteroaryl group having 2 to 60 carbon atoms.

In an exemplary embodiment of the present application, R2 is -(L1)ₐNR4R5, and R1 and R3 are the same as or different from each other, and may be each independently a substituted or unsubstituted aryl group having 6 to 60 carbon atoms; a substituted or unsubstituted heteroaryl group having 2 to 60 carbon atoms.

In an exemplary embodiment of the present application, R3 is -(L1)ₐNR4R5, and R1 and R2 are the same as or different from each other, and may be each independently a substituted or unsubstituted aryl group having 6 to 60 carbon atoms; a substituted or unsubstituted heteroaryl group having 2 to 60 carbon atoms.

In an exemplary embodiment of the present application, R1 is -(L1)ₐNR4R5, and R2 and R3 are the same as or different from each other, and may be each independently a substituted or unsubstituted aryl group having 6 to 40 carbon atoms; or a heteroaryl group having 2 to 40 carbon atoms.

In an exemplary embodiment of the present application, R1 is -(L1)ₐNR4R5, and R2 and R3 are the same as or different from each other, and may be each independently a substituted or unsubstituted aryl group having 6 to 20 carbon atoms; or a heteroaryl group having 2 to 20 carbon atoms.

In an exemplary embodiment of the present application, R1 is -(L1)ₐNR4R5, and R2 and R3 are the same as or different from each other, and may be each independently a substituted or unsubstituted phenyl group; a substituted or unsubstituted biphenyl group; a substituted or unsubstituted terphenyl group; or a substituted or unsubstituted naphthyl group; a substituted or unsubstituted fluorenyl group; a substituted or unsubstituted dibenzofuran group; a substituted or unsubstituted dibenzothiophene group.

In an exemplary embodiment of the present application, R2 is -(L1)ₐNR4R5, and R1 and R3 are the same as or different from each other, and may be each independently a substituted or unsubstituted aryl group having 6 to 40 carbon atoms; or a heteroaryl group having 2 to 40 carbon atoms.

In an exemplary embodiment of the present application, R2 is -(L1)ₐNR4R5, and R1 and R3 are the same as or different from each other, and may be each independently a substituted or unsubstituted aryl group having 6 to 20 carbon atoms; or a heteroaryl group having 2 to 20 carbon atoms.

In an exemplary embodiment of the present application, R2 is -(L1)ₐNR4R5, and R1 and R3 are the same as or different from each other, and may be each independently a substituted or unsubstituted phenyl group; a substituted or unsubstituted biphenyl group; a substituted or unsubstituted terphenyl group; or a substituted or unsubstituted naphthyl group; a substituted or unsubstituted fluorenyl group; a substituted or unsubstituted dibenzofuran group; or a substituted or unsubstituted dibenzothiophene group.

In an exemplary embodiment of the present application, R3 is -(L1)ₐNR4R5, and R1 and R2 are the same as or different from each other, and may be each independently a substituted or unsubstituted aryl group having 6 to 40 carbon atoms; or a heteroaryl group having 2 to 40 carbon atoms.

In an exemplary embodiment of the present application, R3 is -(L1)ₐNR4R5, and R1 and R2 are the same as or different from each other, and may be each independently a substituted or unsubstituted aryl group having 6 to 20 carbon atoms; or a heteroaryl group having 2 to 20 carbon atoms.

In an exemplary embodiment of the present application, R3 is -(L1)ₐNR4R5, and R1 and R2 are the same as or different from each other, and may be each independently a substituted or unsubstituted phenyl group; a substituted or unsubstituted biphenyl group; a substituted or unsubstituted terphenyl group; or a substituted or unsubstituted naphthyl group; a substituted or unsubstituted fluorenyl group; a substituted or unsubstituted dibenzofuran group; or a substituted or unsubstituted dibenzothiophene group.

In an exemplary embodiment of the present application, R1 to R3 may each independently have a substituent substituted with deuterium, and the contents of substituted deuterium of R1 to R3 may be each independently 05 to 100%.

In an exemplary embodiment of the present application, R1 to R3 may each independently have a substituent substituted with deuterium, and the contents of substituted deuterium of R1 to R3 may be each independently 10% to 100%.

In an exemplary embodiment of the present application, R1 to R3 may each independently have a substituent substituted with deuterium, and the contents of substituted deuterium of R1 to R3 may be each independently 20% to 100%.

In an exemplary embodiment of the present application, L1 may be a direct bond; a substituted or unsubstituted arylene group having 6 to 60 carbon atoms or a substituted or unsubstituted heteroarylene group having 2 to 60 carbon atoms.

In an exemplary embodiment of the present application, L1 may be a direct bond; a substituted or unsubstituted arylene group having 6 to 40 carbon atoms or a substituted or unsubstituted heteroarylene group having 2 to 40 carbon atoms.

In an exemplary embodiment of the present application, L1 may be a direct bond; a substituted or unsubstituted arylene group having 6 to 20 carbon atoms or a substituted or unsubstituted heteroarylene group having 2 to 20 carbon atoms.

In an exemplary embodiment of the present application, L1 may be a direct bond; or a substituted or unsubstituted arylene group having 6 to 60 carbon atoms.

In an exemplary embodiment of the present application, L1 may be a direct bond; or a substituted or unsubstituted arylene group having 6 to 40 carbon atoms.

In an exemplary embodiment of the present application, L1 may be a direct bond; or a substituted or unsubstituted arylene group having 6 to 20 carbon atoms.

In another exemplary embodiment, L1 is a direct bond; or a phenylene group.

In still another exemplary embodiment, L1 is a direct bond.

In yet another exemplary embodiment, L1 is a phenylene group.

In an exemplary embodiment of the present application, a of Chemical Formula 1 is an integer from 0 to 3, and when a is 2, L1's may be the same as or different from each other.

In another exemplary embodiment, a is 0.

In still another exemplary embodiment, a is 1.

In yet another exemplary embodiment, a is 2.

In yet another exemplary embodiment, a is 3.

In an exemplary embodiment of the present application, when a is 2 or higher, L1's in the parenthesis may be the same as or different from each other.

In an exemplary embodiment of the present application, R4 and R5 are the same as or different from each other, and may be each independently a substituted or unsubstituted aryl group having 6 to 60 carbon atoms; or a substituted or unsubstituted heteroaryl group having 2 to 60 carbon atoms.

In an exemplary embodiment of the present application, R4 and R5 are the same as or different from each other, and may be each independently a substituted or unsubstituted aryl group having 6 to 40 carbon atoms; or a substituted or unsubstituted heteroaryl group having 2 to 40 carbon atoms.

In an exemplary embodiment of the present application, R4 and R5 are the same as or different from each other, and may be each independently a substituted or unsubstituted aryl group having 6 to 20 carbon atoms; or a substituted or unsubstituted heteroaryl group having 2 to 20 carbon atoms.

In an exemplary embodiment of the present application, R4 and R5 are the same as or different from each other, and may be each independently a substituted or unsubstituted phenyl group; a substituted or unsubstituted biphenyl group; or a substituted or unsubstituted naphthyl group; a substituted or unsubstituted fluorenyl group; a substituted or unsubstituted dibenzofuran group; or a substituted or unsubstituted dibenzothiophene group.

In an exemplary embodiment of the present application, R4 and R5 are the same as or different from each other, and may be each independently a phenyl group which is unsubstituted or substituted with one or more substituents selected from the group consisting of a heteroaryl group having 2 to 20 carbon atoms; a substituted or unsubstituted biphenyl group; a substituted or unsubstituted naphthyl group; a fluorenyl group which is unsubstituted or substituted with one or more substituents selected from the group consisting of an alkyl group having 1 to 10 carbon atoms and a heteroaryl group having 2 to 20 carbon atoms; a substituted or unsubstituted dibenzofuran group; or a substituted or unsubstituted dibenzothiophene group.

In an exemplary embodiment of the present application, R4 and R5 are the same as or different from each other, and may be each independently a phenyl group which is unsubstituted or substituted with one or more dibenzofuran groups; a substituted or unsubstituted biphenyl group; a substituted or unsubstituted naphthyl group; a fluorenyl group which is unsubstituted or substituted with one or more substituents selected from the group consisting of a methyl group and a phenyl group; a substituted or unsubstituted dibenzofuran group; or a substituted or unsubstituted dibenzothiophene group.

In an exemplary embodiment of the present application, Rm and Rn of Chemical Formula 1 are the same as or different from each other, and each independently hydrogen; deuterium; a cyano group; a substituted or unsubstituted aryl group having 6 to 60 carbon atoms; or a substituted or unsubstituted alkyl group having 1 to 60 carbon atoms, m is an integer from 0 to 5, and when m is 2 or higher, Rm's in the parenthesis are the same as or different from each other, and n is an integer from 0 to 2, and when n is 2, Rn's in the parenthesis are the same as or different from each other.

In an exemplary embodiment of the present application, Rm and Rn of Chemical Formula 1 are the same as or different from each other, and may each independently include hydrogen; or deuterium.

In an exemplary embodiment of the present application, Rm and Rn of Chemical Formula 1 are the same as or different from each other, and may be each independently hydrogen; or deuterium.

In an exemplary embodiment of the present application, both Rm and Rn of Chemical Formula 1 are hydrogen.

In an exemplary embodiment of the present application, both Rm and Rn of Chemical Formula 1 are deuterium.

In an exemplary embodiment of the present application, Chemical Formula 1 may be represented by any one of the following Chemical Formulae 1-1 to 1-3.

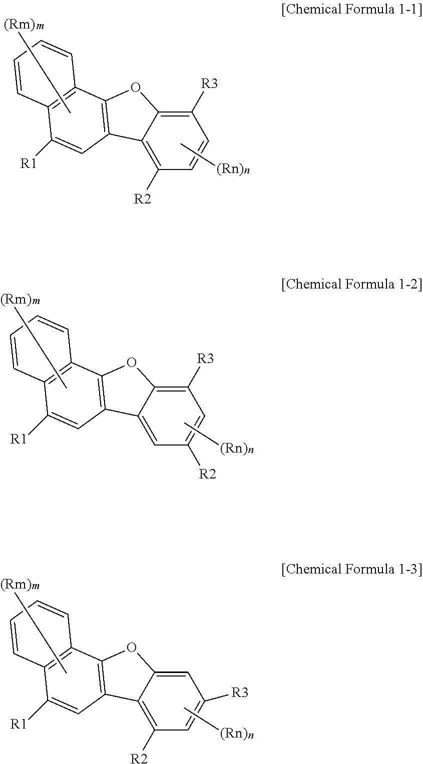

In Chemical Formulae 1-1 to 1-3, the definitions of R1 to R3, Rm, Rn, m and n are the same as those in Chemical Formula 1.

In an exemplary embodiment of the present application, Chemical Formula 1 may be represented by any one of the following Chemical Formulae 1-4 to 1-6.

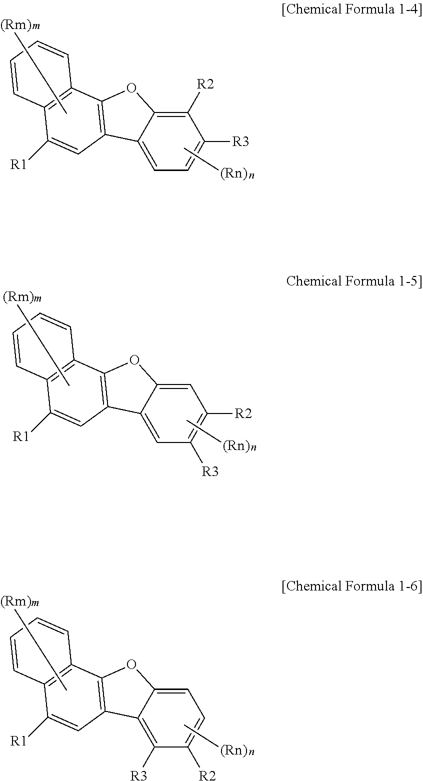

In Chemical Formulae 1-4 to 1-6, the definitions of R1 to R3, Rm, Rn, m and n are the same as those in Chemical Formula 1.

In the present specification, in regard to -(L1)ₐNR4R5, for example, Chemical Formula 1-1 may be represented by any one of the following Chemical Formulae 1-1-1 to 1-1-3.

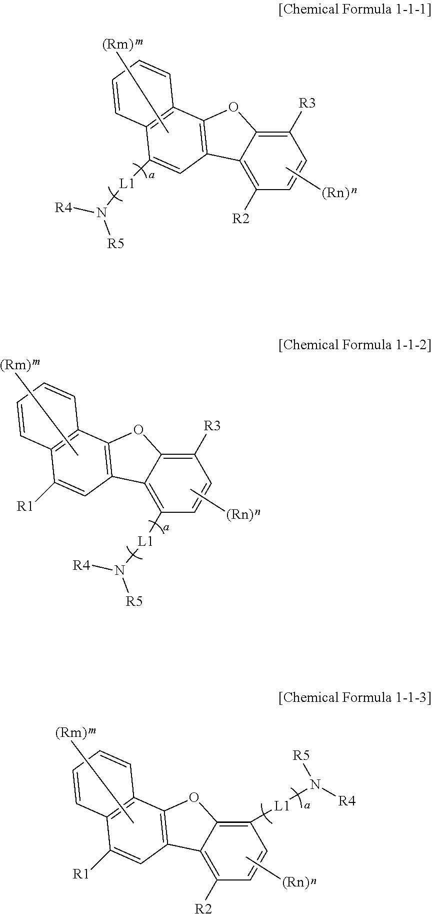

In Chemical Formulae 1-1-1 to 1-1-3, R1 to R3 are the same as or different from each other, and are each independently a substituted or unsubstituted aryl group having 6 to 60 carbon atoms; or a substituted or unsubstituted heteroaryl group having 2 to 60 carbon atoms, and
the definitions of Rm, Rn, m and n are the same as those in Chemical Formula 1.

Furthermore, the content on Chemical Formula 1 may be applied to the definitions of the aryl group and heteroaryl group of Chemical Formulae 1-1-1 to 1-1-3.

According to an exemplary embodiment of the present application, Chemical Formula 1 may be represented by any one of the following compounds, but is not limited thereto.

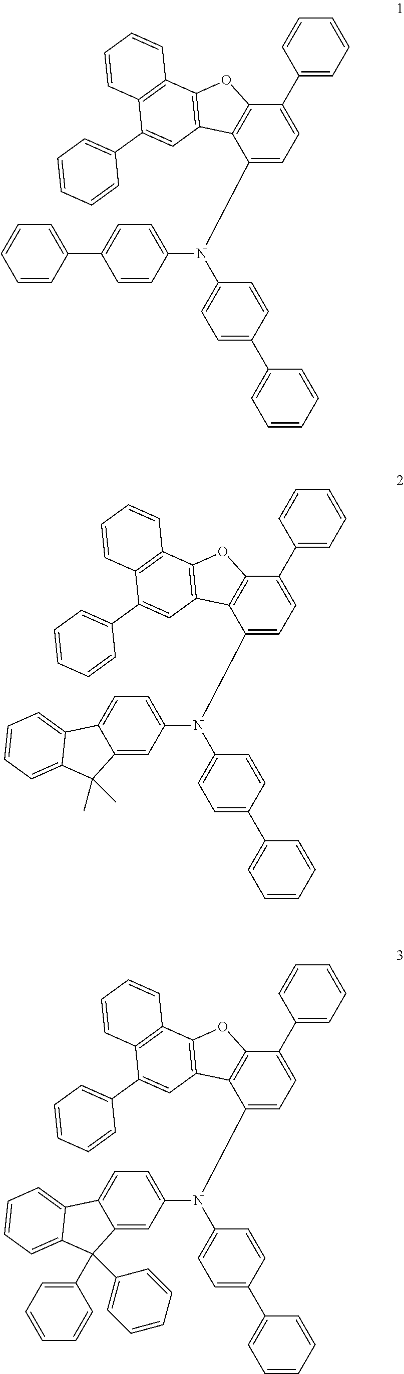 [SMILES: c1ccc(-c2ccc(N(c3ccc(-c4ccccc4)cc3)c3ccc(-c4ccccc4)c4oc5c6ccccc6c(-c6ccccc6)cc5c34)cc2)cc1 ; CC1(C)c2ccccc2-c2ccc(N(c3ccc(-c4ccccc4)cc3)c3ccc(-c4ccccc4)c4oc5c6ccccc6c(-c6ccccc6)cc5c34)cc21 ; c1ccc(-c2ccc(N(c3ccc4c(c3)C(c3ccccc3)(c3ccccc3)c3ccccc3-4)c3ccc(-c4ccccc4)c4oc5c6ccccc6c(-c6ccccc6)cc5c34)cc2)cc1]

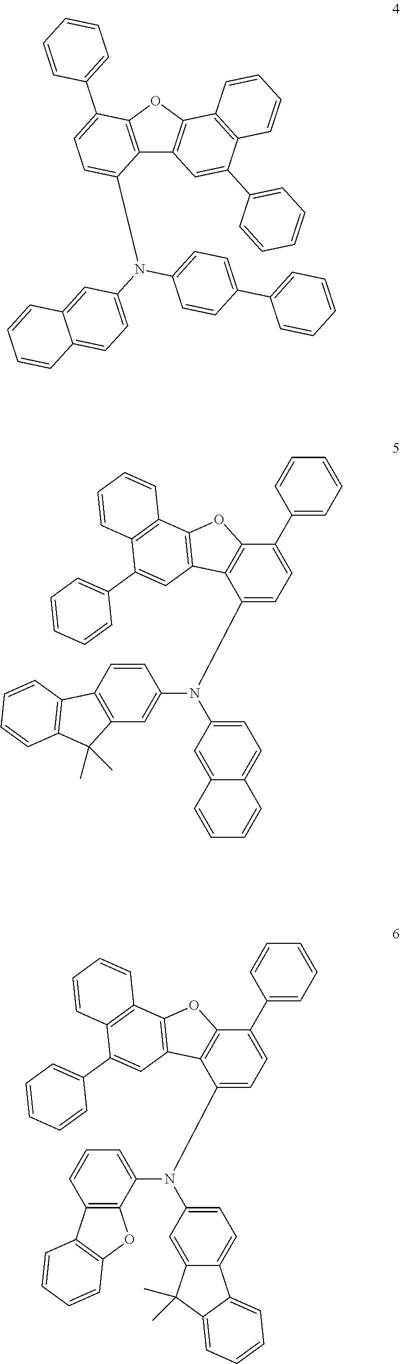 [SMILES: c1ccc(-c2ccc(N(c3ccc4ccccc4c3)c3ccc(-c4ccccc4)c4oc5c6ccccc6c(-c6ccccc6)cc5c34)cc2)cc1 ; CC1(C)c2ccccc2-c2ccc(N(c3ccc4ccccc4c3)c3ccc(-c4ccccc4)c4oc5c6ccccc6c(-c6ccccc6)cc5c34)cc21 ; CC1(C)c2ccccc2-c2ccc(N(c3cccc4c3oc3ccccc34)c3ccc(-c4ccccc4)c4oc5c6ccccc6c(-c6ccccc6)cc5c34)cc21]

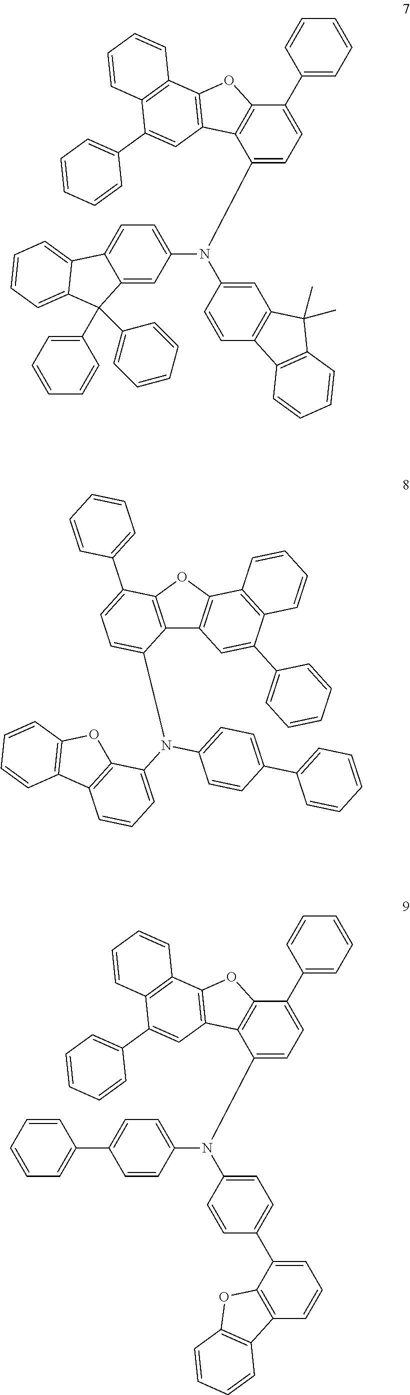 [SMILES: CC1(C)c2ccccc2-c2ccc(N(c3ccc4c(c3)C(c3ccccc3)(c3ccccc3)c3ccccc3-4)c3ccc(-c4ccccc4)c4oc5c6ccccc6c(-c6ccccc6)cc5c34)cc21 ; c1ccc(-c2ccc(N(c3cccc4c3oc3ccccc34)c3ccc(-c4ccccc4)c4oc5c6ccccc6c(-c6ccccc6)cc5c34)cc2)cc1 ; c1ccc(-c2ccc(N(c3ccc(-c4cccc5c4oc4ccccc45)cc3)c3ccc(-c4ccccc4)c4oc5c6ccccc6c(-c6ccccc6)cc5c34)cc2)cc1]

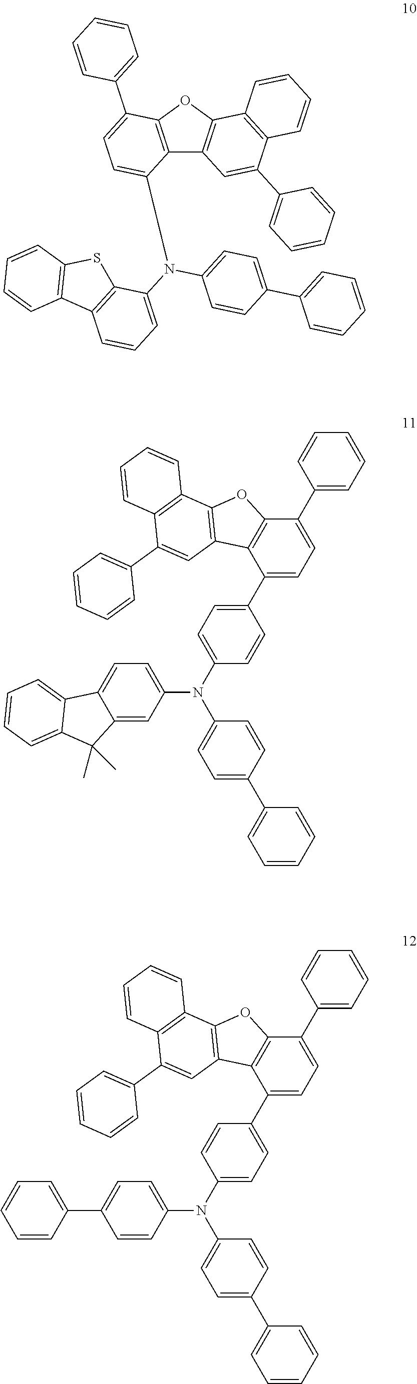 [SMILES: c1ccc(-c2ccc(N(c3cccc4c3sc3ccccc34)c3ccc(-c4ccccc4)c4oc5c6ccccc6c(-c6ccccc6)cc5c34)cc2)cc1 ; CC1(C)c2ccccc2-c2ccc(N(c3ccc(-c4ccccc4)cc3)c3ccc(-c4ccc(-c5ccccc5)c5oc6c7ccccc7c(-c7ccccc7)cc6c45)cc3)cc21 ; c1ccc(-c2ccc(N(c3ccc(-c4ccccc4)cc3)c3ccc(-c4ccc(-c5ccccc5)c5oc6c7ccccc7c(-c7ccccc7)cc6c45)cc3)cc2)cc1]

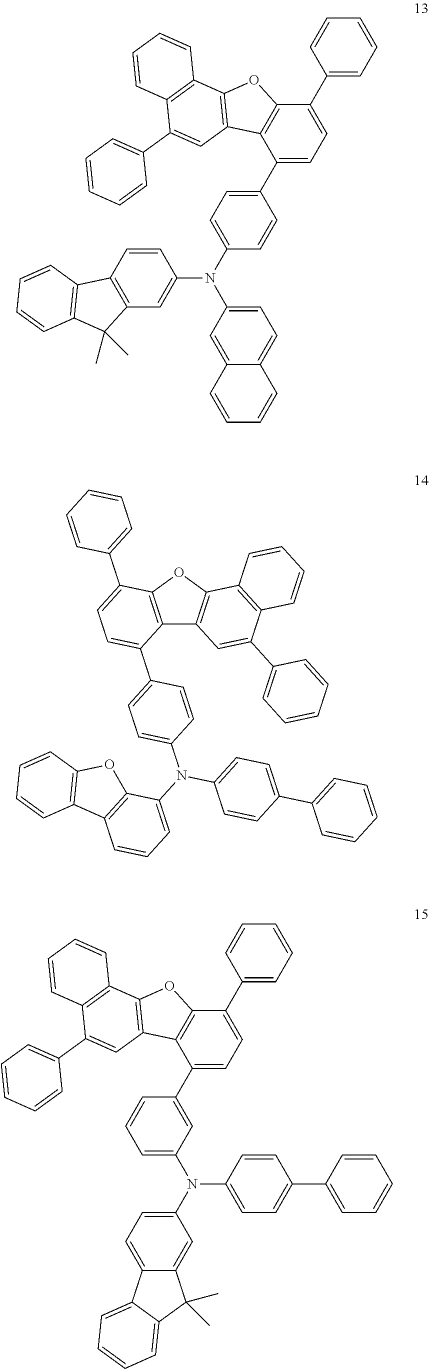 [SMILES: CC1(C)c2ccccc2-c2ccc(N(c3ccc(-c4ccc(-c5ccccc5)c5oc6c7ccccc7c(-c7ccccc7)cc6c45)cc3)c3ccc4ccccc4c3)cc21 ; c1ccc(-c2ccc(N(c3ccc(-c4ccc(-c5ccccc5)c5oc6c7ccccc7c(-c7ccccc7)cc6c45)cc3)c3cccc4c3oc3ccccc34)cc2)cc1 ; CC1(C)c2ccccc2-c2ccc(N(c3ccc(-c4ccccc4)cc3)c3cccc(-c4ccc(-c5ccccc5)c5oc6c7ccccc7c(-c7ccccc7)cc6c45)c3)cc21]

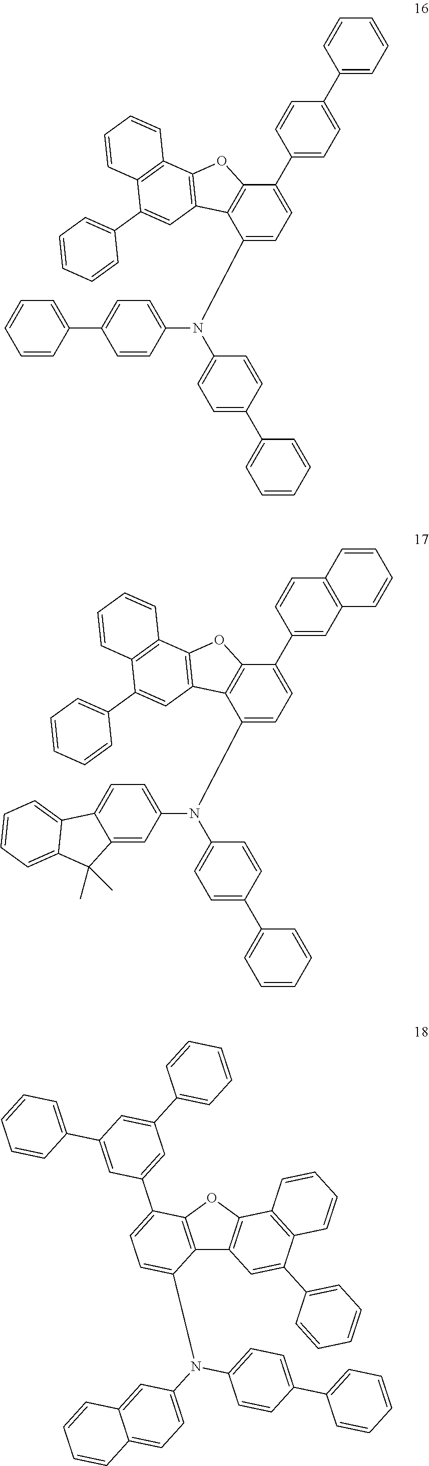 [SMILES: c1ccc(-c2ccc(-c3ccc(N(c4ccc(-c5ccccc5)cc4)c4ccc(-c5ccccc5)cc4)c4c3oc3c5ccccc5c(-c5ccccc5)cc34)cc2)cc1 ; CC1(C)c2ccccc2-c2ccc(N(c3ccc(-c4ccccc4)cc3)c3ccc(-c4ccc5ccccc5c4)c4oc5c6ccccc6c(-c6ccccc6)cc5c34)cc21 ; c1ccc(-c2ccc(N(c3ccc4ccccc4c3)c3ccc(-c4cc(-c5ccccc5)cc(-c5ccccc5)c4)c4oc5c6ccccc6c(-c6ccccc6)cc5c34)cc2)cc1]

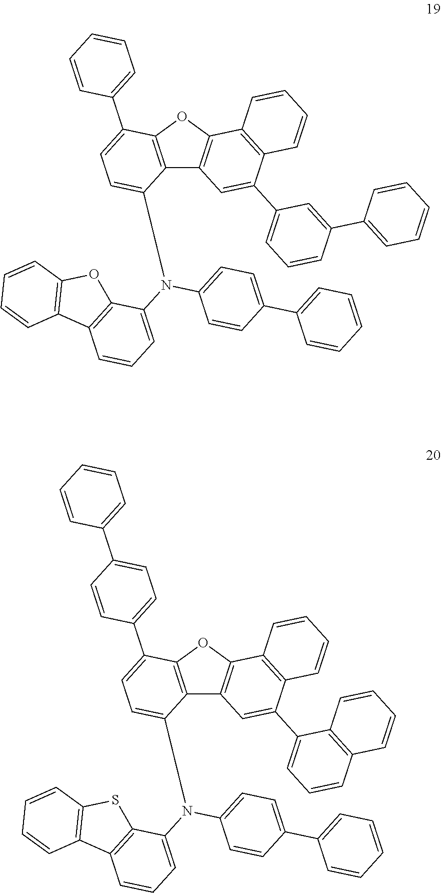 [SMILES: c1ccc(-c2ccc(N(c3cccc4c3oc3ccccc34)c3ccc(-c4ccccc4)c4oc5c6ccccc6c(-c6cccc(-c7ccccc7)c6)cc5c34)cc2)cc1 ; c1ccc(-c2ccc(-c3ccc(N(c4ccc(-c5ccccc5)cc4)c4cccc5c4sc4ccccc45)c4c3oc3c5ccccc5c(-c5cccc6ccccc56)cc34)cc2)cc1]

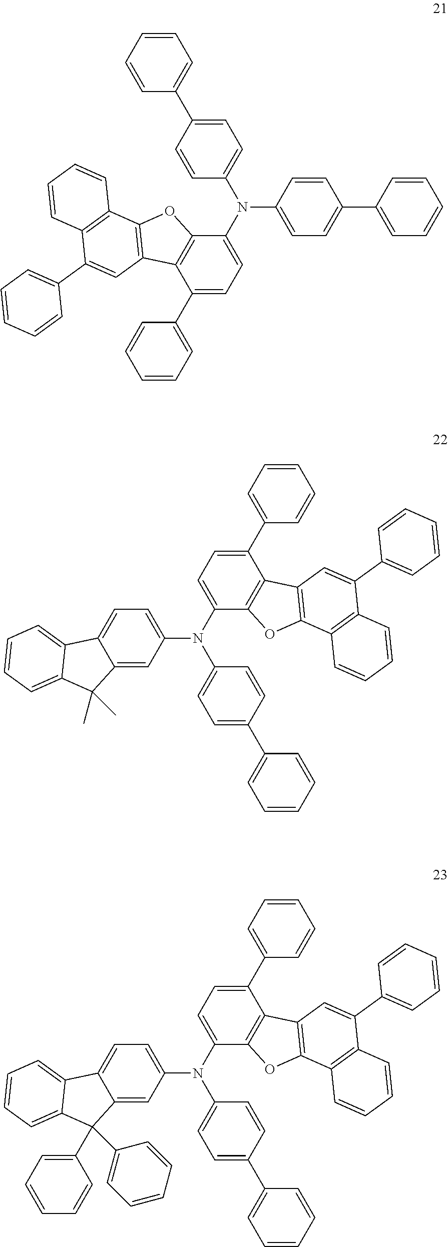 [SMILES: c1ccc(-c2ccc(N(c3ccc(-c4ccccc4)cc3)c3ccc(-c4ccccc4)c4c3oc3c5ccccc5c(-c5ccccc5)cc34)cc2)cc1 ; CC1(C)c2ccccc2-c2ccc(N(c3ccc(-c4ccccc4)cc3)c3ccc(-c4ccccc4)c4c3oc3c5ccccc5c(-c5ccccc5)cc34)cc21 ; c1ccc(-c2ccc(N(c3ccc4c(c3)C(c3ccccc3)(c3ccccc3)c3ccccc3-4)c3ccc(-c4ccccc4)c4c3oc3c5ccccc5c(-c5ccccc5)cc34)cc2)cc1]

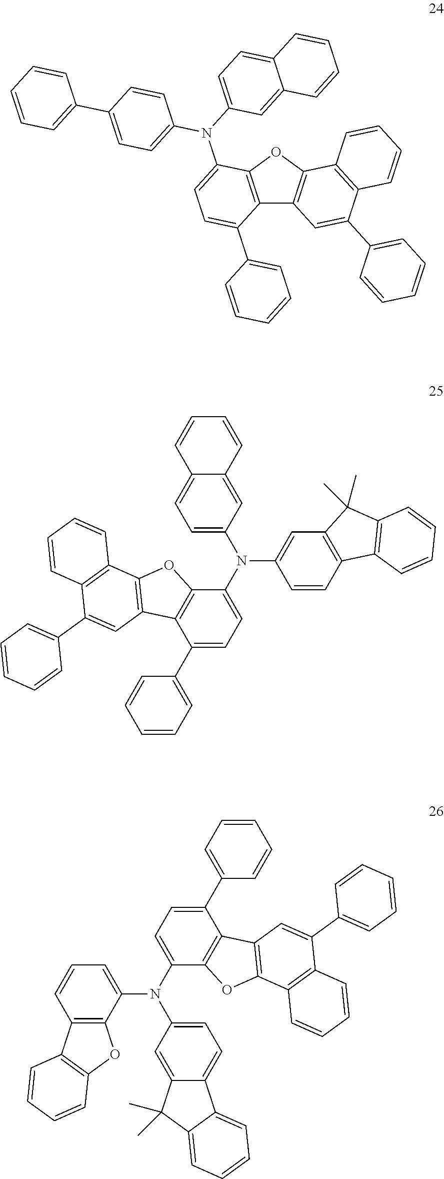 [SMILES: c1ccc(-c2ccc(N(c3ccc4ccccc4c3)c3ccc(-c4ccccc4)c4c3oc3c5ccccc5c(-c5ccccc5)cc34)cc2)cc1 ; CC1(C)c2ccccc2-c2ccc(N(c3ccc4ccccc4c3)c3ccc(-c4ccccc4)c4c3oc3c5ccccc5c(-c5ccccc5)cc34)cc21 ; CC1(C)c2ccccc2-c2ccc(N(c3cccc4c3oc3ccccc34)c3ccc(-c4ccccc4)c4c3oc3c5ccccc5c(-c5ccccc5)cc34)cc21]

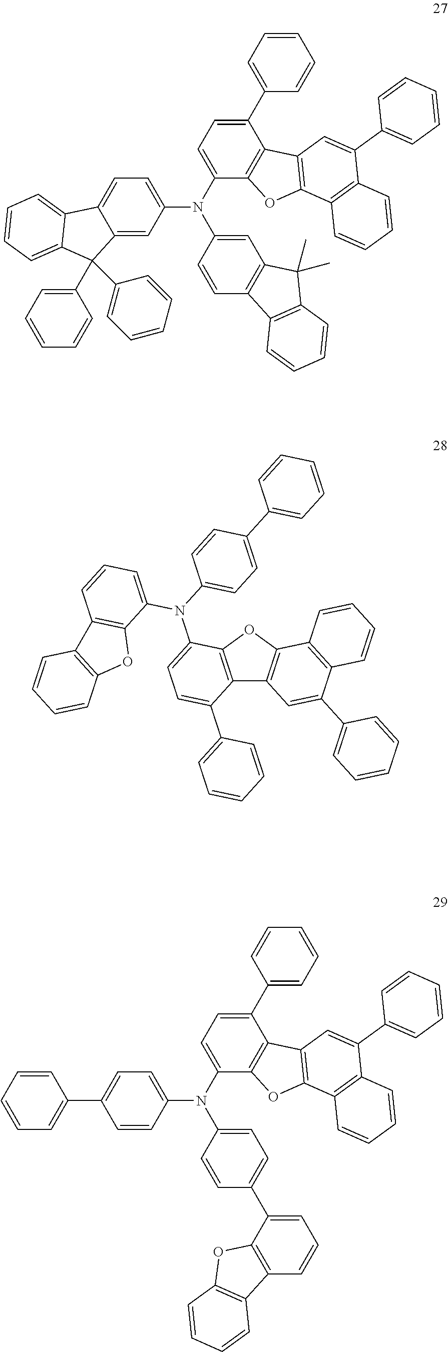 [SMILES: CC1(C)c2ccccc2-c2ccc(N(c3ccc4c(c3)C(c3ccccc3)(c3ccccc3)c3ccccc3-4)c3ccc(-c4ccccc4)c4c3oc3c5ccccc5c(-c5ccccc5)cc34)cc21 ; c1ccc(-c2ccc(N(c3cccc4c3oc3ccccc34)c3ccc(-c4ccccc4)c4c3oc3c5ccccc5c(-c5ccccc5)cc34)cc2)cc1 ; c1ccc(-c2ccc(N(c3ccc(-c4cccc5c4oc4ccccc45)cc3)c3ccc(-c4ccccc4)c4c3oc3c5ccccc5c(-c5ccccc5)cc34)cc2)cc1]

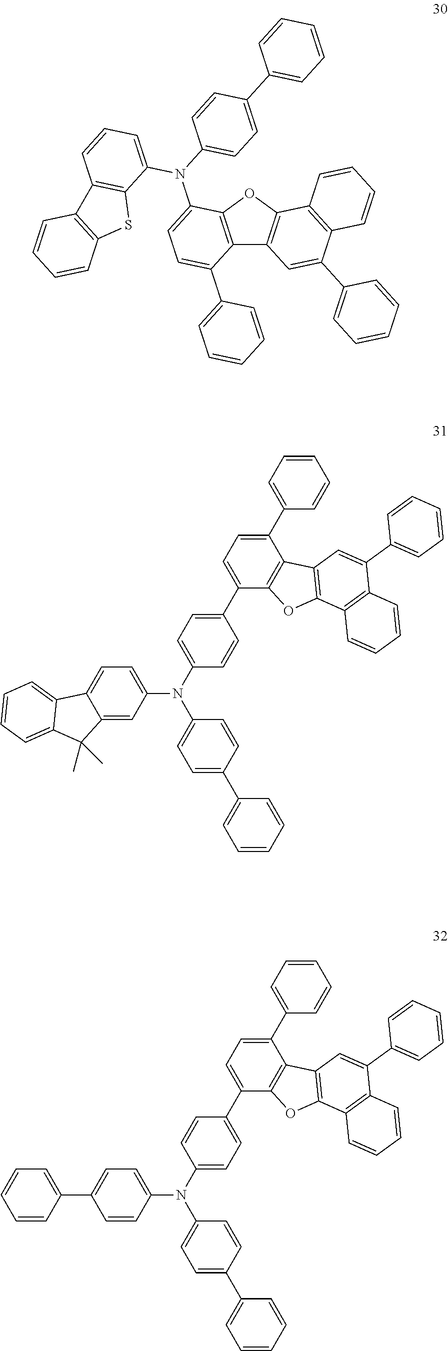 [SMILES: c1ccc(-c2ccc(N(c3ccc(-c4ccccc4)c4c3oc3c5ccccc5c(-c5ccccc5)cc34)c3cccc4c3sc3ccccc34)cc2)cc1 ; CC1(C)c2ccccc2-c2ccc(N(c3ccc(-c4ccccc4)cc3)c3ccc(-c4ccc(-c5ccccc5)c5c4oc4c6ccccc6c(-c6ccccc6)cc45)cc3)cc21 ; c1ccc(-c2ccc(N(c3ccc(-c4ccccc4)cc3)c3ccc(-c4ccc(-c5ccccc5)c5c4oc4c6ccccc6c(-c6ccccc6)cc45)cc3)cc2)cc1]

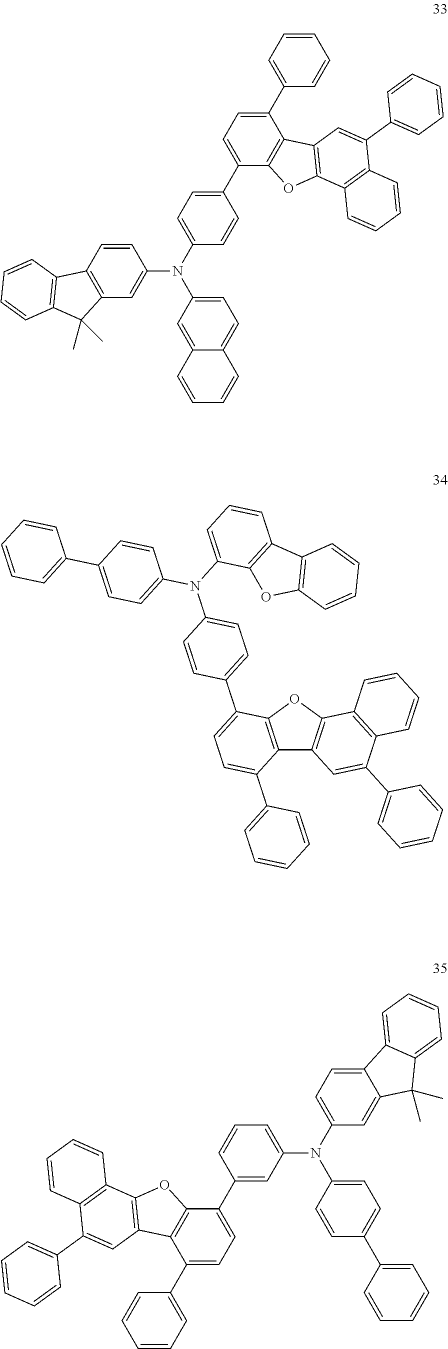 [SMILES: CC1(C)c2ccccc2-c2ccc(N(c3ccc(-c4ccc(-c5ccccc5)c5c4oc4c6ccccc6c(-c6ccccc6)cc45)cc3)c3ccc4ccccc4c3)cc21 ; c1ccc(-c2ccc(N(c3ccc(-c4ccc(-c5ccccc5)c5c4oc4c6ccccc6c(-c6ccccc6)cc45)cc3)c3cccc4c3oc3ccccc34)cc2)cc1 ; CC1(C)c2ccccc2-c2ccc(N(c3ccc(-c4ccccc4)cc3)c3cccc(-c4ccc(-c5ccccc5)c5c4oc4c6ccccc6c(-c6ccccc6)cc45)c3)cc21]

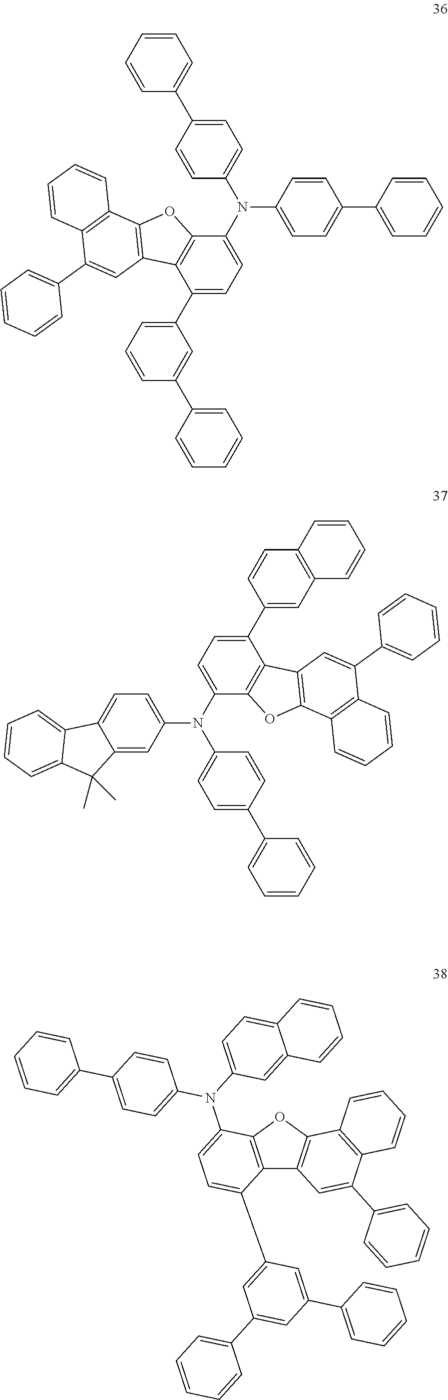 [SMILES: c1ccc(-c2ccc(N(c3ccc(-c4ccccc4)cc3)c3ccc(-c4cccc(-c5ccccc5)c4)c4c3oc3c5ccccc5c(-c5ccccc5)cc34)cc2)cc1 ; CC1(C)c2ccccc2-c2ccc(N(c3ccc(-c4ccccc4)cc3)c3ccc(-c4ccc5ccccc5c4)c4c3oc3c5ccccc5c(-c5ccccc5)cc34)cc21 ; c1ccc(-c2ccc(N(c3ccc4ccccc4c3)c3ccc(-c4cc(-c5ccccc5)cc(-c5ccccc5)c4)c4c3oc3c5ccccc5c(-c5ccccc5)cc34)cc2)cc1]

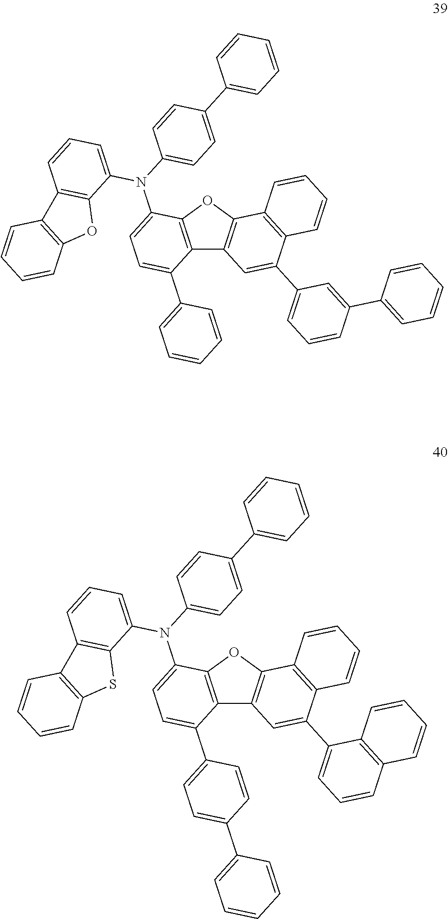 [SMILES: c1ccc(-c2ccc(N(c3cccc4c3oc3ccccc34)c3ccc(-c4ccccc4)c4c3oc3c5ccccc5c(-c5cccc(-c6ccccc6)c5)cc34)cc2)cc1 ; c1ccc(-c2ccc(-c3ccc(N(c4ccc(-c5ccccc5)cc4)c4cccc5c4sc4ccccc45)c4oc5c6ccccc6c(-c6cccc7ccccc67)cc5c34)cc2)cc1]

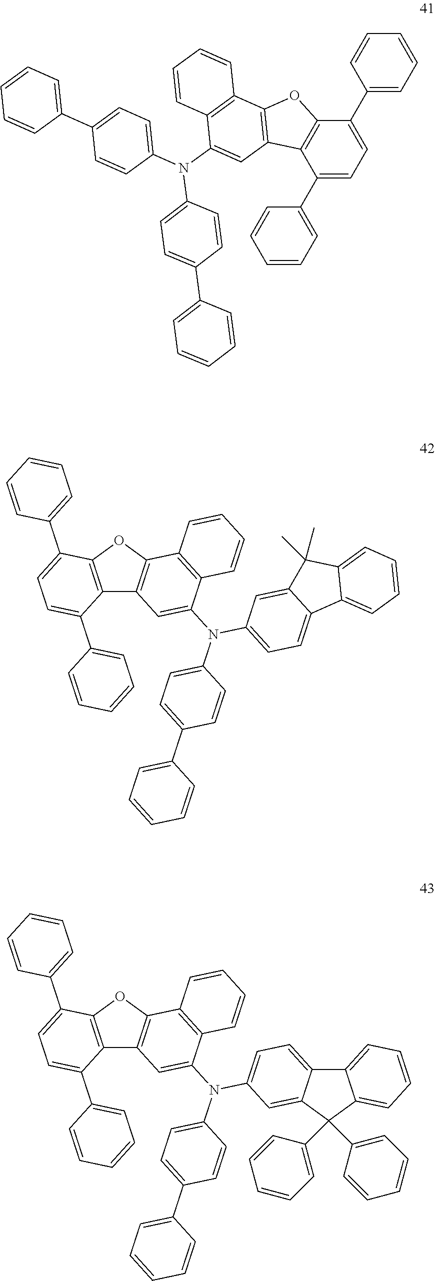 [SMILES: c1ccc(-c2ccc(N(c3ccc(-c4ccccc4)cc3)c3cc4c(oc5c(-c6ccccc6)ccc(-c6ccccc6)c54)c4ccccc34)cc2)cc1 ; CC1(C)c2ccccc2-c2ccc(N(c3ccc(-c4ccccc4)cc3)c3cc4c(oc5c(-c6ccccc6)ccc(-c6ccccc6)c54)c4ccccc34)cc21 ; c1ccc(-c2ccc(N(c3ccc4c(c3)C(c3ccccc3)(c3ccccc3)c3ccccc3-4)c3cc4c(oc5c(-c6ccccc6)ccc(-c6ccccc6)c54)c4ccccc34)cc2)cc1]

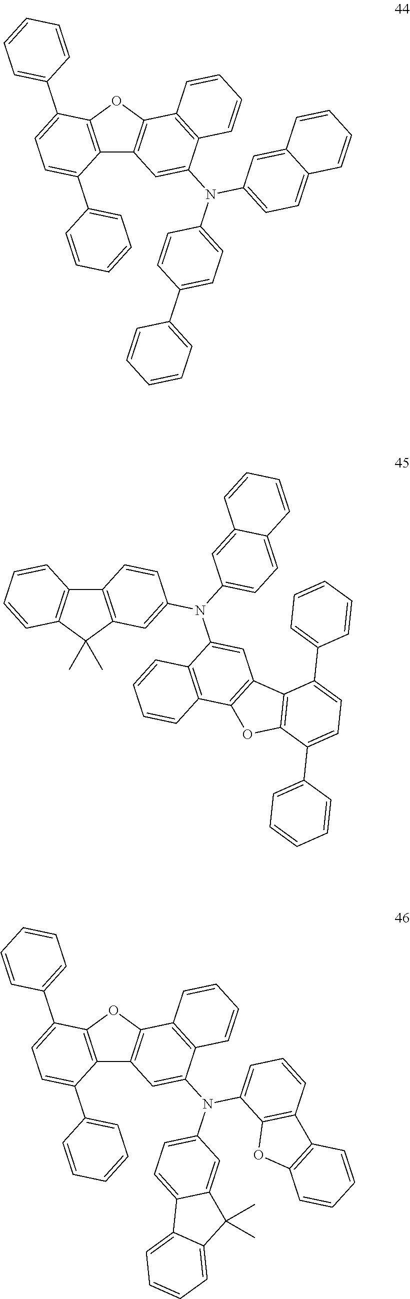 [SMILES: c1ccc(-c2ccc(N(c3ccc4ccccc4c3)c3cc4c(oc5c(-c6ccccc6)ccc(-c6ccccc6)c54)c4ccccc34)cc2)cc1 ; CC1(C)c2ccccc2-c2ccc(N(c3ccc4ccccc4c3)c3cc4c(oc5c(-c6ccccc6)ccc(-c6ccccc6)c54)c4ccccc34)cc21 ; CC1(C)c2ccccc2-c2ccc(N(c3cc4c(oc5c(-c6ccccc6)ccc(-c6ccccc6)c54)c4ccccc34)c3cccc4c3oc3ccccc34)cc21]

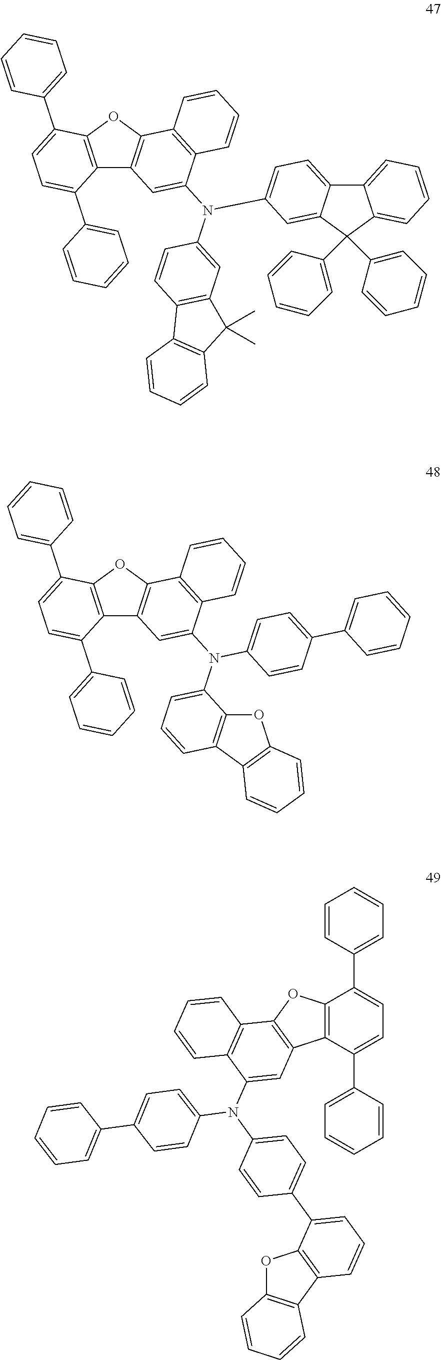 [SMILES: CC1(C)c2ccccc2-c2ccc(N(c3ccc4c(c3)C(c3ccccc3)(c3ccccc3)c3ccccc3-4)c3cc4c(oc5c(-c6ccccc6)ccc(-c6ccccc6)c54)c4ccccc34)cc21 ; c1ccc(-c2ccc(N(c3cc4c(oc5c(-c6ccccc6)ccc(-c6ccccc6)c54)c4ccccc34)c3cccc4c3oc3ccccc34)cc2)cc1 ; c1ccc(-c2ccc(N(c3ccc(-c4cccc5c4oc4ccccc45)cc3)c3cc4c(oc5c(-c6ccccc6)ccc(-c6ccccc6)c54)c4ccccc34)cc2)cc1]

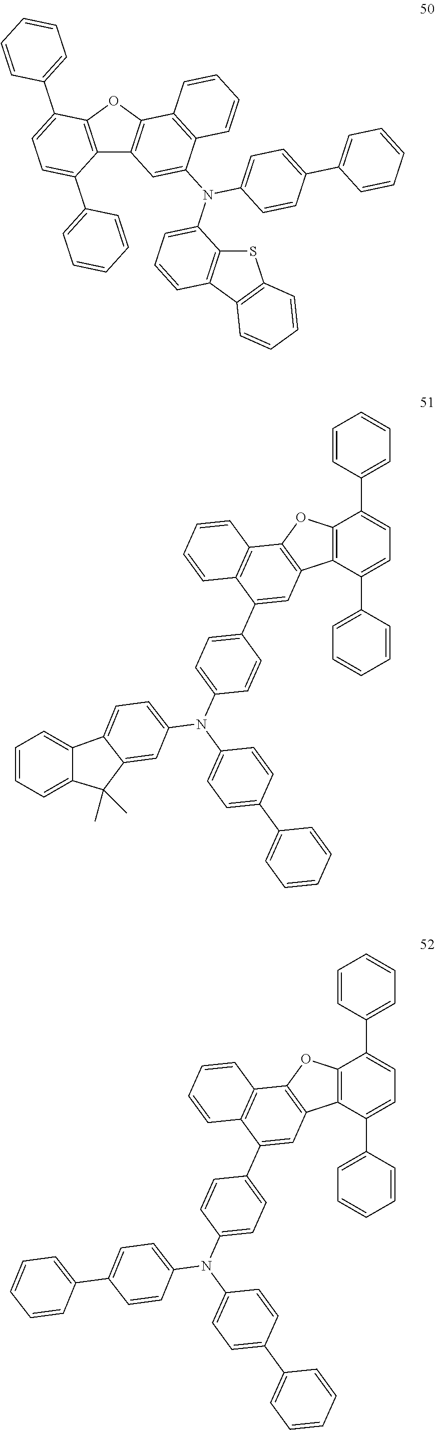 [SMILES: c1ccc(-c2ccc(N(c3cc4c(oc5c(-c6ccccc6)ccc(-c6ccccc6)c54)c4ccccc34)c3cccc4c3sc3ccccc34)cc2)cc1 ; CC1(C)c2ccccc2-c2ccc(N(c3ccc(-c4ccccc4)cc3)c3ccc(-c4cc5c(oc6c(-c7ccccc7)ccc(-c7ccccc7)c65)c5ccccc45)cc3)cc21 ; c1ccc(-c2ccc(N(c3ccc(-c4ccccc4)cc3)c3ccc(-c4cc5c(oc6c(-c7ccccc7)ccc(-c7ccccc7)c65)c5ccccc45)cc3)cc2)cc1]

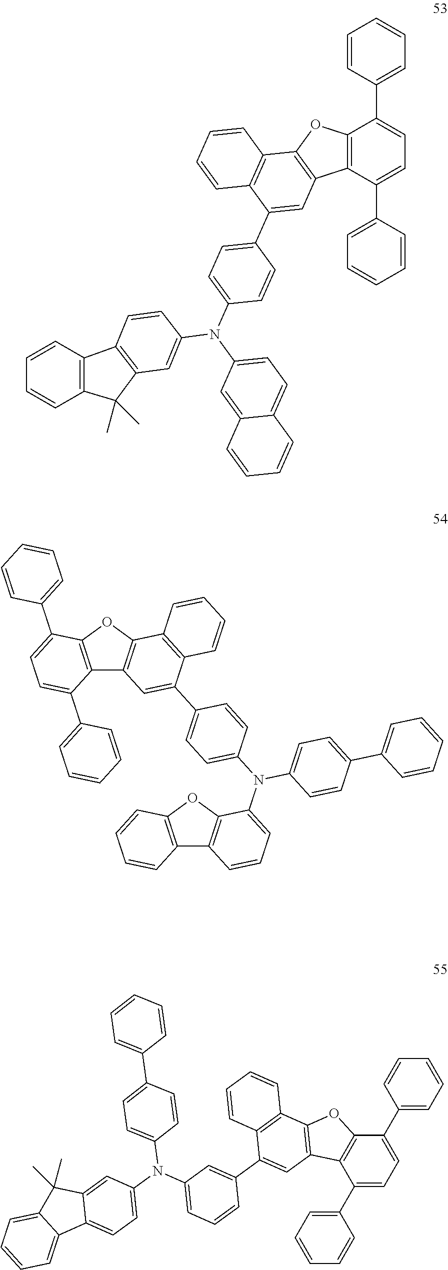 [SMILES: CC1(C)c2ccccc2-c2ccc(N(c3ccc(-c4cc5c(oc6c(-c7ccccc7)ccc(-c7ccccc7)c65)c5ccccc45)cc3)c3ccc4ccccc4c3)cc21 ; c1ccc(-c2ccc(N(c3ccc(-c4cc5c(oc6c(-c7ccccc7)ccc(-c7ccccc7)c65)c5ccccc45)cc3)c3cccc4c3oc3ccccc34)cc2)cc1 ; CC1(C)c2ccccc2-c2ccc(N(c3ccc(-c4ccccc4)cc3)c3cccc(-c4cc5c(oc6c(-c7ccccc7)ccc(-c7ccccc7)c65)c5ccccc45)c3)cc21]

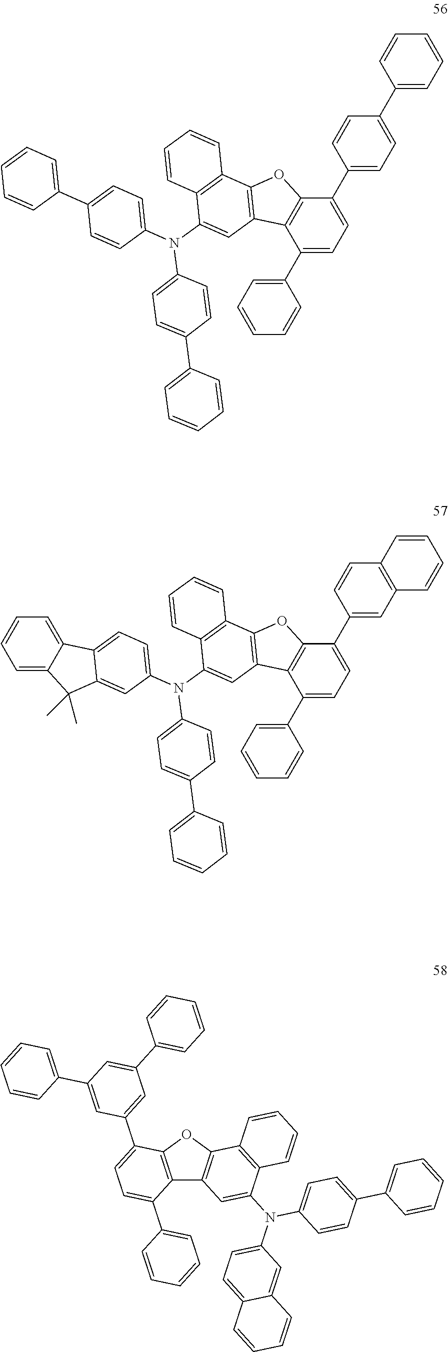 [SMILES: c1ccc(-c2ccc(-c3ccc(-c4ccccc4)c4c3oc3c5ccccc5c(N(c5ccc(-c6ccccc6)cc5)c5ccc(-c6ccccc6)cc5)cc34)cc2)cc1 ; CC1(C)c2ccccc2-c2ccc(N(c3ccc(-c4ccccc4)cc3)c3cc4c(oc5c(-c6ccc7ccccc7c6)ccc(-c6ccccc6)c54)c4ccccc34)cc21 ; c1ccc(-c2ccc(N(c3ccc4ccccc4c3)c3cc4c(oc5c(-c6cc(-c7ccccc7)cc(-c7ccccc7)c6)ccc(-c6ccccc6)c54)c4ccccc34)cc2)cc1]

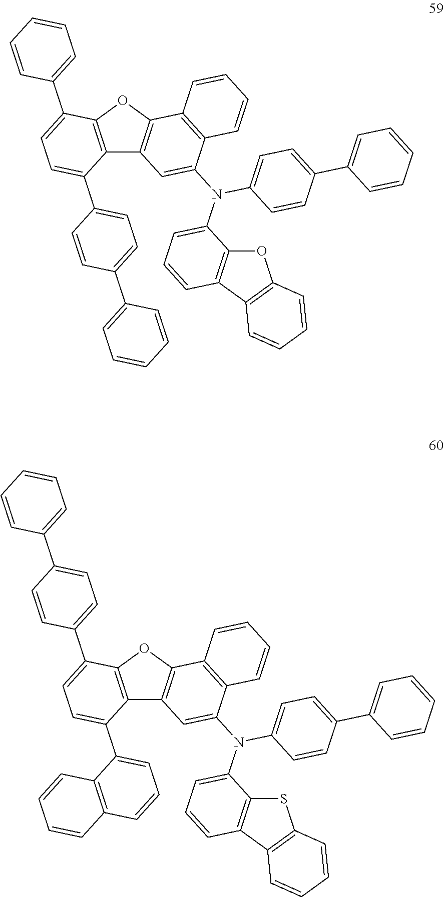 [SMILES: c1ccc(-c2ccc(-c3ccc(-c4ccccc4)c4oc5c6ccccc6c(N(c6ccc(-c7ccccc7)cc6)c6cccc7c6oc6ccccc67)cc5c34)cc2)cc1 ; c1ccc(-c2ccc(-c3ccc(-c4cccc5ccccc45)c4c3oc3c5ccccc5c(N(c5ccc(-c6ccccc6)cc5)c5cccc6c5sc5ccccc56)cc34)cc2)cc1]

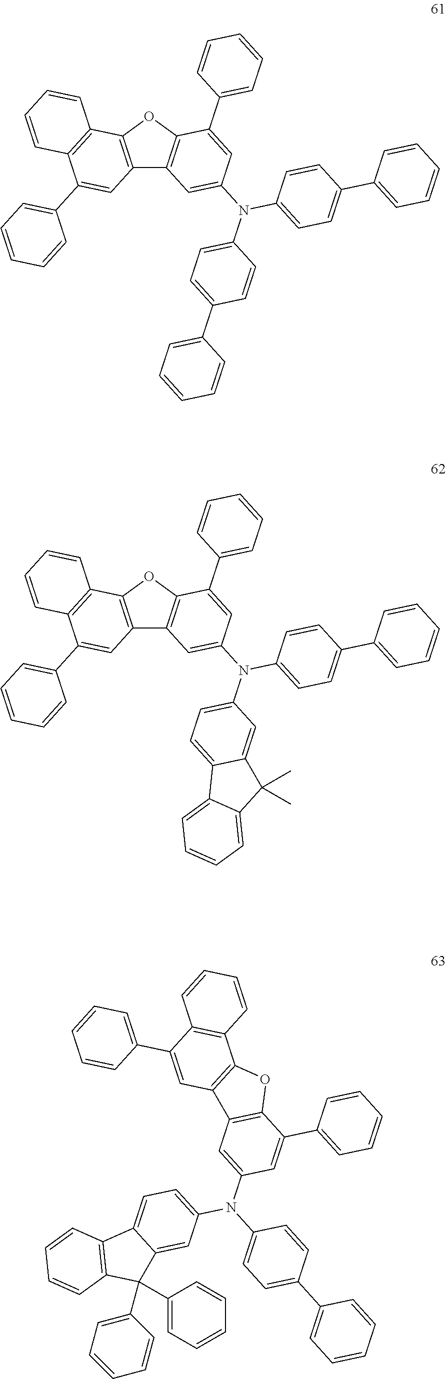 [SMILES: c1ccc(-c2ccc(N(c3ccc(-c4ccccc4)cc3)c3cc(-c4ccccc4)c4oc5c6ccccc6c(-c6ccccc6)cc5c4c3)cc2)cc1 ; CC1(C)c2ccccc2-c2ccc(N(c3ccc(-c4ccccc4)cc3)c3cc(-c4ccccc4)c4oc5c6ccccc6c(-c6ccccc6)cc5c4c3)cc21 ; c1ccc(-c2ccc(N(c3ccc4c(c3)C(c3ccccc3)(c3ccccc3)c3ccccc3-4)c3cc(-c4ccccc4)c4oc5c6ccccc6c(-c6ccccc6)cc5c4c3)cc2)cc1]

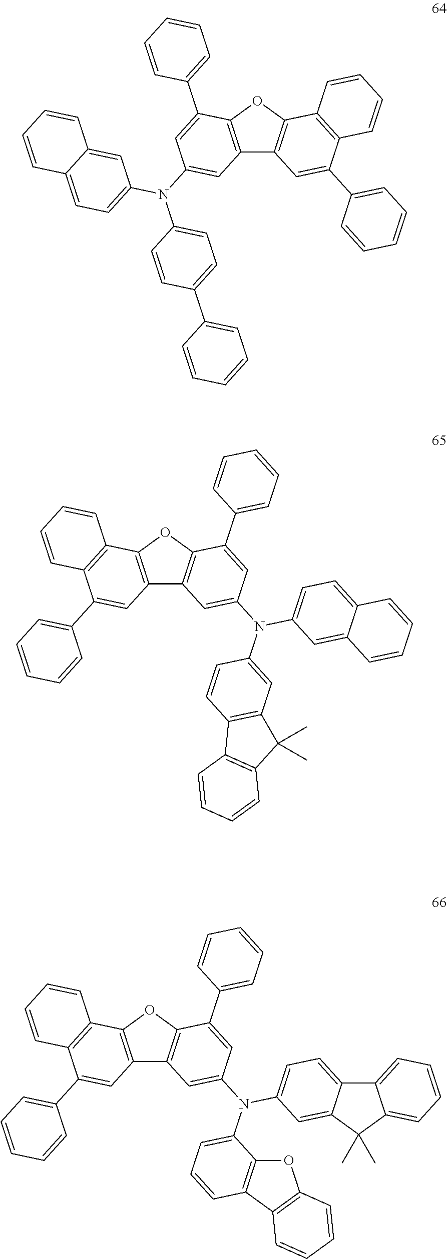 [SMILES: c1ccc(-c2ccc(N(c3ccc4ccccc4c3)c3cc(-c4ccccc4)c4oc5c6ccccc6c(-c6ccccc6)cc5c4c3)cc2)cc1 ; CC1(C)c2ccccc2-c2ccc(N(c3ccc4ccccc4c3)c3cc(-c4ccccc4)c4oc5c6ccccc6c(-c6ccccc6)cc5c4c3)cc21 ; CC1(C)c2ccccc2-c2ccc(N(c3cc(-c4ccccc4)c4oc5c6ccccc6c(-c6ccccc6)cc5c4c3)c3cccc4c3oc3ccccc34)cc21]

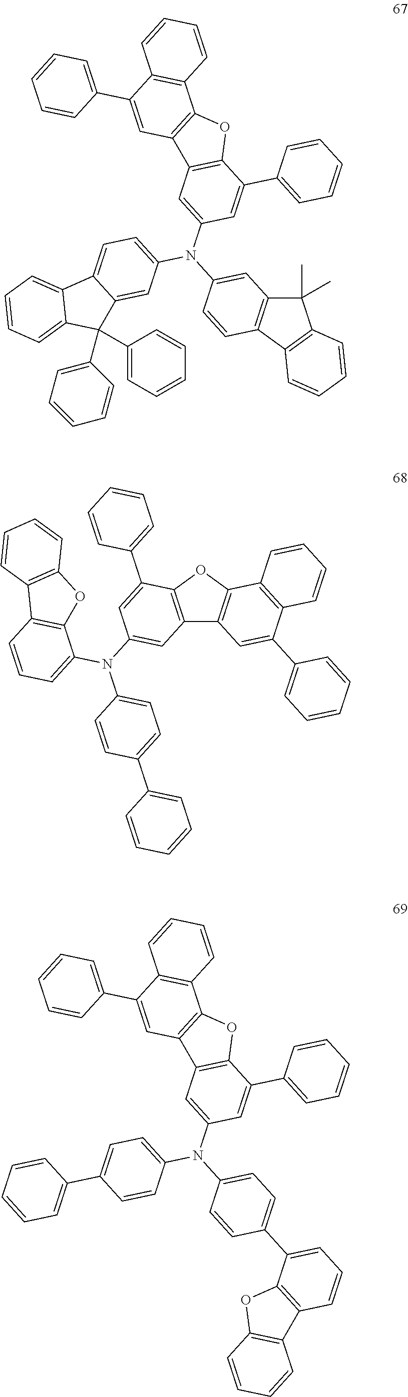 [SMILES: CC1(C)c2ccccc2-c2ccc(N(c3ccc4c(c3)C(c3ccccc3)(c3ccccc3)c3ccccc3-4)c3cc(-c4ccccc4)c4oc5c6ccccc6c(-c6ccccc6)cc5c4c3)cc21 ; c1ccc(-c2ccc(N(c3cc(-c4ccccc4)c4oc5c6ccccc6c(-c6ccccc6)cc5c4c3)c3cccc4c3oc3ccccc34)cc2)cc1 ; c1ccc(-c2ccc(N(c3ccc(-c4cccc5c4oc4ccccc45)cc3)c3cc(-c4ccccc4)c4oc5c6ccccc6c(-c6ccccc6)cc5c4c3)cc2)cc1]

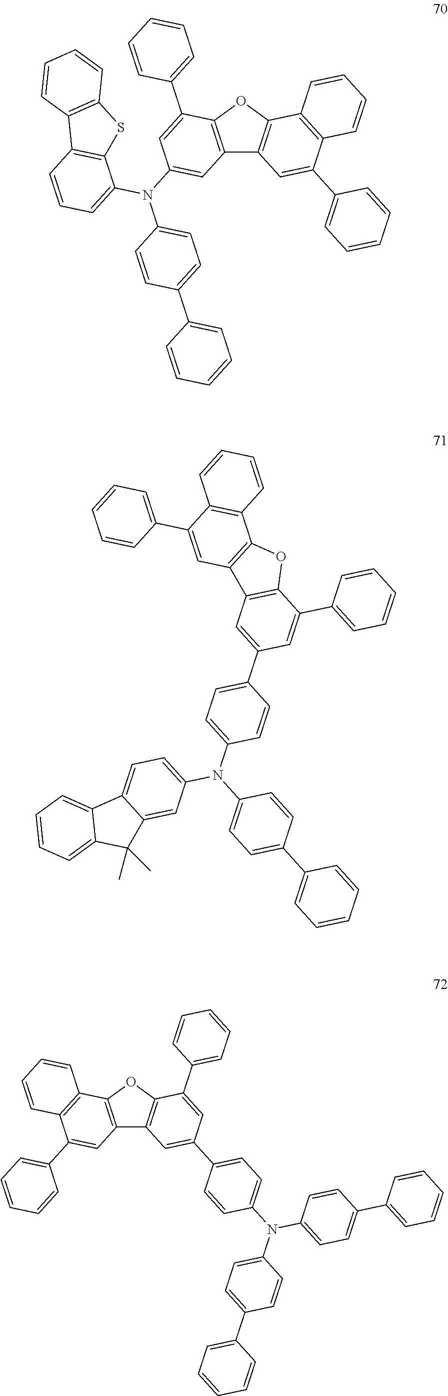 [SMILES: c1ccc(-c2ccc(N(c3cc(-c4ccccc4)c4oc5c6ccccc6c(-c6ccccc6)cc5c4c3)c3cccc4c3sc3ccccc34)cc2)cc1 ; CC1(C)c2ccccc2-c2ccc(N(c3ccc(-c4ccccc4)cc3)c3ccc(-c4cc(-c5ccccc5)c5oc6c7ccccc7c(-c7ccccc7)cc6c5c4)cc3)cc21 ; c1ccc(-c2ccc(N(c3ccc(-c4ccccc4)cc3)c3ccc(-c4cc(-c5ccccc5)c5oc6c7ccccc7c(-c7ccccc7)cc6c5c4)cc3)cc2)cc1]

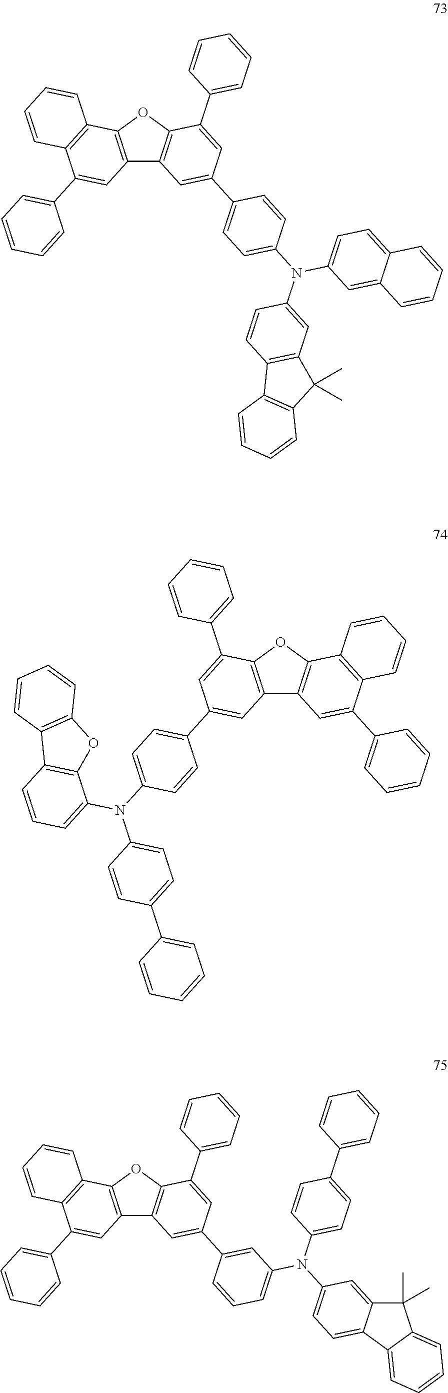 [SMILES: CC1(C)c2ccccc2-c2ccc(N(c3ccc(-c4cc(-c5ccccc5)c5oc6c7ccccc7c(-c7ccccc7)cc6c5c4)cc3)c3ccc4ccccc4c3)cc21 ; c1ccc(-c2ccc(N(c3ccc(-c4cc(-c5ccccc5)c5oc6c7ccccc7c(-c7ccccc7)cc6c5c4)cc3)c3cccc4c3oc3ccccc34)cc2)cc1 ; CC1(C)c2ccccc2-c2ccc(N(c3ccc(-c4ccccc4)cc3)c3cccc(-c4cc(-c5ccccc5)c5oc6c7ccccc7c(-c7ccccc7)cc6c5c4)c3)cc21]

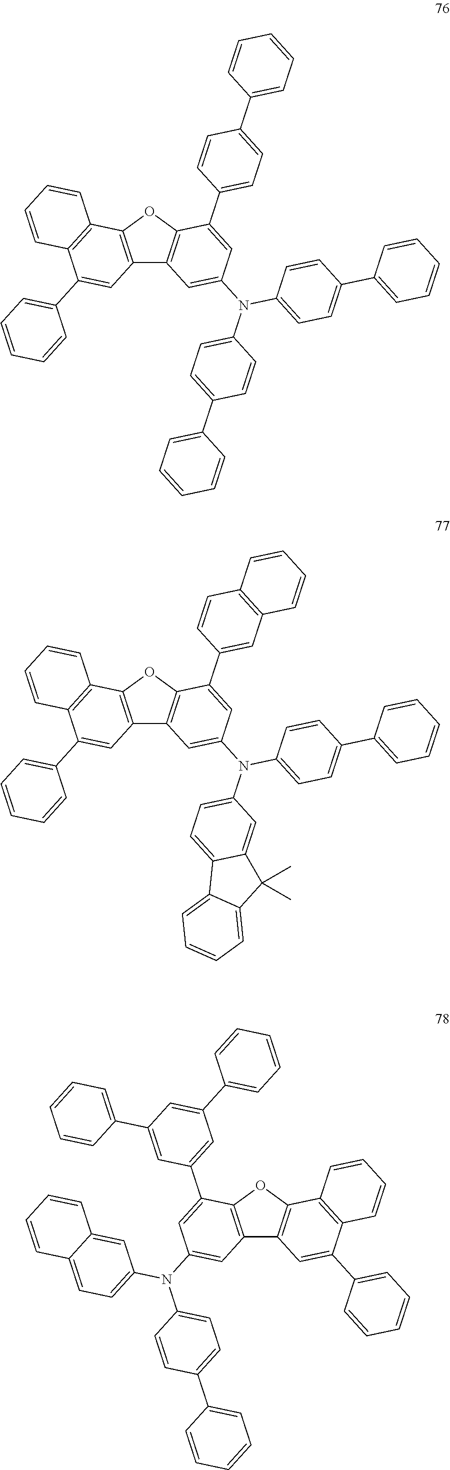 [SMILES: c1ccc(-c2ccc(-c3cc(N(c4ccc(-c5ccccc5)cc4)c4ccc(-c5ccccc5)cc4)cc4c3oc3c5ccccc5c(-c5ccccc5)cc43)cc2)cc1 ; CC1(C)c2ccccc2-c2ccc(N(c3ccc(-c4ccccc4)cc3)c3cc(-c4ccc5ccccc5c4)c4oc5c6ccccc6c(-c6ccccc6)cc5c4c3)cc21 ; c1ccc(-c2ccc(N(c3ccc4ccccc4c3)c3cc(-c4cc(-c5ccccc5)cc(-c5ccccc5)c4)c4oc5c6ccccc6c(-c6ccccc6)cc5c4c3)cc2)cc1]

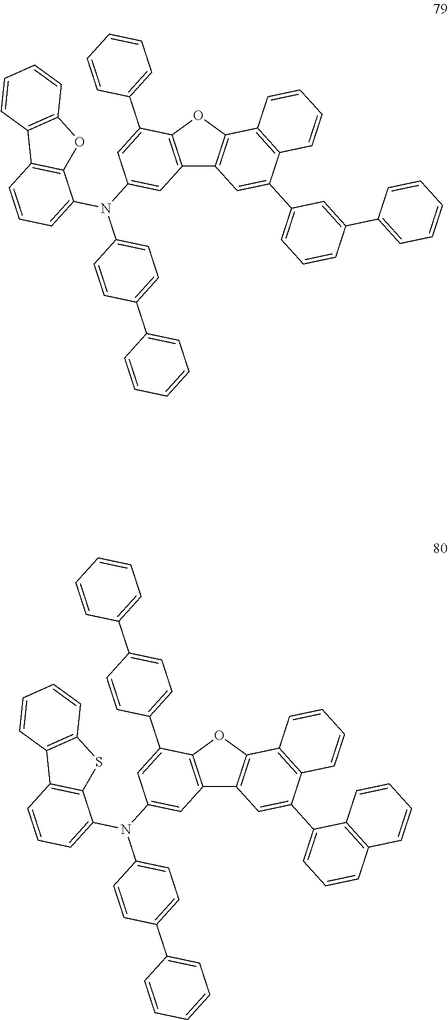 [SMILES: c1ccc(-c2ccc(N(c3cc(-c4ccccc4)c4oc5c6ccccc6c(-c6cccc(-c7ccccc7)c6)cc5c4c3)c3cccc4c3oc3ccccc34)cc2)cc1 ; c1ccc(-c2ccc(-c3cc(N(c4ccc(-c5ccccc5)cc4)c4cccc5c4sc4ccccc45)cc4c3oc3c5ccccc5c(-c5cccc6ccccc56)cc43)cc2)cc1]

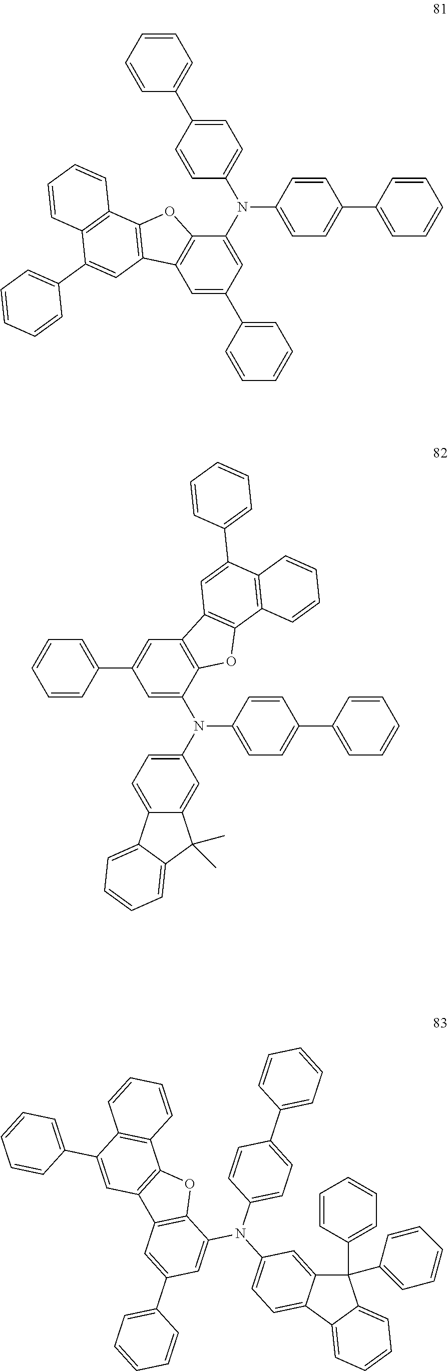 [SMILES: c1ccc(-c2ccc(N(c3ccc(-c4ccccc4)cc3)c3cc(-c4ccccc4)cc4c3oc3c5ccccc5c(-c5ccccc5)cc43)cc2)cc1 ; CC1(C)c2ccccc2-c2ccc(N(c3ccc(-c4ccccc4)cc3)c3cc(-c4ccccc4)cc4c3oc3c5ccccc5c(-c5ccccc5)cc43)cc21 ; c1ccc(-c2ccc(N(c3ccc4c(c3)C(c3ccccc3)(c3ccccc3)c3ccccc3-4)c3cc(-c4ccccc4)cc4c3oc3c5ccccc5c(-c5ccccc5)cc43)cc2)cc1]

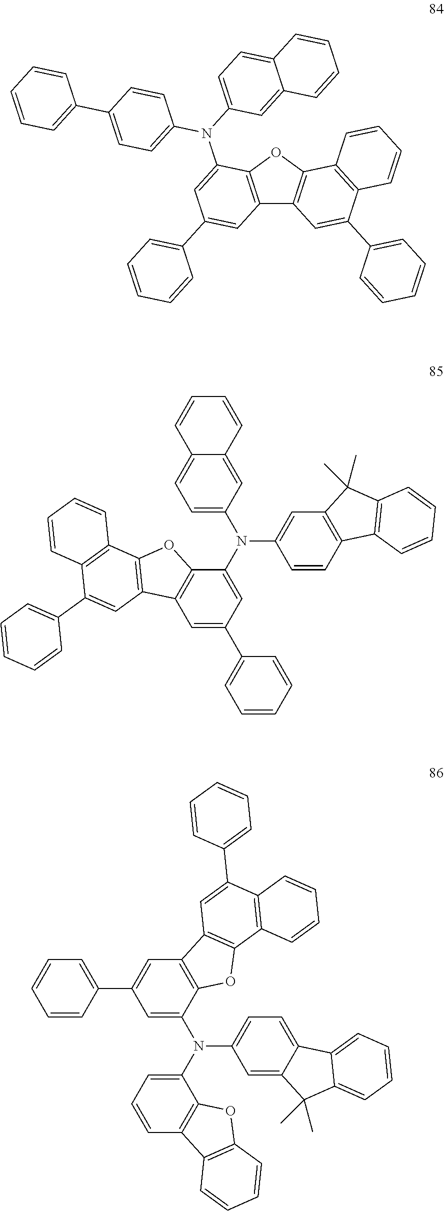 [SMILES: c1ccc(-c2ccc(N(c3ccc4ccccc4c3)c3cc(-c4ccccc4)cc4c3oc3c5ccccc5c(-c5ccccc5)cc43)cc2)cc1 ; CC1(C)c2ccccc2-c2ccc(N(c3ccc4ccccc4c3)c3cc(-c4ccccc4)cc4c3oc3c5ccccc5c(-c5ccccc5)cc43)cc21 ; CC1(C)c2ccccc2-c2ccc(N(c3cccc4c3oc3ccccc34)c3cc(-c4ccccc4)cc4c3oc3c5ccccc5c(-c5ccccc5)cc43)cc21]

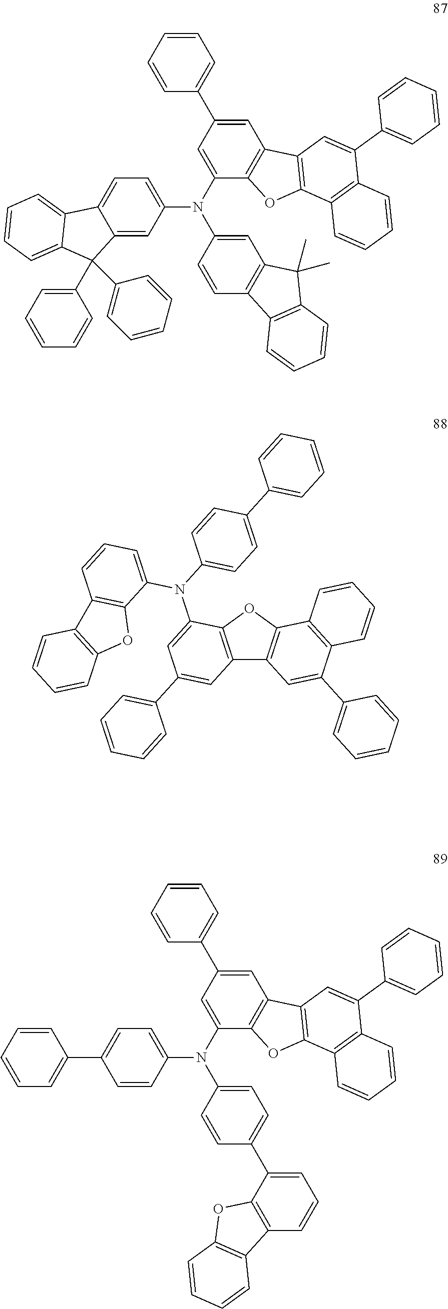 [SMILES: CC1(C)c2ccccc2-c2ccc(N(c3ccc4c(c3)C(c3ccccc3)(c3ccccc3)c3ccccc3-4)c3cc(-c4ccccc4)cc4c3oc3c5ccccc5c(-c5ccccc5)cc43)cc21 ; c1ccc(-c2ccc(N(c3cccc4c3oc3ccccc34)c3cc(-c4ccccc4)cc4c3oc3c5ccccc5c(-c5ccccc5)cc43)cc2)cc1 ; c1ccc(-c2ccc(N(c3ccc(-c4cccc5c4oc4ccccc45)cc3)c3cc(-c4ccccc4)cc4c3oc3c5ccccc5c(-c5ccccc5)cc43)cc2)cc1]

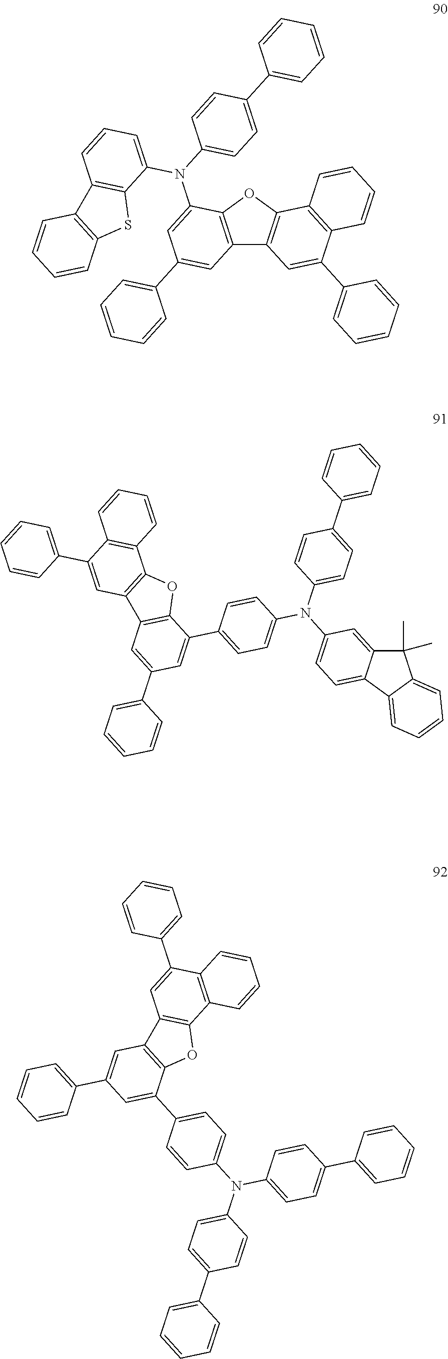 [SMILES: c1ccc(-c2ccc(N(c3cc(-c4ccccc4)cc4c3oc3c5ccccc5c(-c5ccccc5)cc43)c3cccc4c3sc3ccccc34)cc2)cc1 ; CC1(C)c2ccccc2-c2ccc(N(c3ccc(-c4ccccc4)cc3)c3ccc(-c4cc(-c5ccccc5)cc5c4oc4c6ccccc6c(-c6ccccc6)cc54)cc3)cc21 ; c1ccc(-c2ccc(N(c3ccc(-c4ccccc4)cc3)c3ccc(-c4cc(-c5ccccc5)cc5c4oc4c6ccccc6c(-c6ccccc6)cc54)cc3)cc2)cc1]

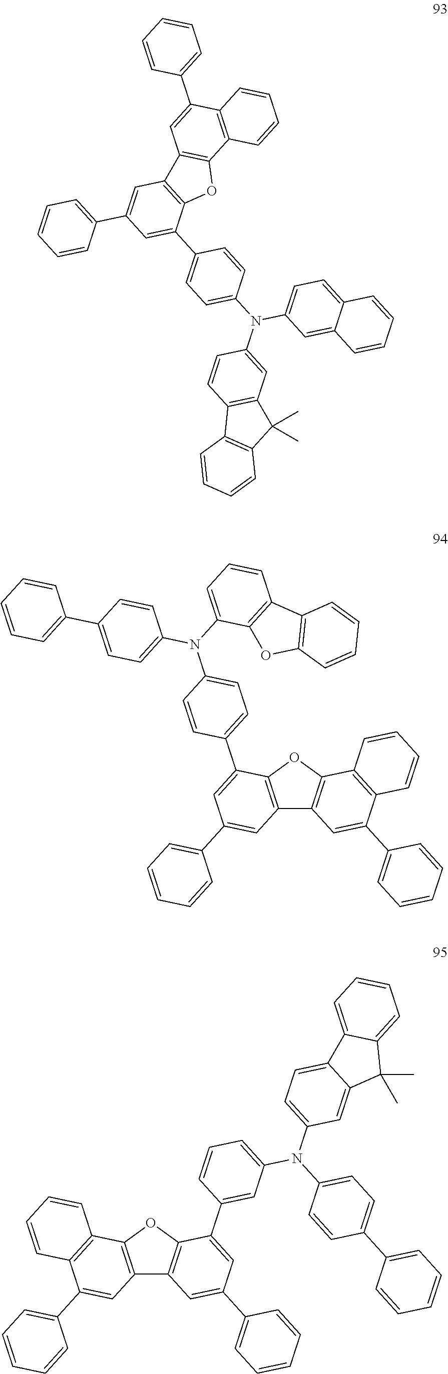 [SMILES: CC1(C)c2ccccc2-c2ccc(N(c3ccc(-c4cc(-c5ccccc5)cc5c4oc4c6ccccc6c(-c6ccccc6)cc54)cc3)c3ccc4ccccc4c3)cc21 ; c1ccc(-c2ccc(N(c3ccc(-c4cc(-c5ccccc5)cc5c4oc4c6ccccc6c(-c6ccccc6)cc54)cc3)c3cccc4c3oc3ccccc34)cc2)cc1 ; CC1(C)c2ccccc2-c2ccc(N(c3ccc(-c4ccccc4)cc3)c3cccc(-c4cc(-c5ccccc5)cc5c4oc4c6ccccc6c(-c6ccccc6)cc54)c3)cc21]

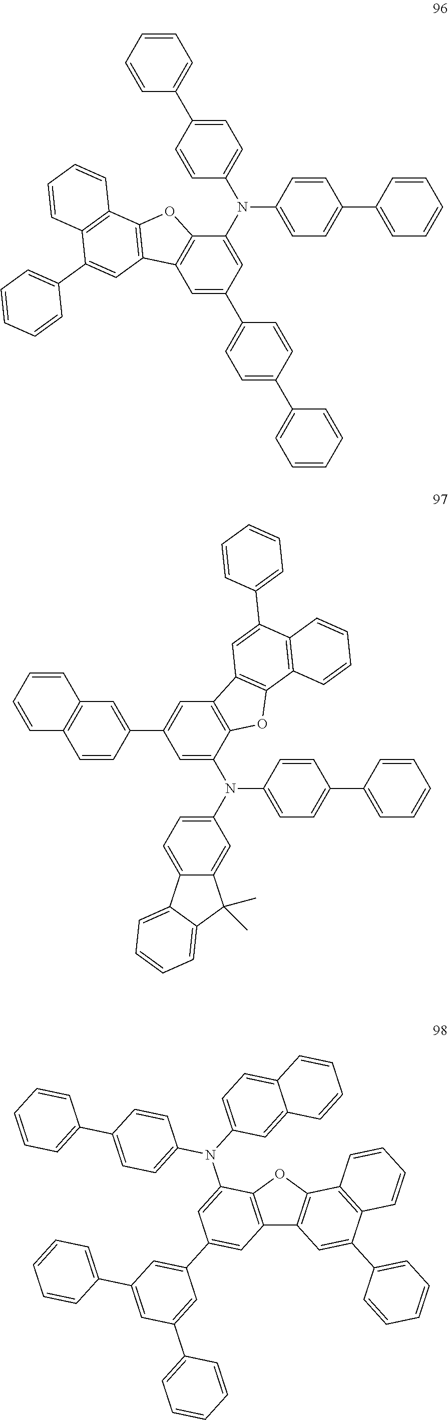 [SMILES: c1ccc(-c2ccc(-c3cc(N(c4ccc(-c5ccccc5)cc4)c4ccc(-c5ccccc5)cc4)c4oc5c6ccccc6c(-c6ccccc6)cc5c4c3)cc2)cc1 ; CC1(C)c2ccccc2-c2ccc(N(c3ccc(-c4ccccc4)cc3)c3cc(-c4ccc5ccccc5c4)cc4c3oc3c5ccccc5c(-c5ccccc5)cc43)cc21 ; c1ccc(-c2ccc(N(c3ccc4ccccc4c3)c3cc(-c4cc(-c5ccccc5)cc(-c5ccccc5)c4)cc4c3oc3c5ccccc5c(-c5ccccc5)cc43)cc2)cc1]

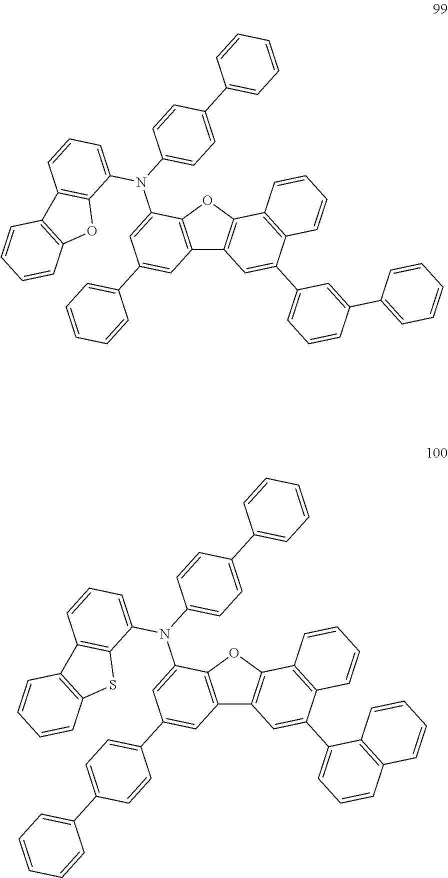 [SMILES: c1ccc(-c2ccc(N(c3cccc4c3oc3ccccc34)c3cc(-c4ccccc4)cc4c3oc3c5ccccc5c(-c5cccc(-c6ccccc6)c5)cc43)cc2)cc1 ; c1ccc(-c2ccc(-c3cc(N(c4ccc(-c5ccccc5)cc4)c4cccc5c4sc4ccccc45)c4oc5c6ccccc6c(-c6cccc7ccccc67)cc5c4c3)cc2)cc1]

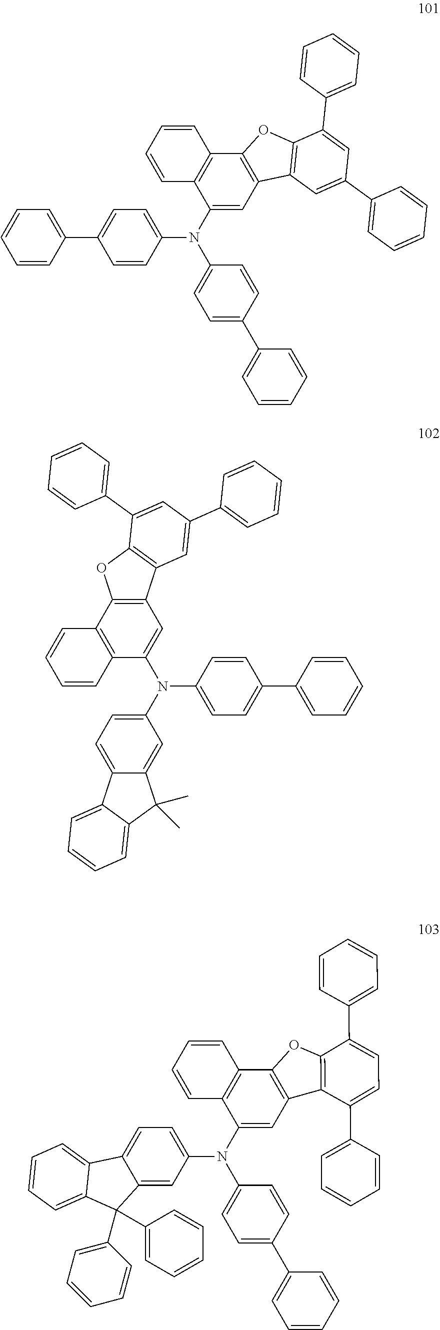 [SMILES: c1ccc(-c2ccc(N(c3ccc(-c4ccccc4)cc3)c3cc4c5cc(-c6ccccc6)cc(-c6ccccc6)c5oc4c4ccccc34)cc2)cc1 ; CC1(C)c2ccccc2-c2ccc(N(c3ccc(-c4ccccc4)cc3)c3cc4c5cc(-c6ccccc6)cc(-c6ccccc6)c5oc4c4ccccc34)cc21 ; c1ccc(-c2ccc(N(c3ccc4c(c3)C(c3ccccc3)(c3ccccc3)c3ccccc3-4)c3cc4c(oc5c(-c6ccccc6)ccc(-c6ccccc6)c54)c4ccccc34)cc2)cc1]

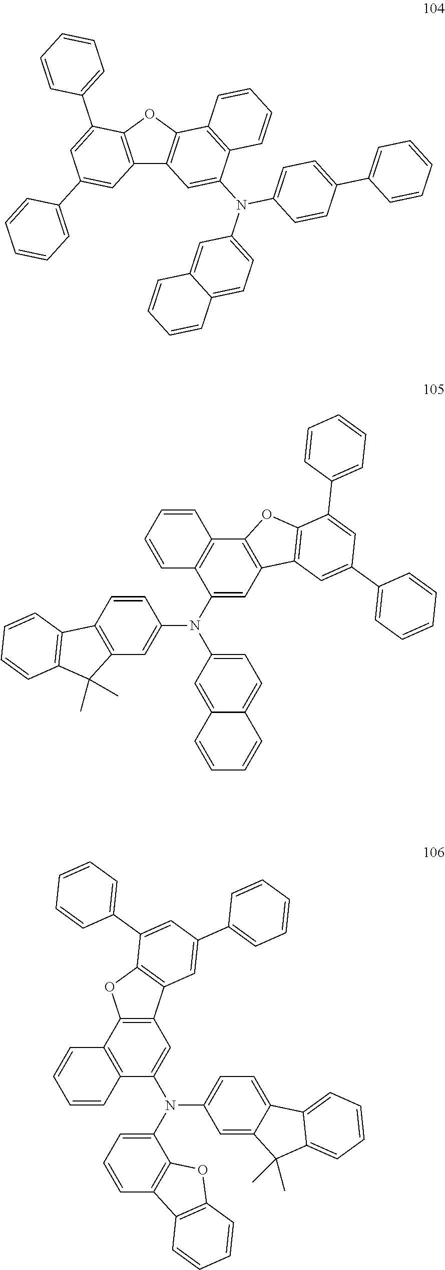 [SMILES: c1ccc(-c2ccc(N(c3ccc4ccccc4c3)c3cc4c5cc(-c6ccccc6)cc(-c6ccccc6)c5oc4c4ccccc34)cc2)cc1 ; CC1(C)c2ccccc2-c2ccc(N(c3ccc4ccccc4c3)c3cc4c5cc(-c6ccccc6)cc(-c6ccccc6)c5oc4c4ccccc34)cc21 ; CC1(C)c2ccccc2-c2ccc(N(c3cc4c5cc(-c6ccccc6)cc(-c6ccccc6)c5oc4c4ccccc34)c3cccc4c3oc3ccccc34)cc21]

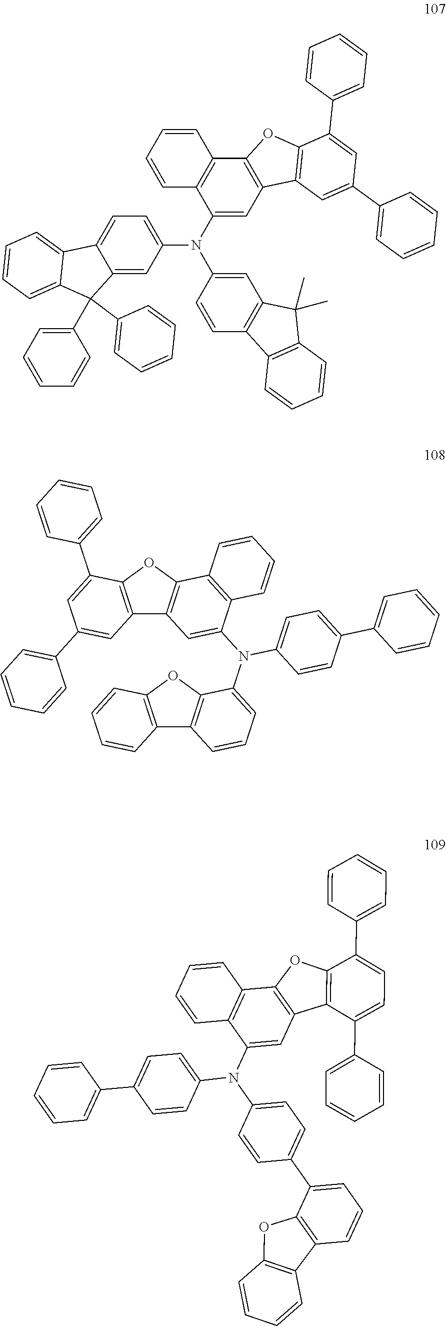 [SMILES: CC1(C)c2ccccc2-c2ccc(N(c3ccc4c(c3)C(c3ccccc3)(c3ccccc3)c3ccccc3-4)c3cc4c5cc(-c6ccccc6)cc(-c6ccccc6)c5oc4c4ccccc34)cc21 ; c1ccc(-c2ccc(N(c3cc4c5cc(-c6ccccc6)cc(-c6ccccc6)c5oc4c4ccccc34)c3cccc4c3oc3ccccc34)cc2)cc1 ; c1ccc(-c2ccc(N(c3ccc(-c4cccc5c4oc4ccccc45)cc3)c3cc4c(oc5c(-c6ccccc6)ccc(-c6ccccc6)c54)c4ccccc34)cc2)cc1]

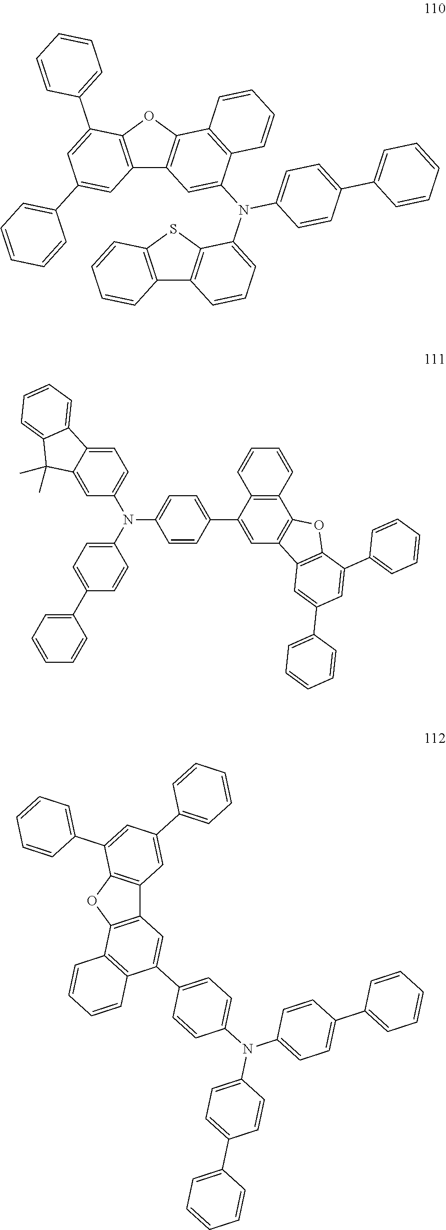 [SMILES: c1ccc(-c2ccc(N(c3cc4c5cc(-c6ccccc6)cc(-c6ccccc6)c5oc4c4ccccc34)c3cccc4c3sc3ccccc34)cc2)cc1 ; CC1(C)c2ccccc2-c2ccc(N(c3ccc(-c4ccccc4)cc3)c3ccc(-c4cc5c6cc(-c7ccccc7)cc(-c7ccccc7)c6oc5c5ccccc45)cc3)cc21 ; c1ccc(-c2ccc(N(c3ccc(-c4ccccc4)cc3)c3ccc(-c4cc5c6cc(-c7ccccc7)cc(-c7ccccc7)c6oc5c5ccccc45)cc3)cc2)cc1]

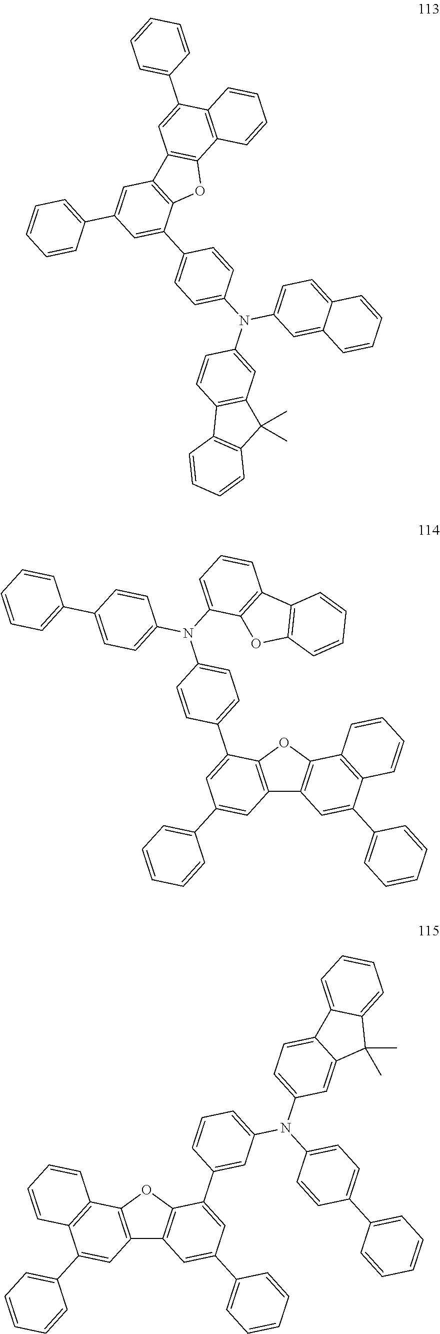 [SMILES: CC1(C)c2ccccc2-c2ccc(N(c3ccc(-c4cc(-c5ccccc5)cc5c4oc4c6ccccc6c(-c6ccccc6)cc54)cc3)c3ccc4ccccc4c3)cc21 ; c1ccc(-c2ccc(N(c3ccc(-c4cc(-c5ccccc5)cc5c4oc4c6ccccc6c(-c6ccccc6)cc54)cc3)c3cccc4c3oc3ccccc34)cc2)cc1 ; CC1(C)c2ccccc2-c2ccc(N(c3ccc(-c4ccccc4)cc3)c3cccc(-c4cc(-c5ccccc5)cc5c4oc4c6ccccc6c(-c6ccccc6)cc54)c3)cc21]

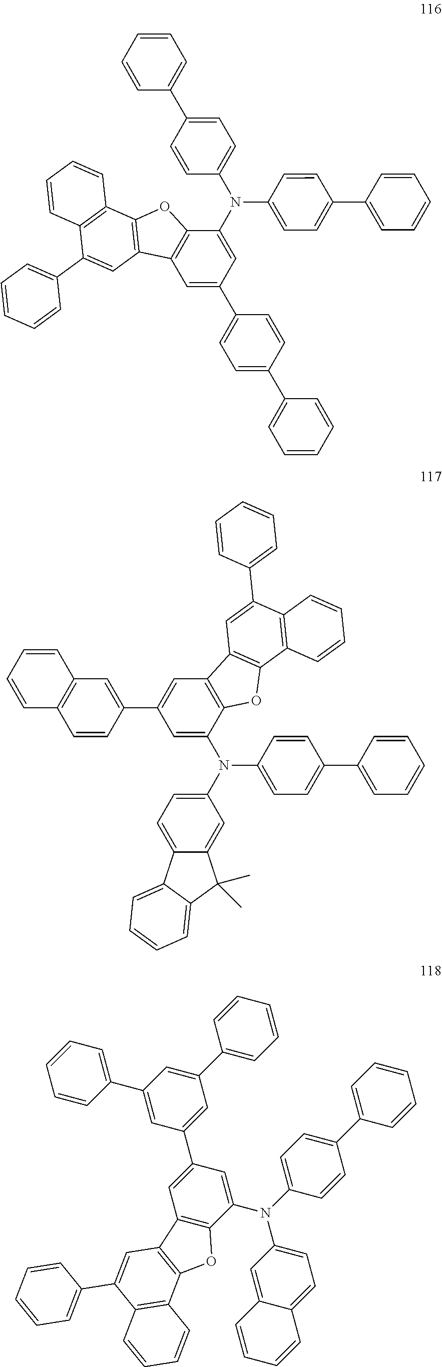 [SMILES: c1ccc(-c2ccc(-c3cc(N(c4ccc(-c5ccccc5)cc4)c4ccc(-c5ccccc5)cc4)c4oc5c6ccccc6c(-c6ccccc6)cc5c4c3)cc2)cc1 ; CC1(C)c2ccccc2-c2ccc(N(c3ccc(-c4ccccc4)cc3)c3cc(-c4ccc5ccccc5c4)cc4c3oc3c5ccccc5c(-c5ccccc5)cc43)cc21 ; c1ccc(-c2ccc(N(c3ccc4ccccc4c3)c3cc(-c4cc(-c5ccccc5)cc(-c5ccccc5)c4)cc4c3oc3c5ccccc5c(-c5ccccc5)cc43)cc2)cc1]

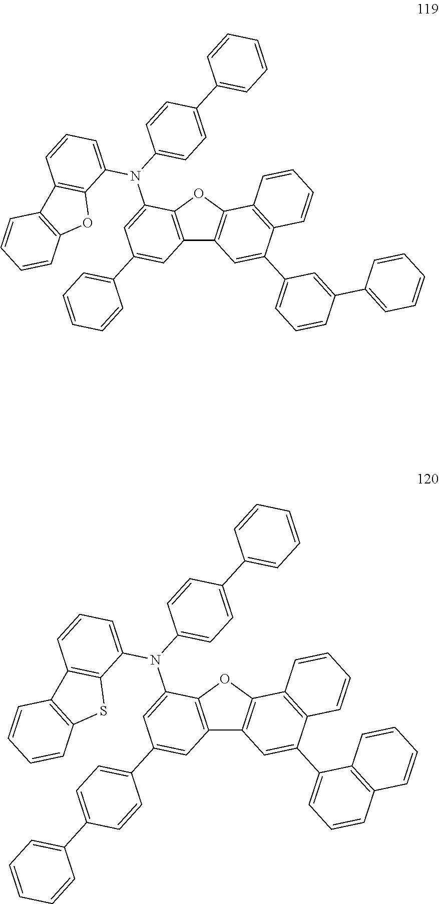 [SMILES: c1ccc(-c2ccc(N(c3cccc4c3oc3ccccc34)c3cc(-c4ccccc4)cc4c3oc3c5ccccc5c(-c5cccc(-c6ccccc6)c5)cc43)cc2)cc1 ; c1ccc(-c2ccc(-c3cc(N(c4ccc(-c5ccccc5)cc4)c4cccc5c4sc4ccccc45)c4oc5c6ccccc6c(-c6cccc7ccccc67)cc5c4c3)cc2)cc1]

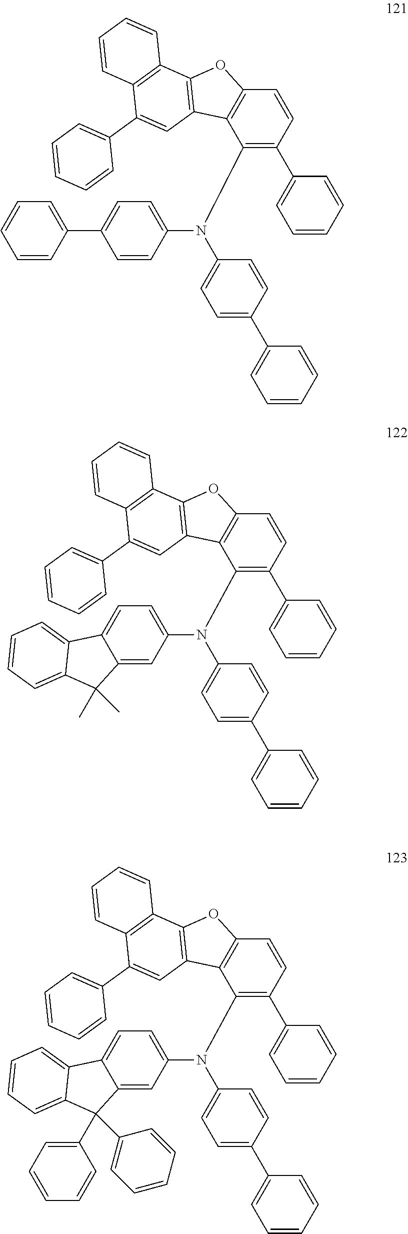 [SMILES: c1ccc(-c2ccc(N(c3ccc(-c4ccccc4)cc3)c3c(-c4ccccc4)ccc4oc5c6ccccc6c(-c6ccccc6)cc5c34)cc2)cc1 ; CC1(C)c2ccccc2-c2ccc(N(c3ccc(-c4ccccc4)cc3)c3c(-c4ccccc4)ccc4oc5c6ccccc6c(-c6ccccc6)cc5c34)cc21 ; c1ccc(-c2ccc(N(c3ccc4c(c3)C(c3ccccc3)(c3ccccc3)c3ccccc3-4)c3c(-c4ccccc4)ccc4oc5c6ccccc6c(-c6ccccc6)cc5c34)cc2)cc1]

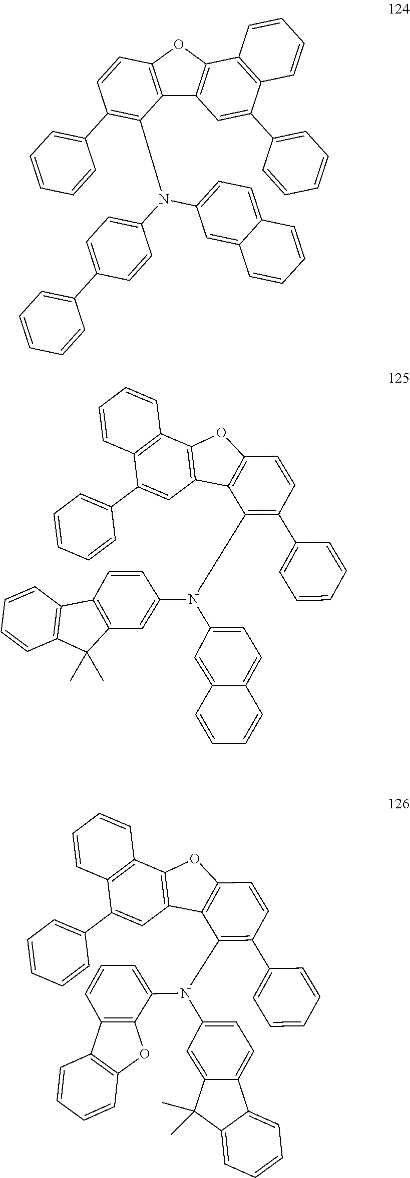 [SMILES: c1ccc(-c2ccc(N(c3ccc4ccccc4c3)c3c(-c4ccccc4)ccc4oc5c6ccccc6c(-c6ccccc6)cc5c34)cc2)cc1 ; CC1(C)c2ccccc2-c2ccc(N(c3ccc4ccccc4c3)c3c(-c4ccccc4)ccc4oc5c6ccccc6c(-c6ccccc6)cc5c34)cc21 ; CC1(C)c2ccccc2-c2ccc(N(c3cccc4c3oc3ccccc34)c3c(-c4ccccc4)ccc4oc5c6ccccc6c(-c6ccccc6)cc5c34)cc21]

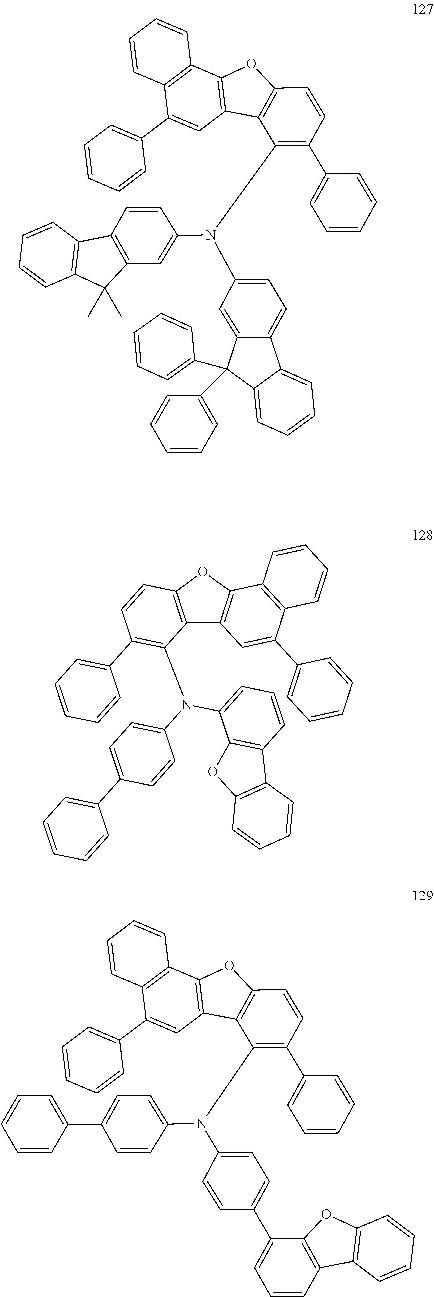 [SMILES: CC1(C)c2ccccc2-c2ccc(N(c3ccc4c(c3)C(c3ccccc3)(c3ccccc3)c3ccccc3-4)c3c(-c4ccccc4)ccc4oc5c6ccccc6c(-c6ccccc6)cc5c34)cc21 ; c1ccc(-c2ccc(N(c3cccc4c3oc3ccccc34)c3c(-c4ccccc4)ccc4oc5c6ccccc6c(-c6ccccc6)cc5c34)cc2)cc1 ; c1ccc(-c2ccc(N(c3ccc(-c4cccc5c4oc4ccccc45)cc3)c3c(-c4ccccc4)ccc4oc5c6ccccc6c(-c6ccccc6)cc5c34)cc2)cc1]

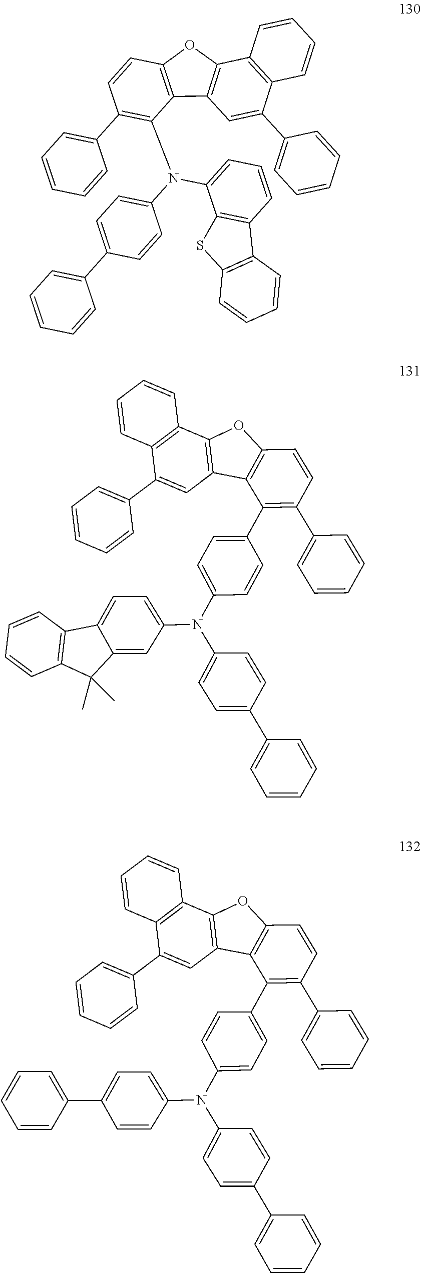 [SMILES: c1ccc(-c2ccc(N(c3cccc4c3sc3ccccc34)c3c(-c4ccccc4)ccc4oc5c6ccccc6c(-c6ccccc6)cc5c34)cc2)cc1 ; CC1(C)c2ccccc2-c2ccc(N(c3ccc(-c4ccccc4)cc3)c3ccc(-c4c(-c5ccccc5)ccc5oc6c7ccccc7c(-c7ccccc7)cc6c45)cc3)cc21 ; c1ccc(-c2ccc(N(c3ccc(-c4ccccc4)cc3)c3ccc(-c4c(-c5ccccc5)ccc5oc6c7ccccc7c(-c7ccccc7)cc6c45)cc3)cc2)cc1]

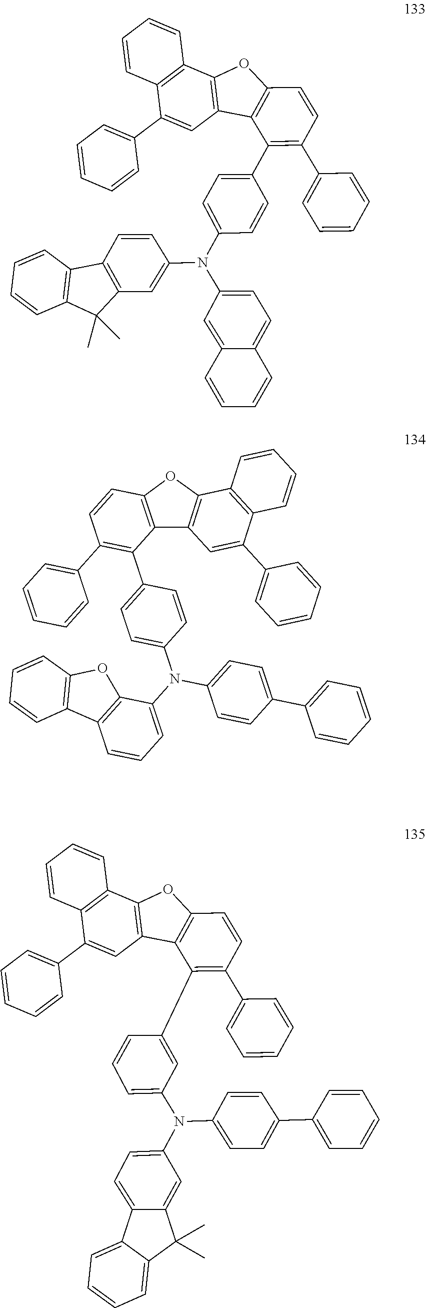

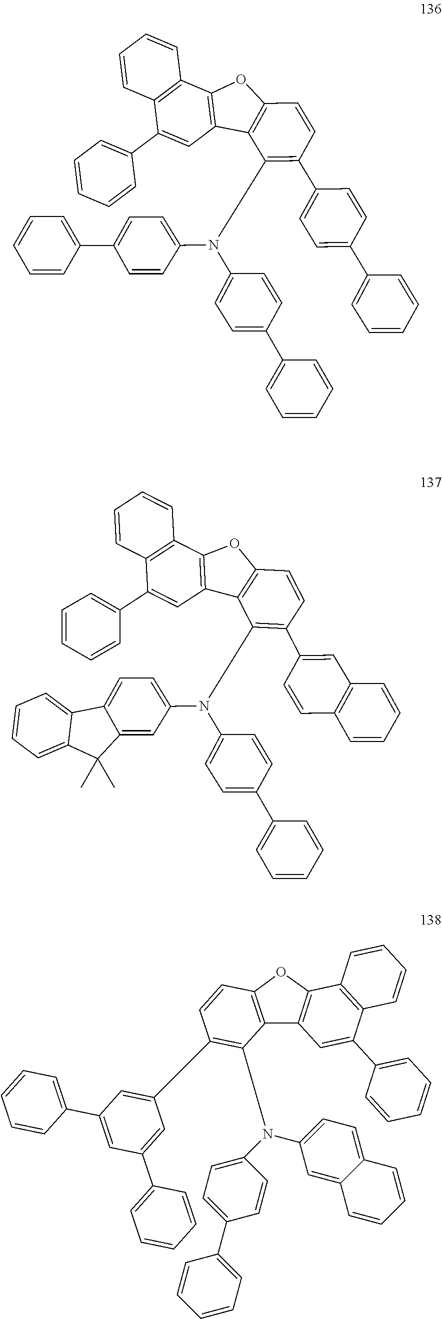 [SMILES: c1ccc(-c2ccc(-c3ccc4oc5c6ccccc6c(-c6ccccc6)cc5c4c3N(c3ccc(-c4ccccc4)cc3)c3ccc(-c4ccccc4)cc3)cc2)cc1 ; CC1(C)c2ccccc2-c2ccc(N(c3ccc(-c4ccccc4)cc3)c3c(-c4ccc5ccccc5c4)ccc4oc5c6ccccc6c(-c6ccccc6)cc5c34)cc21 ; c1ccc(-c2ccc(N(c3ccc4ccccc4c3)c3c(-c4cc(-c5ccccc5)cc(-c5ccccc5)c4)ccc4oc5c6ccccc6c(-c6ccccc6)cc5c34)cc2)cc1]

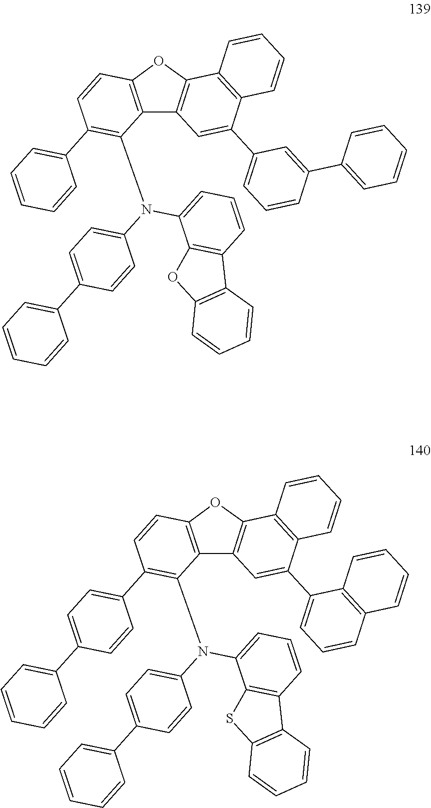 [SMILES: c1ccc(-c2ccc(N(c3cccc4c3oc3ccccc34)c3c(-c4ccccc4)ccc4oc5c6ccccc6c(-c6cccc(-c7ccccc7)c6)cc5c34)cc2)cc1 ; c1ccc(-c2ccc(-c3ccc4oc5c6ccccc6c(-c6cccc7ccccc67)cc5c4c3N(c3ccc(-c4ccccc4)cc3)c3cccc4c3sc3ccccc34)cc2)cc1]

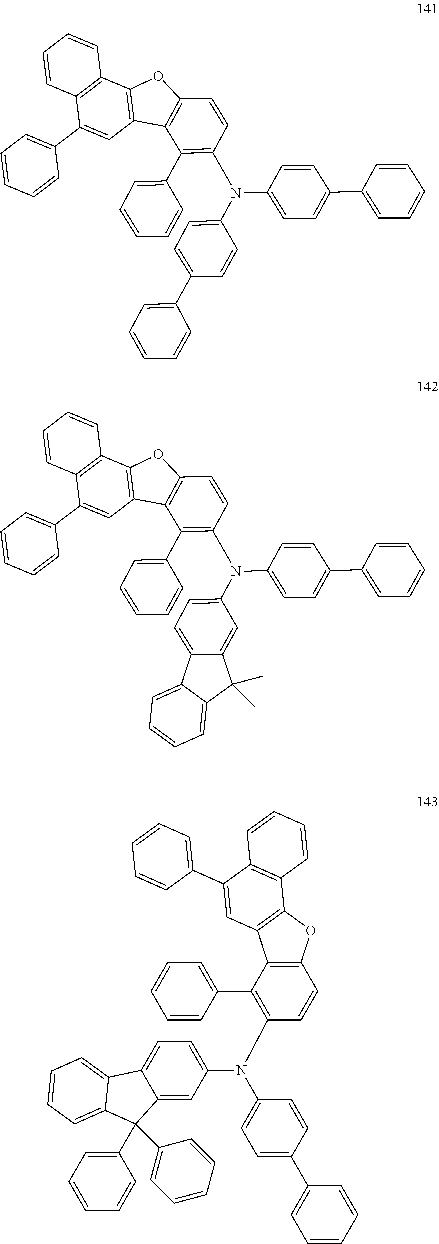 [SMILES: c1ccc(-c2ccc(N(c3ccc(-c4ccccc4)cc3)c3ccc4oc5c6ccccc6c(-c6ccccc6)cc5c4c3-c3ccccc3)cc2)cc1 ; CC1(C)c2ccccc2-c2ccc(N(c3ccc(-c4ccccc4)cc3)c3ccc4oc5c6ccccc6c(-c6ccccc6)cc5c4c3-c3ccccc3)cc21 ; c1ccc(-c2ccc(N(c3ccc4c(c3)C(c3ccccc3)(c3ccccc3)c3ccccc3-4)c3ccc4oc5c6ccccc6c(-c6ccccc6)cc5c4c3-c3ccccc3)cc2)cc1]

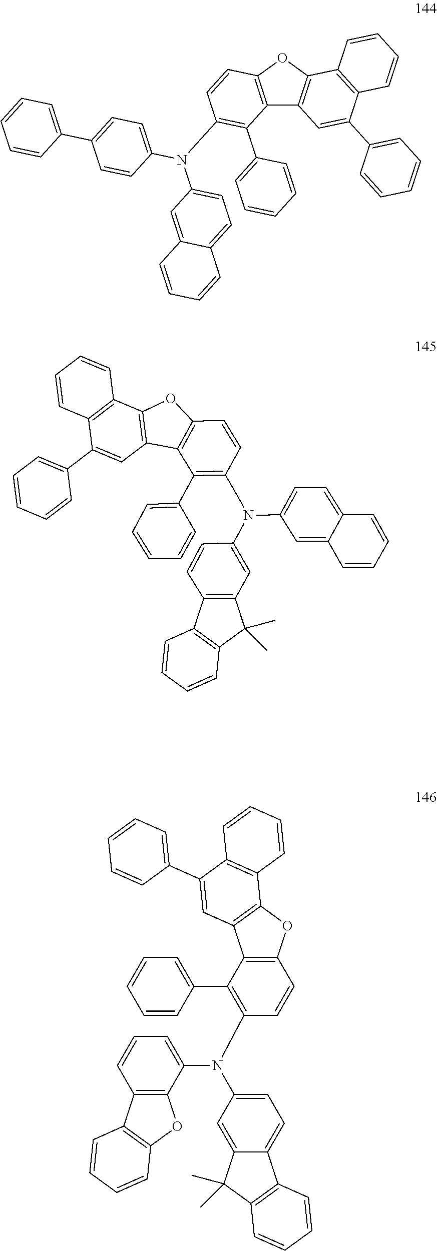 [SMILES: c1ccc(-c2ccc(N(c3ccc4ccccc4c3)c3ccc4oc5c6ccccc6c(-c6ccccc6)cc5c4c3-c3ccccc3)cc2)cc1 ; CC1(C)c2ccccc2-c2ccc(N(c3ccc4ccccc4c3)c3ccc4oc5c6ccccc6c(-c6ccccc6)cc5c4c3-c3ccccc3)cc21 ; CC1(C)c2ccccc2-c2ccc(N(c3ccc4oc5c6ccccc6c(-c6ccccc6)cc5c4c3-c3ccccc3)c3cccc4c3oc3ccccc34)cc21]

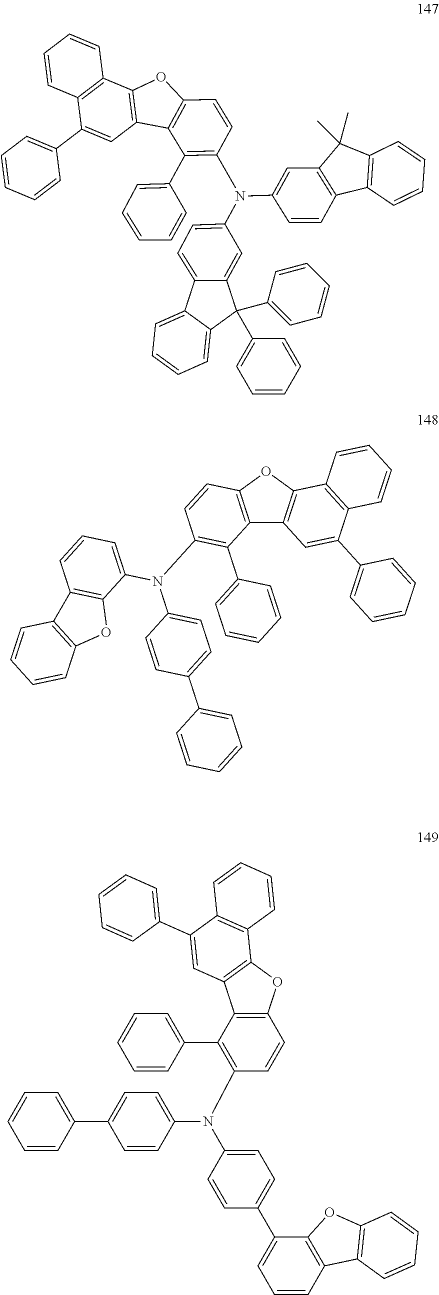 [SMILES: CC1(C)c2ccccc2-c2ccc(N(c3ccc4c(c3)C(c3ccccc3)(c3ccccc3)c3ccccc3-4)c3ccc4oc5c6ccccc6c(-c6ccccc6)cc5c4c3-c3ccccc3)cc21 ; c1ccc(-c2ccc(N(c3ccc4oc5c6ccccc6c(-c6ccccc6)cc5c4c3-c3ccccc3)c3cccc4c3oc3ccccc34)cc2)cc1 ; c1ccc(-c2ccc(N(c3ccc(-c4cccc5c4oc4ccccc45)cc3)c3ccc4oc5c6ccccc6c(-c6ccccc6)cc5c4c3-c3ccccc3)cc2)cc1]

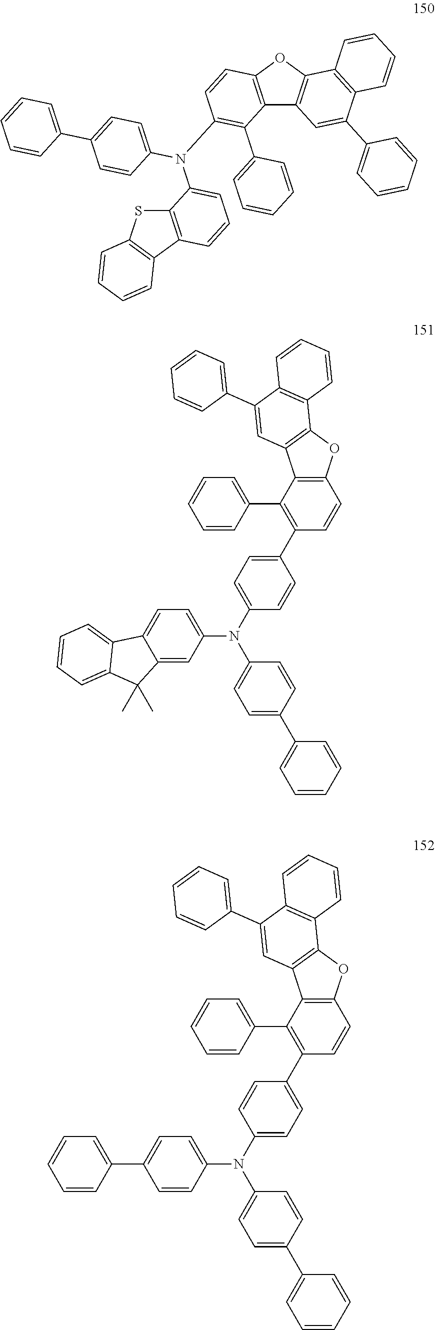 [SMILES: c1ccc(-c2ccc(N(c3ccc4oc5c6ccccc6c(-c6ccccc6)cc5c4c3-c3ccccc3)c3cccc4c3sc3ccccc34)cc2)cc1 ; CC1(C)c2ccccc2-c2ccc(N(c3ccc(-c4ccccc4)cc3)c3ccc(-c4ccc5oc6c7ccccc7c(-c7ccccc7)cc6c5c4-c4ccccc4)cc3)cc21 ; c1ccc(-c2ccc(N(c3ccc(-c4ccccc4)cc3)c3ccc(-c4ccc5oc6c7ccccc7c(-c7ccccc7)cc6c5c4-c4ccccc4)cc3)cc2)cc1]

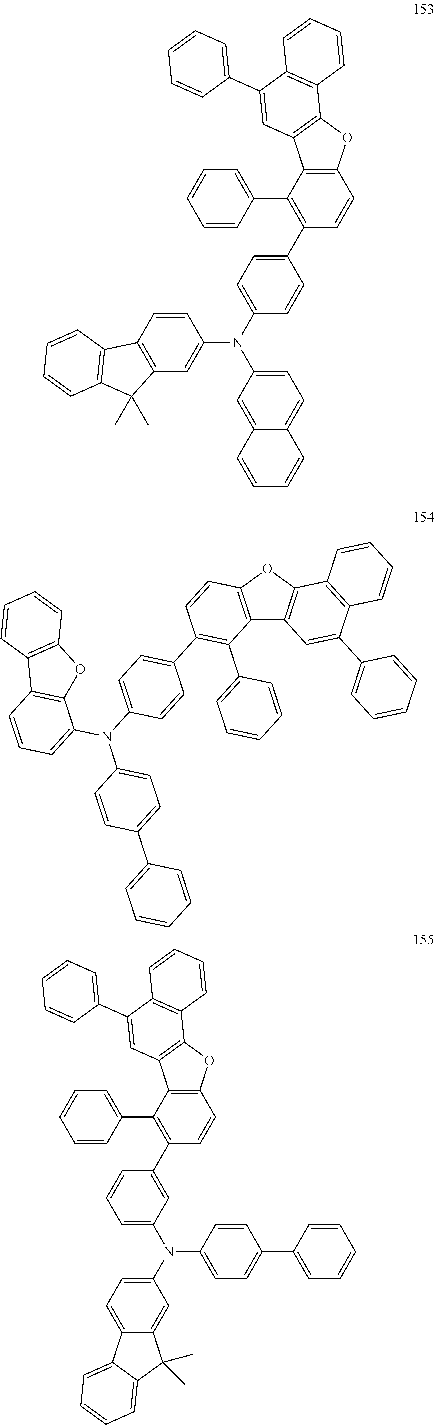

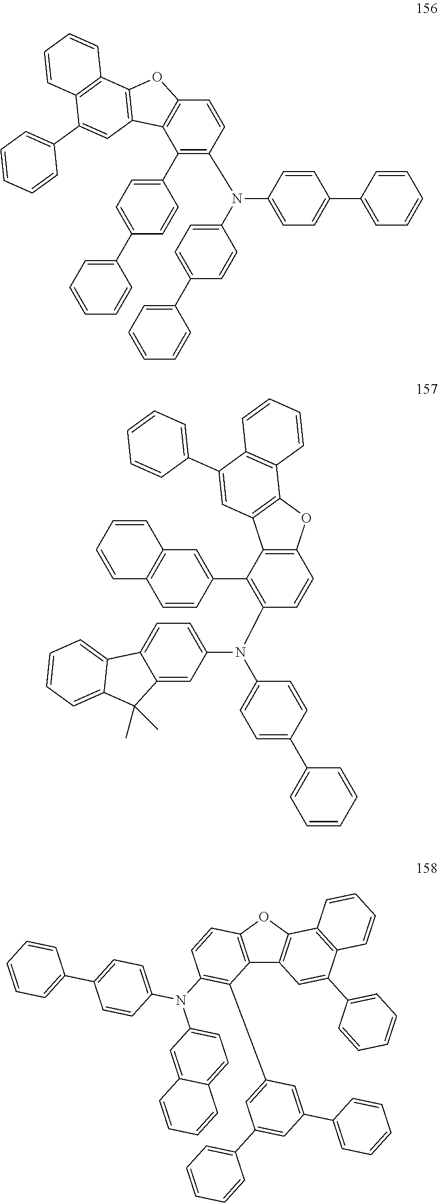 [SMILES: c1ccc(-c2ccc(-c3c(N(c4ccc(-c5ccccc5)cc4)c4ccc(-c5ccccc5)cc4)ccc4oc5c6ccccc6c(-c6ccccc6)cc5c34)cc2)cc1 ; CC1(C)c2ccccc2-c2ccc(N(c3ccc(-c4ccccc4)cc3)c3ccc4oc5c6ccccc6c(-c6ccccc6)cc5c4c3-c3ccc4ccccc4c3)cc21 ; c1ccc(-c2ccc(N(c3ccc4ccccc4c3)c3ccc4oc5c6ccccc6c(-c6ccccc6)cc5c4c3-c3cc(-c4ccccc4)cc(-c4ccccc4)c3)cc2)cc1]

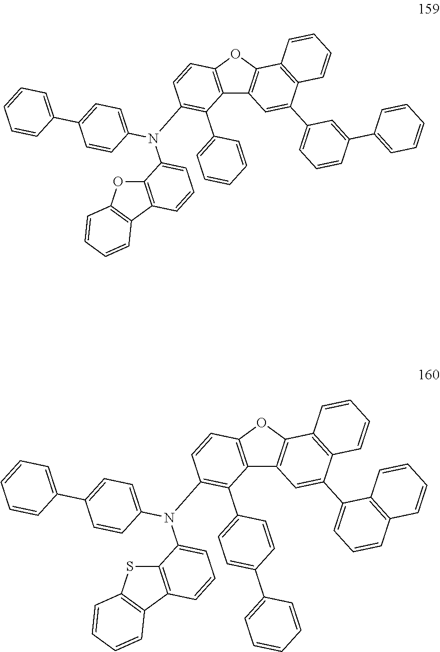 [SMILES: c1ccc(-c2ccc(N(c3ccc4oc5c6ccccc6c(-c6cccc(-c7ccccc7)c6)cc5c4c3-c3ccccc3)c3cccc4c3oc3ccccc34)cc2)cc1 ; c1ccc(-c2ccc(-c3c(N(c4ccc(-c5ccccc5)cc4)c4cccc5c4sc4ccccc45)ccc4oc5c6ccccc6c(-c6cccc7ccccc67)cc5c34)cc2)cc1]

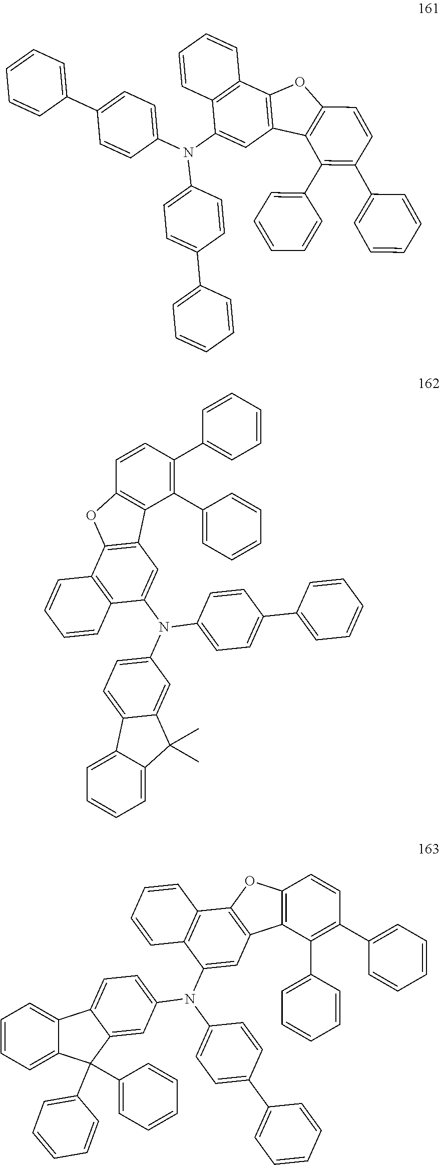 [SMILES: c1ccc(-c2ccc(N(c3ccc(-c4ccccc4)cc3)c3cc4c(oc5ccc(-c6ccccc6)c(-c6ccccc6)c54)c4ccccc34)cc2)cc1 ; CC1(C)c2ccccc2-c2ccc(N(c3ccc(-c4ccccc4)cc3)c3cc4c(oc5ccc(-c6ccccc6)c(-c6ccccc6)c54)c4ccccc34)cc21 ; c1ccc(-c2ccc(N(c3ccc4c(c3)C(c3ccccc3)(c3ccccc3)c3ccccc3-4)c3cc4c(oc5ccc(-c6ccccc6)c(-c6ccccc6)c54)c4ccccc34)cc2)cc1]

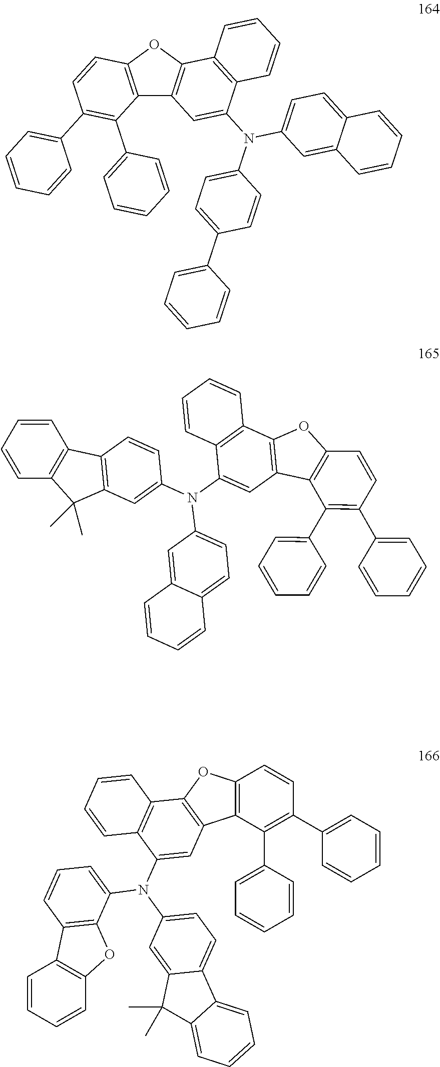 [SMILES: c1ccc(-c2ccc(N(c3ccc4ccccc4c3)c3cc4c(oc5ccc(-c6ccccc6)c(-c6ccccc6)c54)c4ccccc34)cc2)cc1 ; CC1(C)c2ccccc2-c2ccc(N(c3ccc4ccccc4c3)c3cc4c(oc5ccc(-c6ccccc6)c(-c6ccccc6)c54)c4ccccc34)cc21 ; CC1(C)c2ccccc2-c2ccc(N(c3cc4c(oc5ccc(-c6ccccc6)c(-c6ccccc6)c54)c4ccccc34)c3cccc4c3oc3ccccc34)cc21]

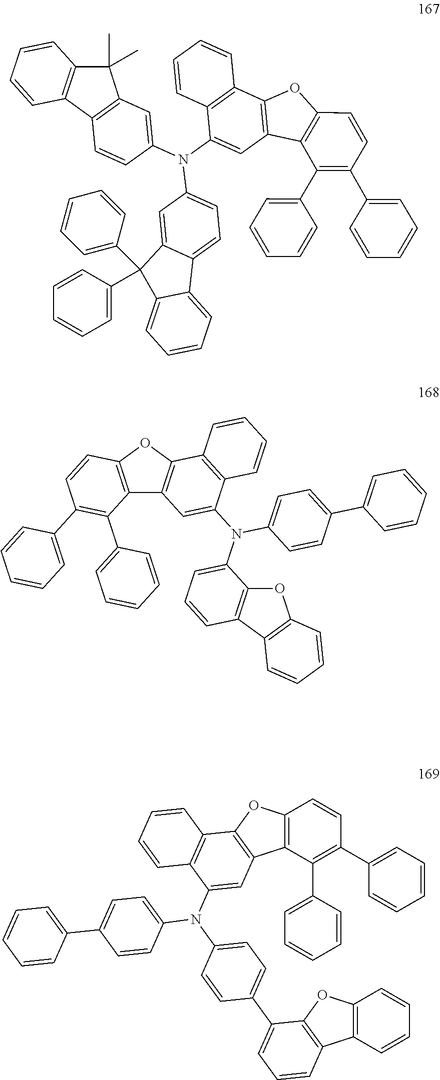 [SMILES: CC1(C)c2ccccc2-c2ccc(N(c3ccc4c(c3)C(c3ccccc3)(c3ccccc3)c3ccccc3-4)c3cc4c(oc5ccc(-c6ccccc6)c(-c6ccccc6)c54)c4ccccc34)cc21 ; c1ccc(-c2ccc(N(c3cc4c(oc5ccc(-c6ccccc6)c(-c6ccccc6)c54)c4ccccc34)c3cccc4c3oc3ccccc34)cc2)cc1 ; c1ccc(-c2ccc(N(c3ccc(-c4cccc5c4oc4ccccc45)cc3)c3cc4c(oc5ccc(-c6ccccc6)c(-c6ccccc6)c54)c4ccccc34)cc2)cc1]

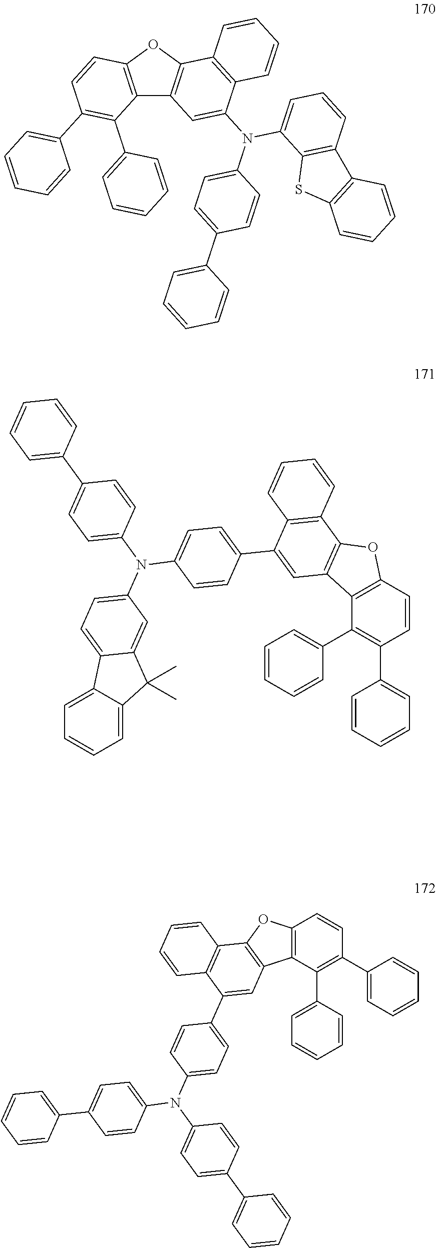 [SMILES: c1ccc(-c2ccc(N(c3cc4c(oc5ccc(-c6ccccc6)c(-c6ccccc6)c54)c4ccccc34)c3cccc4c3sc3ccccc34)cc2)cc1 ; CC1(C)c2ccccc2-c2ccc(N(c3ccc(-c4ccccc4)cc3)c3ccc(-c4cc5c(oc6ccc(-c7ccccc7)c(-c7ccccc7)c65)c5ccccc45)cc3)cc21 ; c1ccc(-c2ccc(N(c3ccc(-c4ccccc4)cc3)c3ccc(-c4cc5c(oc6ccc(-c7ccccc7)c(-c7ccccc7)c65)c5ccccc45)cc3)cc2)cc1]

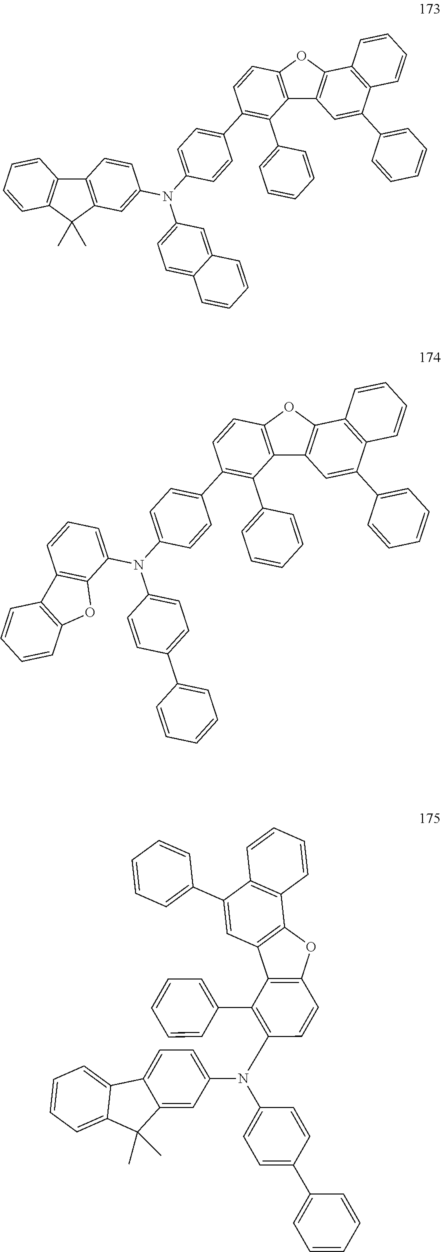 [SMILES: CC1(C)c2ccccc2-c2ccc(N(c3ccc(-c4ccc5oc6c7ccccc7c(-c7ccccc7)cc6c5c4-c4ccccc4)cc3)c3ccc4ccccc4c3)cc21 ; c1ccc(-c2ccc(N(c3ccc(-c4ccc5oc6c7ccccc7c(-c7ccccc7)cc6c5c4-c4ccccc4)cc3)c3cccc4c3oc3ccccc34)cc2)cc1 ; CC1(C)c2ccccc2-c2ccc(N(c3ccc(-c4ccccc4)cc3)c3ccc4oc5c6ccccc6c(-c6ccccc6)cc5c4c3-c3ccccc3)cc21]

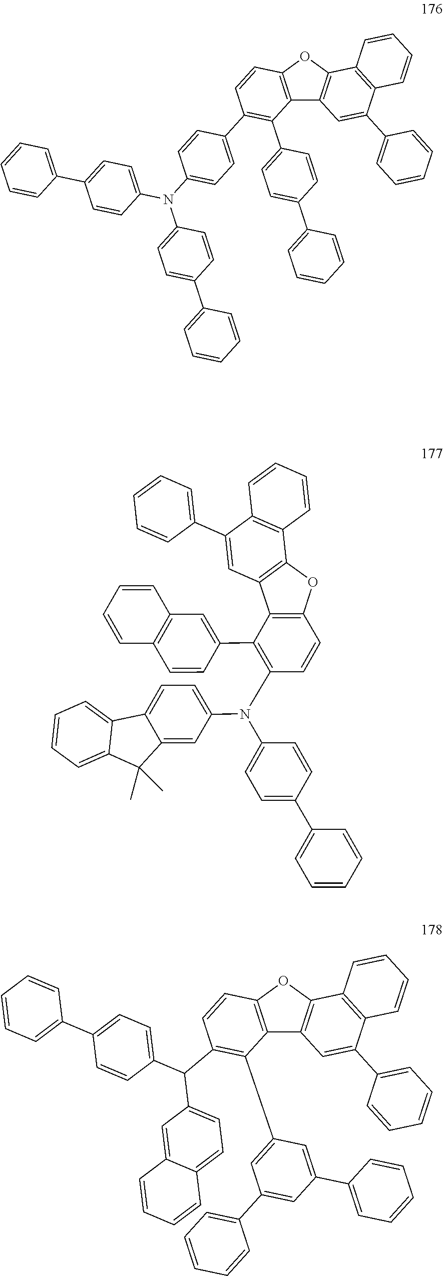 [SMILES: c1ccc(-c2ccc(-c3c(-c4ccc(N(c5ccc(-c6ccccc6)cc5)c5ccc(-c6ccccc6)cc5)cc4)ccc4oc5c6ccccc6c(-c6ccccc6)cc5c34)cc2)cc1 ; CC1(C)c2ccccc2-c2ccc(N(c3ccc(-c4ccccc4)cc3)c3ccc4oc5c6ccccc6c(-c6ccccc6)cc5c4c3-c3ccc4ccccc4c3)cc21 ; c1ccc(-c2ccc(C(c3ccc4ccccc4c3)c3ccc4oc5c6ccccc6c(-c6ccccc6)cc5c4c3-c3cc(-c4ccccc4)cc(-c4ccccc4)c3)cc2)cc1]

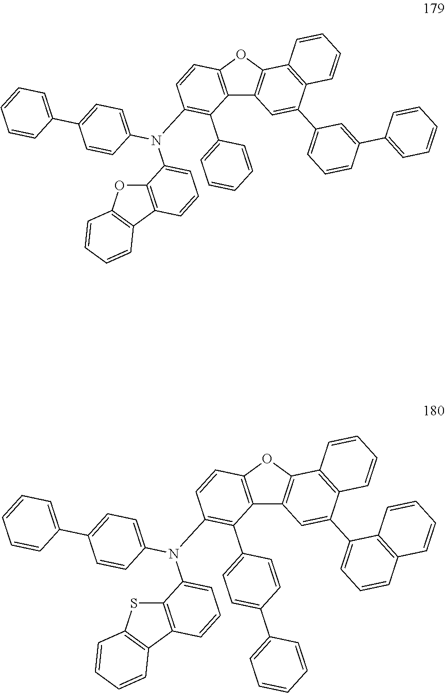 [SMILES: c1ccc(-c2ccc(N(c3ccc4oc5c6ccccc6c(-c6cccc(-c7ccccc7)c6)cc5c4c3-c3ccccc3)c3cccc4c3oc3ccccc34)cc2)cc1 ; c1ccc(-c2ccc(-c3c(N(c4ccc(-c5ccccc5)cc4)c4cccc5c4sc4ccccc45)ccc4oc5c6ccccc6c(-c6cccc7ccccc67)cc5c34)cc2)cc1]

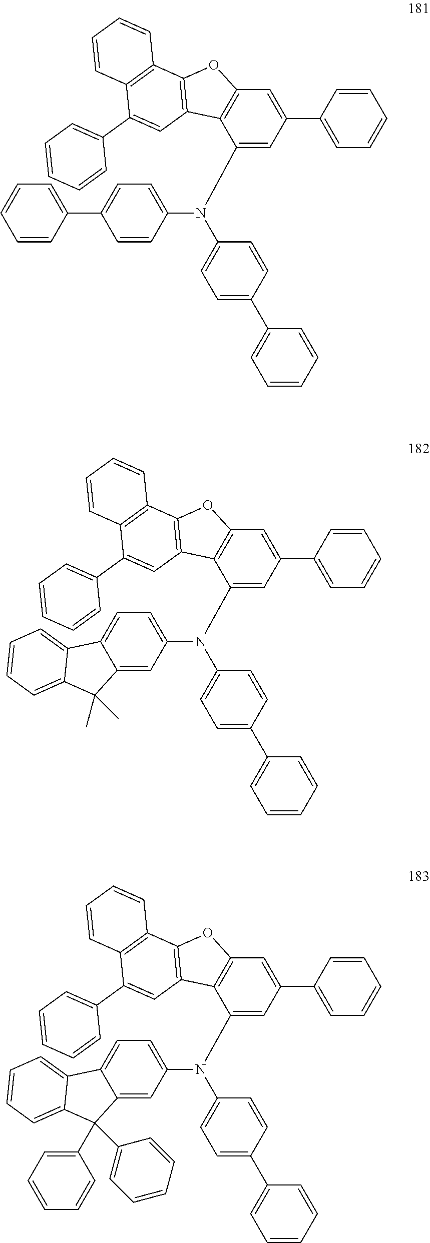 [SMILES: c1ccc(-c2ccc(N(c3ccc(-c4ccccc4)cc3)c3cc(-c4ccccc4)cc4oc5c6ccccc6c(-c6ccccc6)cc5c34)cc2)cc1 ; CC1(C)c2ccccc2-c2ccc(N(c3ccc(-c4ccccc4)cc3)c3cc(-c4ccccc4)cc4oc5c6ccccc6c(-c6ccccc6)cc5c34)cc21 ; c1ccc(-c2ccc(N(c3ccc4c(c3)C(c3ccccc3)(c3ccccc3)c3ccccc3-4)c3cc(-c4ccccc4)cc4oc5c6ccccc6c(-c6ccccc6)cc5c34)cc2)cc1]

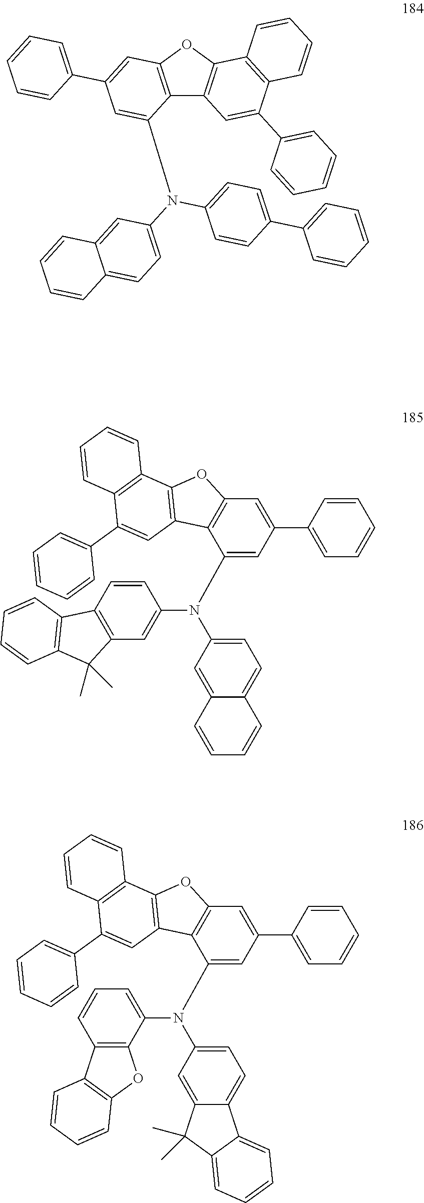 [SMILES: c1ccc(-c2ccc(N(c3ccc4ccccc4c3)c3cc(-c4ccccc4)cc4oc5c6ccccc6c(-c6ccccc6)cc5c34)cc2)cc1 ; CC1(C)c2ccccc2-c2ccc(N(c3ccc4ccccc4c3)c3cc(-c4ccccc4)cc4oc5c6ccccc6c(-c6ccccc6)cc5c34)cc21 ; CC1(C)c2ccccc2-c2ccc(N(c3cccc4c3oc3ccccc34)c3cc(-c4ccccc4)cc4oc5c6ccccc6c(-c6ccccc6)cc5c34)cc21]

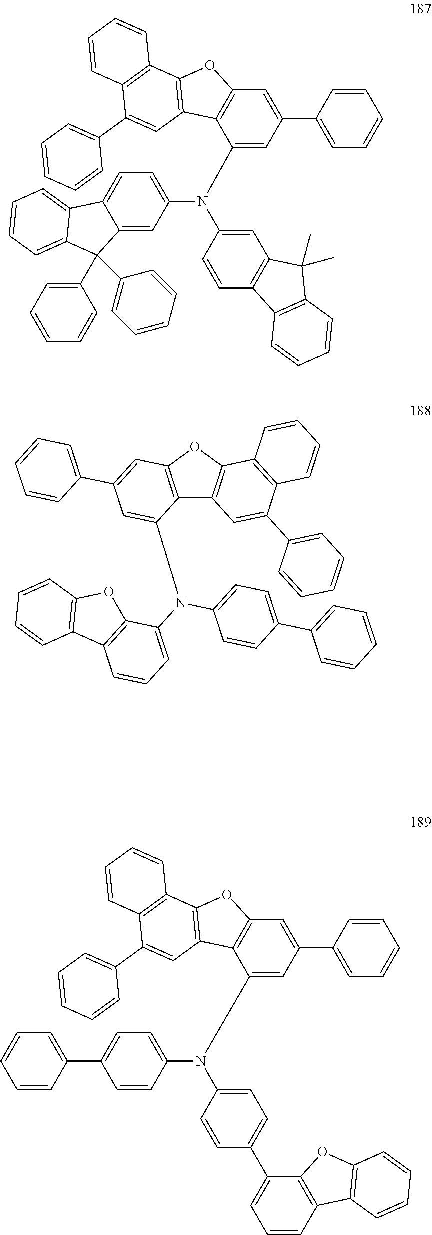 [SMILES: CC1(C)c2ccccc2-c2ccc(N(c3ccc4c(c3)C(c3ccccc3)(c3ccccc3)c3ccccc3-4)c3cc(-c4ccccc4)cc4oc5c6ccccc6c(-c6ccccc6)cc5c34)cc21 ; c1ccc(-c2ccc(N(c3cccc4c3oc3ccccc34)c3cc(-c4ccccc4)cc4oc5c6ccccc6c(-c6ccccc6)cc5c34)cc2)cc1 ; c1ccc(-c2ccc(N(c3ccc(-c4cccc5c4oc4ccccc45)cc3)c3cc(-c4ccccc4)cc4oc5c6ccccc6c(-c6ccccc6)cc5c34)cc2)cc1]

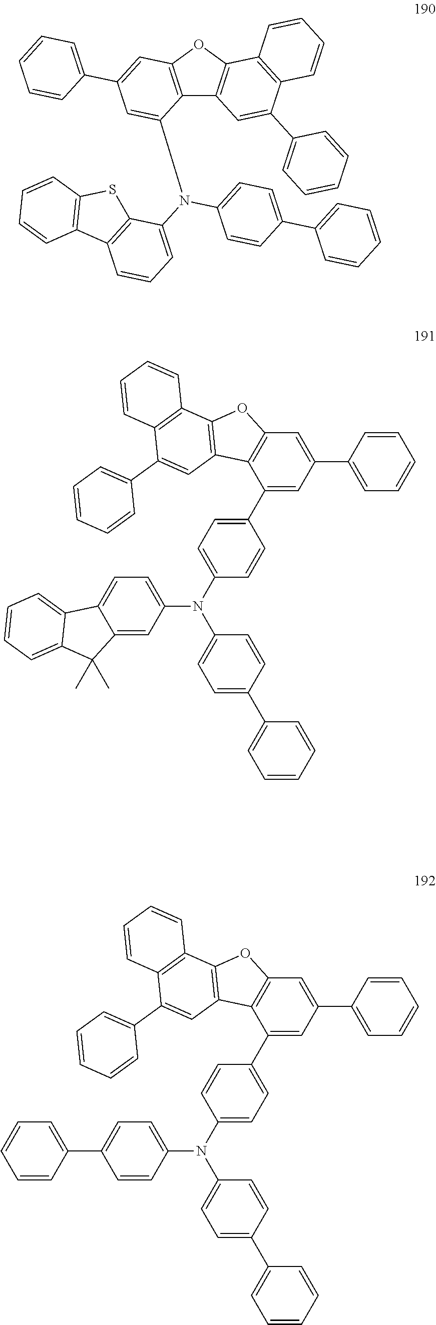 [SMILES: c1ccc(-c2ccc(N(c3cccc4c3sc3ccccc34)c3cc(-c4ccccc4)cc4oc5c6ccccc6c(-c6ccccc6)cc5c34)cc2)cc1 ; CC1(C)c2ccccc2-c2ccc(N(c3ccc(-c4ccccc4)cc3)c3ccc(-c4cc(-c5ccccc5)cc5oc6c7ccccc7c(-c7ccccc7)cc6c45)cc3)cc21 ; c1ccc(-c2ccc(N(c3ccc(-c4ccccc4)cc3)c3ccc(-c4cc(-c5ccccc5)cc5oc6c7ccccc7c(-c7ccccc7)cc6c45)cc3)cc2)cc1]

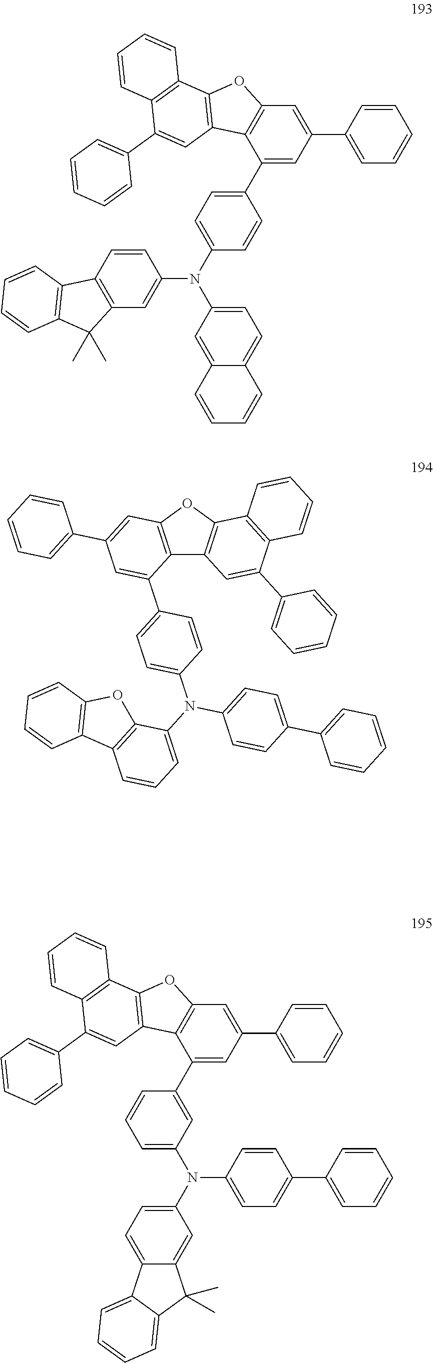 [SMILES: CC1(C)c2ccccc2-c2ccc(N(c3ccc(-c4cc(-c5ccccc5)cc5oc6c7ccccc7c(-c7ccccc7)cc6c45)cc3)c3ccc4ccccc4c3)cc21 ; c1ccc(-c2ccc(N(c3ccc(-c4cc(-c5ccccc5)cc5oc6c7ccccc7c(-c7ccccc7)cc6c45)cc3)c3cccc4c3oc3ccccc34)cc2)cc1 ; CC1(C)c2ccccc2-c2ccc(N(c3ccc(-c4ccccc4)cc3)c3cccc(-c4cc(-c5ccccc5)cc5oc6c7ccccc7c(-c7ccccc7)cc6c45)c3)cc21]

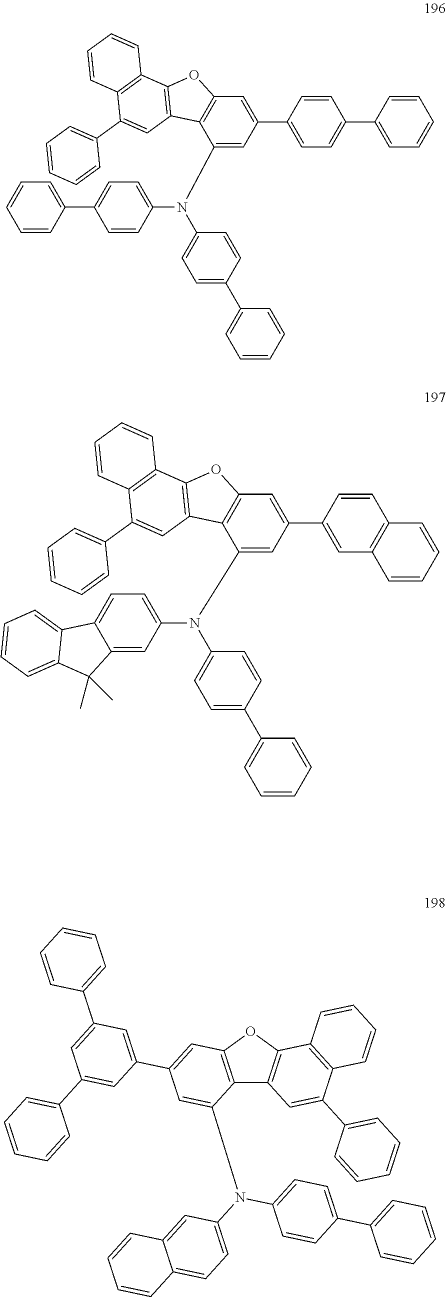 [SMILES: c1ccc(-c2ccc(-c3cc(N(c4ccc(-c5ccccc5)cc4)c4ccc(-c5ccccc5)cc4)c4c(c3)oc3c5ccccc5c(-c5ccccc5)cc34)cc2)cc1 ; CC1(C)c2ccccc2-c2ccc(N(c3ccc(-c4ccccc4)cc3)c3cc(-c4ccc5ccccc5c4)cc4oc5c6ccccc6c(-c6ccccc6)cc5c34)cc21 ; c1ccc(-c2ccc(N(c3ccc4ccccc4c3)c3cc(-c4cc(-c5ccccc5)cc(-c5ccccc5)c4)cc4oc5c6ccccc6c(-c6ccccc6)cc5c34)cc2)cc1]

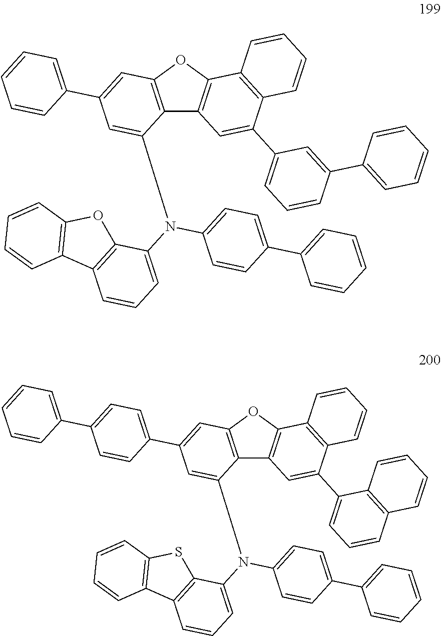 [SMILES: c1ccc(-c2ccc(N(c3cccc4c3oc3ccccc34)c3cc(-c4ccccc4)cc4oc5c6ccccc6c(-c6cccc(-c7ccccc7)c6)cc5c34)cc2)cc1 ; c1ccc(-c2ccc(-c3cc(N(c4ccc(-c5ccccc5)cc4)c4cccc5c4sc4ccccc45)c4c(c3)oc3c5ccccc5c(-c5cccc6ccccc56)cc34)cc2)cc1]

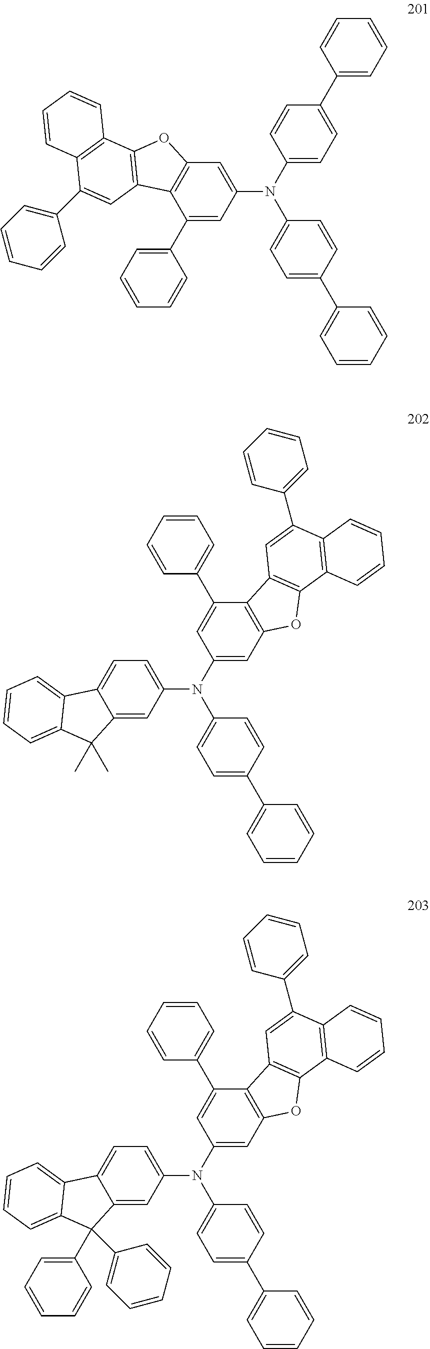 [SMILES: c1ccc(-c2ccc(N(c3ccc(-c4ccccc4)cc3)c3cc(-c4ccccc4)c4c(c3)oc3c5ccccc5c(-c5ccccc5)cc34)cc2)cc1 ; CC1(C)c2ccccc2-c2ccc(N(c3ccc(-c4ccccc4)cc3)c3cc(-c4ccccc4)c4c(c3)oc3c5ccccc5c(-c5ccccc5)cc34)cc21 ; c1ccc(-c2ccc(N(c3ccc4c(c3)C(c3ccccc3)(c3ccccc3)c3ccccc3-4)c3cc(-c4ccccc4)c4c(c3)oc3c5ccccc5c(-c5ccccc5)cc34)cc2)cc1]

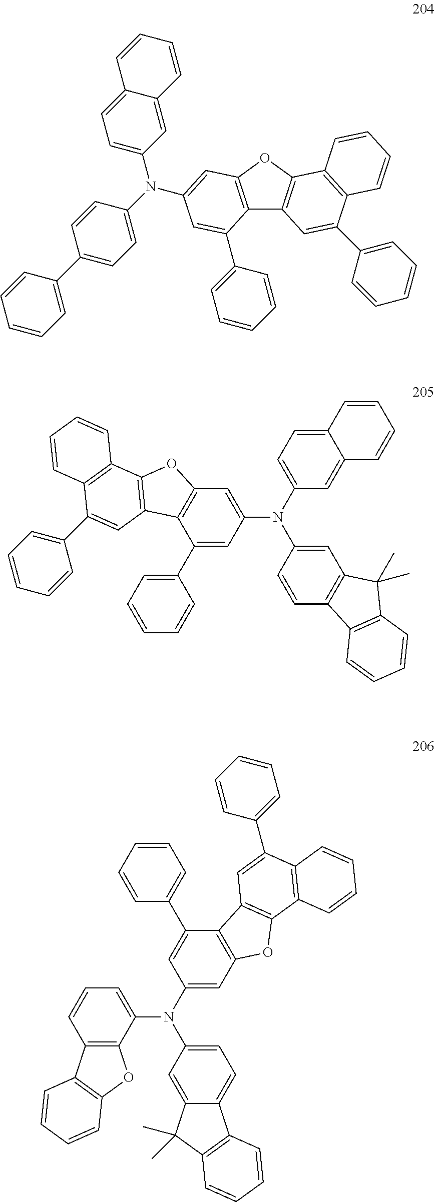 [SMILES: c1ccc(-c2ccc(N(c3ccc4ccccc4c3)c3cc(-c4ccccc4)c4c(c3)oc3c5ccccc5c(-c5ccccc5)cc34)cc2)cc1 ; CC1(C)c2ccccc2-c2ccc(N(c3ccc4ccccc4c3)c3cc(-c4ccccc4)c4c(c3)oc3c5ccccc5c(-c5ccccc5)cc34)cc21 ; CC1(C)c2ccccc2-c2ccc(N(c3cc(-c4ccccc4)c4c(c3)oc3c5ccccc5c(-c5ccccc5)cc34)c3cccc4c3oc3ccccc34)cc21]

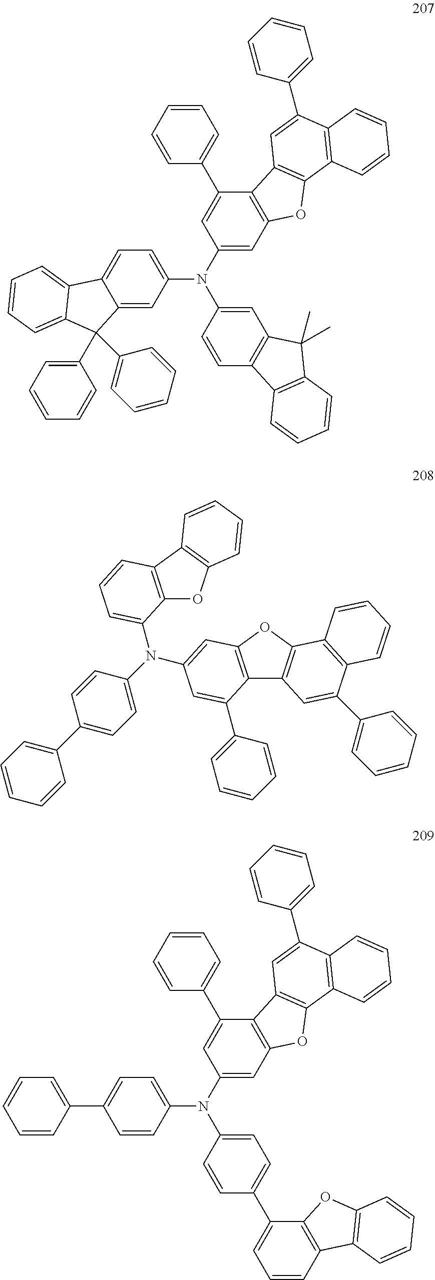 [SMILES: CC1(C)c2ccccc2-c2ccc(N(c3ccc4c(c3)C(c3ccccc3)(c3ccccc3)c3ccccc3-4)c3cc(-c4ccccc4)c4c(c3)oc3c5ccccc5c(-c5ccccc5)cc34)cc21 ; c1ccc(-c2ccc(N(c3cc(-c4ccccc4)c4c(c3)oc3c5ccccc5c(-c5ccccc5)cc34)c3cccc4c3oc3ccccc34)cc2)cc1 ; c1ccc(-c2ccc(N(c3ccc(-c4cccc5c4oc4ccccc45)cc3)c3cc(-c4ccccc4)c4c(c3)oc3c5ccccc5c(-c5ccccc5)cc34)cc2)cc1]

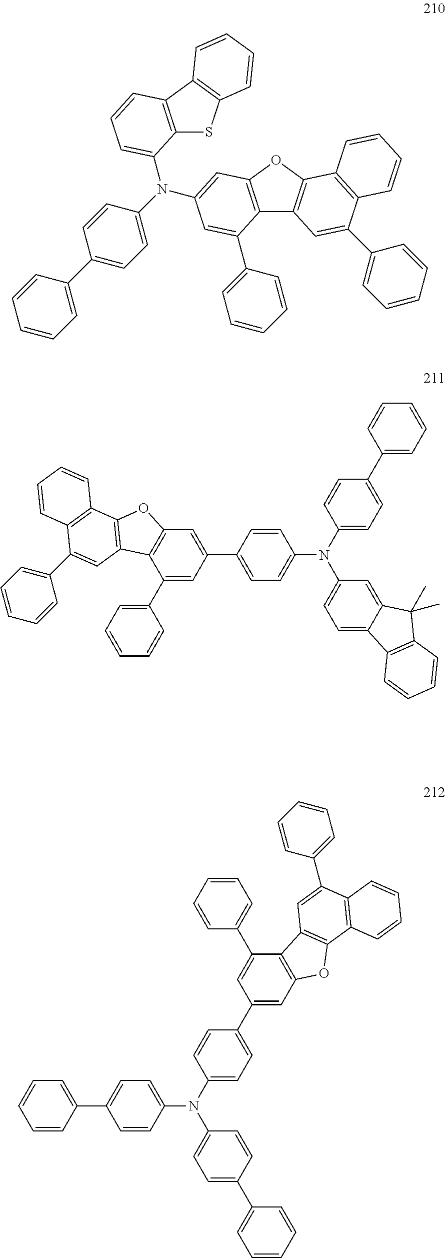 [SMILES: c1ccc(-c2ccc(N(c3cc(-c4ccccc4)c4c(c3)oc3c5ccccc5c(-c5ccccc5)cc34)c3cccc4c3sc3ccccc34)cc2)cc1 ; CC1(C)c2ccccc2-c2ccc(N(c3ccc(-c4ccccc4)cc3)c3ccc(-c4cc(-c5ccccc5)c5c(c4)oc4c6ccccc6c(-c6ccccc6)cc45)cc3)cc21 ; c1ccc(-c2ccc(N(c3ccc(-c4ccccc4)cc3)c3ccc(-c4cc(-c5ccccc5)c5c(c4)oc4c6ccccc6c(-c6ccccc6)cc45)cc3)cc2)cc1]

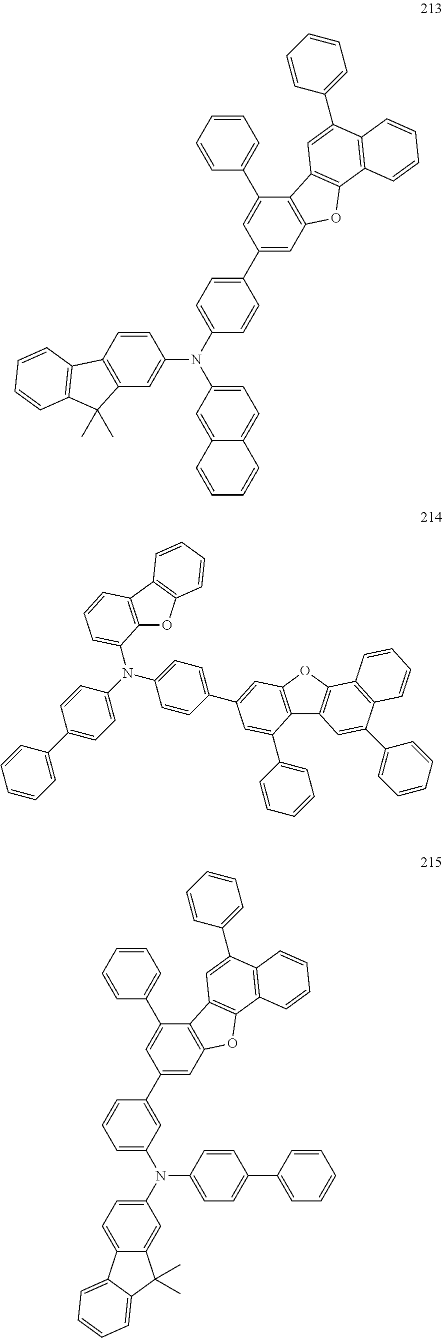 [SMILES: CC1(C)c2ccccc2-c2ccc(N(c3ccc(-c4cc(-c5ccccc5)c5c(c4)oc4c6ccccc6c(-c6ccccc6)cc45)cc3)c3ccc4ccccc4c3)cc21 ; c1ccc(-c2ccc(N(c3ccc(-c4cc(-c5ccccc5)c5c(c4)oc4c6ccccc6c(-c6ccccc6)cc45)cc3)c3cccc4c3oc3ccccc34)cc2)cc1 ; CC1(C)c2ccccc2-c2ccc(N(c3ccc(-c4ccccc4)cc3)c3cccc(-c4cc(-c5ccccc5)c5c(c4)oc4c6ccccc6c(-c6ccccc6)cc45)c3)cc21]

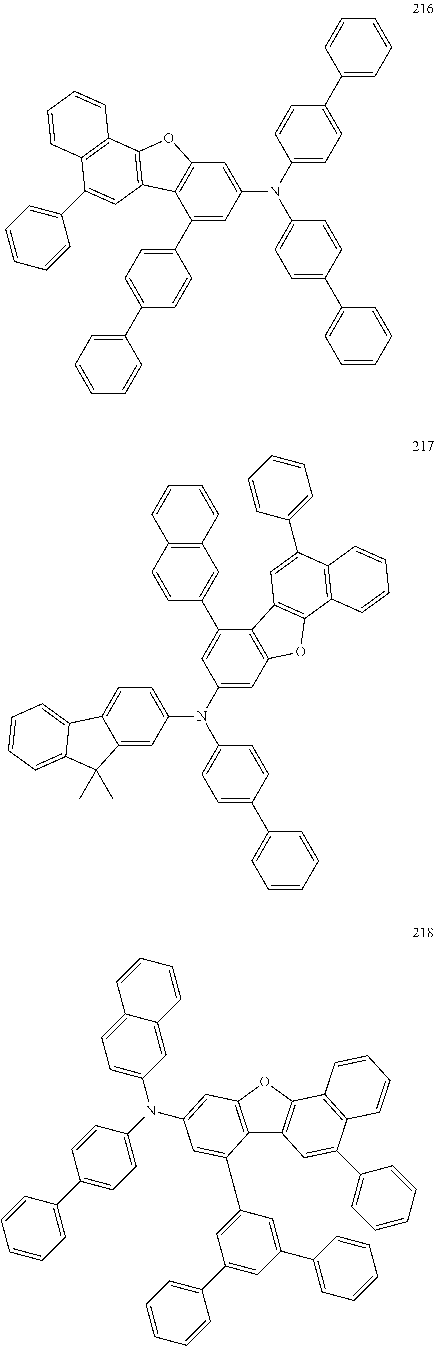 [SMILES: c1ccc(-c2ccc(-c3cc(N(c4ccc(-c5ccccc5)cc4)c4ccc(-c5ccccc5)cc4)cc4oc5c6ccccc6c(-c6ccccc6)cc5c34)cc2)cc1 ; CC1(C)c2ccccc2-c2ccc(N(c3ccc(-c4ccccc4)cc3)c3cc(-c4ccc5ccccc5c4)c4c(c3)oc3c5ccccc5c(-c5ccccc5)cc34)cc21 ; c1ccc(-c2ccc(N(c3ccc4ccccc4c3)c3cc(-c4cc(-c5ccccc5)cc(-c5ccccc5)c4)c4c(c3)oc3c5ccccc5c(-c5ccccc5)cc34)cc2)cc1]

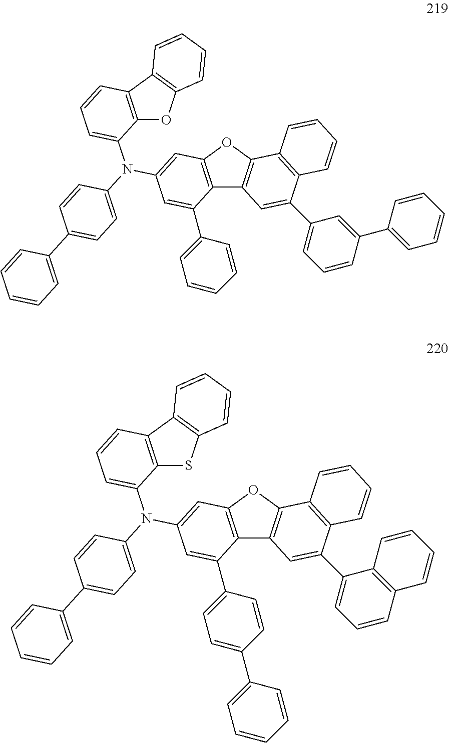 [SMILES: c1ccc(-c2ccc(N(c3cc(-c4ccccc4)c4c(c3)oc3c5ccccc5c(-c5cccc(-c6ccccc6)c5)cc34)c3cccc4c3oc3ccccc34)cc2)cc1 ; c1ccc(-c2ccc(-c3cc(N(c4ccc(-c5ccccc5)cc4)c4cccc5c4sc4ccccc45)cc4oc5c6ccccc6c(-c6cccc7ccccc67)cc5c34)cc2)cc1]

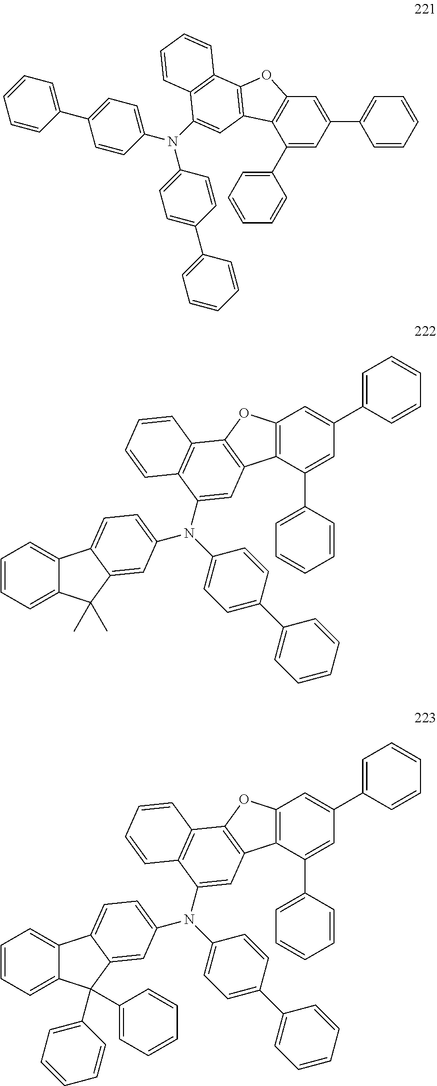 [SMILES: c1ccc(-c2ccc(N(c3ccc(-c4ccccc4)cc3)c3cc4c(oc5cc(-c6ccccc6)cc(-c6ccccc6)c54)c4ccccc34)cc2)cc1 ; CC1(C)c2ccccc2-c2ccc(N(c3ccc(-c4ccccc4)cc3)c3cc4c(oc5cc(-c6ccccc6)cc(-c6ccccc6)c54)c4ccccc34)cc21 ; c1ccc(-c2ccc(N(c3ccc4c(c3)C(c3ccccc3)(c3ccccc3)c3ccccc3-4)c3cc4c(oc5cc(-c6ccccc6)cc(-c6ccccc6)c54)c4ccccc34)cc2)cc1]

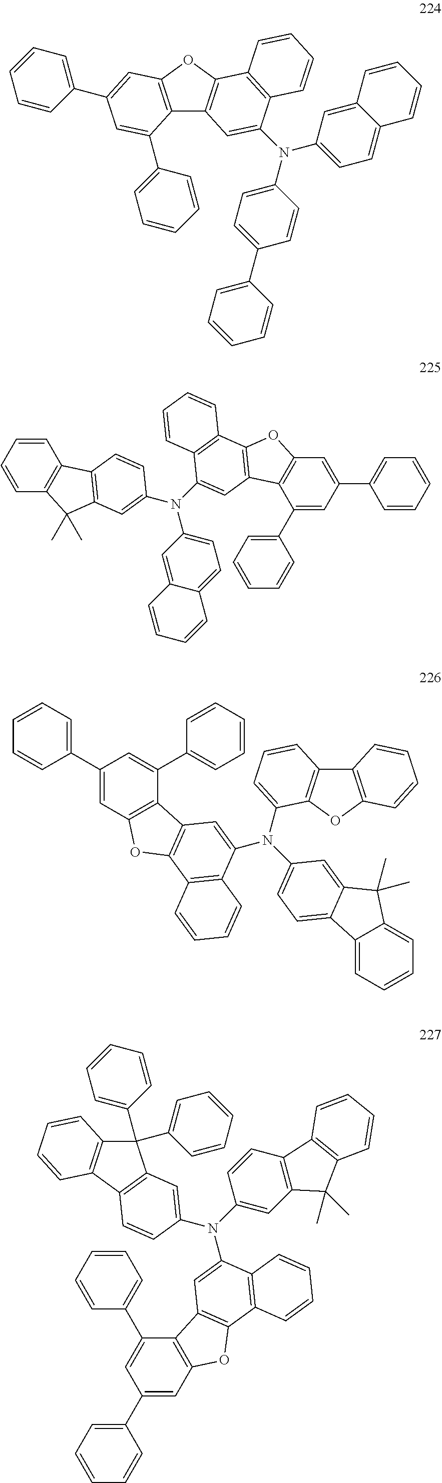 [SMILES: c1ccc(-c2ccc(N(c3ccc4ccccc4c3)c3cc4c(oc5cc(-c6ccccc6)cc(-c6ccccc6)c54)c4ccccc34)cc2)cc1 ; CC1(C)c2ccccc2-c2ccc(N(c3ccc4ccccc4c3)c3cc4c(oc5cc(-c6ccccc6)cc(-c6ccccc6)c54)c4ccccc34)cc21 ; CC1(C)c2ccccc2-c2ccc(N(c3cc4c(oc5cc(-c6ccccc6)cc(-c6ccccc6)c54)c4ccccc34)c3cccc4c3oc3ccccc34)cc21 ; CC1(C)c2ccccc2-c2ccc(N(c3ccc4c(c3)C(c3ccccc3)(c3ccccc3)c3ccccc3-4)c3cc4c(oc5cc(-c6ccccc6)cc(-c6ccccc6)c54)c4ccccc34)cc21]

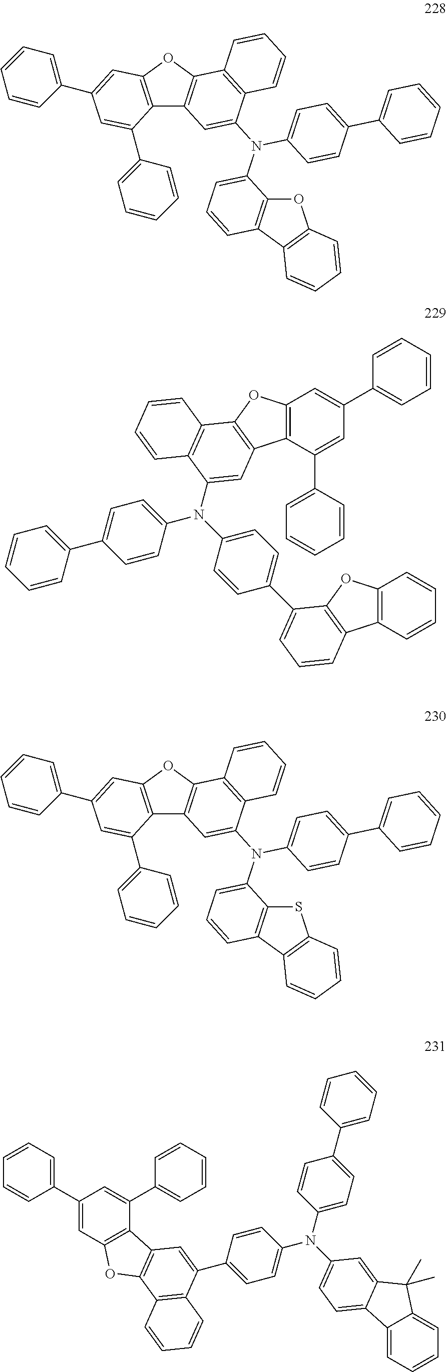 [SMILES: c1ccc(-c2ccc(N(c3cc4c(oc5cc(-c6ccccc6)cc(-c6ccccc6)c54)c4ccccc34)c3cccc4c3oc3ccccc34)cc2)cc1 ; c1ccc(-c2ccc(N(c3ccc(-c4cccc5c4oc4ccccc45)cc3)c3cc4c(oc5cc(-c6ccccc6)cc(-c6ccccc6)c54)c4ccccc34)cc2)cc1 ; c1ccc(-c2ccc(N(c3cc4c(oc5cc(-c6ccccc6)cc(-c6ccccc6)c54)c4ccccc34)c3cccc4c3sc3ccccc34)cc2)cc1 ; CC1(C)c2ccccc2-c2ccc(N(c3ccc(-c4ccccc4)cc3)c3ccc(-c4cc5c(oc6cc(-c7ccccc7)cc(-c7ccccc7)c65)c5ccccc45)cc3)cc21]

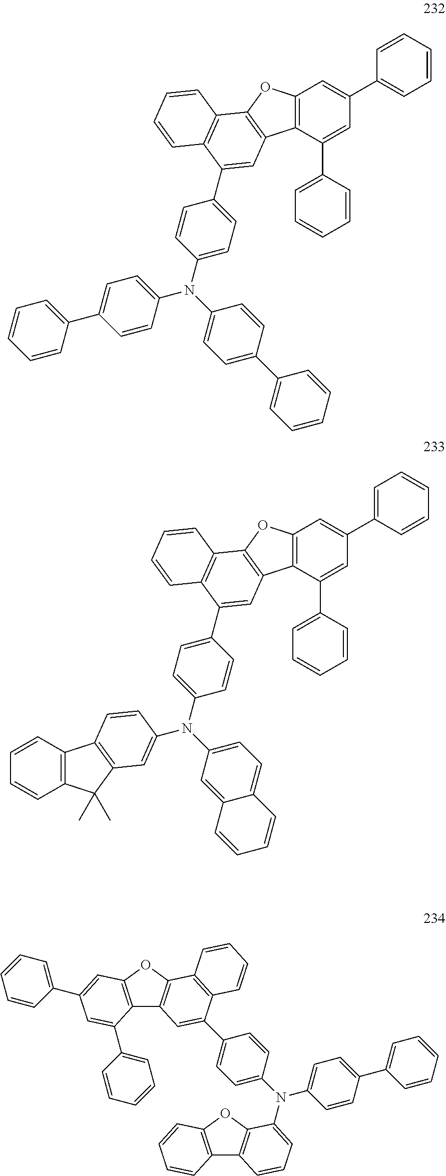 [SMILES: c1ccc(-c2ccc(N(c3ccc(-c4ccccc4)cc3)c3ccc(-c4cc5c(oc6cc(-c7ccccc7)cc(-c7ccccc7)c65)c5ccccc45)cc3)cc2)cc1 ; CC1(C)c2ccccc2-c2ccc(N(c3ccc(-c4cc5c(oc6cc(-c7ccccc7)cc(-c7ccccc7)c65)c5ccccc45)cc3)c3ccc4ccccc4c3)cc21 ; c1ccc(-c2ccc(N(c3ccc(-c4cc5c(oc6cc(-c7ccccc7)cc(-c7ccccc7)c65)c5ccccc45)cc3)c3cccc4c3oc3ccccc34)cc2)cc1]

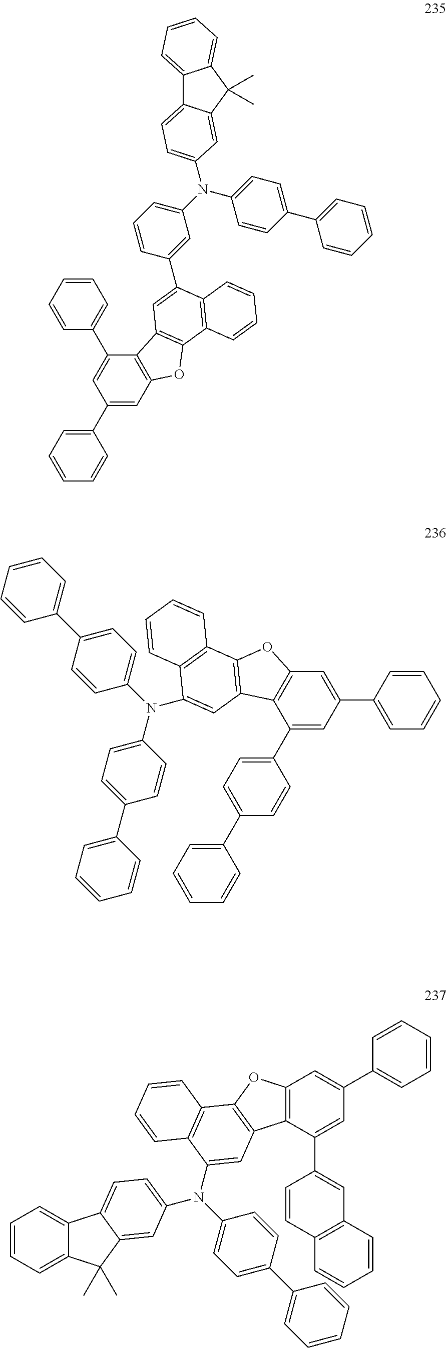 [SMILES: CC1(C)c2ccccc2-c2ccc(N(c3ccc(-c4ccccc4)cc3)c3cccc(-c4cc5c(oc6cc(-c7ccccc7)cc(-c7ccccc7)c65)c5ccccc45)c3)cc21 ; c1ccc(-c2ccc(-c3cc(-c4ccccc4)cc4oc5c6ccccc6c(N(c6ccc(-c7ccccc7)cc6)c6ccc(-c7ccccc7)cc6)cc5c34)cc2)cc1 ; CC1(C)c2ccccc2-c2ccc(N(c3ccc(-c4ccccc4)cc3)c3cc4c(oc5cc(-c6ccccc6)cc(-c6ccc7ccccc7c6)c54)c4ccccc34)cc21]

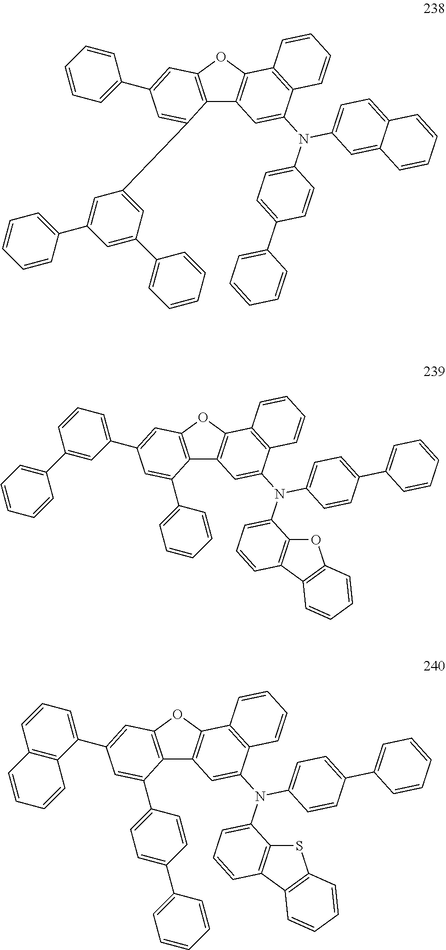 [SMILES: c1ccc(-c2ccc(N(c3ccc4ccccc4c3)c3cc4c(oc5cc(-c6ccccc6)cc(-c6cc(-c7ccccc7)cc(-c7ccccc7)c6)c54)c4ccccc34)cc2)cc1 ; c1ccc(-c2ccc(N(c3cc4c(oc5cc(-c6cccc(-c7ccccc7)c6)cc(-c6ccccc6)c54)c4ccccc34)c3cccc4c3oc3ccccc34)cc2)cc1 ; c1ccc(-c2ccc(-c3cc(-c4cccc5ccccc45)cc4oc5c6ccccc6c(N(c6ccc(-c7ccccc7)cc6)c6cccc7c6sc6ccccc67)cc5c34)cc2)cc1]

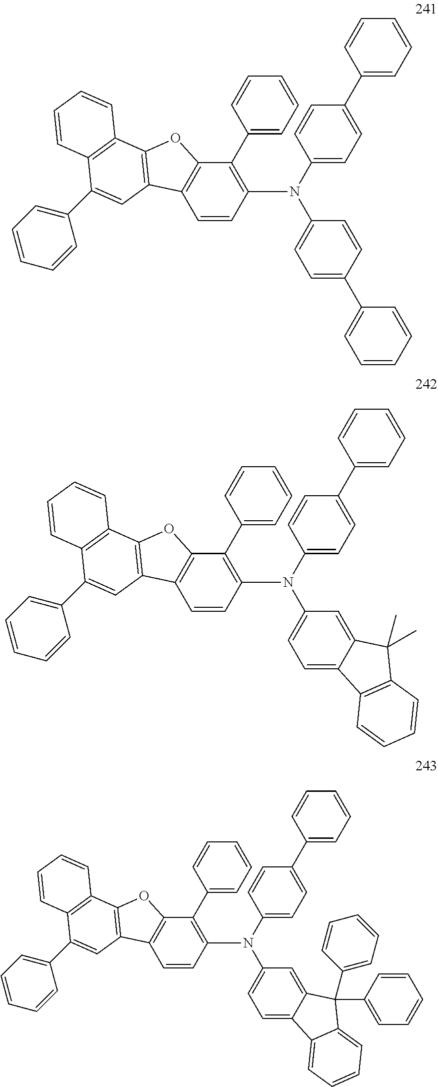

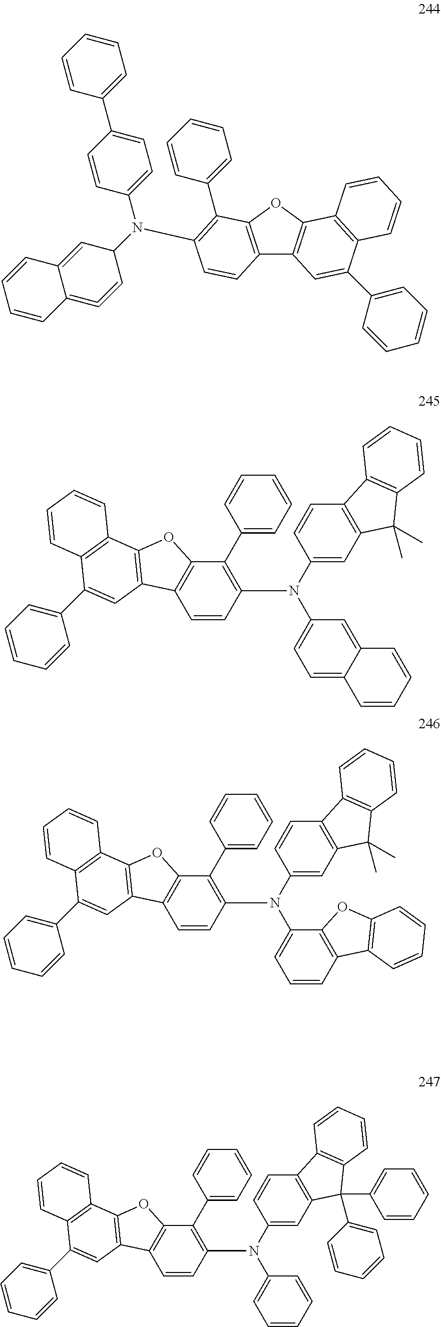

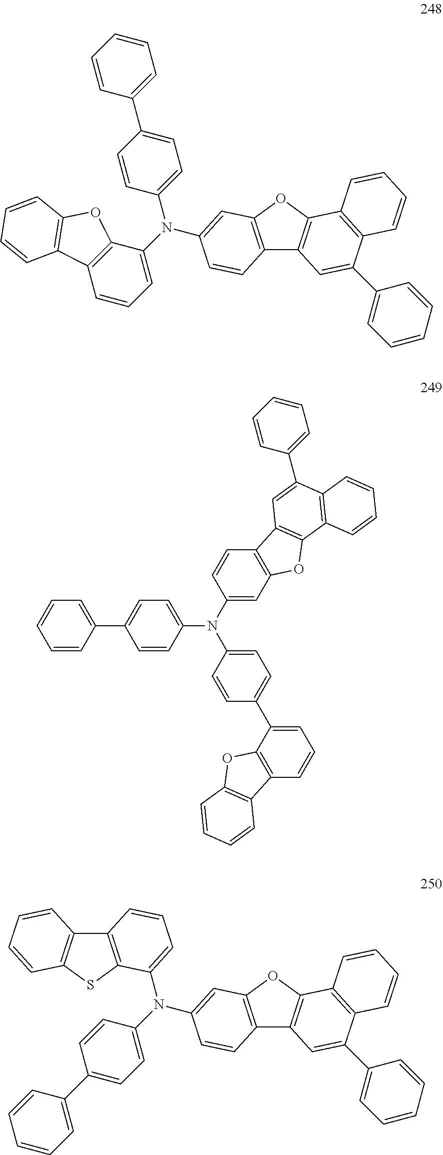 [SMILES: c1ccc(-c2ccc(N(c3ccc4c(c3)oc3c5ccccc5c(-c5ccccc5)cc43)c3cccc4c3oc3ccccc34)cc2)cc1 ; c1ccc(-c2ccc(N(c3ccc(-c4cccc5c4oc4ccccc45)cc3)c3ccc4c(c3)oc3c5ccccc5c(-c5ccccc5)cc43)cc2)cc1 ; c1ccc(-c2ccc(N(c3ccc4c(c3)oc3c5ccccc5c(-c5ccccc5)cc43)c3cccc4c3sc3ccccc34)cc2)cc1]

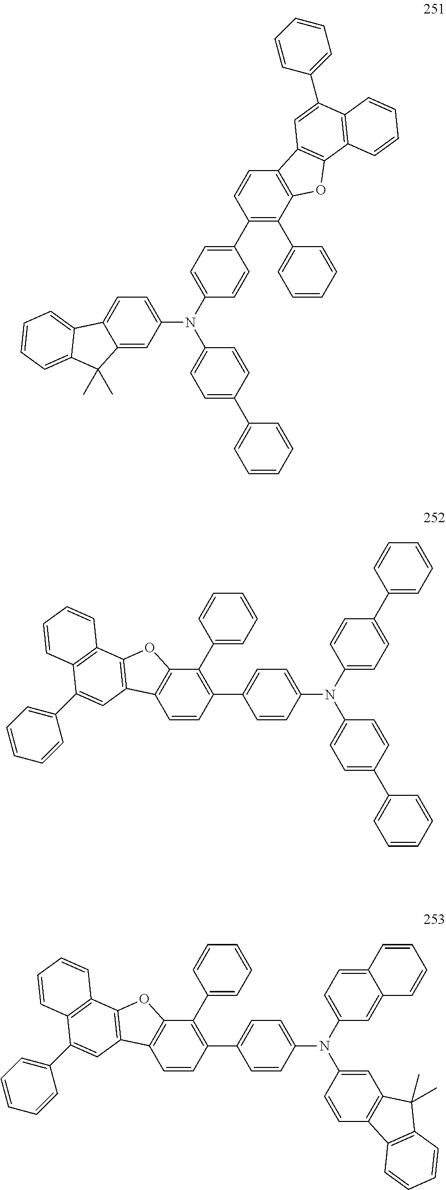 [SMILES: CC1(C)c2ccccc2-c2ccc(N(c3ccc(-c4ccccc4)cc3)c3ccc(-c4ccc5c(oc6c7ccccc7c(-c7ccccc7)cc56)c4-c4ccccc4)cc3)cc21 ; c1ccc(-c2ccc(N(c3ccc(-c4ccccc4)cc3)c3ccc(-c4ccc5c(oc6c7ccccc7c(-c7ccccc7)cc56)c4-c4ccccc4)cc3)cc2)cc1 ; CC1(C)c2ccccc2-c2ccc(N(c3ccc(-c4ccc5c(oc6c7ccccc7c(-c7ccccc7)cc56)c4-c4ccccc4)cc3)c3ccc4ccccc4c3)cc21]

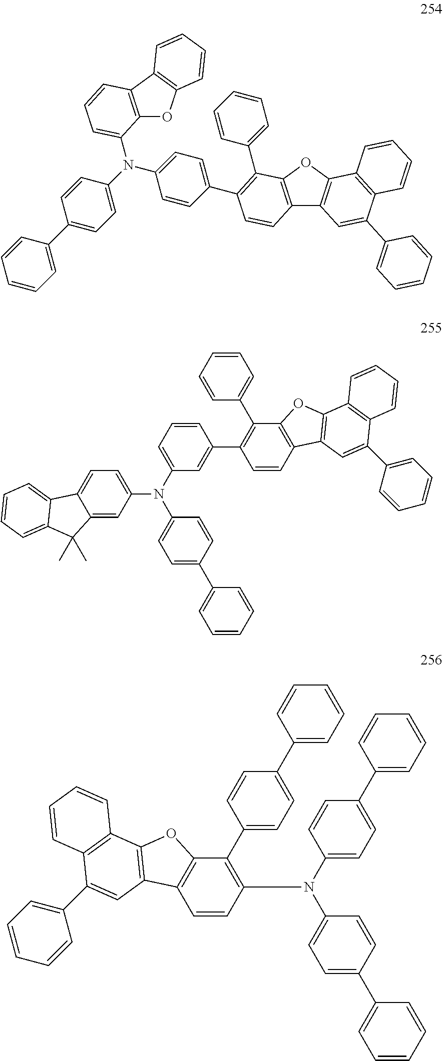 [SMILES: c1ccc(-c2ccc(N(c3ccc(-c4ccc5c(oc6c7ccccc7c(-c7ccccc7)cc56)c4-c4ccccc4)cc3)c3cccc4c3oc3ccccc34)cc2)cc1 ; CC1(C)c2ccccc2-c2ccc(N(c3ccc(-c4ccccc4)cc3)c3cccc(-c4ccc5c(oc6c7ccccc7c(-c7ccccc7)cc56)c4-c4ccccc4)c3)cc21 ; c1ccc(-c2ccc(-c3c(N(c4ccc(-c5ccccc5)cc4)c4ccc(-c5ccccc5)cc4)ccc4c3oc3c5ccccc5c(-c5ccccc5)cc43)cc2)cc1]

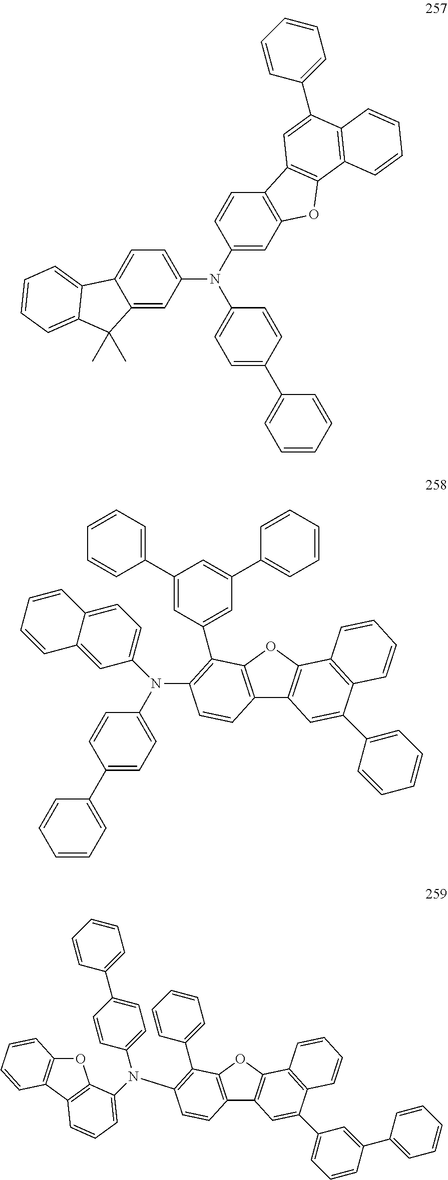 [SMILES: CC1(C)c2ccccc2-c2ccc(N(c3ccc(-c4ccccc4)cc3)c3ccc4c(c3)oc3c5ccccc5c(-c5ccccc5)cc43)cc21 ; c1ccc(-c2ccc(N(c3ccc4ccccc4c3)c3ccc4c(oc5c6ccccc6c(-c6ccccc6)cc45)c3-c3cc(-c4ccccc4)cc(-c4ccccc4)c3)cc2)cc1 ; c1ccc(-c2ccc(N(c3ccc4c(oc5c6ccccc6c(-c6cccc(-c7ccccc7)c6)cc45)c3-c3ccccc3)c3cccc4c3oc3ccccc34)cc2)cc1]

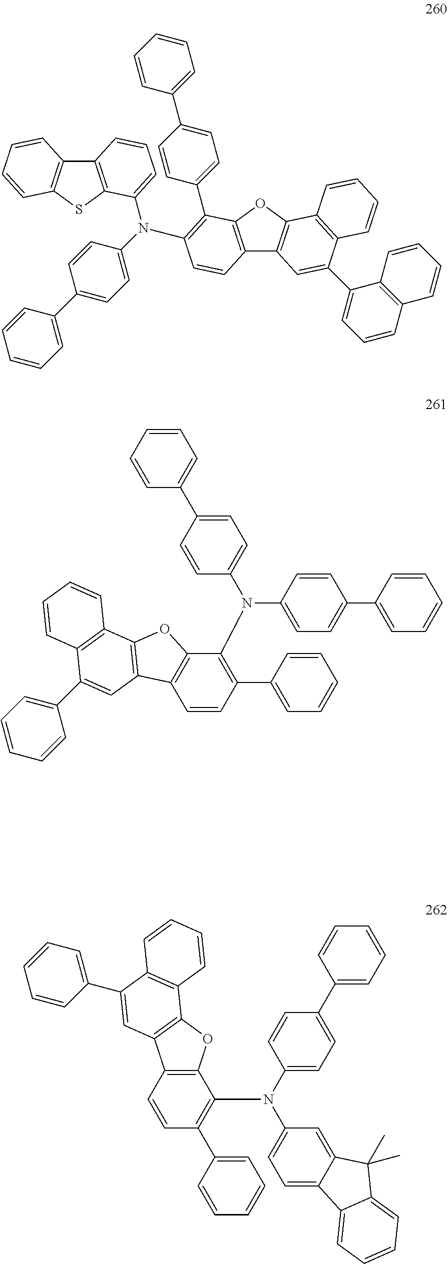 [SMILES: c1ccc(-c2ccc(-c3c(N(c4ccc(-c5ccccc5)cc4)c4cccc5c4sc4ccccc45)ccc4c3oc3c5ccccc5c(-c5cccc6ccccc56)cc43)cc2)cc1 ; c1ccc(-c2ccc(N(c3ccc(-c4ccccc4)cc3)c3c(-c4ccccc4)ccc4c3oc3c5ccccc5c(-c5ccccc5)cc43)cc2)cc1 ; CC1(C)c2ccccc2-c2ccc(N(c3ccc(-c4ccccc4)cc3)c3c(-c4ccccc4)ccc4c3oc3c5ccccc5c(-c5ccccc5)cc43)cc21]

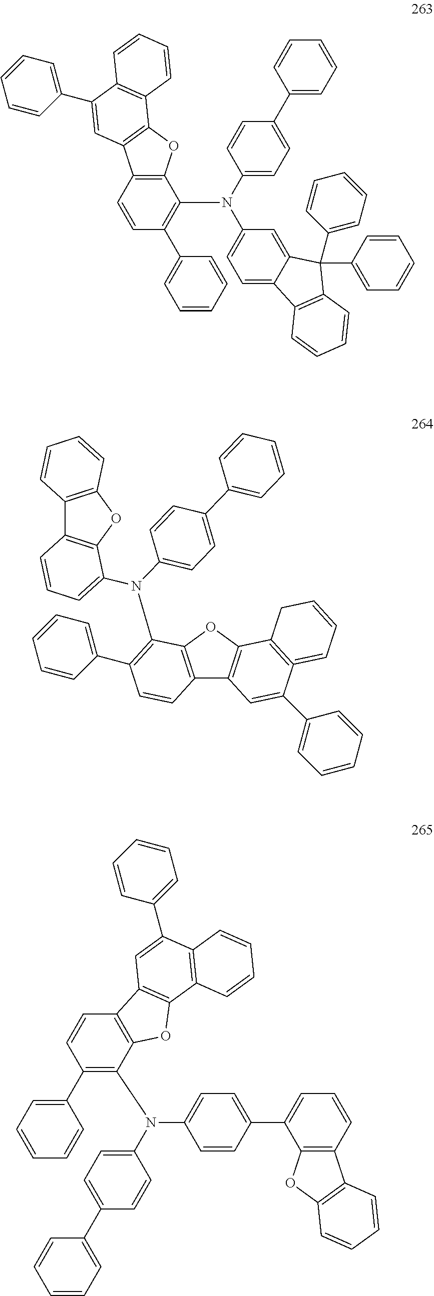

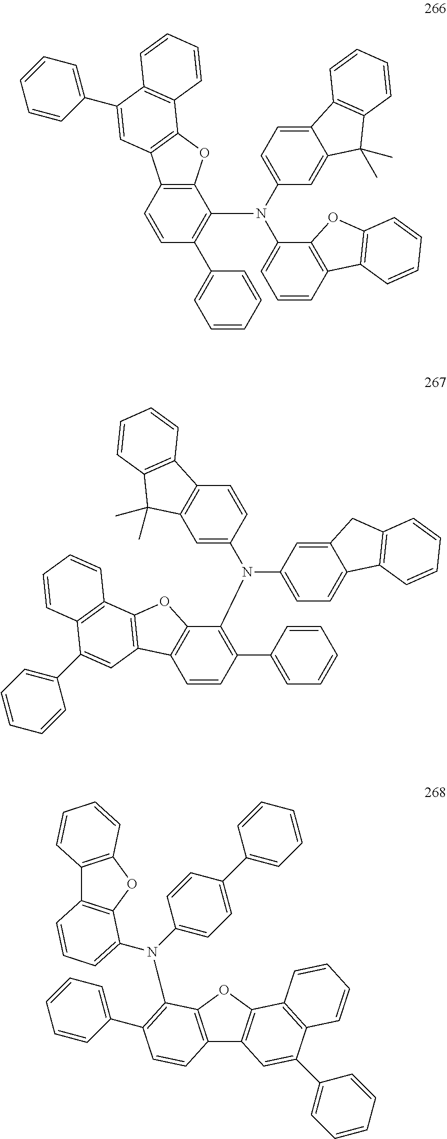 [SMILES: CC1(C)c2ccccc2-c2ccc(N(c3cccc4c3oc3ccccc34)c3c(-c4ccccc4)ccc4c3oc3c5ccccc5c(-c5ccccc5)cc43)cc21 ; CC1(C)c2ccccc2-c2ccc(N(c3ccc4c(c3)Cc3ccccc3-4)c3c(-c4ccccc4)ccc4c3oc3c5ccccc5c(-c5ccccc5)cc43)cc21 ; c1ccc(-c2ccc(N(c3cccc4c3oc3ccccc34)c3c(-c4ccccc4)ccc4c3oc3c5ccccc5c(-c5ccccc5)cc43)cc2)cc1]

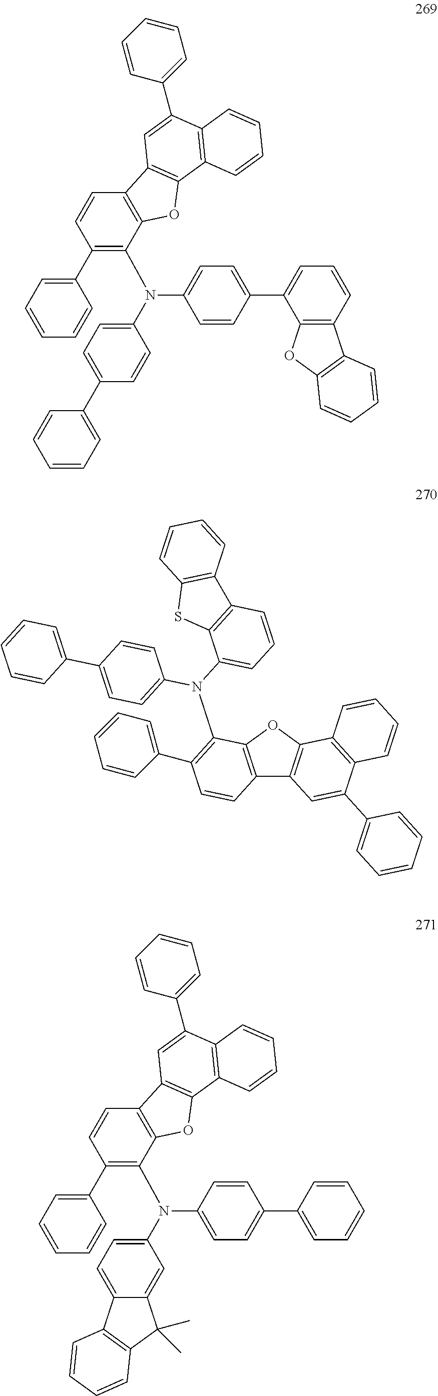 [SMILES: c1ccc(-c2ccc(N(c3ccc(-c4cccc5c4oc4ccccc45)cc3)c3c(-c4ccccc4)ccc4c3oc3c5ccccc5c(-c5ccccc5)cc43)cc2)cc1 ; c1ccc(-c2ccc(N(c3c(-c4ccccc4)ccc4c3oc3c5ccccc5c(-c5ccccc5)cc43)c3cccc4c3sc3ccccc34)cc2)cc1 ; CC1(C)c2ccccc2-c2ccc(N(c3ccc(-c4ccccc4)cc3)c3c(-c4ccccc4)ccc4c3oc3c5ccccc5c(-c5ccccc5)cc43)cc21]

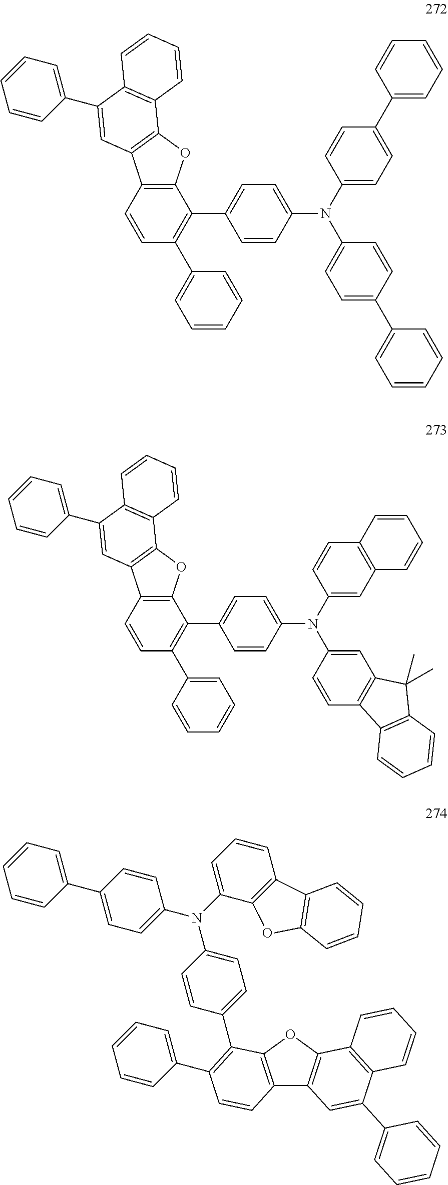 [SMILES: c1ccc(-c2ccc(N(c3ccc(-c4ccccc4)cc3)c3ccc(-c4c(-c5ccccc5)ccc5c4oc4c6ccccc6c(-c6ccccc6)cc54)cc3)cc2)cc1 ; CC1(C)c2ccccc2-c2ccc(N(c3ccc(-c4c(-c5ccccc5)ccc5c4oc4c6ccccc6c(-c6ccccc6)cc54)cc3)c3ccc4ccccc4c3)cc21 ; c1ccc(-c2ccc(N(c3ccc(-c4c(-c5ccccc5)ccc5c4oc4c6ccccc6c(-c6ccccc6)cc54)cc3)c3cccc4c3oc3ccccc34)cc2)cc1]

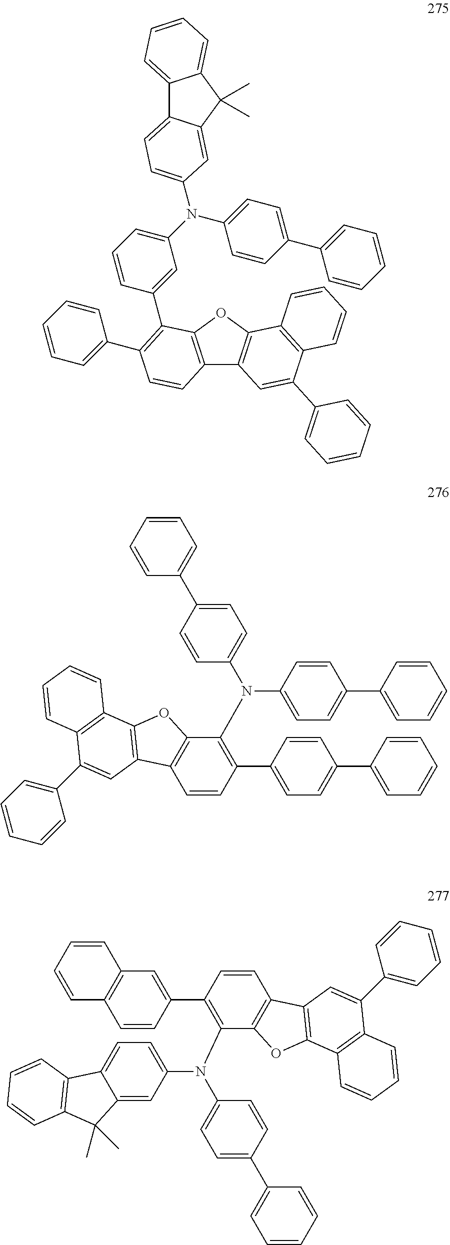 [SMILES: CC1(C)c2ccccc2-c2ccc(N(c3ccc(-c4ccccc4)cc3)c3cccc(-c4c(-c5ccccc5)ccc5c4oc4c6ccccc6c(-c6ccccc6)cc54)c3)cc21 ; c1ccc(-c2ccc(-c3ccc4c(oc5c6ccccc6c(-c6ccccc6)cc45)c3N(c3ccc(-c4ccccc4)cc3)c3ccc(-c4ccccc4)cc3)cc2)cc1 ; CC1(C)c2ccccc2-c2ccc(N(c3ccc(-c4ccccc4)cc3)c3c(-c4ccc5ccccc5c4)ccc4c3oc3c5ccccc5c(-c5ccccc5)cc43)cc21]

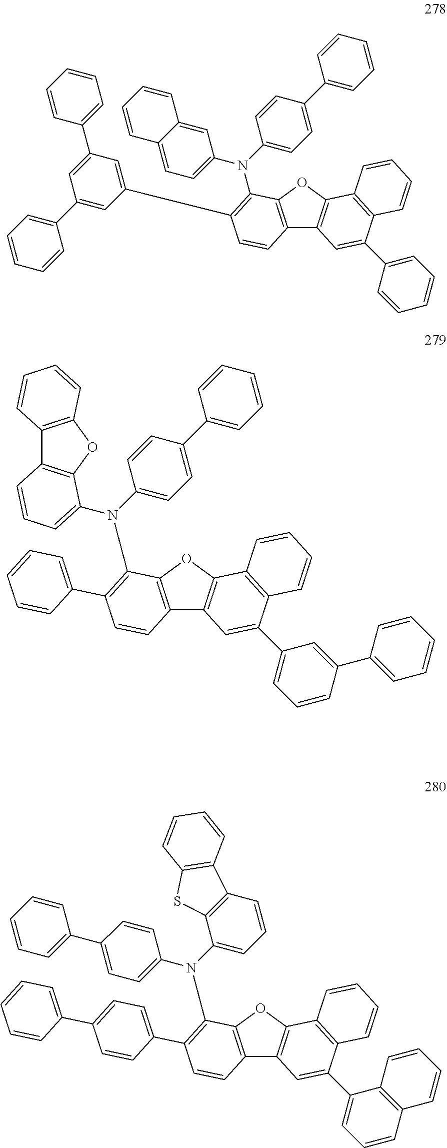 [SMILES: c1ccc(-c2ccc(N(c3ccc4ccccc4c3)c3c(-c4cc(-c5ccccc5)cc(-c5ccccc5)c4)ccc4c3oc3c5ccccc5c(-c5ccccc5)cc43)cc2)cc1 ; c1ccc(-c2ccc(N(c3cccc4c3oc3ccccc34)c3c(-c4ccccc4)ccc4c3oc3c5ccccc5c(-c5cccc(-c6ccccc6)c5)cc43)cc2)cc1 ; c1ccc(-c2ccc(-c3ccc4c(oc5c6ccccc6c(-c6cccc7ccccc67)cc45)c3N(c3ccc(-c4ccccc4)cc3)c3cccc4c3sc3ccccc34)cc2)cc1]

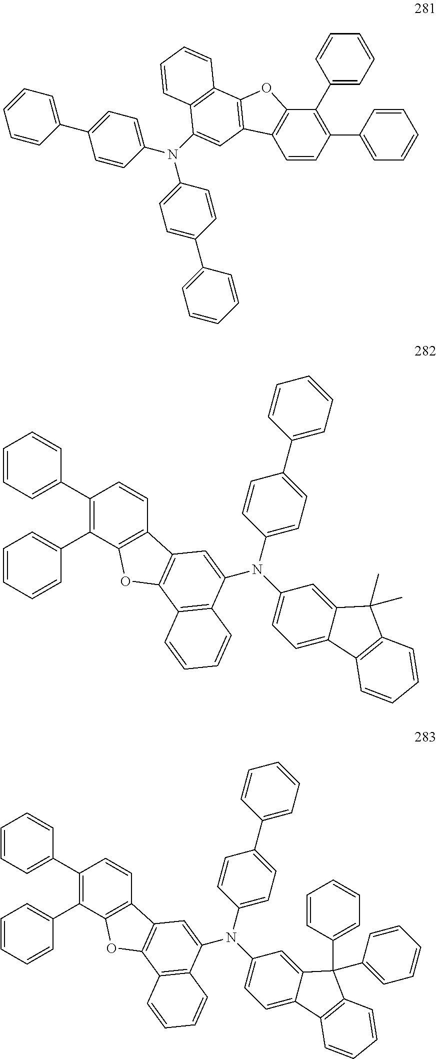 [SMILES: c1ccc(-c2ccc(N(c3ccc(-c4ccccc4)cc3)c3cc4c5ccc(-c6ccccc6)c(-c6ccccc6)c5oc4c4ccccc34)cc2)cc1 ; CC1(C)c2ccccc2-c2ccc(N(c3ccc(-c4ccccc4)cc3)c3cc4c5ccc(-c6ccccc6)c(-c6ccccc6)c5oc4c4ccccc34)cc21 ; c1ccc(-c2ccc(N(c3ccc4c(c3)C(c3ccccc3)(c3ccccc3)c3ccccc3-4)c3cc4c5ccc(-c6ccccc6)c(-c6ccccc6)c5oc4c4ccccc34)cc2)cc1]

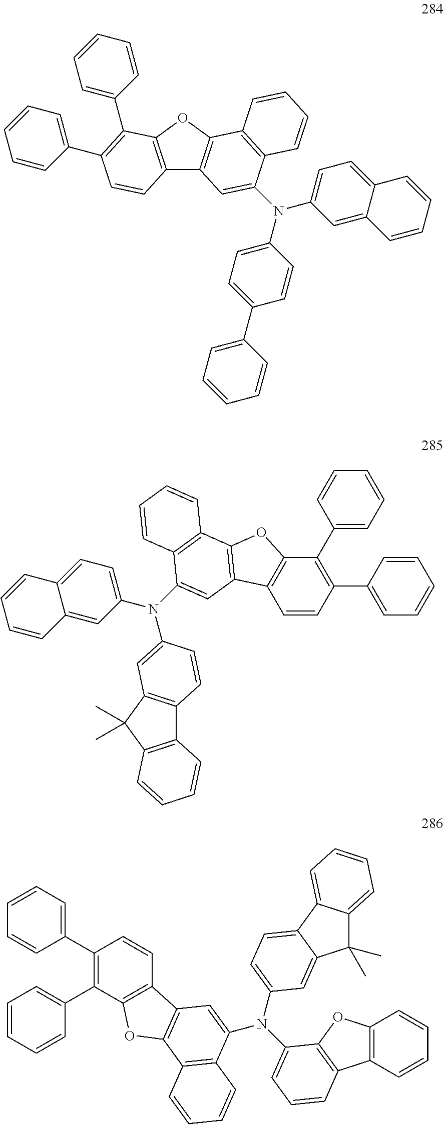 [SMILES: c1ccc(-c2ccc(N(c3ccc4ccccc4c3)c3cc4c5ccc(-c6ccccc6)c(-c6ccccc6)c5oc4c4ccccc34)cc2)cc1 ; CC1(C)c2ccccc2-c2ccc(N(c3ccc4ccccc4c3)c3cc4c5ccc(-c6ccccc6)c(-c6ccccc6)c5oc4c4ccccc34)cc21 ; CC1(C)c2ccccc2-c2ccc(N(c3cc4c5ccc(-c6ccccc6)c(-c6ccccc6)c5oc4c4ccccc34)c3cccc4c3oc3ccccc34)cc21]

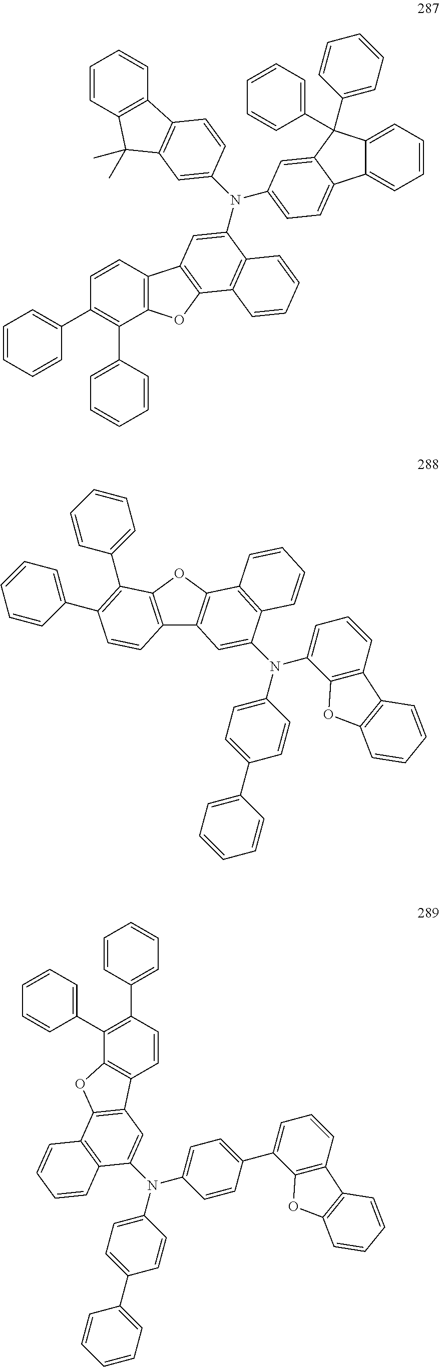 [SMILES: CC1(C)c2ccccc2-c2ccc(N(c3ccc4c(c3)C(c3ccccc3)(c3ccccc3)c3ccccc3-4)c3cc4c5ccc(-c6ccccc6)c(-c6ccccc6)c5oc4c4ccccc34)cc21 ; c1ccc(-c2ccc(N(c3cc4c5ccc(-c6ccccc6)c(-c6ccccc6)c5oc4c4ccccc34)c3cccc4c3oc3ccccc34)cc2)cc1 ; c1ccc(-c2ccc(N(c3ccc(-c4cccc5c4oc4ccccc45)cc3)c3cc4c5ccc(-c6ccccc6)c(-c6ccccc6)c5oc4c4ccccc34)cc2)cc1]

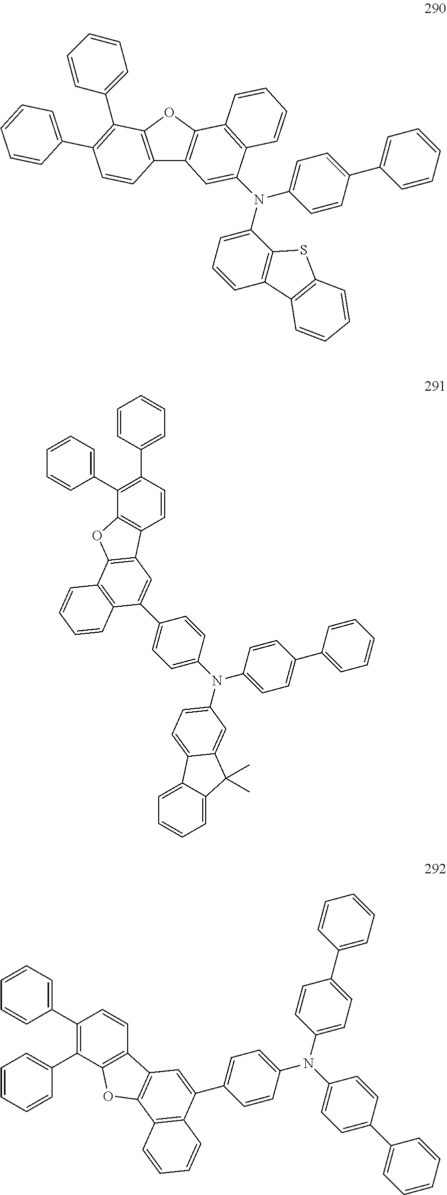 [SMILES: c1ccc(-c2ccc(N(c3cc4c5ccc(-c6ccccc6)c(-c6ccccc6)c5oc4c4ccccc34)c3cccc4c3sc3ccccc34)cc2)cc1 ; CC1(C)c2ccccc2-c2ccc(N(c3ccc(-c4ccccc4)cc3)c3ccc(-c4cc5c6ccc(-c7ccccc7)c(-c7ccccc7)c6oc5c5ccccc45)cc3)cc21 ; c1ccc(-c2ccc(N(c3ccc(-c4ccccc4)cc3)c3ccc(-c4cc5c6ccc(-c7ccccc7)c(-c7ccccc7)c6oc5c5ccccc45)cc3)cc2)cc1]

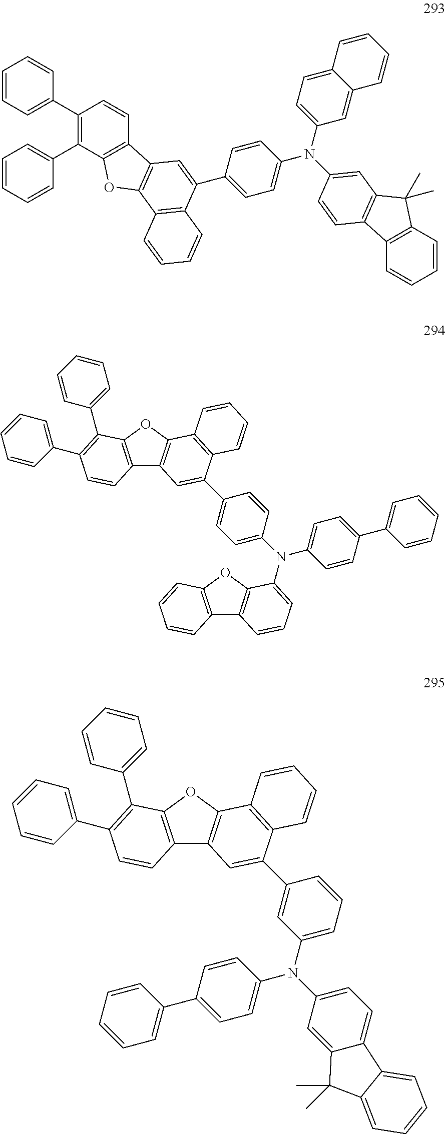 [SMILES: CC1(C)c2ccccc2-c2ccc(N(c3ccc(-c4cc5c6ccc(-c7ccccc7)c(-c7ccccc7)c6oc5c5ccccc45)cc3)c3ccc4ccccc4c3)cc21 ; c1ccc(-c2ccc(N(c3ccc(-c4cc5c6ccc(-c7ccccc7)c(-c7ccccc7)c6oc5c5ccccc45)cc3)c3cccc4c3oc3ccccc34)cc2)cc1 ; CC1(C)c2ccccc2-c2ccc(N(c3ccc(-c4ccccc4)cc3)c3cccc(-c4cc5c6ccc(-c7ccccc7)c(-c7ccccc7)c6oc5c5ccccc45)c3)cc21]

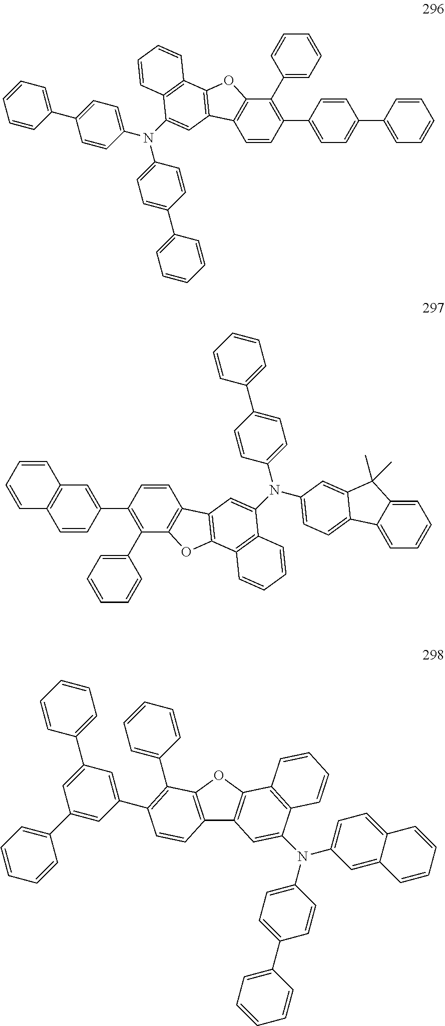 [SMILES: c1ccc(-c2ccc(-c3ccc4c(oc5c6ccccc6c(N(c6ccc(-c7ccccc7)cc6)c6ccc(-c7ccccc7)cc6)cc45)c3-c3ccccc3)cc2)cc1 ; CC1(C)c2ccccc2-c2ccc(N(c3ccc(-c4ccccc4)cc3)c3cc4c5ccc(-c6ccc7ccccc7c6)c(-c6ccccc6)c5oc4c4ccccc34)cc21 ; c1ccc(-c2ccc(N(c3ccc4ccccc4c3)c3cc4c5ccc(-c6cc(-c7ccccc7)cc(-c7ccccc7)c6)c(-c6ccccc6)c5oc4c4ccccc34)cc2)cc1]

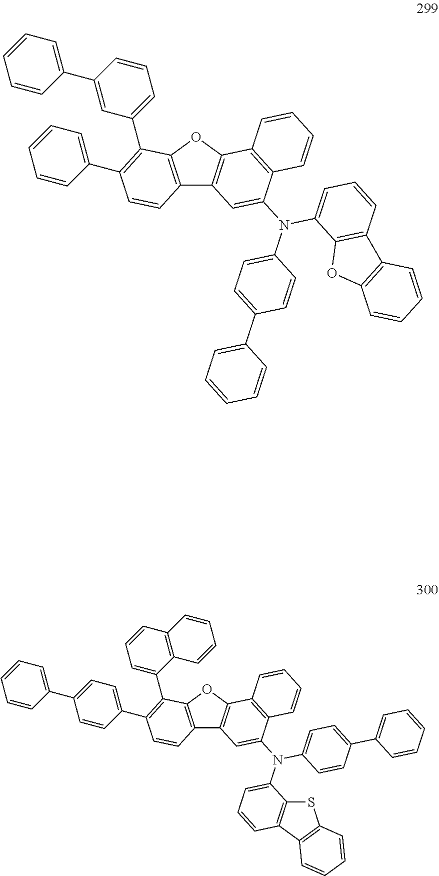 [SMILES: c1ccc(-c2ccc(N(c3cc4c5ccc(-c6ccccc6)c(-c6cccc(-c7ccccc7)c6)c5oc4c4ccccc34)c3cccc4c3oc3ccccc34)cc2)cc1 ; c1ccc(-c2ccc(-c3ccc4c(oc5c6ccccc6c(N(c6ccc(-c7ccccc7)cc6)c6cccc7c6sc6ccccc67)cc45)c3-c3cccc4ccccc34)cc2)cc1]

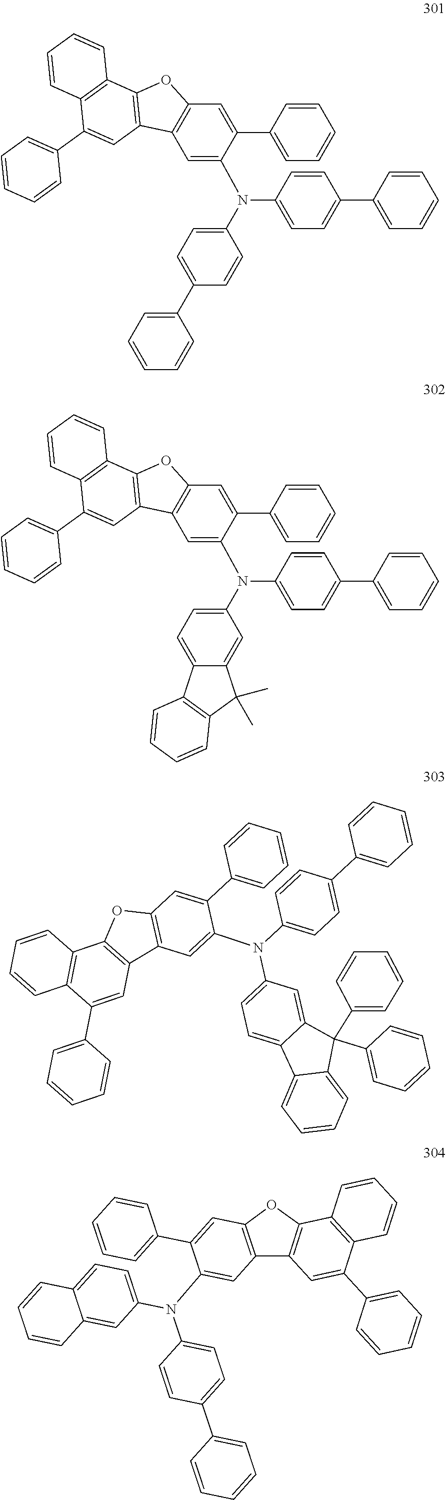 [SMILES: c1ccc(-c2ccc(N(c3ccc(-c4ccccc4)cc3)c3cc4c(cc3-c3ccccc3)oc3c5ccccc5c(-c5ccccc5)cc43)cc2)cc1 ; CC1(C)c2ccccc2-c2ccc(N(c3ccc(-c4ccccc4)cc3)c3cc4c(cc3-c3ccccc3)oc3c5ccccc5c(-c5ccccc5)cc43)cc21 ; c1ccc(-c2ccc(N(c3ccc4c(c3)C(c3ccccc3)(c3ccccc3)c3ccccc3-4)c3cc4c(cc3-c3ccccc3)oc3c5ccccc5c(-c5ccccc5)cc43)cc2)cc1 ; c1ccc(-c2ccc(N(c3ccc4ccccc4c3)c3cc4c(cc3-c3ccccc3)oc3c5ccccc5c(-c5ccccc5)cc43)cc2)cc1]

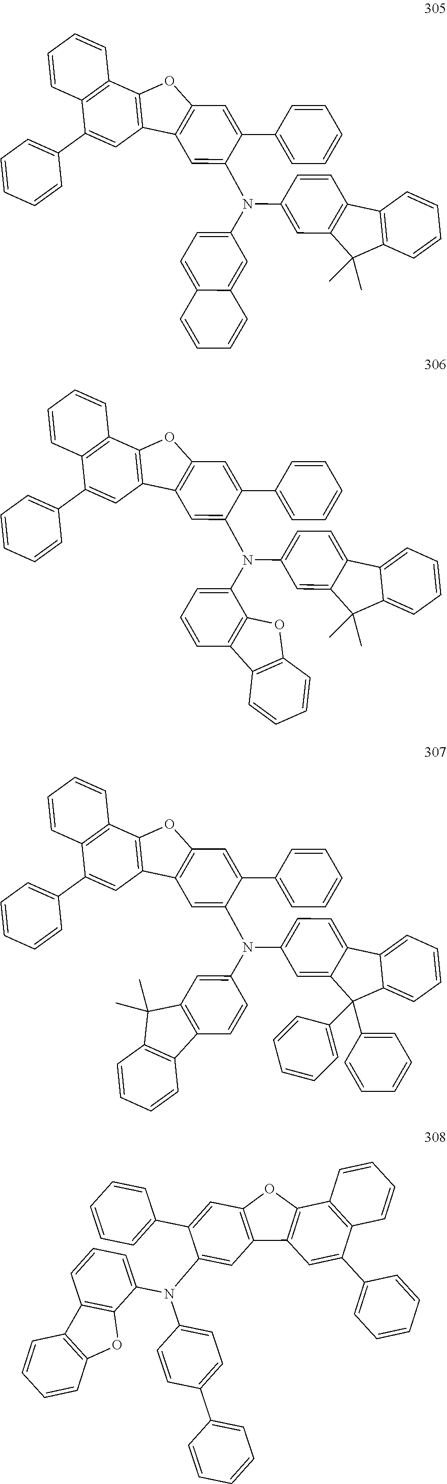 [SMILES: CC1(C)c2ccccc2-c2ccc(N(c3ccc4ccccc4c3)c3cc4c(cc3-c3ccccc3)oc3c5ccccc5c(-c5ccccc5)cc43)cc21 ; CC1(C)c2ccccc2-c2ccc(N(c3cc4c(cc3-c3ccccc3)oc3c5ccccc5c(-c5ccccc5)cc43)c3cccc4c3oc3ccccc34)cc21 ; CC1(C)c2ccccc2-c2ccc(N(c3ccc4c(c3)C(c3ccccc3)(c3ccccc3)c3ccccc3-4)c3cc4c(cc3-c3ccccc3)oc3c5ccccc5c(-c5ccccc5)cc43)cc21 ; c1ccc(-c2ccc(N(c3cc4c(cc3-c3ccccc3)oc3c5ccccc5c(-c5ccccc5)cc43)c3cccc4c3oc3ccccc34)cc2)cc1]

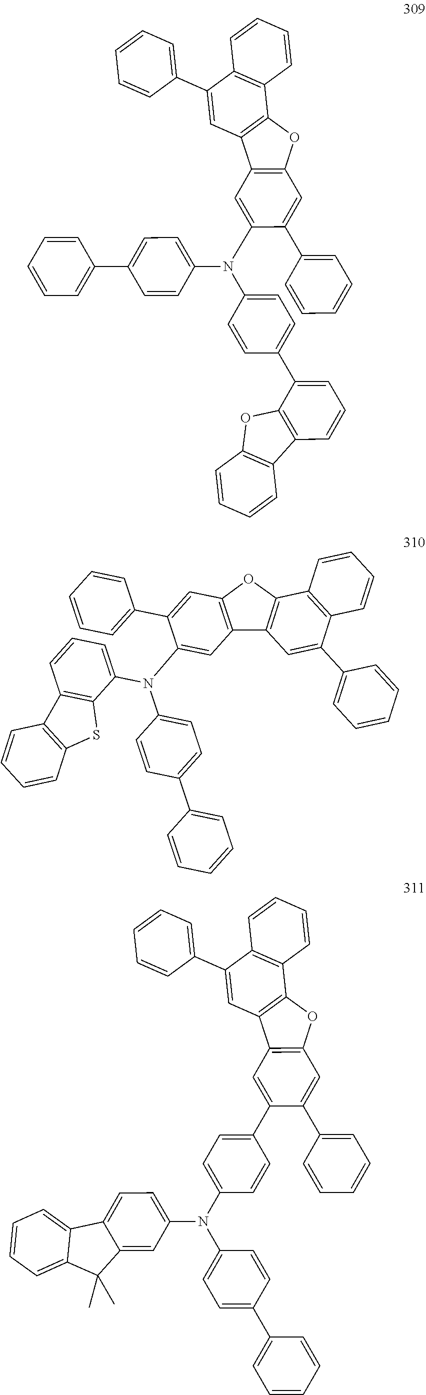 [SMILES: c1ccc(-c2ccc(N(c3ccc(-c4cccc5c4oc4ccccc45)cc3)c3cc4c(cc3-c3ccccc3)oc3c5ccccc5c(-c5ccccc5)cc43)cc2)cc1 ; c1ccc(-c2ccc(N(c3cc4c(cc3-c3ccccc3)oc3c5ccccc5c(-c5ccccc5)cc43)c3cccc4c3sc3ccccc34)cc2)cc1 ; CC1(C)c2ccccc2-c2ccc(N(c3ccc(-c4ccccc4)cc3)c3ccc(-c4cc5c(cc4-c4ccccc4)oc4c6ccccc6c(-c6ccccc6)cc54)cc3)cc21]

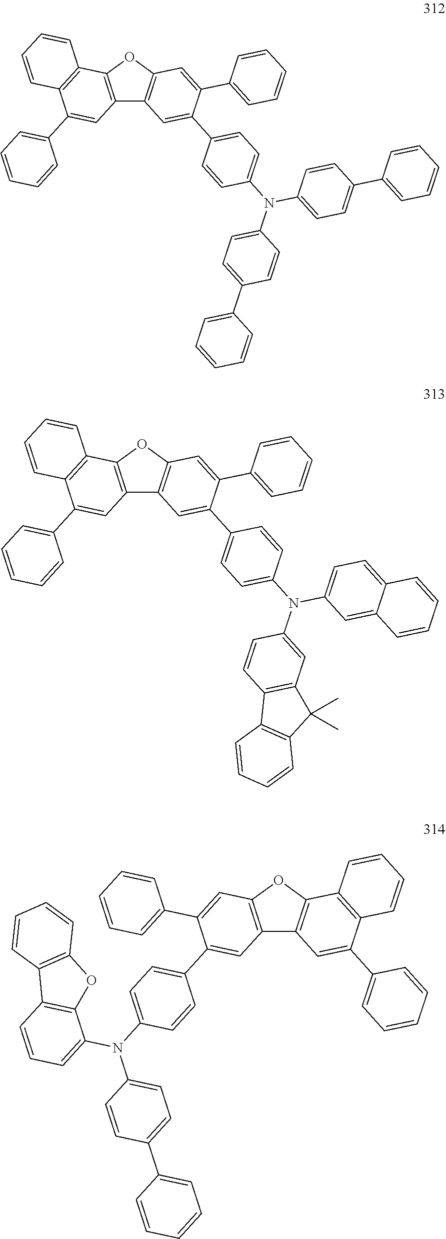 [SMILES: c1ccc(-c2ccc(N(c3ccc(-c4ccccc4)cc3)c3ccc(-c4cc5c(cc4-c4ccccc4)oc4c6ccccc6c(-c6ccccc6)cc54)cc3)cc2)cc1 ; CC1(C)c2ccccc2-c2ccc(N(c3ccc(-c4cc5c(cc4-c4ccccc4)oc4c6ccccc6c(-c6ccccc6)cc54)cc3)c3ccc4ccccc4c3)cc21 ; c1ccc(-c2ccc(N(c3ccc(-c4cc5c(cc4-c4ccccc4)oc4c6ccccc6c(-c6ccccc6)cc54)cc3)c3cccc4c3oc3ccccc34)cc2)cc1]

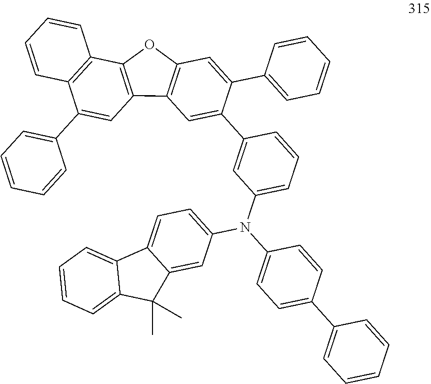 [SMILES: CC1(C)c2ccccc2-c2ccc(N(c3ccc(-c4ccccc4)cc3)c3cccc(-c4cc5c(cc4-c4ccccc4)oc4c6ccccc6c(-c6ccccc6)cc54)c3)cc21]

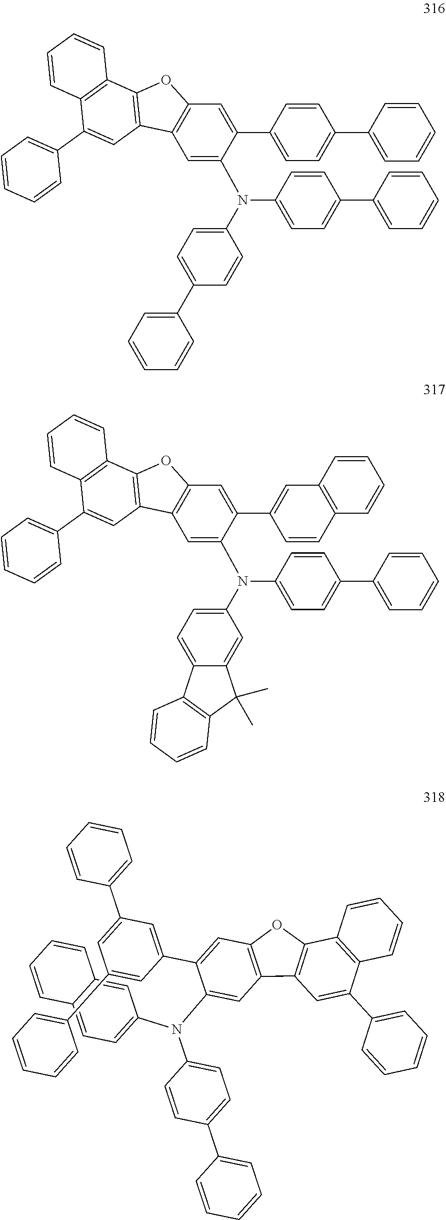 [SMILES: c1ccc(-c2ccc(-c3cc4oc5c6ccccc6c(-c6ccccc6)cc5c4cc3N(c3ccc(-c4ccccc4)cc3)c3ccc(-c4ccccc4)cc3)cc2)cc1 ; CC1(C)c2ccccc2-c2ccc(N(c3ccc(-c4ccccc4)cc3)c3cc4c(cc3-c3ccc5ccccc5c3)oc3c5ccccc5c(-c5ccccc5)cc43)cc21 ; c1ccc(-c2ccc(N(c3ccc4ccccc4c3)c3cc4c(cc3-c3cc(-c5ccccc5)cc(-c5ccccc5)c3)oc3c5ccccc5c(-c5ccccc5)cc43)cc2)cc1]

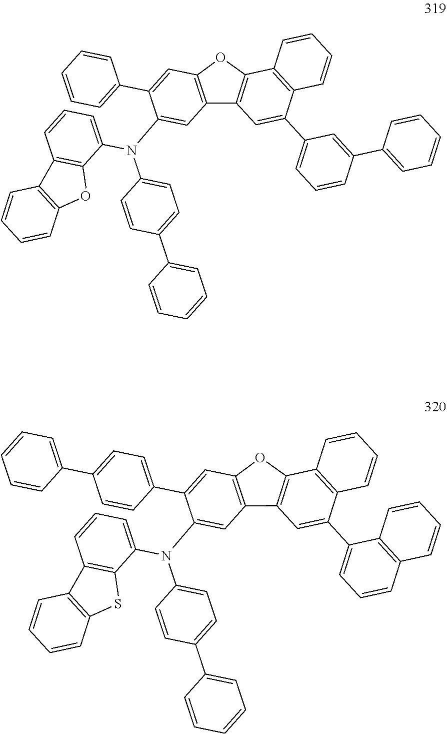 [SMILES: c1ccc(-c2ccc(N(c3cc4c(cc3-c3ccccc3)oc3c5ccccc5c(-c5cccc(-c6ccccc6)c5)cc43)c3cccc4c3oc3ccccc34)cc2)cc1 ; c1ccc(-c2ccc(-c3cc4oc5c6ccccc6c(-c6cccc7ccccc67)cc5c4cc3N(c3ccc(-c4ccccc4)cc3)c3cccc4c3sc3ccccc34)cc2)cc1]

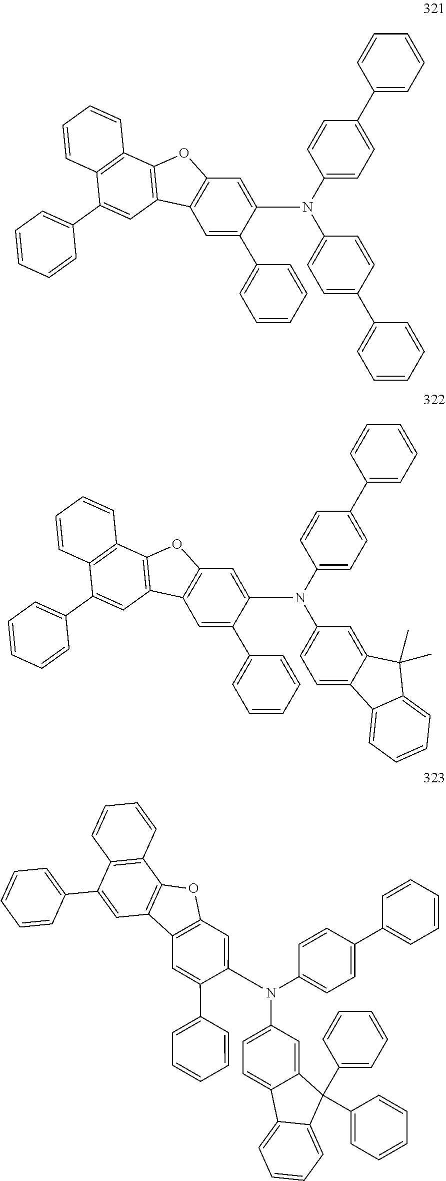

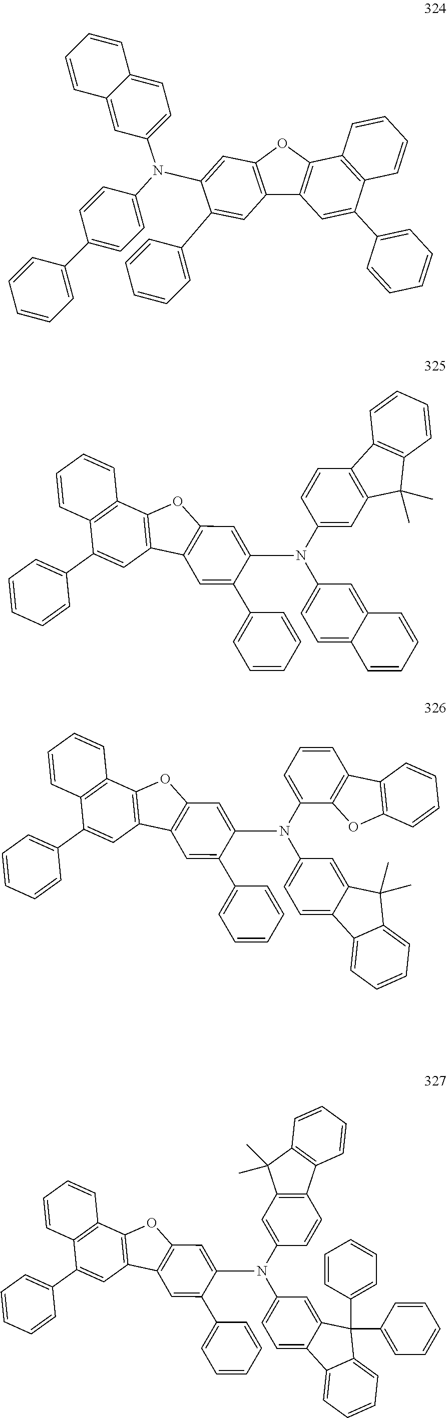 [SMILES: c1ccc(-c2ccc(N(c3ccc4ccccc4c3)c3cc4oc5c6ccccc6c(-c6ccccc6)cc5c4cc3-c3ccccc3)cc2)cc1 ; CC1(C)c2ccccc2-c2ccc(N(c3ccc4ccccc4c3)c3cc4oc5c6ccccc6c(-c6ccccc6)cc5c4cc3-c3ccccc3)cc21 ; CC1(C)c2ccccc2-c2ccc(N(c3cc4oc5c6ccccc6c(-c6ccccc6)cc5c4cc3-c3ccccc3)c3cccc4c3oc3ccccc34)cc21 ; CC1(C)c2ccccc2-c2ccc(N(c3ccc4c(c3)C(c3ccccc3)(c3ccccc3)c3ccccc3-4)c3cc4oc5c6ccccc6c(-c6ccccc6)cc5c4cc3-c3ccccc3)cc21]

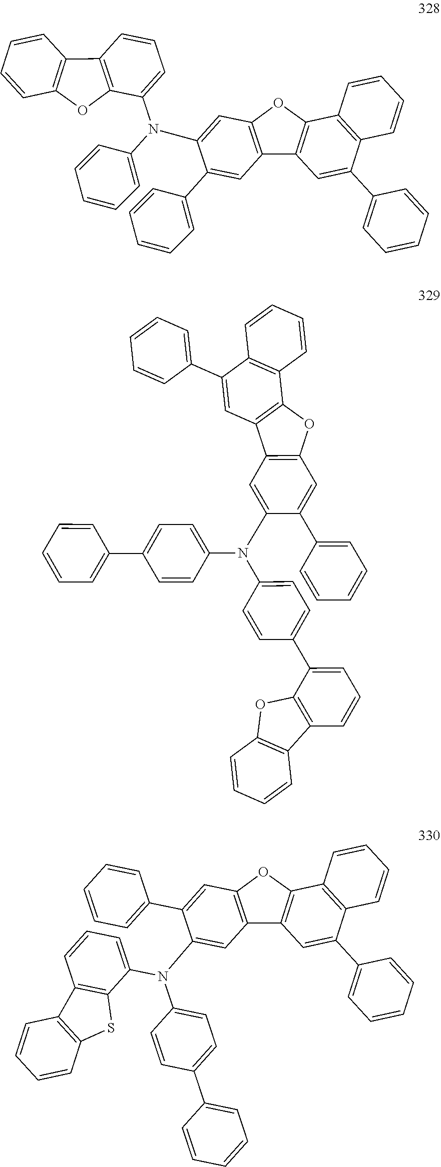 [SMILES: c1ccc(-c2cc3c(cc2N(c2ccccc2)c2cccc4c2oc2ccccc24)oc2c4ccccc4c(-c4ccccc4)cc32)cc1 ; c1ccc(-c2ccc(N(c3ccc(-c4cccc5c4oc4ccccc45)cc3)c3cc4c(cc3-c3ccccc3)oc3c5ccccc5c(-c5ccccc5)cc43)cc2)cc1 ; c1ccc(-c2ccc(N(c3cc4c(cc3-c3ccccc3)oc3c5ccccc5c(-c5ccccc5)cc43)c3cccc4c3sc3ccccc34)cc2)cc1]

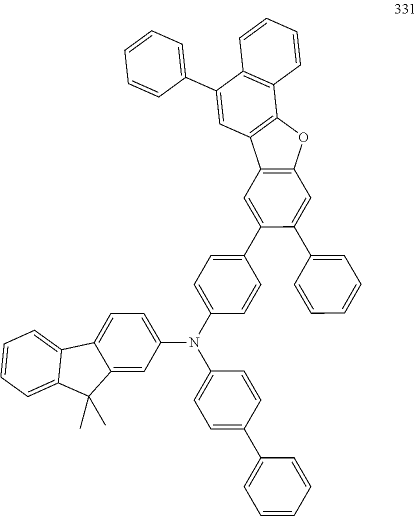 [SMILES: CC1(C)c2ccccc2-c2ccc(N(c3ccc(-c4ccccc4)cc3)c3ccc(-c4cc5c(cc4-c4ccccc4)oc4c6ccccc6c(-c6ccccc6)cc54)cc3)cc21]

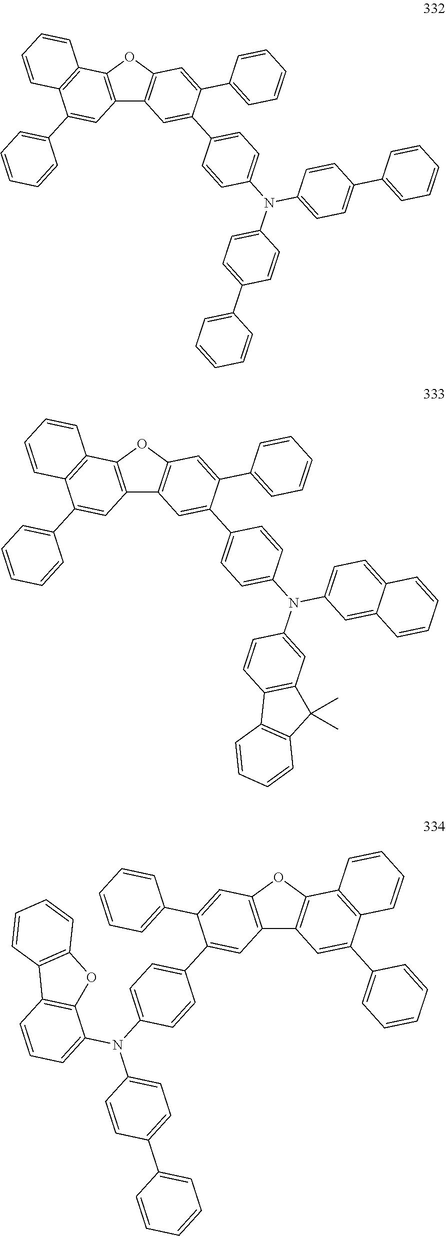 [SMILES: c1ccc(-c2ccc(N(c3ccc(-c4ccccc4)cc3)c3ccc(-c4cc5c(cc4-c4ccccc4)oc4c6ccccc6c(-c6ccccc6)cc54)cc3)cc2)cc1 ; CC1(C)c2ccccc2-c2ccc(N(c3ccc(-c4cc5c(cc4-c4ccccc4)oc4c6ccccc6c(-c6ccccc6)cc54)cc3)c3ccc4ccccc4c3)cc21 ; c1ccc(-c2ccc(N(c3ccc(-c4cc5c(cc4-c4ccccc4)oc4c6ccccc6c(-c6ccccc6)cc54)cc3)c3cccc4c3oc3ccccc34)cc2)cc1]

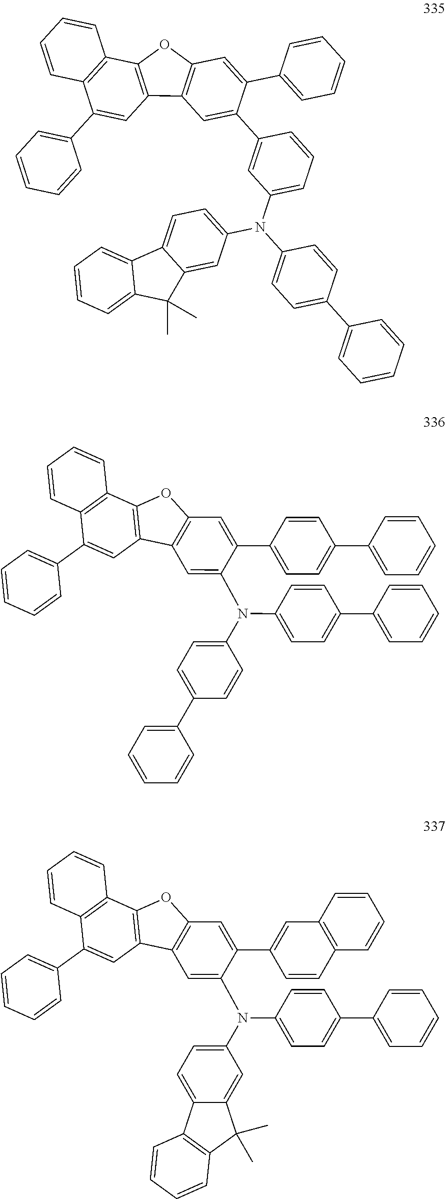 [SMILES: CC1(C)c2ccccc2-c2ccc(N(c3ccc(-c4ccccc4)cc3)c3cccc(-c4cc5c(cc4-c4ccccc4)oc4c6ccccc6c(-c6ccccc6)cc54)c3)cc21 ; c1ccc(-c2ccc(-c3cc4oc5c6ccccc6c(-c6ccccc6)cc5c4cc3N(c3ccc(-c4ccccc4)cc3)c3ccc(-c4ccccc4)cc3)cc2)cc1 ; CC1(C)c2ccccc2-c2ccc(N(c3ccc(-c4ccccc4)cc3)c3cc4c(cc3-c3ccc5ccccc5c3)oc3c5ccccc5c(-c5ccccc5)cc43)cc21]

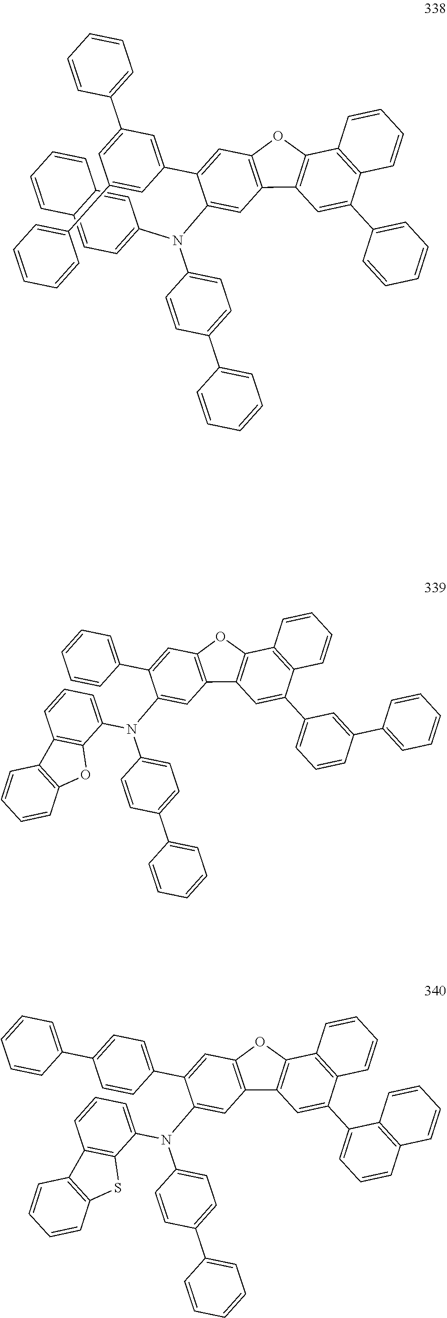 [SMILES: c1ccc(-c2ccc(N(c3ccc4ccccc4c3)c3cc4c(cc3-c3cc(-c5ccccc5)cc(-c5ccccc5)c3)oc3c5ccccc5c(-c5ccccc5)cc43)cc2)cc1 ; c1ccc(-c2ccc(N(c3cc4c(cc3-c3ccccc3)oc3c5ccccc5c(-c5cccc(-c6ccccc6)c5)cc43)c3cccc4c3oc3ccccc34)cc2)cc1 ; c1ccc(-c2ccc(-c3cc4oc5c6ccccc6c(-c6cccc7ccccc67)cc5c4cc3N(c3ccc(-c4ccccc4)cc3)c3cccc4c3sc3ccccc34)cc2)cc1]

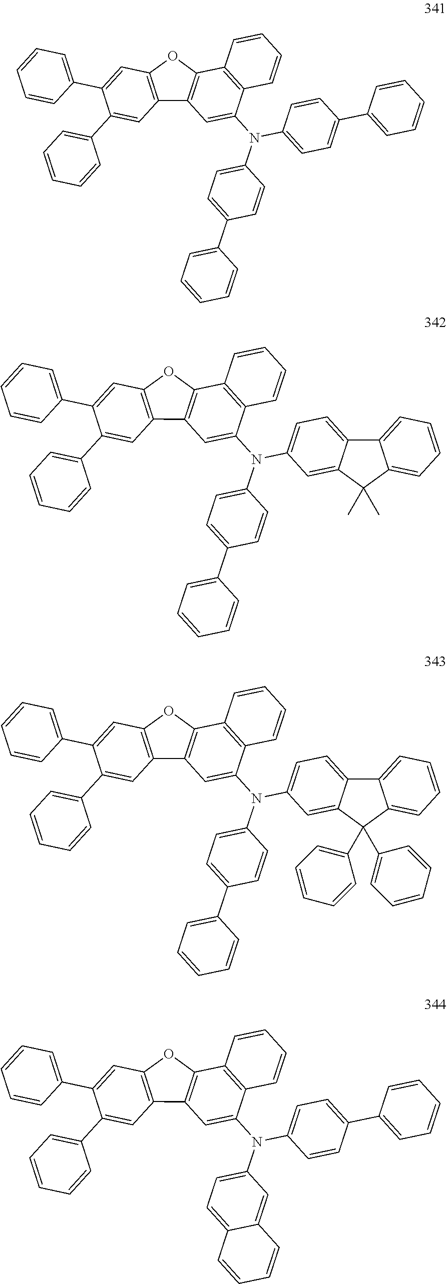 [SMILES: c1ccc(-c2ccc(N(c3ccc(-c4ccccc4)cc3)c3cc4c5cc(-c6ccccc6)c(-c6ccccc6)cc5oc4c4ccccc34)cc2)cc1 ; CC1(C)c2ccccc2-c2ccc(N(c3ccc(-c4ccccc4)cc3)c3cc4c5cc(-c6ccccc6)c(-c6ccccc6)cc5oc4c4ccccc34)cc21 ; c1ccc(-c2ccc(N(c3ccc4c(c3)C(c3ccccc3)(c3ccccc3)c3ccccc3-4)c3cc4c5cc(-c6ccccc6)c(-c6ccccc6)cc5oc4c4ccccc34)cc2)cc1 ; c1ccc(-c2ccc(N(c3ccc4ccccc4c3)c3cc4c5cc(-c6ccccc6)c(-c6ccccc6)cc5oc4c4ccccc34)cc2)cc1]

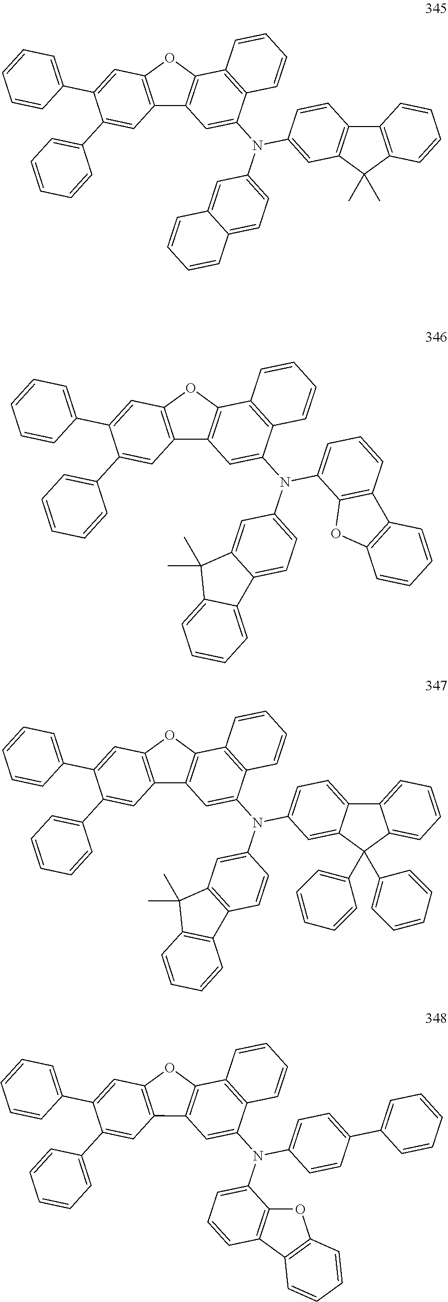 [SMILES: CC1(C)c2ccccc2-c2ccc(N(c3ccc4ccccc4c3)c3cc4c5cc(-c6ccccc6)c(-c6ccccc6)cc5oc4c4ccccc34)cc21 ; CC1(C)c2ccccc2-c2ccc(N(c3cc4c5cc(-c6ccccc6)c(-c6ccccc6)cc5oc4c4ccccc34)c3cccc4c3oc3ccccc34)cc21 ; CC1(C)c2ccccc2-c2ccc(N(c3ccc4c(c3)C(c3ccccc3)(c3ccccc3)c3ccccc3-4)c3cc4c5cc(-c6ccccc6)c(-c6ccccc6)cc5oc4c4ccccc34)cc21 ; c1ccc(-c2ccc(N(c3cc4c5cc(-c6ccccc6)c(-c6ccccc6)cc5oc4c4ccccc34)c3cccc4c3oc3ccccc34)cc2)cc1]

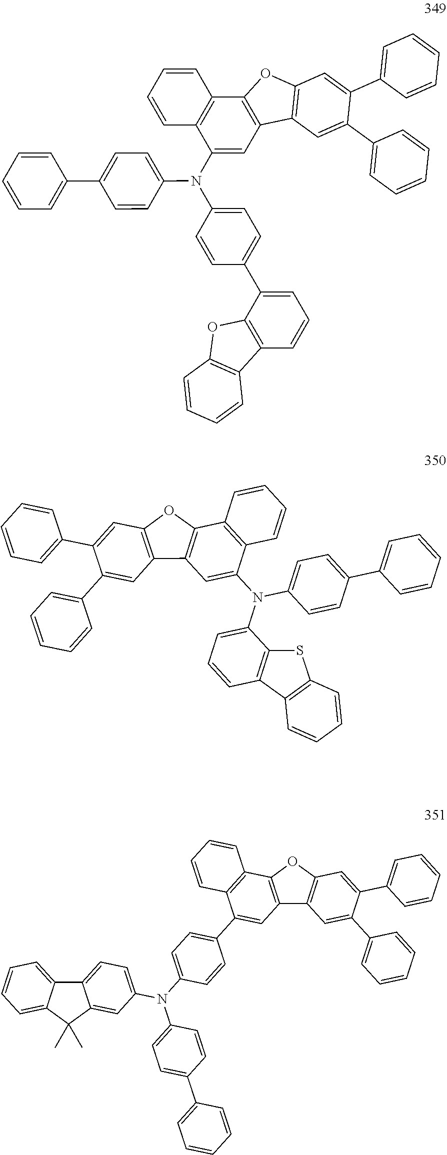 [SMILES: c1ccc(-c2ccc(N(c3ccc(-c4cccc5c4oc4ccccc45)cc3)c3cc4c5cc(-c6ccccc6)c(-c6ccccc6)cc5oc4c4ccccc34)cc2)cc1 ; c1ccc(-c2ccc(N(c3cc4c5cc(-c6ccccc6)c(-c6ccccc6)cc5oc4c4ccccc34)c3cccc4c3sc3ccccc34)cc2)cc1 ; CC1(C)c2ccccc2-c2ccc(N(c3ccc(-c4ccccc4)cc3)c3ccc(-c4cc5c6cc(-c7ccccc7)c(-c7ccccc7)cc6oc5c5ccccc45)cc3)cc21]

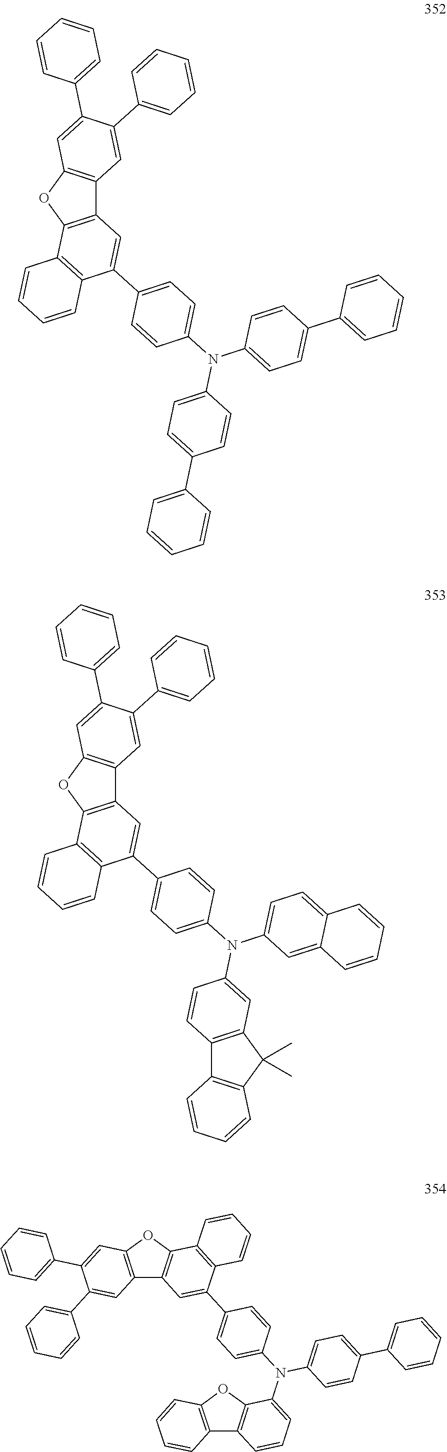 [SMILES: c1ccc(-c2ccc(N(c3ccc(-c4ccccc4)cc3)c3ccc(-c4cc5c6cc(-c7ccccc7)c(-c7ccccc7)cc6oc5c5ccccc45)cc3)cc2)cc1 ; CC1(C)c2ccccc2-c2ccc(N(c3ccc(-c4cc5c6cc(-c7ccccc7)c(-c7ccccc7)cc6oc5c5ccccc45)cc3)c3ccc4ccccc4c3)cc21 ; c1ccc(-c2ccc(N(c3ccc(-c4cc5c6cc(-c7ccccc7)c(-c7ccccc7)cc6oc5c5ccccc45)cc3)c3cccc4c3oc3ccccc34)cc2)cc1]

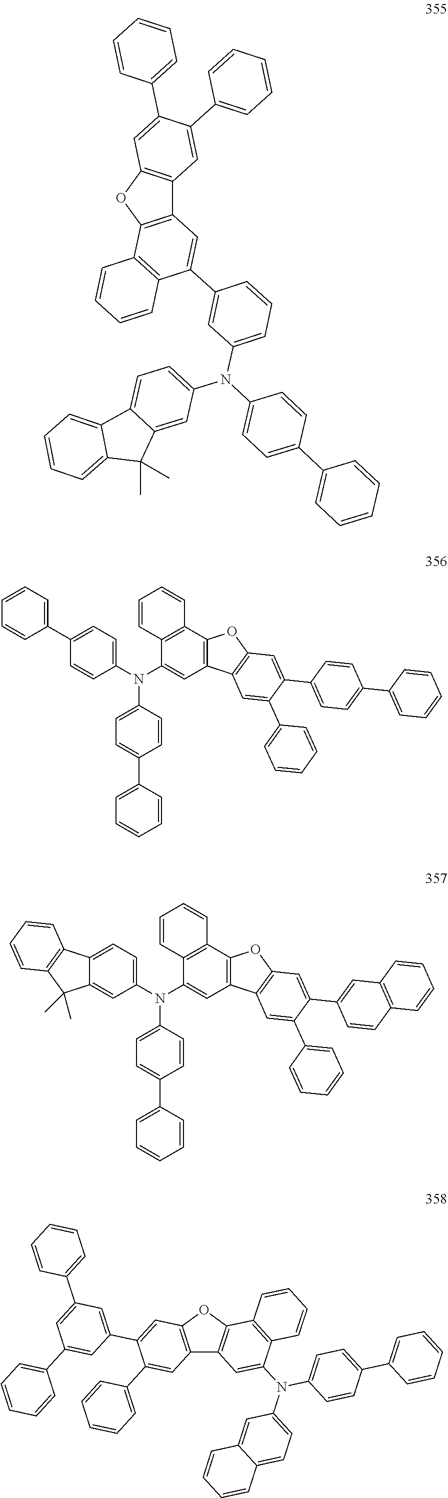 [SMILES: CC1(C)c2ccccc2-c2ccc(N(c3ccc(-c4ccccc4)cc3)c3cccc(-c4cc5c6cc(-c7ccccc7)c(-c7ccccc7)cc6oc5c5ccccc45)c3)cc21 ; c1ccc(-c2ccc(-c3cc4oc5c6ccccc6c(N(c6ccc(-c7ccccc7)cc6)c6ccc(-c7ccccc7)cc6)cc5c4cc3-c3ccccc3)cc2)cc1 ; CC1(C)c2ccccc2-c2ccc(N(c3ccc(-c4ccccc4)cc3)c3cc4c5cc(-c6ccccc6)c(-c6ccc7ccccc7c6)cc5oc4c4ccccc34)cc21 ; c1ccc(-c2ccc(N(c3ccc4ccccc4c3)c3cc4c5cc(-c6ccccc6)c(-c6cc(-c7ccccc7)cc(-c7ccccc7)c6)cc5oc4c4ccccc34)cc2)cc1]

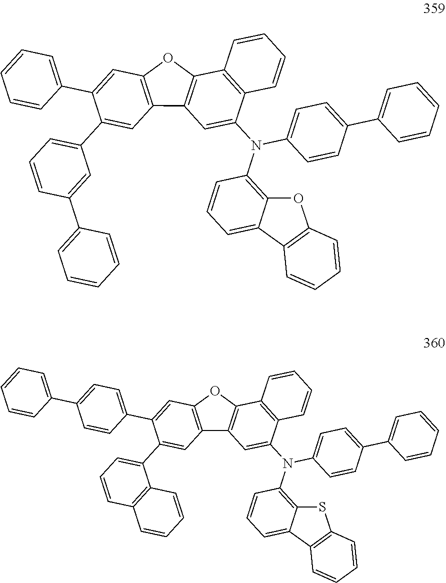 [SMILES: c1ccc(-c2ccc(N(c3cc4c5cc(-c6cccc(-c7ccccc7)c6)c(-c6ccccc6)cc5oc4c4ccccc34)c3cccc4c3oc3ccccc34)cc2)cc1 ; c1ccc(-c2ccc(-c3cc4oc5c6ccccc6c(N(c6ccc(-c7ccccc7)cc6)c6cccc7c6sc6ccccc67)cc5c4cc3-c3cccc4ccccc34)cc2)cc1]

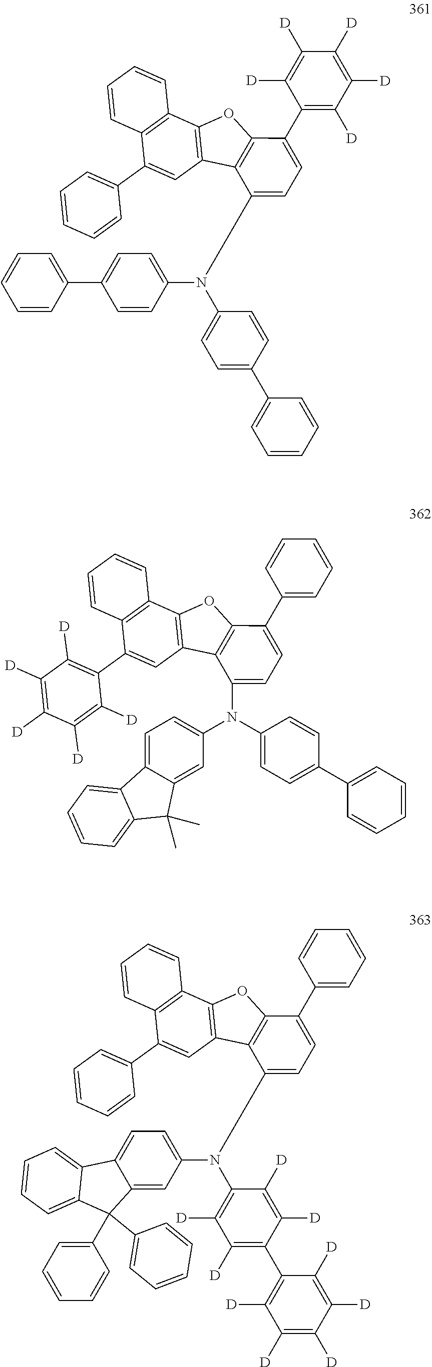 [SMILES: [2H]c1c([2H])c([2H])c(-c2ccc(N(c3ccc(-c4ccccc4)cc3)c3ccc(-c4ccccc4)cc3)c3c2oc2c4ccccc4c(-c4ccccc4)cc23)c([2H])c1[2H] ; [2H]c1c([2H])c([2H])c(-c2cc3c(oc4c(-c5ccccc5)ccc(N(c5ccc(-c6ccccc6)cc5)c5ccc6c(c5)C(C)(C)c5ccccc5-6)c43)c3ccccc23)c([2H])c1[2H] ; [2H]c1c([2H])c([2H])c(-c2c([2H])c([2H])c(N(c3ccc4c(c3)C(c3ccccc3)(c3ccccc3)c3ccccc3-4)c3ccc(-c4ccccc4)c4oc5c6ccccc6c(-c6ccccc6)cc5c34)c([2H])c2[2H])c([2H])c1[2H]]

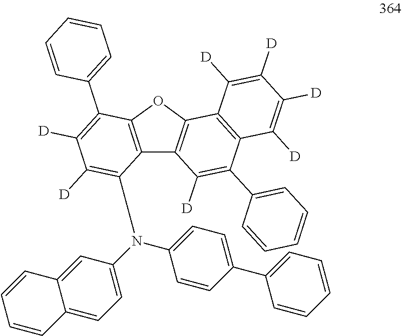 [SMILES: [2H]c1c([2H])c(N(c2ccc(-c3ccccc3)cc2)c2ccc3ccccc3c2)c2c(oc3c4c([2H])c([2H])c([2H])c([2H])c4c(-c4ccccc4)c([2H])c32)c1-c1ccccc1]

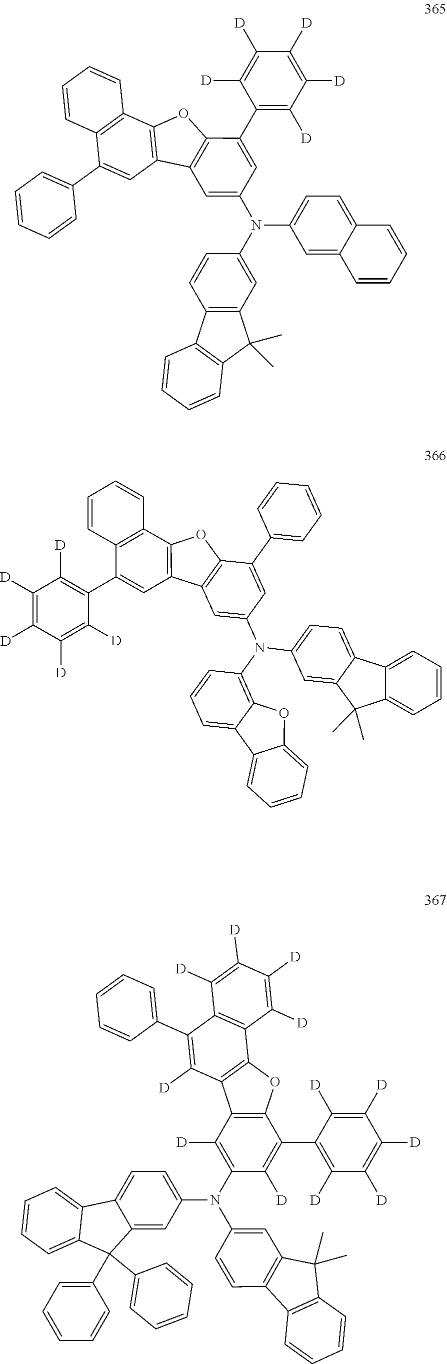 [SMILES: [2H]c1c([2H])c([2H])c(-c2cc(N(c3ccc4c(c3)C(C)(C)c3ccccc3-4)c3ccc4ccccc4c3)cc3c2oc2c4ccccc4c(-c4ccccc4)cc32)c([2H])c1[2H] ; [2H]c1c([2H])c([2H])c(-c2cc3c4cc(N(c5ccc6c(c5)C(C)(C)c5ccccc5-6)c5cccc6c5oc5ccccc56)cc(-c5ccccc5)c4oc3c3ccccc23)c([2H])c1[2H] ; [2H]c1c([2H])c([2H])c(-c2c([2H])c(N(c3ccc4c(c3)C(C)(C)c3ccccc3-4)c3ccc4c(c3)C(c3ccccc3)(c3ccccc3)c3ccccc3-4)c([2H])c3c2oc2c4c([2H])c([2H])c([2H])c([2H])c4c(-c4ccccc4)c([2H])c23)c([2H])c1[2H]]

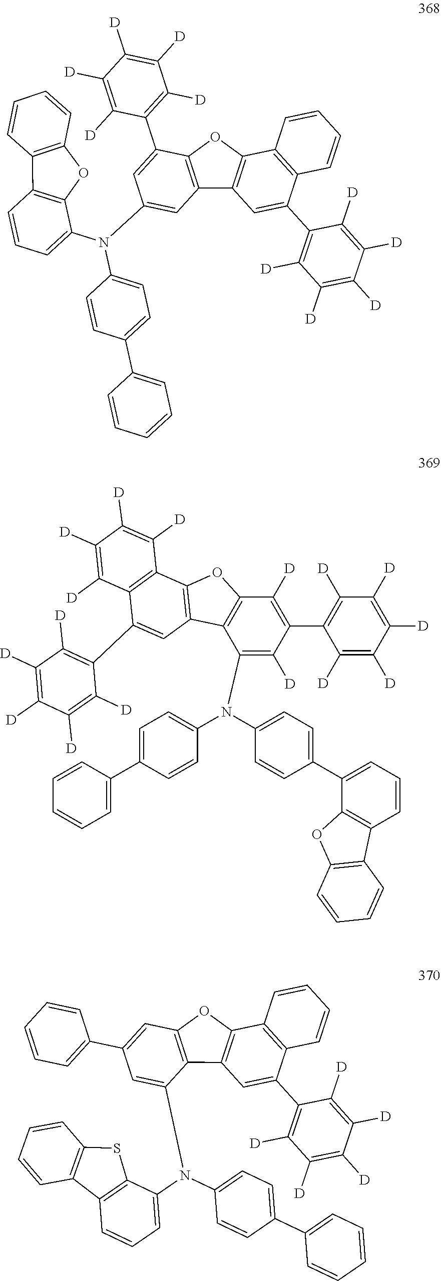 [SMILES: [2H]c1c([2H])c([2H])c(-c2cc3c4cc(N(c5ccc(-c6ccccc6)cc5)c5cccc6c5oc5ccccc56)cc(-c5c([2H])c([2H])c([2H])c([2H])c5[2H])c4oc3c3ccccc23)c([2H])c1[2H] ; [2H]c1c([2H])c([2H])c(-c2c([2H])c(N(c3ccc(-c4ccccc4)cc3)c3ccc(-c4cccc5c4oc4ccccc45)cc3)c3c(oc4c3cc(-c3c([2H])c([2H])c([2H])c([2H])c3[2H])c3c([2H])c([2H])c([2H])c([2H])c34)c2[2H])c([2H])c1[2H] ; [2H]c1c([2H])c([2H])c(-c2cc3c(oc4cc(-c5ccccc5)cc(N(c5ccc(-c6ccccc6)cc5)c5cccc6c5sc5ccccc56)c43)c3ccccc23)c([2H])c1[2H]]

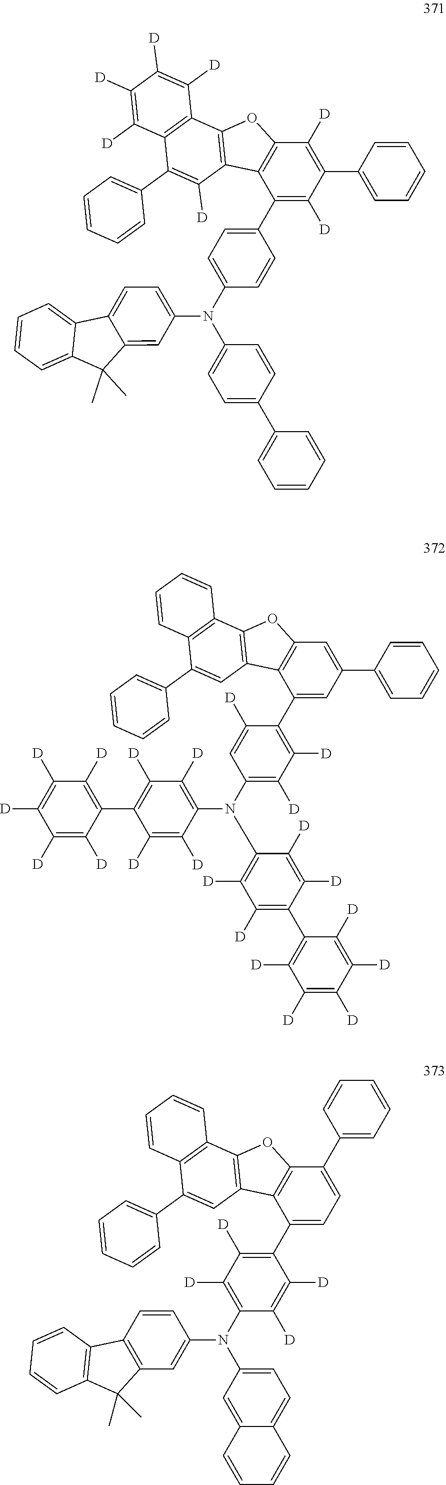 [SMILES: [2H]c1c([2H])c([2H])c2c(c1[2H])c(-c1ccccc1)c([2H])c1c2oc2c([2H])c(-c3ccccc3)c([2H])c(-c3ccc(N(c4ccc(-c5ccccc5)cc4)c4ccc5c(c4)C(C)(C)c4ccccc4-5)cc3)c21 ; [2H]c1cc(N(c2c([2H])c([2H])c(-c3c([2H])c([2H])c([2H])c([2H])c3[2H])c([2H])c2[2H])c2c([2H])c([2H])c(-c3c([2H])c([2H])c([2H])c([2H])c3[2H])c([2H])c2[2H])c([2H])c([2H])c1-c1cc(-c2ccccc2)cc2oc3c4ccccc4c(-c4ccccc4)cc3c12 ; [2H]c1c([2H])c(N(c2ccc3c(c2)C(C)(C)c2ccccc2-3)c2ccc3ccccc3c2)c([2H])c([2H])c1-c1ccc(-c2ccccc2)c2oc3c4ccccc4c(-c4ccccc4)cc3c12]

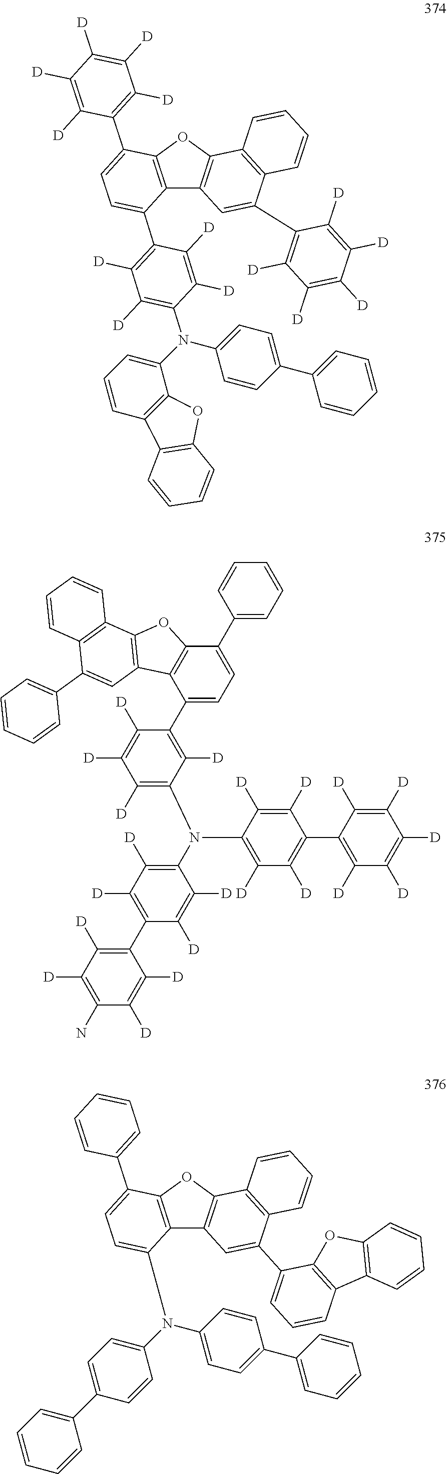

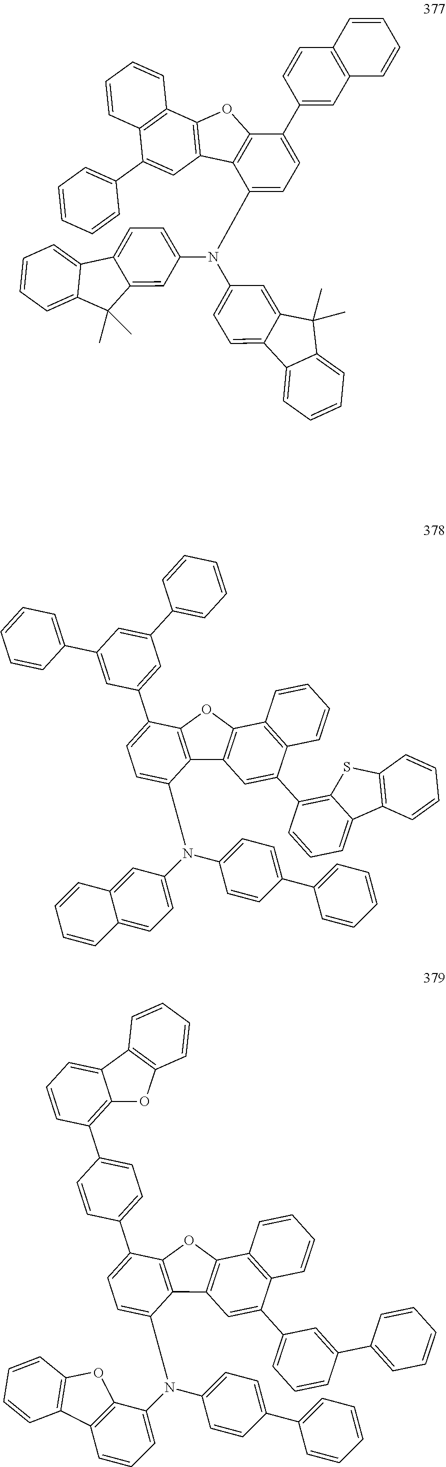 [SMILES: CC1(C)c2ccccc2-c2ccc(N(c3ccc4c(c3)C(C)(C)c3ccccc3-4)c3ccc(-c4ccc5ccccc5c4)c4oc5c6ccccc6c(-c6ccccc6)cc5c34)cc21 ; c1ccc(-c2ccc(N(c3ccc4ccccc4c3)c3ccc(-c4cc(-c5ccccc5)cc(-c5ccccc5)c4)c4oc5c6ccccc6c(-c6cccc7c6sc6ccccc67)cc5c34)cc2)cc1 ; c1ccc(-c2ccc(N(c3cccc4c3oc3ccccc34)c3ccc(-c4ccc(-c5cccc6c5oc5ccccc56)cc4)c4oc5c6ccccc6c(-c6cccc(-c7ccccc7)c6)cc5c34)cc2)cc1]

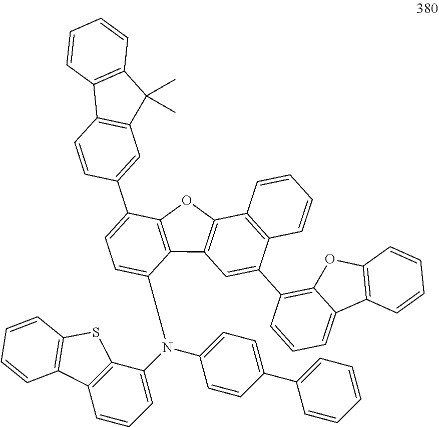 [SMILES: CC1(C)c2ccccc2-c2ccc(-c3ccc(N(c4ccc(-c5ccccc5)cc4)c4cccc5c4sc4ccccc45)c4c3oc3c5ccccc5c(-c5cccc6c5oc5ccccc56)cc34)cc21]

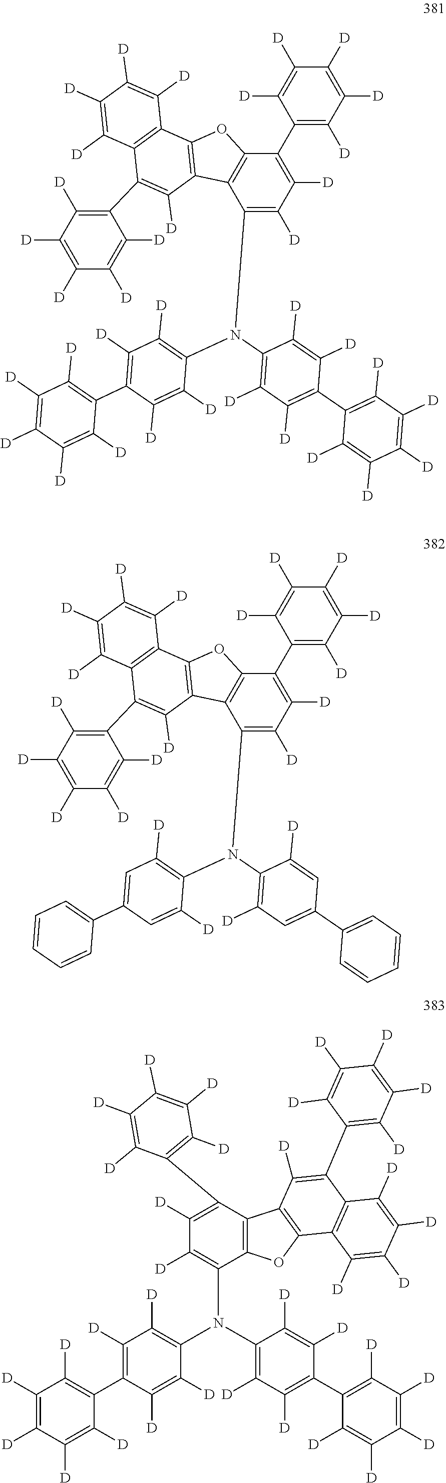

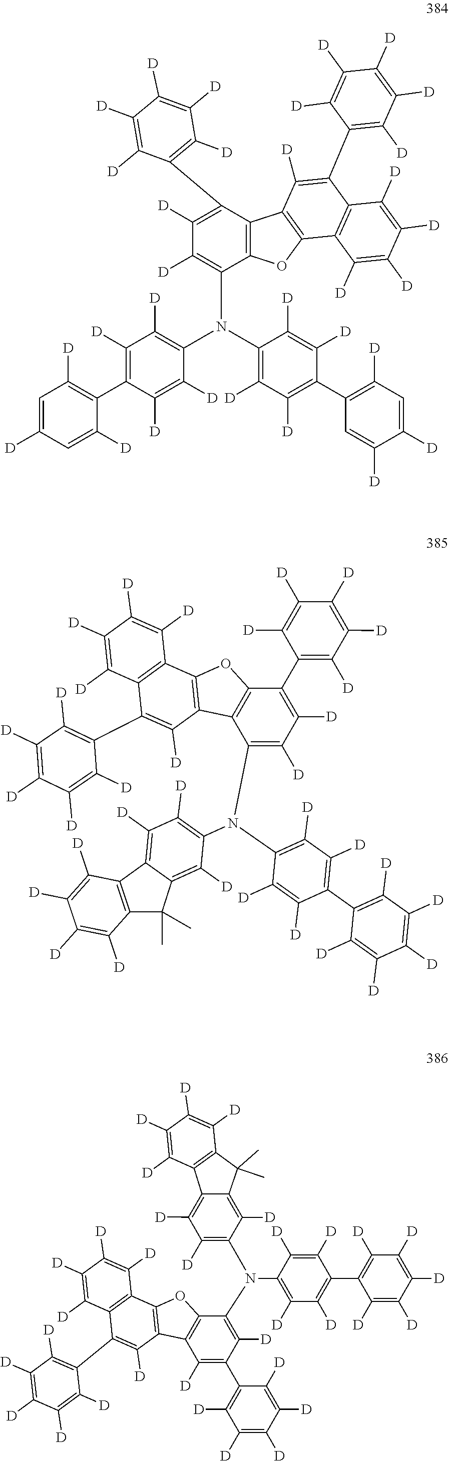

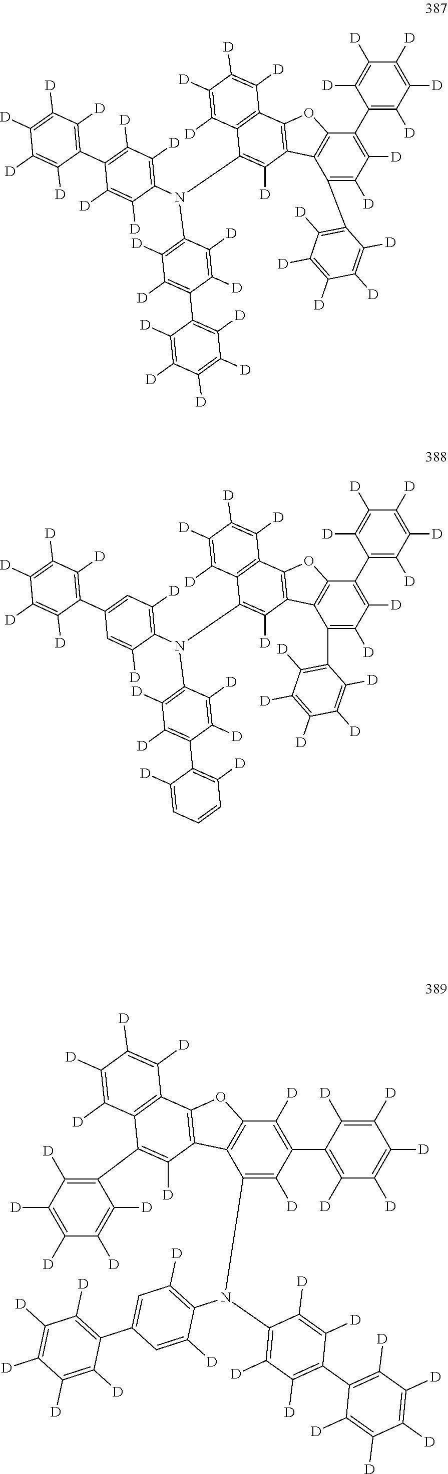

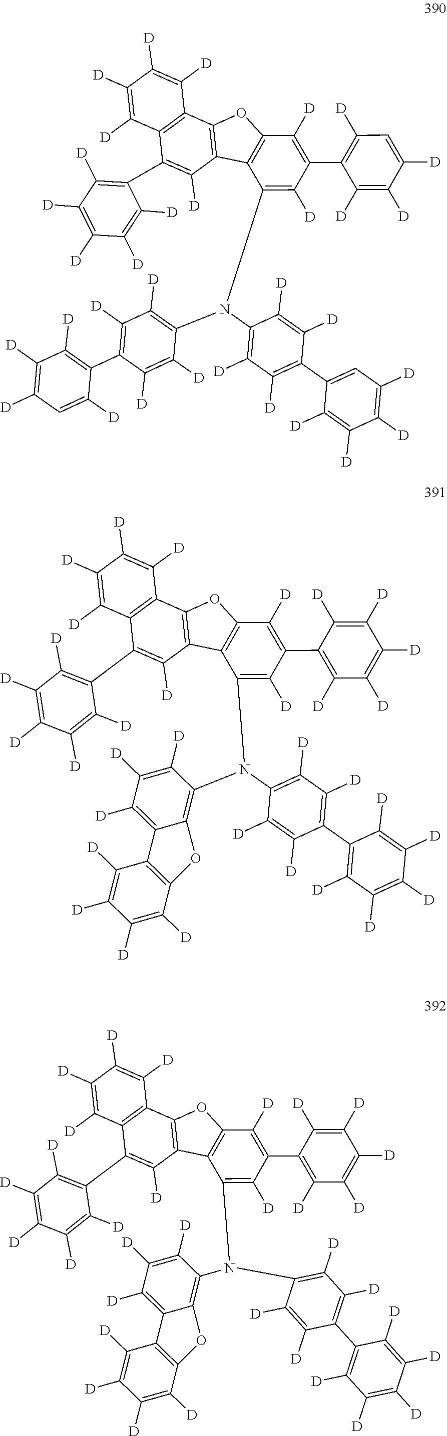

Further, various substituents may be introduced into the structure of Chemical Formula 1 to synthesize a compound having inherent characteristics of a substituent introduced. For example, it is possible to synthesize a material which satisfies conditions required for each organic material layer by introducing a substituent usually used for a hole injection layer material, a material for transporting holes, a light emitting layer material, an electron transport layer material, and a charge generation layer material, which are used for preparing an organic light emitting device, into the core structure.

In addition, it is possible to finely adjust an energy band gap by introducing various substituents into the structure of Chemical Formula 1, and meanwhile, it is possible to improve characteristics at the interface between organic materials and diversify the use of the material.

Meanwhile, the heterocyclic compound has a high glass transition temperature (Tg) and thus has excellent thermal stability. The increase in thermal stability becomes an important factor for providing a device with driving stability.

The heterocyclic compound according to an exemplary embodiment of the present application may be prepared by a multi-step chemical reaction. Some intermediate compounds are first prepared, and the compound of Chemical Formula 1 may be prepared from the intermediate compounds. More specifically, the heterocyclic compound according to an exemplary embodiment of the present application may be prepared based on the Preparation Examples to be described below.

Another exemplary embodiment of the present application provides an organic light emitting device including the heterocyclic compound represented by Chemical Formula 1. The ‘organic light emitting device” may be expressed by terms such as “organic light emitting diode”, “organic light emitting diodes (OLEDs)”, “OLED device”, and “organic electroluminescence device”.

The heterocyclic compound may be formed as an organic material layer by not only a vacuum deposition method, but also a solution application method when an organic light emitting device is manufactured. Here, the solution application method means spin coating, dip coating, inkjet printing, screen printing, a spray method, roll coating, and the like, but is not limited thereto.

Specifically, the organic light emitting device according to an exemplary embodiment of the present application includes a first electrode, a second electrode and an organic material layer having one or more layers provided between the first electrode and the second electrode, in which one or more layers of the organic material layer include the heterocyclic compound represented by Chemical Formula 1. When the heterocyclic compound represented by Chemical Formula 1 is included in the organic material layer, the light emitting efficiency and life time of the organic light emitting device are excellent.

In an exemplary embodiment of the present application, the first electrode may be a positive electrode, and the second electrode may be a negative electrode.

In another exemplary embodiment, the first electrode may be a negative electrode, and the second electrode may be a positive electrode.

In an exemplary embodiment of the present application, the organic light emitting device may be a green organic light emitting device, and the heterocyclic compound according to Chemical Formula 1 may be used as a material for the green organic light emitting device.

In an exemplary embodiment of the present application, the organic light emitting device may be a red organic light emitting device, and the heterocyclic compound according to Chemical Formula 1 may be used as a material for the red organic light emitting device.

In an exemplary embodiment of the present application, the organic light emitting device may be a blue organic light emitting device, and the heterocyclic compound according to Chemical Formula 1 may be used as a material for the blue organic light emitting device.

Further, the organic material layer includes a hole transport layer, and the hole transport layer includes the heterocyclic compound represented by Chemical Formula 1. When the heterocyclic compound represented by Chemical Formula 1 is included in the hole transport layer among the organic material layers, the light emitting efficiency and life time of the organic light emitting device are better.

Further, the organic material layer includes an electron blocking layer, and the electron blocking layer includes the heterocyclic compound represented by Chemical Formula 1. When the heterocyclic compound represented by Chemical Formula 1 is included in the electron blocking layer among the organic material layers, the light emitting efficiency and life time of the organic light emitting device are better.

The organic light emitting device of the present invention may further include one or two or more layers selected from the group consisting of a light emitting layer, a hole injection layer, a hole transport layer, an electron injection layer, an electron transport layer, a hole auxiliary layer, and a hole blocking layer.

The organic light emitting device according to an exemplary embodiment of the present application may be manufactured by typical manufacturing methods and materials of the organic light emitting device, except that the above-described heterocyclic compound is used to form an organic material layer.

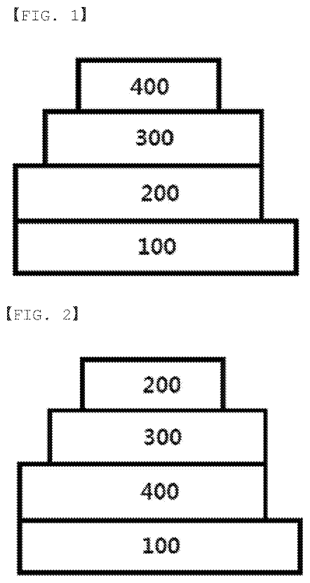

FIGS. 1 to 3 exemplify the stacking sequence of the electrodes and the organic material layer of the organic light emitting device according to an exemplary embodiment of the present application. However, the scope of the present application is not intended to be limited by these drawings, and the structure of the organic light emitting device known in the art may also be applied to the present application.

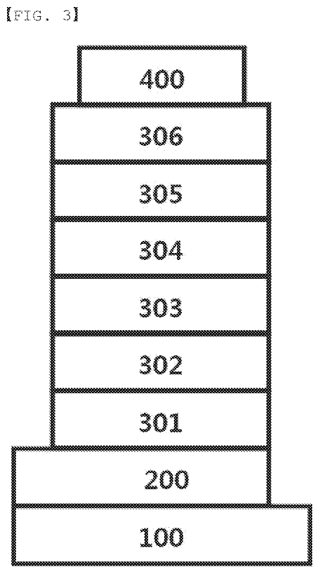

According to FIG. 1, an organic light emitting device in which a positive electrode 200, an organic material layer 300, and a negative electrode 400 are sequentially stacked on a substrate 100 is illustrated. However, the organic light emitting device is not limited only to such a structure, and as in FIG. 2, an organic light emitting device in which a negative electrode, an organic material layer, and a positive electrode are sequentially stacked on a substrate may also be implemented.

FIG. 3 exemplifies a case where an organic material layer is a multilayer. An organic light emitting device according to FIG. 3 includes a hole injection layer 301, a hole transport layer 302, a light emitting layer 303, a hole blocking layer 304, an electron transport layer 305, and an electron injection layer 306. However, the scope of the present application is not limited by the stacking structure as described above, and if necessary, the other layers except for the light emitting layer may be omitted, and another necessary functional layer may be further added.

In the organic light emitting device according to an exemplary embodiment of the present application, materials other than the heterocyclic compound of Chemical Formula 1 will be exemplified below, but these materials are illustrative only and are not for limiting the scope of the present application, and may be replaced with materials publicly known in the art.

As a positive electrode material, materials having a relatively high work function may be used, and a transparent conductive oxide, a metal or a conductive polymer, and the like may be used. Specific examples of the positive electrode material include: a metal such as vanadium, chromium, copper, zinc, and gold, or an alloy thereof; a metal oxide such as zinc oxide, indium oxide, indium tin oxide (ITO), and indium zinc oxide (IZO); a combination of a metal and an oxide, such as ZnO:Al or SnO₂:Sb; a conductive polymer such as poly(3-methylthiophene), poly[3,4-(ethylene-1,2-dioxy)thiophene](PEDOT), polypyrrole, and polyaniline; and the like, but are not limited thereto.

As a negative electrode material, materials having a relatively low work function may be used, and a metal, a metal oxide, or a conductive polymer, and the like may be used. Specific examples of the negative electrode material include: a metal such as magnesium, calcium, sodium, potassium, titanium, indium, yttrium, lithium, gadolinium, aluminum, silver, tin, and lead, or an alloy thereof; a multi-layer structured material, such as LiF/Al or LiO₂/Al; and the like, but are not limited thereto.

As a hole injection material, a publicly-known hole injection material may also be used, and it is possible to use, for example, a phthalocyanine compound such as copper phthalocyanine disclosed in U.S. Pat. No. 4,356,429 or starburst-type amine derivatives described in the document [Advanced Material, 6, p. 677 (1994)], for example, tris(4-carbazoyl-9-ylphenyl)amine (TCTA), 4,4′,4″-tri[phenyl (m-tolyl)amino]triphenylamine (m-MTDATA), 1,3,5-tris[4-(3-methylphenylphenylamino)phenyl]benzene (m-MTDAPB), polyaniline/dodecylbenzenesulfonic acid or poly(3,4-ethylenedioxythiophene)/poly(4-styrenesulfonate), which is a soluble conductive polymer, polyaniline/camphor sulfonic acid or polyaniline/poly(4-styrene-sulfonate), and the like.

As a hole transporting material, a pyrazoline derivative, an arylamine-based derivative, a stilbene derivative, a triphenyldiamine derivative, and the like may be used, and a low-molecular weight or polymer material may also be used.

As an electron transporting material, it is possible to use an oxadiazole derivative, anthraquinodimethane and a derivative thereof, benzoquinone and a derivative thereof, naphthoquinone and a derivative thereof, anthraquinone and a derivative thereof, tetracyanoanthraquinodimethane and a derivative thereof, a fluorenone derivative, diphenyldicyanoethylene and a derivative thereof, a diphenoquinone derivative, a metal complex of 8-hydroxyquinoline and a derivative thereof, and the like, and a low-molecular weight material and a polymer material may also be used.

As an electron injection material, for example, LiF is representatively used in the art, but the present application is not limited thereto.

As a light emitting material, a red, green, or blue light emitting material may be used, and if necessary, two or more light emitting materials may be mixed and used. Further, as the light emitting material, a fluorescent material may also be used, but a phosphorescent material may also be used. As the light emitting material, it is also possible to use alone a material which emits light by combining holes and electrons each injected from a positive electrode and a negative electrode, but materials in which a host material and a dopant material are involved in light emission together may also be used.

The organic light emitting device according to an exemplary embodiment of the present application may be a top emission type, a bottom emission type, or a dual emission type according to the material to be used.

The heterocyclic compound according to an exemplary embodiment of the present application may act even in organic electronic devices including organic solar cells, organic photoconductors, organic transistors, and the like, based on the principle similar to those applied to organic light emitting devices.

Hereinafter, the present specification will be described in more detail through Examples, but these Examples are provided only for exemplifying the present application, and are not intended to limit the scope of the present application.

### <Preparation Example 1> Preparation of Compound 1

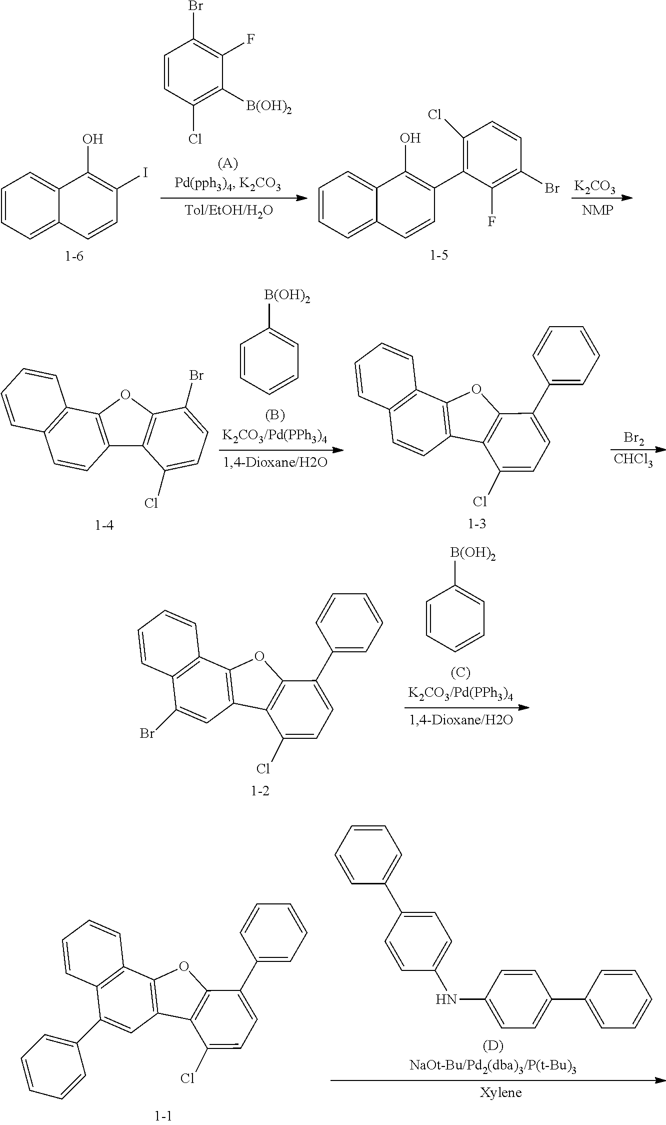

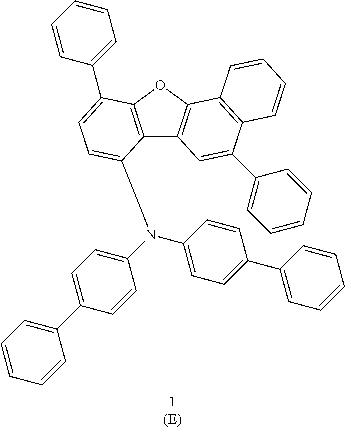 [SMILES: c1ccc(-c2ccc(N(c3ccc(-c4ccccc4)cc3)c3ccc(-c4ccccc4)c4oc5c6ccccc6c(-c6ccccc6)cc5c34)cc2)cc1]

### (1) Preparation of Compound 1-5

After 3-bromo-6-chloro-2-fluoro-phenyl)boronic acid (Intermediate A) (33.76 g, 133.3 mmol), 2-iodonaphthalen-1-ol (30.0 g, 111.08 mmol), Pd(pph₃)₄(6.42 g, 5.55 mmol) and potassium carbonate (46.06 g, 333.25 mmol) were added to toluene (600 mL), ethanol (150 mL) and water (150 mL) and dispersed, the resulting dispersion was stirred under reflux for 6 hours. Thereafter, the temperature was lowered to room temperature, an aqueous layer was separated, and an organic layer was separated by washing the organic layer once more with water. A slurry was produced by introducing anhydrous magnesium sulfate into the collected organic layer, and then filtered and concentrated under reduced pressure. A compound in an oil state was separated by silica chromatography with a combination of hexane and ethyl acetate to prepare Compound 1-5 (30 g, 85.32 mmol, 76% yield).

### (2) Preparation of Compound 1-4

2-(3-Bromo-6-chloro-2-fluoro-phenyl)naphthalen-1-ol (Compound 1-5) (30.0 g, 85.32 mmol) and potassium carbonate (23.59 g, 170.65 mmol) were added to N-methyl-2-pyrrolidone (NMP) (600 mL), and the resulting mixture was stirred at 140° C. for 3 hours. After about 1 hour, the reaction solution was cooled to room temperature and slowly introduced into 500 ml of water. The precipitated solid was filtered, and after the filtered solid was dissolved in tetrahydrofuran (THF), the resulting solution was treated with anhydrous magnesium sulfate, filtered, and then concentrated under reduced pressure. The concentrated compound was slurried with a small amount of tetrahydrofuran and an excessive amount of hexane and filtered. In order to purify the filtered compound, the filtered compound was separated by silica chromatography with hexane and ethyl acetate to prepare Compound 1-4 (28 g, 84.442 mmol, 98.966% yield).

### (3) Preparation of Compound 1-3

10-Bromo-7-chloro-naphtho[1,2-b]benzofuran (Compound 1-4) (28.0 g, 84.44 mmol), phenyl boronic acid (Intermediate B), (11.56 g, 92.89 mmol), Pd(pph₃)₄ (4.88 g, 4.22 mmol) and potassium carbonate (23.59 g, 170.65 mmol) were added to 1,4-dioxane (600 mL) and water (150 mL), and the resulting mixture was reacted under reflux for 5 hours. After the reaction was completed, 100 ml of methylene chloride (MC) and 150 ml of water were added to the reaction solution to complete the reaction (work up), and then the resulting product was put into a separatory funnel to separate the organic layer. The organic layer was dried over anhydrous MgSO₄, the solvent was removed by a rotary evaporator, and then acetone was added thereto to obtain Compound 1-3 (25 g, 76.036 mmol, 90.046% yield) in a slurry state.

### (4) Preparation of Compound 1-2

After 7-chloro-10-phenyl-naphtho[1,2-b]benzofuran (Compound 1-3) (25.0 g, 76.04 mmol) was dissolved in chloroform (250 mL), the resulting solution was stirred in an ice water bath, and then bromine (4.21 mL, 91.24 mmol) was added dropwise thereto using a needle, and then the resulting mixture was stirred at room temperature for 3 hours to obtain Compound 1-2 (30 g, 73.585 mmol, 96.776% yield).

### (5) Preparation of Compound 1-1

5-Bromo-7-chloro-10-phenyl-naphtho[1,2-b]benzofuran (Compound 1-2) (30.0 g, 73.59 mmol), phenyl boronic acid (Intermediate C) (10.07 g, 80.94 mmol), Pd(pph₃)₄ (4.25 g, 3.68 mmol) and potassium carbonate (30.51 g, 220.76 mmol) were added to 1,4-dioxane (300 mL) and water (150 mL), and the resulting mixture was reacted under reflux for 5 hours. After the reaction was completed, 100 ml of methylene chloride (MC) and 150 ml of water were added to the reaction solution to complete the reaction (work up), and then the resulting product was put into a separatory funnel to separate the organic layer. The organic layer was dried over anhydrous MgSO₄, the solvent was removed by a rotary evaporator, and then acetone and hexane were added thereto to obtain Compound 1-1 (25 g, 61.745 mmol, 83.91% yield) in a slurry state.

### (6) Preparation of Compound 1

7-Chloro-5,10-diphenyl-naphtho[1,2-b]benzofuran (Compound 1-1) (25.0 g, 61.75 mmol), bis(4-biphenylyl)amine) (Intermediate D) (21.26 g, 64.83 mmol), Pd₂(dba)₃ (2.83 g, 3.09 mmol), t-Bu₃P (1.47 mL, 6.17 mmol) and NaOt-Bu (17.8 g, 185.24 mmol) were added to xylene (400 mL), and the resulting mixture was stirred under reflux at 125° C. for 5 hours. After the reaction was completed, methylene chloride (MC) was added to the reaction solution and dissolved, then extraction was performed with distilled water, the organic layer was dried over anhydrous magnesium sulfate, the solvent was removed by a rotary evaporator, and then the residue was purified with column chromatography (MC:hexane=1:3) to obtain Compound 1 (30 g, 43.488 mmol, 70.432% yield).

A target compound E in the following Table 1 was synthesized in the same manner as in Preparation Example 1, except that Intermediates A to D in the following Table 1 were used instead of 3-bromo-6-chloro-2-fluoro-phenyl)boronic acid (Intermediate A), phenyl boronic acid (Intermediate B), phenyl boronic acid (Intermediate C) and bis(4-biphenylyl)amine (Intermediate D), respectively, in Preparation Example 1.

TABLE 1

| Com- | | | | | | |
|---|---|---|---|---|---|---|
| pound | Intermediate | Intermediate | Intermediate | Intermediate | Target compound | |
| No. | A | B | C | D | E | yield |
| 1 | 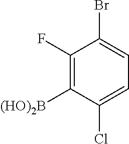 [SMILES: OB(O)c1c(Cl)ccc(Br)c1F] | 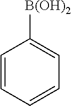 | 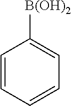 | 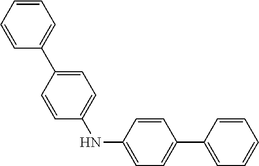 [SMILES: c1ccc(-c2ccc(Nc3ccc(-c4ccccc4)cc3)cc2)cc1] | 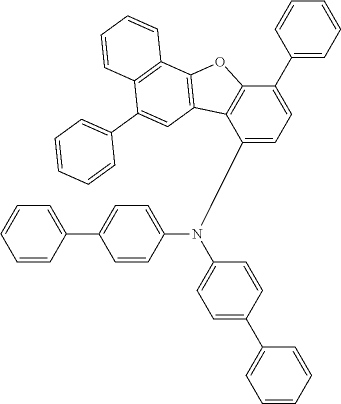 [SMILES: c1ccc(-c2ccc(N(c3ccc(-c4ccccc4)cc3)c3ccc(-c4ccccc4)c4oc5c6ccccc6c(-c6ccccc6)cc5c34)cc2)cc1] | 82% |
| 2 | 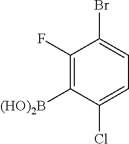 [SMILES: OB(O)c1c(Cl)ccc(Br)c1F] | 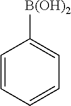 | 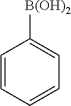 | 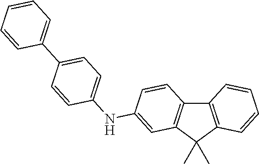 [SMILES: CC1(C)c2ccccc2-c2ccc(Nc3ccc(-c4ccccc4)cc3)cc21] | 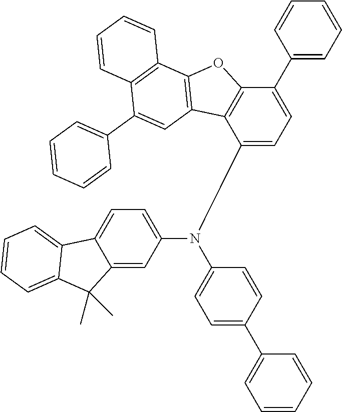 [SMILES: CC1(C)c2ccccc2-c2ccc(N(c3ccc(-c4ccccc4)cc3)c3ccc(-c4ccccc4)c4oc5c6ccccc6c(-c6ccccc6)cc5c34)cc21] | 73% |
| 21 | 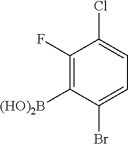 [SMILES: OB(O)c1c(Br)ccc(Cl)c1F] | 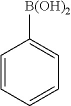 | 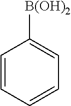 | 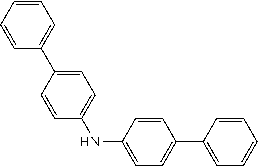 [SMILES: c1ccc(-c2ccc(Nc3ccc(-c4ccccc4)cc3)cc2)cc1] | 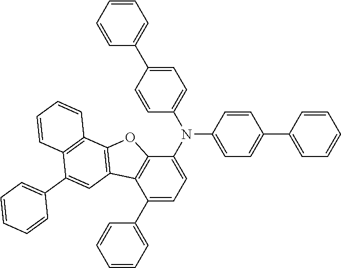 [SMILES: c1ccc(-c2ccc(N(c3ccc(-c4ccccc4)cc3)c3ccc(-c4ccccc4)c4c3oc3c5ccccc5c(-c5ccccc5)cc34)cc2)cc1] | 75% |
| 36 | 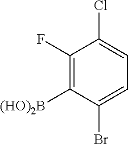 [SMILES: OB(O)c1c(Br)ccc(Cl)c1F] | 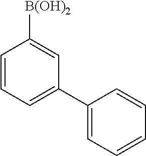 [SMILES: OB(O)c1cccc(-c2ccccc2)c1] | 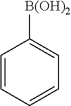 | 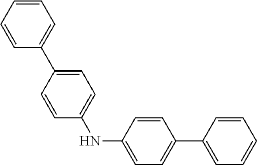 [SMILES: c1ccc(-c2ccc(Nc3ccc(-c4ccccc4)cc3)cc2)cc1] | 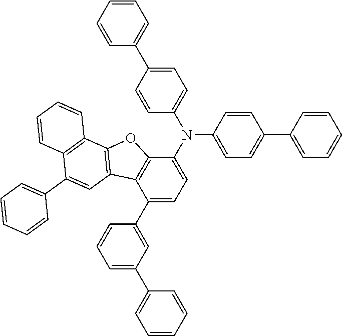 [SMILES: c1ccc(-c2ccc(N(c3ccc(-c4ccccc4)cc3)c3ccc(-c4cccc(-c5ccccc5)c4)c4c3oc3c5ccccc5c(-c5ccccc5)cc34)cc2)cc1] | 62% |
| 78 | 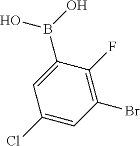 [SMILES: OB(O)c1cc(Cl)cc(Br)c1F] | 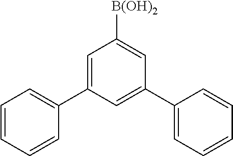 [SMILES: OB(O)c1cc(-c2ccccc2)cc(-c2ccccc2)c1] | 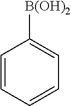 | 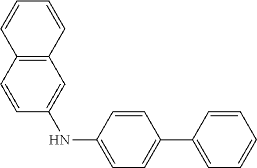 [SMILES: c1ccc(-c2ccc(Nc3ccc4ccccc4c3)cc2)cc1] | 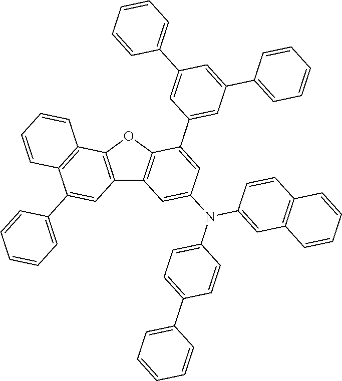 [SMILES: c1ccc(-c2ccc(N(c3ccc4ccccc4c3)c3cc(-c4cc(-c5ccccc5)cc(-c5ccccc5)c4)c4oc5c6ccccc6c(-c6ccccc6)cc5c4c3)cc2)cc1] | 55% |
| 96 | 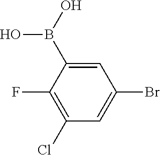 [SMILES: OB(O)c1cc(Br)cc(Cl)c1F] | 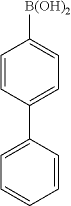 [SMILES: OB(O)c1ccc(-c2ccccc2)cc1] | 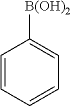 | 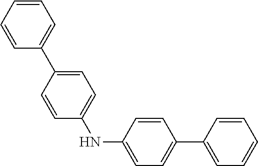 [SMILES: c1ccc(-c2ccc(Nc3ccc(-c4ccccc4)cc3)cc2)cc1] | 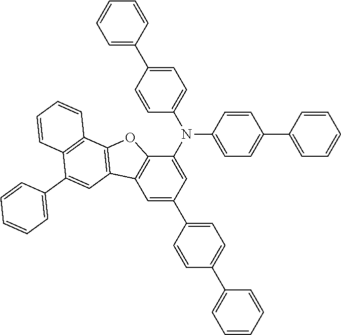 [SMILES: c1ccc(-c2ccc(-c3cc(N(c4ccc(-c5ccccc5)cc4)c4ccc(-c5ccccc5)cc4)c4oc5c6ccccc6c(-c6ccccc6)cc5c4c3)cc2)cc1] | 52% |
| 97 | 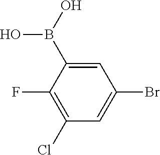 [SMILES: OB(O)c1cc(Br)cc(Cl)c1F] | 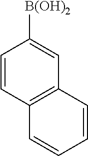 [SMILES: OB(O)c1ccc2ccccc2c1] | 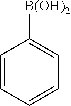 | 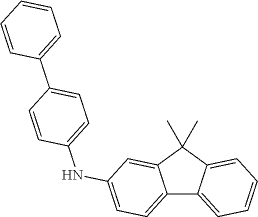 [SMILES: CC1(C)c2ccccc2-c2ccc(Nc3ccc(-c4ccccc4)cc3)cc21] | 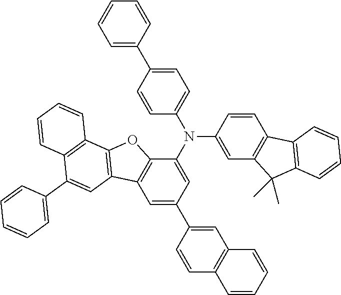 [SMILES: CC1(C)c2ccccc2-c2ccc(N(c3ccc(-c4ccccc4)cc3)c3cc(-c4ccc5ccccc5c4)cc4c3oc3c5ccccc5c(-c5ccccc5)cc43)cc21] | 47% |
| 116 | 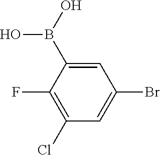 [SMILES: OB(O)c1cc(Br)cc(Cl)c1F] | 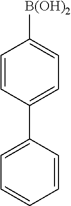 [SMILES: OB(O)c1ccc(-c2ccccc2)cc1] | 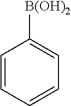 | 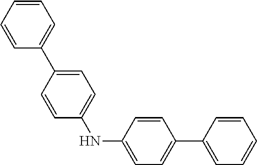 [SMILES: c1ccc(-c2ccc(Nc3ccc(-c4ccccc4)cc3)cc2)cc1] | 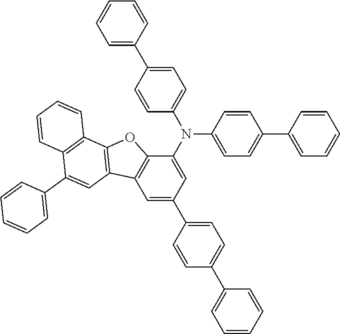 [SMILES: c1ccc(-c2ccc(-c3cc(N(c4ccc(-c5ccccc5)cc4)c4ccc(-c5ccccc5)cc4)c4oc5c6ccccc6c(-c6ccccc6)cc5c4c3)cc2)cc1] | 43% |
| 136 | 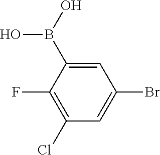 [SMILES: OB(O)c1cc(Br)cc(Cl)c1F] | 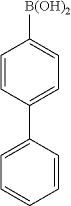 [SMILES: OB(O)c1ccc(-c2ccccc2)cc1] | 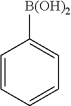 | 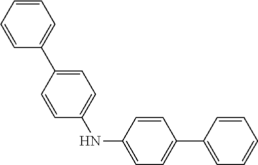 [SMILES: c1ccc(-c2ccc(Nc3ccc(-c4ccccc4)cc3)cc2)cc1] | 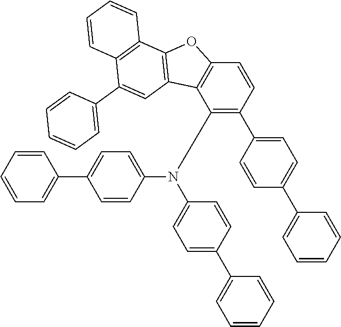 [SMILES: c1ccc(-c2ccc(-c3ccc4oc5c6ccccc6c(-c6ccccc6)cc5c4c3N(c3ccc(-c4ccccc4)cc3)c3ccc(-c4ccccc4)cc3)cc2)cc1] | 48% |
| 175 | 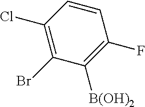 [SMILES: OB(O)c1c(F)ccc(Cl)c1Br] | 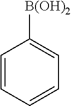 | 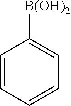 | 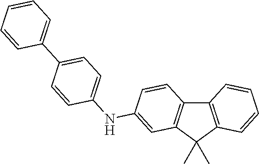 [SMILES: CC1(C)c2ccccc2-c2ccc(Nc3ccc(-c4ccccc4)cc3)cc21] | 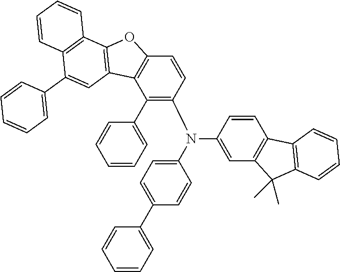 [SMILES: CC1(C)c2ccccc2-c2ccc(N(c3ccc(-c4ccccc4)cc3)c3ccc4oc5c6ccccc6c(-c6ccccc6)cc5c4c3-c3ccccc3)cc21] | 56% |
| 181 | 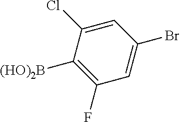 [SMILES: OB(O)c1c(F)cc(Br)cc1Cl] | 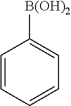 | 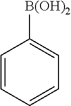 | 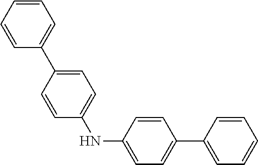 [SMILES: c1ccc(-c2ccc(Nc3ccc(-c4ccccc4)cc3)cc2)cc1] | 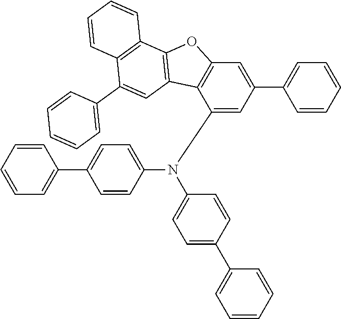 [SMILES: c1ccc(-c2ccc(N(c3ccc(-c4ccccc4)cc3)c3cc(-c4ccccc4)cc4oc5c6ccccc6c(-c6ccccc6)cc5c34)cc2)cc1] | 54% |
| 267 | 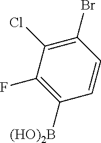 [SMILES: OB(O)c1ccc(Br)c(Cl)c1F] | 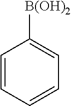 | 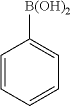 | 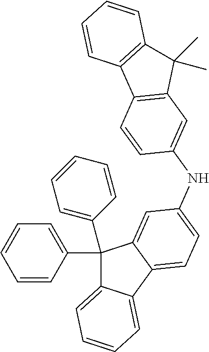 [SMILES: CC1(C)c2ccccc2-c2ccc(Nc3ccc4c(c3)C(c3ccccc3)(c3ccccc3)c3ccccc3-4)cc21] | 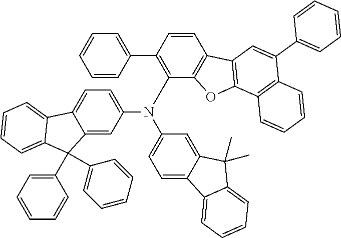 [SMILES: CC1(C)c2ccccc2-c2ccc(N(c3ccc4c(c3)C(c3ccccc3)(c3ccccc3)c3ccccc3-4)c3c(-c4ccccc4)ccc4c3oc3c5ccccc5c(-c5ccccc5)cc43)cc21] | 78% |
| 279 | 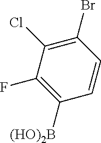 [SMILES: OB(O)c1ccc(Br)c(Cl)c1F] | 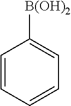 | 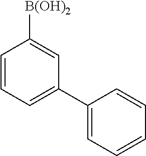 [SMILES: OB(O)c1cccc(-c2ccccc2)c1] | 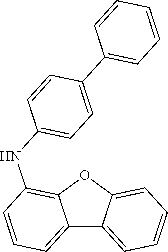 [SMILES: c1ccc(-c2ccc(Nc3cccc4c3oc3ccccc34)cc2)cc1] | 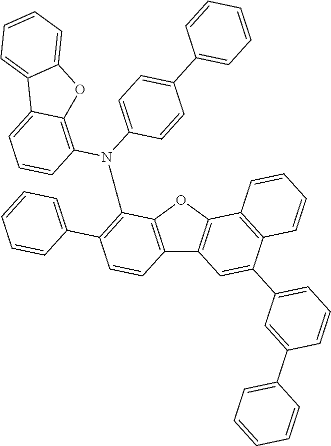 [SMILES: c1ccc(-c2ccc(N(c3cccc4c3oc3ccccc34)c3c(-c4ccccc4)ccc4c3oc3c5ccccc5c(-c5cccc(-c6ccccc6)c5)cc43)cc2)cc1] | 56% |
| 319 | 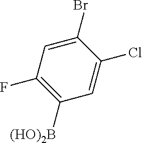 [SMILES: OB(O)c1cc(Cl)c(Br)cc1F] | 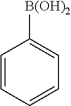 | 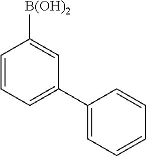 [SMILES: OB(O)c1cccc(-c2ccccc2)c1] | 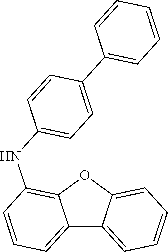 [SMILES: c1ccc(-c2ccc(Nc3cccc4c3oc3ccccc34)cc2)cc1] | 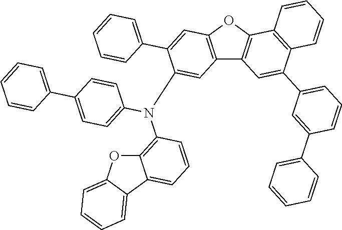 [SMILES: c1ccc(-c2ccc(N(c3cc4c(cc3-c3ccccc3)oc3c5ccccc5c(-c5cccc(-c6ccccc6)c5)cc43)c3cccc4c3oc3ccccc34)cc2)cc1] | 90% |
| 336 | 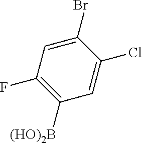 [SMILES: OB(O)c1cc(Cl)c(Br)cc1F] | 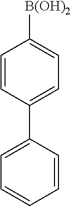 [SMILES: OB(O)c1ccc(-c2ccccc2)cc1] | 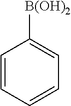 | 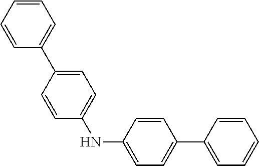 [SMILES: c1ccc(-c2ccc(Nc3ccc(-c4ccccc4)cc3)cc2)cc1] | 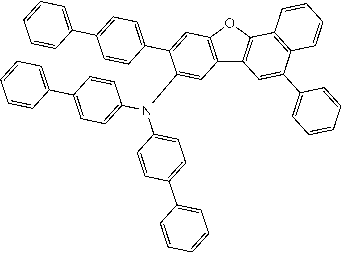 [SMILES: c1ccc(-c2ccc(-c3cc4oc5c6ccccc6c(-c6ccccc6)cc5c4cc3N(c3ccc(-c4ccccc4)cc3)c3ccc(-c4ccccc4)cc3)cc2)cc1] | 65% |
| 361 | 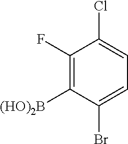 [SMILES: OB(O)c1c(Br)ccc(Cl)c1F] | 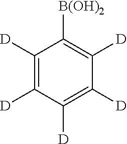 [SMILES: [2H]c1c([2H])c([2H])c(B(O)O)c([2H])c1[2H]] | 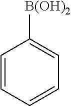 | 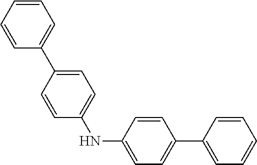 [SMILES: c1ccc(-c2ccc(Nc3ccc(-c4ccccc4)cc3)cc2)cc1] | 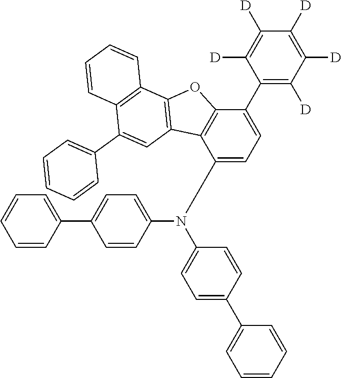 [SMILES: [2H]c1c([2H])c([2H])c(-c2ccc(N(c3ccc(-c4ccccc4)cc3)c3ccc(-c4ccccc4)cc3)c3c2oc2c4ccccc4c(-c4ccccc4)cc23)c([2H])c1[2H]] | 72% |
| 370 | 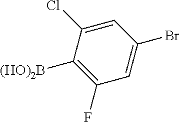 [SMILES: OB(O)c1c(F)cc(Br)cc1Cl] | 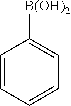 | 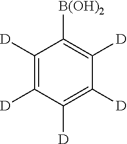 [SMILES: [2H]c1c([2H])c([2H])c(B(O)O)c([2H])c1[2H]] | 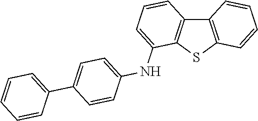 [SMILES: c1ccc(-c2ccc(Nc3cccc4c3sc3ccccc34)cc2)cc1] | 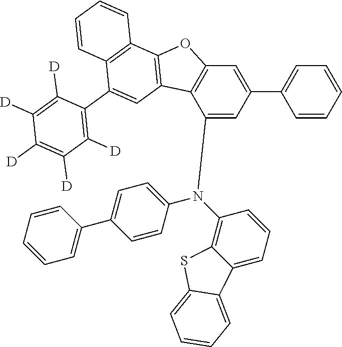 [SMILES: [2H]c1c([2H])c([2H])c(-c2cc3c(oc4cc(-c5ccccc5)cc(N(c5ccc(-c6ccccc6)cc5)c5cccc6c5sc5ccccc56)c43)c3ccccc23)c([2H])c1[2H]] | 54% |
| 376 | 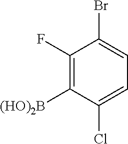 [SMILES: OB(O)c1c(Cl)ccc(Br)c1F] | 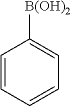 | 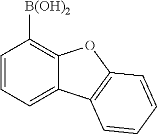 [SMILES: OB(O)c1cccc2c1oc1ccccc12] | 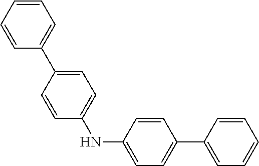 [SMILES: c1ccc(-c2ccc(Nc3ccc(-c4ccccc4)cc3)cc2)cc1] | 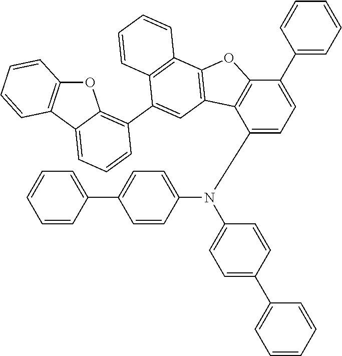 [SMILES: c1ccc(-c2ccc(N(c3ccc(-c4ccccc4)cc3)c3ccc(-c4ccccc4)c4oc5c6ccccc6c(-c6cccc7c6oc6ccccc67)cc5c34)cc2)cc1] | 82% |
| 380 | 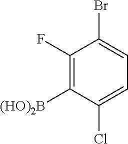 [SMILES: OB(O)c1c(Cl)ccc(Br)c1F] | 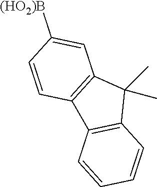 | 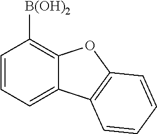 [SMILES: OB(O)c1cccc2c1oc1ccccc12] | 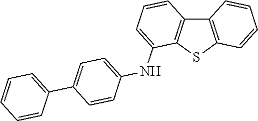 [SMILES: c1ccc(-c2ccc(Nc3cccc4c3sc3ccccc34)cc2)cc1] | 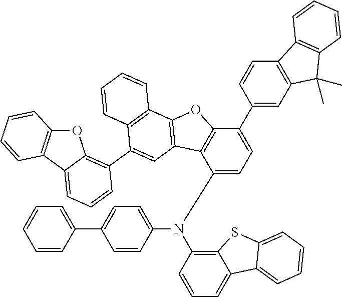 [SMILES: CC1(C)c2ccccc2-c2ccc(-c3ccc(N(c4ccc(-c5ccccc5)cc4)c4cccc5c4sc4ccccc45)c4c3oc3c5ccccc5c(-c5cccc6c5oc5ccccc56)cc34)cc21] | 82% |

### <Preparation Example 2> Preparation of Compound 41

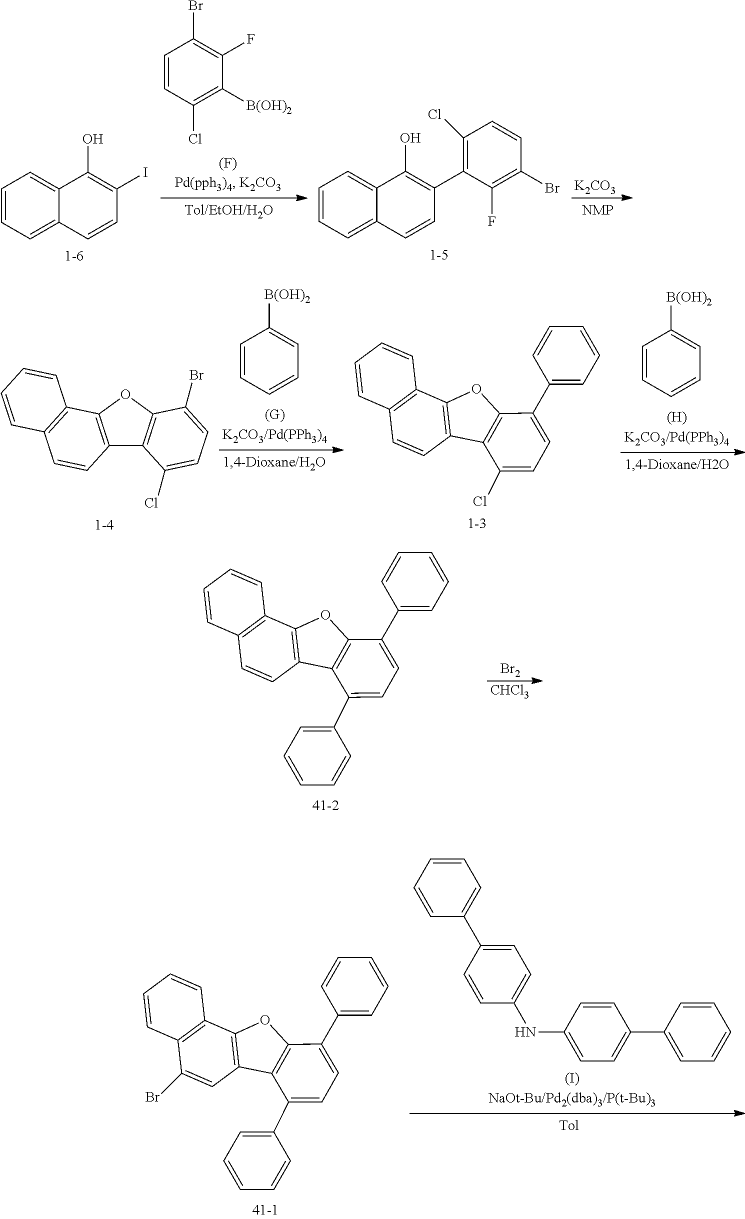

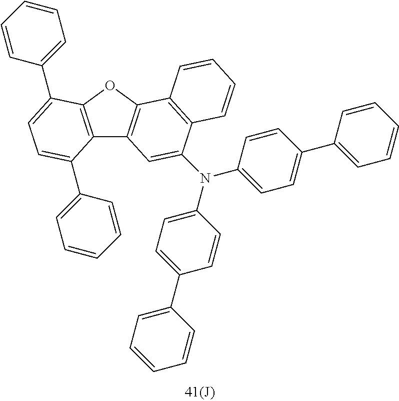 [SMILES: c1ccc(-c2ccc(N(c3ccc(-c4ccccc4)cc3)c3cc4c(oc5c(-c6ccccc6)ccc(-c6ccccc6)c54)c4ccccc34)cc2)cc1]

### (1) Preparation of Compound 41-2

7-Chloro-10-phenyl-naphtho[1,2-b]benzofuran (Compound 1-3) (25.0 g, 76.04 mmol), phenyl boronic acid (Intermediate H) (10.41 g, 83.64 mmol), Pd(dba)₂ (1.3 g, 3.8 mmol), xphos (3.62 g, 7.6 mmol) and potassium carbonate (31.53 g, 228.11 mmol) were added to 1,4-dioxane (250 mL) and water (50 mL), and the resulting mixture was stirred under reflux for 2 hours. Thereafter, the obtained solid was washed with water and filtered to obtain Compound 41-2 (25 g, 67.487 mmol, 88.757% yield).

For reference, the preparation process up to Compound 1-3 is the same as in Preparation Example 1, and 3-bromo-6-chloro-2-fluoro-phenyl)boronic acid and phenylboronic acid, which are the intermediates used in this case, are represented by Intermediate F and Intermediate G, respectively, in Chemical Reaction Formula of Preparation Example 2.

### (2) Preparation of Compound 41-1

After 7,10-diphenylnaphtho[1,2-b]benzofuran (Compound 41-2) (25.0 g, 67.49 mmol) was added to chloroform (250 mL) and dissolved, the resulting solution was stirred in a bath at −78° C. for 5 minutes, then bromine (10.89 g, 67.49 mmol) was added thereto, and the resulting mixture was stirred at room temperature for 3 hours. Thereafter, a solid was precipitated, and then quenched with methanol (MeOH), and filtered to obtain Compound 41-1 (30 g, 66.765 mmol, 98.929% yield).

### (3) Preparation of Compound 41

5-Bromo-7,10-diphenyl-naphtho[1,2-b]benzofuran (Compound 41-1) (25.0 g, 55.64 mmol), bis(4-biphenylyl)amine) (Intermediate I) (20.07 g, 61.2 mmol), Pd₂(dba)₃ (2.55 g, 2.78 mmol), t-Bu₃P (1.32 mL, 5.56 mmol) and sodium tert butoxide (16.04 g, 166.91 mmol) were added to toluene (500 mL), and the resulting mixture was stirred at 120° C. for 2 hours. After the reaction was completed, methylene chloride (MC) was added to the reaction solution and dissolved, then extraction was performed with distilled water, the organic layer was dried over anhydrous magnesium sulfate, the solvent was removed by a rotary evaporator, and then the residue was purified with column chromatography (MC:hexane=1:3) to obtain Compound 41 (25 g, 36.24 mmol, 65.137% yield).

A target compound J in the following Table 2 was synthesized in the same manner as in preparation Example 2, except that Intermediates F to I in the following Table 2 were used instead of 3-bromo-6-chloro-2-fluoro-phenyl)boronic acid (Intermediate F), phenyl boronic acid (Intermediate G), phenyl boronic acid (Intermediate H) and bis(4-biphenylyl)amine (Intermediate I), respectively, in Preparation Example 2.

TABLE 2

| Compound | Intermediate | Intermediate | Intermediate | Intermediate | Target compound | |
|---|---|---|---|---|---|---|
| No. | F | G | H | I | J | yield |
| 41 | 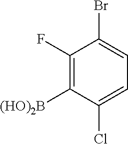 | 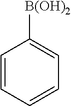 | 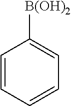 | 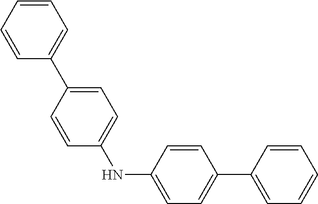 | 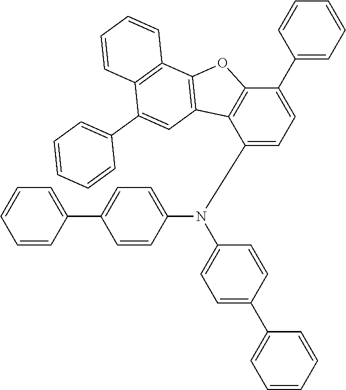 | 72% |
| 59 | 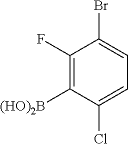 | 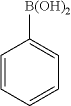 | 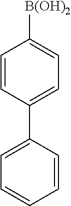 | 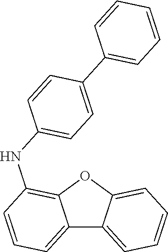 | 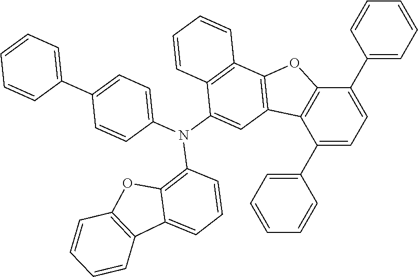 | 64% |
| 109 | 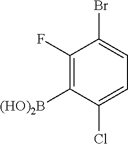 | 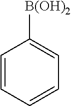 | 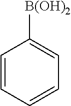 | 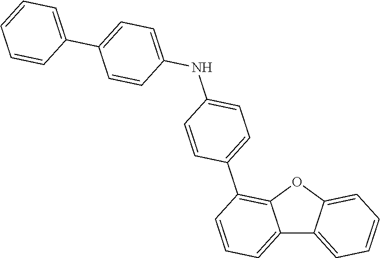 | 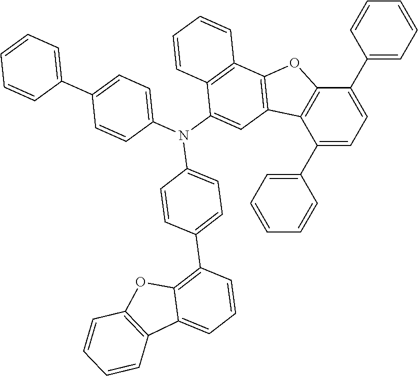 | 59% |
| 161 | 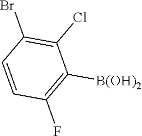 | 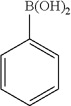 | 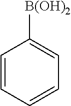 | 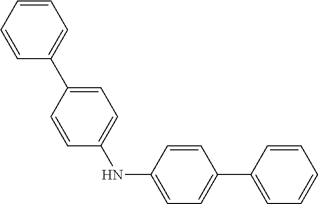 | 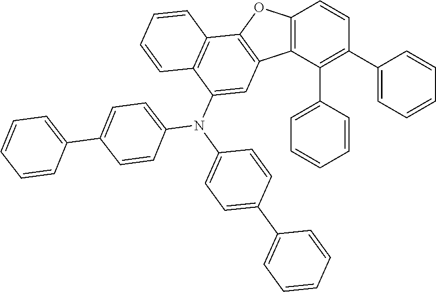 | 49% |
| 169 | 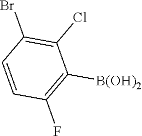 | 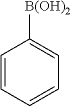 | 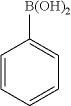 | 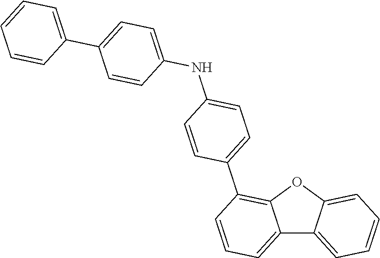 | 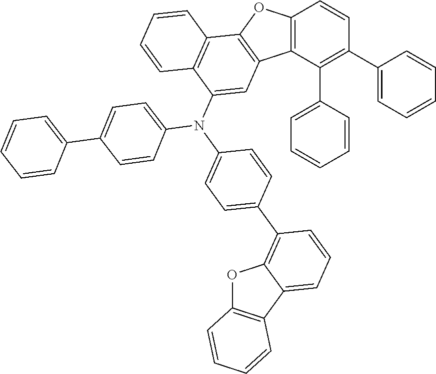 | 75% |
| 282 | 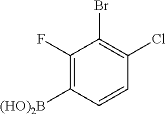 | 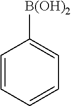 | 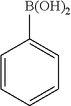 | 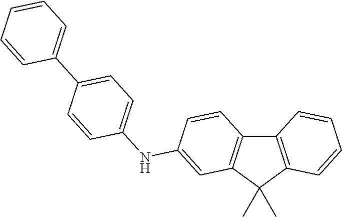 | 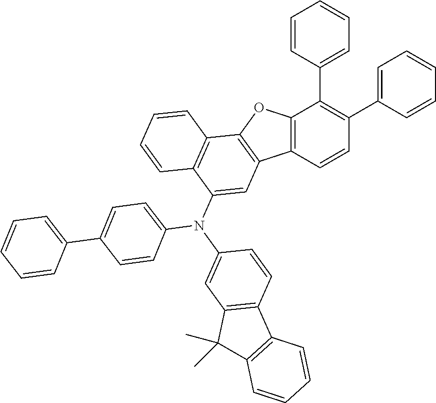 | 85% |
| 350 | 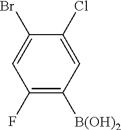 | 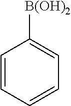 | 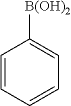 | 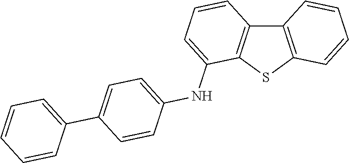 | 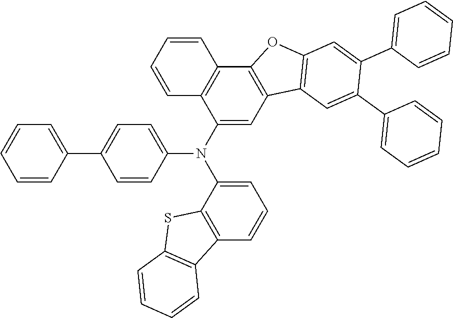 | 72% |

### <Preparation Example 3> Preparation of Compound 12

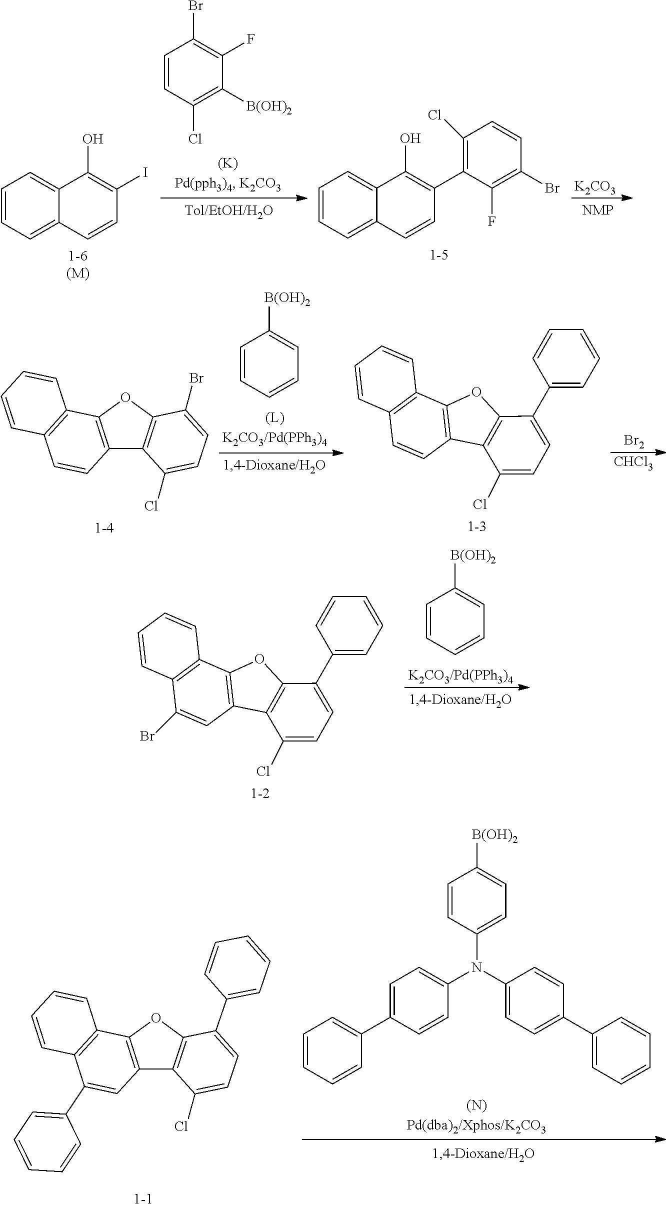

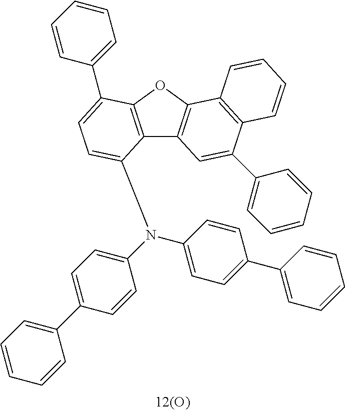 [SMILES: c1ccc(-c2ccc(N(c3ccc(-c4ccccc4)cc3)c3ccc(-c4ccccc4)c4oc5c6ccccc6c(-c6ccccc6)cc5c34)cc2)cc1]

### (1) Preparation of Compound 12

7-Chloro-5,10-diphenyl-naphtho[1,2-b]benzofuran (Compound 1-1) (25.0 g, 55.64 mmol), 4-(dibiphenyl-4-ylamino)phenylboronic acid (Intermediate N) (27.28 g, 61.2 mmol), Pd(dba)₂ (1.6 g, 2.78 mmol), Xphos (2.65 g, 5.56 mmol) and potassium carbonate (23.07 g, 166.91 mmol) were added to 1,4-dioxane (500 mL) and water (100 mL), and the resulting mixture was stirred under reflux at 125° C. for 5 hours. After the reaction was completed, methylene chloride (MC) was added to the reaction solution and dissolved, then extraction was performed with distilled water, the organic layer was dried over anhydrous magnesium sulfate, the solvent was removed by a rotary evaporator, and then the residue was purified with column chromatography (MC:hexane=1:3) to obtain Compound 12 (35 g, 45.695 mmol, 82.131% yield).

For reference, the preparation process up to Compound 1-1 is the same as in Preparation Example 1, and 3-bromo-6-chloro-2-fluoro-phenyl)boronic acid and phenylboronic acid, which are the intermediates used in this case, are represented by Intermediate K, Intermediate L and Intermediate M, respectively, in Chemical Reaction Formula of Preparation Example 3.

A target compound O in the following Table 3 was synthesized in the same manner as in Preparation Example 3, except that Intermediates K to N in the following Table 3 were used instead of 3-bromo-6-chloro-2-fluoro-phenyl)boronic acid (Intermediate K), phenyl boronic acid (Intermediate L), phenyl boronic acid (Intermediate M) and 4-(dibiphenyl-4-ylamino)phenylboronic acid (Intermediate N), respectively, in Preparation Example 3.

TABLE 3

| Com- | | | | | | |
|---|---|---|---|---|---|---|
| pound | Intermediate | Intermediate | Intermediate | Intermediate | Target compound | |
| No. | K | L | M | N | O | yield |
| 12 | 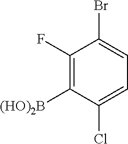 [SMILES: OB(O)c1c(Cl)ccc(Br)c1F] | 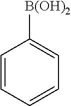 | 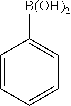 | 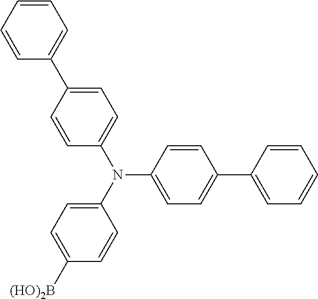 [SMILES: OB(O)c1ccc(N(c2ccc(-c3ccccc3)cc2)c2ccc(-c3ccccc3)cc2)cc1] | 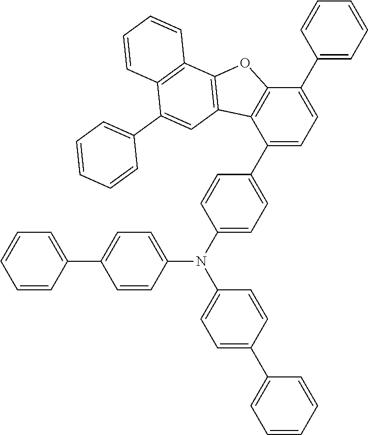 [SMILES: c1ccc(-c2ccc(N(c3ccc(-c4ccccc4)cc3)c3ccc(-c4ccc(-c5ccccc5)c5oc6c7ccccc7c(-c7ccccc7)cc6c45)cc3)cc2)cc1] | 82% |
| 132 | 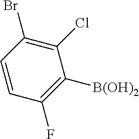 [SMILES: OB(O)c1c(F)ccc(Br)c1Cl] | 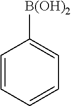 | 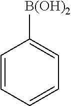 | 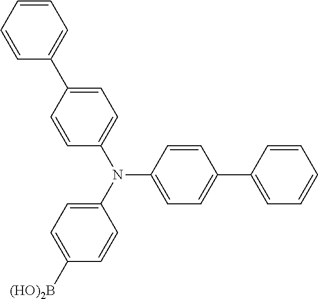 [SMILES: OB(O)c1ccc(N(c2ccc(-c3ccccc3)cc2)c2ccc(-c3ccccc3)cc2)cc1] | 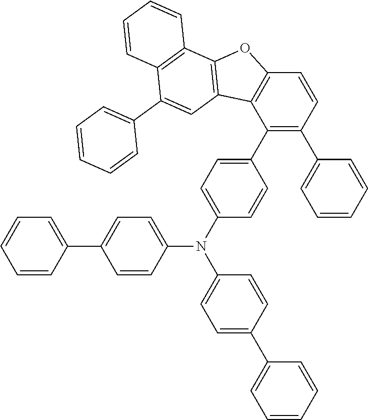 [SMILES: c1ccc(-c2ccc(N(c3ccc(-c4ccccc4)cc3)c3ccc(-c4c(-c5ccccc5)ccc5oc6c7ccccc7c(-c7ccccc7)cc6c45)cc3)cc2)cc1] | 73% |
| 154 | 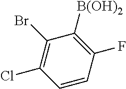 [SMILES: OB(O)c1c(F)ccc(Cl)c1Br] | 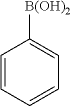 | 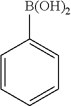 | 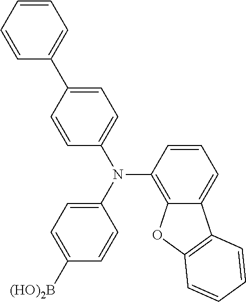 [SMILES: OB(O)c1ccc(N(c2ccc(-c3ccccc3)cc2)c2cccc3c2oc2ccccc23)cc1] | 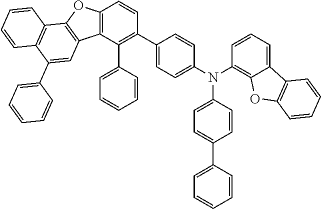 [SMILES: c1ccc(-c2ccc(N(c3ccc(-c4ccc5oc6c7ccccc7c(-c7ccccc7)cc6c5c4-c4ccccc4)cc3)c3cccc4c3oc3ccccc34)cc2)cc1] | 68% |
| 252 | 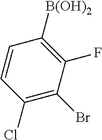 [SMILES: OB(O)c1ccc(Cl)c(Br)c1F] | 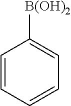 | 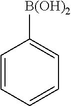 | 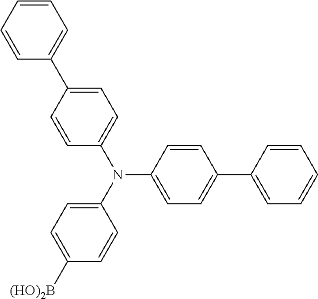 [SMILES: OB(O)c1ccc(N(c2ccc(-c3ccccc3)cc2)c2ccc(-c3ccccc3)cc2)cc1] | 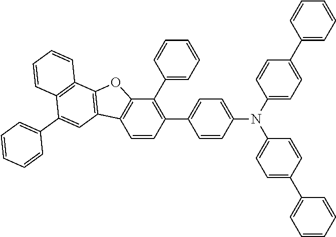 [SMILES: c1ccc(-c2ccc(N(c3ccc(-c4ccccc4)cc3)c3ccc(-c4ccc5c(oc6c7ccccc7c(-c7ccccc7)cc56)c4-c4ccccc4)cc3)cc2)cc1] | 85% |
| 335 | 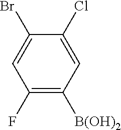 [SMILES: OB(O)c1cc(Cl)c(Br)cc1F] | 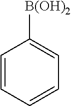 | 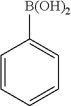 | 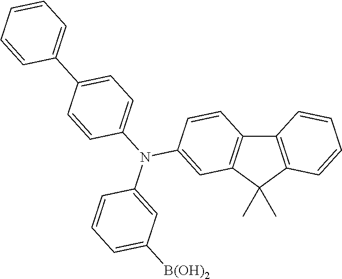 [SMILES: CC1(C)c2ccccc2-c2ccc(N(c3ccc(-c4ccccc4)cc3)c3cccc(B(O)O)c3)cc21] | 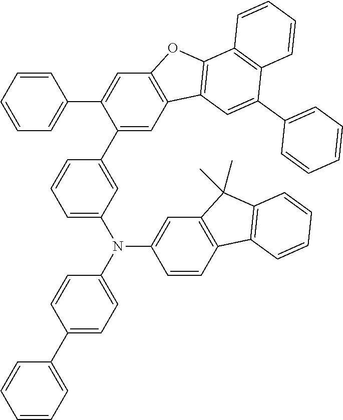 [SMILES: CC1(C)c2ccccc2-c2ccc(N(c3ccc(-c4ccccc4)cc3)c3cccc(-c4cc5c(cc4-c4ccccc4)oc4c6ccccc6c(-c6ccccc6)cc54)c3)cc21] | 69% |
| 372 | 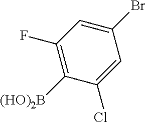 [SMILES: OB(O)c1c(F)cc(Br)cc1Cl] | 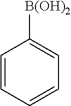 | 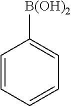 | 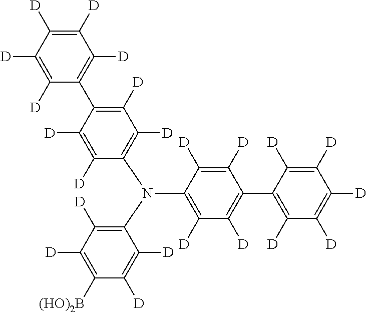 [SMILES: [2H]c1c([2H])c([2H])c(-c2c([2H])c([2H])c(N(c3c([2H])c([2H])c(B(O)O)c([2H])c3[2H])c3c([2H])c([2H])c(-c4c([2H])c([2H])c([2H])c([2H])c4[2H])c([2H])c3[2H])c([2H])c2[2H])c([2H])c1[2H]] | 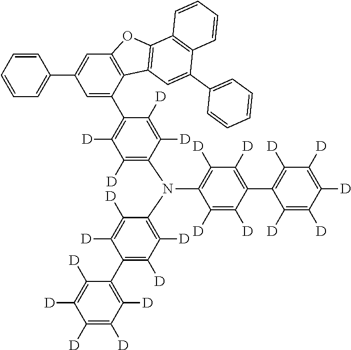 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c(-c4cc(-c5ccccc5)cc5oc6c7ccccc7c(-c7ccccc7)cc6c45)c([2H])c3[2H])c([2H])c2[2H])c([2H])c1[2H]] | 71% |

### <Preparation Example 4> Preparation of Compound 381

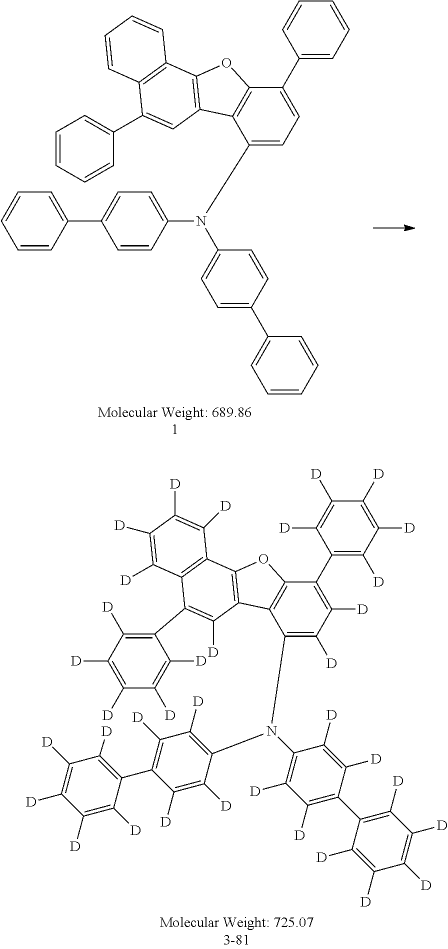 [SMILES: c1ccc(-c2ccc(N(c3ccc(-c4ccccc4)cc3)c3ccc(-c4ccccc4)c4oc5c6ccccc6c(-c6ccccc6)cc5c34)cc2)cc1 ; [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c(-c4c([2H])c([2H])c([2H])c([2H])c4[2H])c4oc5c6c([2H])c([2H])c([2H])c([2H])c6c(-c6c([2H])c([2H])c([2H])c([2H])c6[2H])c([2H])c5c34)c([2H])c2[2H])c([2H])c1[2H]]

After 10.0 g (14.4 mM) of Compound 1 and 15.4 g (102.7 mM) of trifluoromethanesulfonic acid were dissolved in D6 benzene (100 ml), the resulting solution was stirred at 60° C. for 4 hours. After the reaction was completed, the resulting product was neutralized with an aqueous K₃PO₄ solution at room temperature, and then extracted with dichloromethane/H₂O. The reaction solution was purified by column chromatography (DCM:Hex=1:1) and recrystallized with methanol to obtain 7.5 g (72.1%) of a target compound 381.

### <Preparation Example 5> Preparation of Compound 387

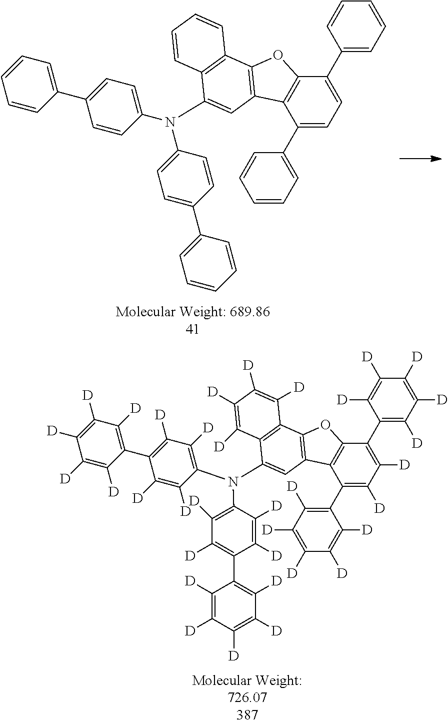 [SMILES: c1ccc(-c2ccc(N(c3ccc(-c4ccccc4)cc3)c3cc4c(oc5c(-c6ccccc6)ccc(-c6ccccc6)c54)c4ccccc34)cc2)cc1 ; [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3cc4c(oc5c(-c6c([2H])c([2H])c([2H])c([2H])c6[2H])c([2H])c([2H])c(-c6c([2H])c([2H])c([2H])c([2H])c6[2H])c54)c4c([2H])c([2H])c([2H])c([2H])c34)c([2H])c2[2H])c([2H])c1[2H]]

After 10.0 g (14.4 mM) of Compound 41 and 24.9 g (165.9 mM) of trifluoromethanesulfonic acid were dissolved in D6 benzene (150 ml), the resulting solution was stirred at 60° C. for 3 hours. After the reaction was completed, the resulting product was neutralized with an aqueous K₃PO₄ solution at room temperature, and then extracted with dichloromethane/H₂O. The reaction solution was purified by column chromatography (DCM:Hex=1:1) and recrystallized with methanol to obtain 9 g (86.5%) of a target compound 387.

Compounds were prepared in the same manner as in the Preparation Examples, and the synthesis confirmation results thereof are shown in the following Tables 4 and 5. Table 4 shows the measured values of ¹H NMR (CDCl₃, 200 MHz), and Table 5 shows the measured values of field desorption mass spectrometry (FD-MS).

TABLE 4

| Compound | ¹H NMR(CDCl₃, 200 MHz) |
|---|---|
| 1 | δ = 8.97(1H, d), 8.18(1H, d), 7.75~7.84(5H, m), 7.37~7.64(25H, m), 7.10(1H, d) |
| 2 | δ = 8.97(1H, d), 8.18(1H, d), 7.75~7.90(7H, m), 7.38~7.51(22H, m), 7.10~7.16(2H, m) |
| 21 | δ = 8.97(1H, d), 8.18(1H, d), 7.75~7.79(9H, m), 7.37~7.64(25H, m), 7.16(1H, d) |
| 36 | δ = 8.97(1H, d), 8.18(1H, d), 7.97(1H, s), 7.37~7.75(35H, m), 7.16(1H, d) |
| 41 | δ = 8.01~8.18(5H, m), 7.75~7.79(6H, m), 7.37~7.55(24H, m), 7.16~7.55(30H, m), |
| | 1.69(6H, s) |
| 47 | δ = 8.01~8.22(5H, m), 7.79~7.90(6H, m), 7.64(1H, t), 7.16~7.55(30H, m), 1.69(6H, s) |
| 59 | δ = 7.98~8.22(6H, m), 7.64(1H, d), 7.25~7.64(26H, m), 6.97(1H, d) |
| 68 | δ = 8.97(1H, d), 8.32(1H, d), 8.18(1H, d), 7.98(1H, d), 7.75~7.79(4H, m), |
| | 7.28~7.68(23H, m) |
| 78 | δ = 8.97(1H, d), 8.32(1H, d), 8.18(1H, d), 8.04(1H, s), 7.37~7.78(33H, m), 7.11(1H, t) |
| 96 | δ = 8.97(1H, d), 8.26(1H, s), 8.18(1H, d), 7.75~7.79(8H, m), 7.37~7.55(22H, m), |
| | 7.25(4H, s) |
| 97 | δ = 8.97(1H, d), 8.26(1H, s), 8.18(1H, d), 7.75~7.90(6H, m), 7.37~7.63(22H, m), |
| | 7.16(1H, d) |
| 109 | δ = 7.98~8.18(8H, m), 7.75~7.79(4H, m), 7.31~7.55(25H, m) |
| 116 | δ = 8.97(1H, d), 8.26(1H, s), 8.18(1H, d), 7.75~7.79(8H, m), 7.37~7.64(24H, m), |
| | 7.25(4H, s) |
| 136 | δ = 8.97(1H, d), 8.18(1H, s), 7.75~7.84(9H, m), 7.37~7.59(24H, m), 7.25(4H, s) |
| 161 | δ = 8.07~8.18(4H, m), 7.89(1H, d), 7.79(1H, m), 7.31~7.69(24H, m) |
| 175 | δ = 8.97(1H, d), 8.18(1H, s), 7.75~7.90(6H, m), 7.28~7.64(24H, m), 7.16(2H, d) |
| 181 | δ = 8.97(1H, d), 8.18~8.20(2H, d), 7.75~7.79(8H, m), 7.37~7.55(24H, m) |
| 267 | δ = 8.97(1H, d), 8.18(1H, s), 7.75~7.79(7H, m), 7.18~7.64(32H, m) |
| 279 | δ = 8.97(1H, d), 8.18(1H, s), 7.94~7.98(2H, d), 7.31~7.75(30H, m), 7.08(2H, m), |
| | 6.97(1H, s) |
| 282 | δ = 8.12~8.22(4H, m), 8.01(1H, d), 7.75~7.90(6H, m), 7.64(1H, t), 7.28~7.55(20H, m), |
| | 7.16(1H, d), 1.69(6H, s) |
| 286 | δ = 8.12~8.22(4H, m), 7.98~8.01(2H, m), 7.79~7.90(4H, m), 7.64(2H, m), |
| | 7.28~7.51(17H, m), 7.16(1H, d), 6.97(1H, d), 1.69(6H, s) |
| 301 | δ = 8.97(1H, d), 8.41(1H, s), 8.18(1H, d), 7.72~7.79(7H, m), 7.08~7.55(23H, m), |
| | 7.08(1H, d) |
| 319 | δ = 8.97(1H, d), 8.41(1H, s), 8.18(1H, s), 7.94~7.98(2H, m), 7.31~7.64(28H, m), |
| | 7.08(1H, d), 6.97(1H, d) |
| 336 | δ = 8.97(1H, d), 8.41(1H, s), 8.18(1H, d), 7.72~7.75(9H, m), 7.31~7.64(23H, m), |
| | 7.25(4H, s) |
| 350 | δ = 8.45(1H, d), 8.11~8.22(4H, m), 8.02(1H, s), 7.97~7.95(2H, m), 7.75~7.79(6H, m), |
| | 7.37~7.64(19H, m) |
| 361 | δ = 8.97(1H, d), 8.18(1H, d), 7.75~7.84(7H, d), 7.31~7.64(20H, m), 7.01(1H, d) |
| 372 | δ = 8.97(1H, d), 8.18(1H, d), 7.75~7.86(6H, m), 7.37~7.64(10H, m) |
| 380 | δ = 8.97(1H, d), 8.45(1H, d), 7.75~8.11(12H, m), 7.31~7.64(19H, m), 7.10(1H, d), |
| | 1.69(6H, s) |

TABLE 5

| Compound | FD-MS | Compound | FD-MS |
|---|---|---|---|
| 1 | MW: 689.86 | 2 | MW: 729.92 |
| 21 | MW: 689.86 | 36 | MW: 765.96 |
| 47 | MW: 894.13 | 68 | MW: 703.84 |
| 78 | MW: 816.02 | 96 | MW: 765.96 |
| 97 | MW: 779.98 | 116 | MW: 765.96 |
| 136 | MW: 765.96 | 175 | MW: 729.92 |
| 181 | MW: 689.86 | 267 | MW: 894.13 |
| 279 | MW: 779.94 | 286 | MW: 743.91 |
| 301 | MW: 689.86 | 319 | MW: 779.94 |
| 336 | MW: 689.86 | 361 | MW: 694.89 |
| 372 | MW: 801.11 | 380 | MW: 926.15 |
| 41 | MW: 689.86 | 59 | MW: 779.94 |
| 109 | MW: 779.94 | 161 | MW: 689.86 |
| 282 | MW: 729.92 | 350 | MW: 719.90 |

### Experimental Example 1

### 1) Manufacture of Organic Light Emitting Device

### Comparative Example 1

Trichloroethylene, acetone, ethanol, and distilled water were each sequentially used to ultrasonically wash a transparent electrode indium tin oxide (ITO) thin film obtained from glass for OLED (manufactured by Samsung-Corning Co., Ltd.) for 5 minutes, and then the ITO thin film was placed in isopropanol, stored, and then used. Next, the ITO substrate was disposed in a substrate folder of a vacuum deposition apparatus, and the following 4,4′,4″-tris(N,N-(2-naphthyl)-phenylamino)triphenyl amine (2-TNATA) was placed in a cell in the vacuum deposition apparatus.

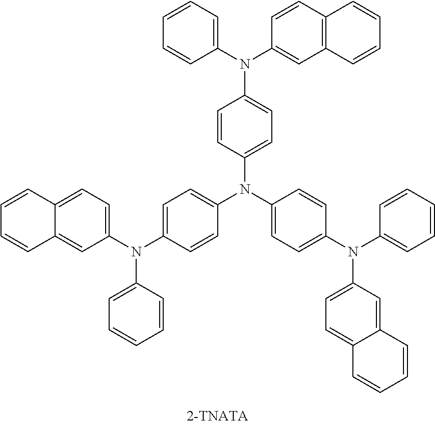 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

Subsequently, air in the chamber was evacuated until the degree of vacuum in the chamber reached 10-6 torr, and then a hole injection layer having a thickness of 600 Å was deposited on the ITO substrate by applying current to the cell to evaporate 2-TNATA. A hole transport layer having a thickness of 300 Å was deposited on the hole injection layer by placing the following N,N′-bis(α-naphthyl)-N,N′-diphenyl-4,4′-diamine (NPB) in another cell in the vacuum deposition apparatus and applying current to the cell to evaporate NPB.

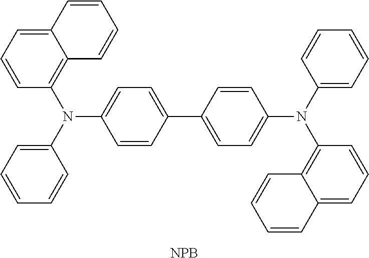 [SMILES: c1ccc(N(c2ccc(-c3ccc(N(c4ccccc4)c4cccc5ccccc45)cc3)cc2)c2cccc3ccccc23)cc1 ; "NPB"]

The hole injection layer and the hole transport layer were formed as described above, and then a blue light emitting material having the following structure as a light emitting layer was deposited thereon. Specifically, a blue light emitting host material H1 was vacuum deposited to have a thickness of 200 Å on one cell in the vacuum deposition apparatus, and a blue light emitting dopant material D1 was vacuum deposited thereon in an amount of 55 based on the host material.

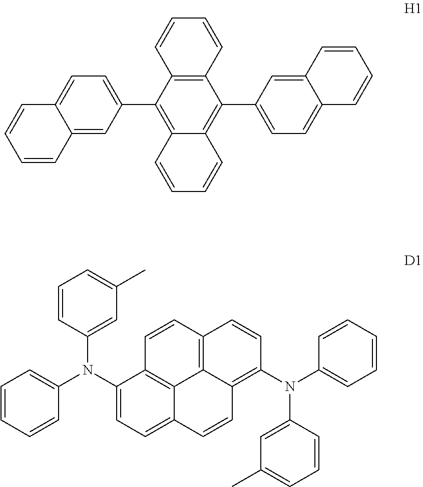

Subsequently, a compound having the following structural formula E1 as an electron transport layer was deposited to have a thickness of 300 Å.

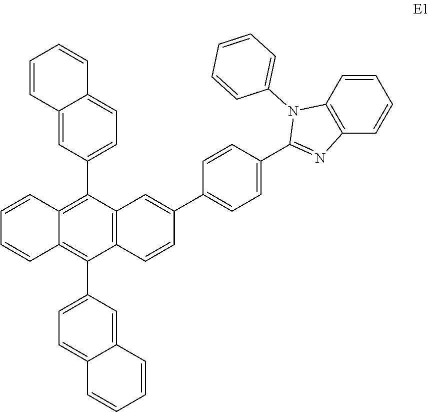 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

An OLED device was manufactured by depositing lithium fluoride (LiF) as an electron injection layer to have a thickness of 10 Å and allowing the Al negative electrode to have a thickness of 1,000 Å. Meanwhile, all the organic compounds required for manufacturing an OLED device were subjected to vacuum sublimed purification under 10⁻⁶ to 10⁻⁸ torr for each material, and used for the manufacture of OLED.

### Comparative Examples 2 to 7 and Examples 1 to 24

An organic electroluminescence device was manufactured in the same manner as in Comparative Example 1, except that the compound in the following Table 6 was used instead of NPB used when a hole transport layer was formed in Comparative Example 1.

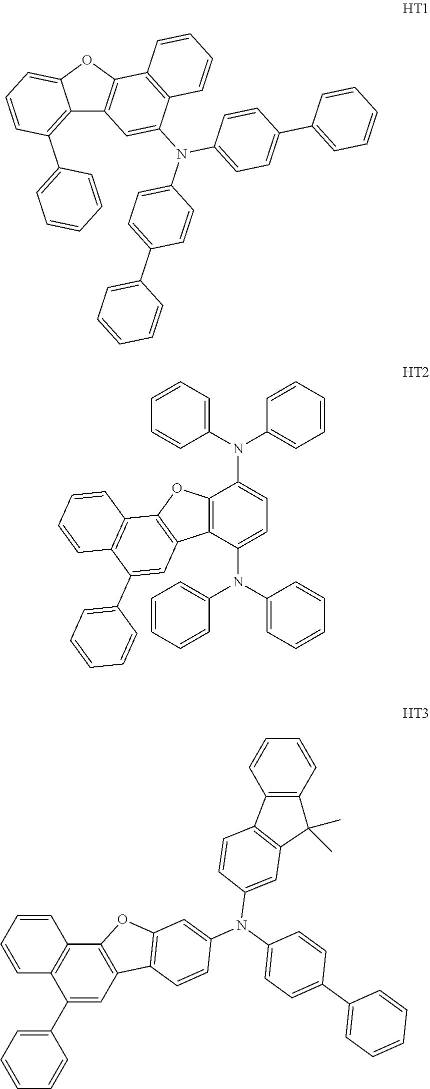 [SMILES: c1ccc(-c2ccc(N(c3ccc(-c4ccccc4)cc3)c3cc4c(oc5cccc(-c6ccccc6)c54)c4ccccc34)cc2)cc1 ; c1ccc(-c2cc3c(oc4c(N(c5ccccc5)c5ccccc5)ccc(N(c5ccccc5)c5ccccc5)c43)c3ccccc23)cc1 ; CC1(C)c2ccccc2-c2ccc(N(c3ccc(-c4ccccc4)cc3)c3ccc4c(c3)oc3c5ccccc5c(-c5ccccc5)cc43)cc21]

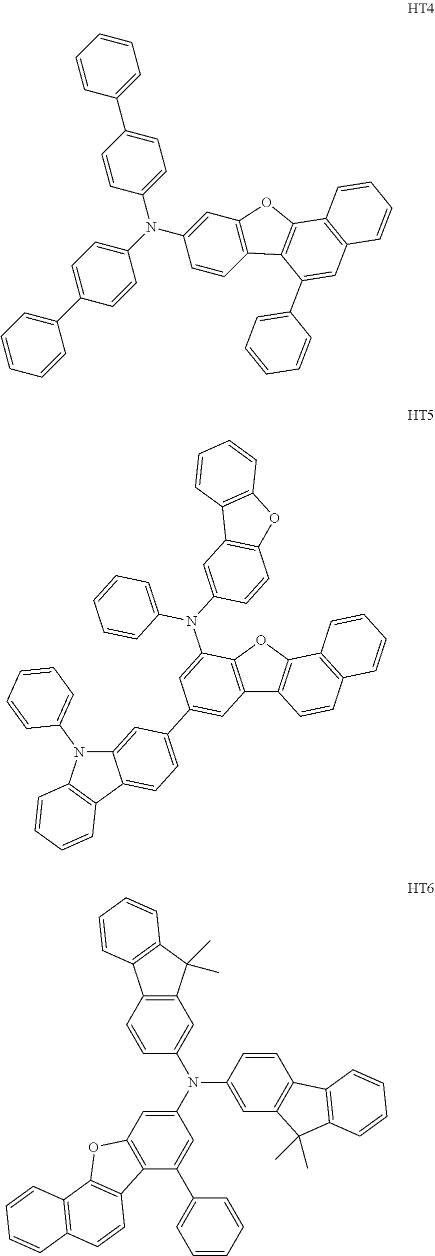 [SMILES: c1ccc(-c2ccc(N(c3ccc(-c4ccccc4)cc3)c3ccc4c(c3)oc3c5ccccc5cc(-c5ccccc5)c43)cc2)cc1 ; c1ccc(N(c2ccc3oc4ccccc4c3c2)c2cc(-c3ccc4c5ccccc5n(-c5ccccc5)c4c3)cc3c2oc2c4ccccc4ccc32)cc1 ; CC1(C)c2ccccc2-c2ccc(N(c3ccc4c(c3)C(C)(C)c3ccccc3-4)c3cc(-c4ccccc4)c4c(c3)oc3c5ccccc5ccc34)cc21]

### 2) Evaluation of Organic Light Emitting Device

For the organic light emitting device manufactured as described above, the electroluminescence (EL) characteristics were measured using M7000 manufactured by McScience Inc., and the measurement results were used to measure T₉₅ through a lifetime measurement device (M6000) manufactured by McScience Inc., when the reference luminance was 700 cd/m².

The results of measuring the driving voltage, light emitting efficiency, color coordinate (CIE), and life time (T₉₅) of the blue organic light emitting device manufactured according to the present invention are shown as in Table 6.

TABLE 6

| | | Driving | | | Life |
|---|---|---|---|---|---|
| | | voltage | Efficiency | CIE | time |
| | Compound | (V) | (cd/A) | (x, y) | (T₉₅) |
| Example 1 | 1 | 4.80 | 6.60 | (0.134, 0.100) | 53 |
| Example 2 | 2 | 4.78 | 6.64 | (0.134, 0.100) | 49 |
| Example 3 | 21 | 4.75 | 6.69 | (0.134, 0.100) | 47 |
| Example 4 | 36 | 4.73 | 6.70 | (0.134, 0.101) | 48 |
| Example 5 | 78 | 4.79 | 6.68 | (0.134, 0.101) | 50 |
| Example 6 | 96 | 4.78 | 6.73 | (0.134, 0.100) | 49 |
| Example 7 | 97 | 4.81 | 6.70 | (0.134, 0.100) | 49 |
| Example 8 | 116 | 4.84 | 6.68 | (0.134, 0.100) | 46 |
| Example 9 | 136 | 4.79 | 6.60 | (0.134, 0.100) | 47 |
| Example 10 | 175 | 4.76 | 6.81 | (0.134, 0.101) | 49 |
| Example 11 | 181 | 4.88 | 6.74 | (0.134, 0.101) | 50 |
| Example 12 | 267 | 4.83 | 6.70 | (0.134, 0.100) | 46 |
| Example 13 | 279 | 4.75 | 6.69 | (0.134, 0.100) | 44 |
| Example 14 | 319 | 4.69 | 6.68 | (0.134, 0.101) | 49 |
| Example 15 | 336 | 4.61 | 6.83 | (0.134, 0.101) | 50 |
| Example 16 | 361 | 4.59 | 6.82 | (0.134, 0.101) | 41 |
| Example 17 | 372 | 4.67 | 6.68 | (0.134, 0.101) | 49 |
| Example 18 | 380 | 4.75 | 6.86 | (0.134, 0.101) | 43 |
| Example 19 | 41 | 4.69 | 6.78 | (0.134, 0.101) | 49 |
| Example 20 | 59 | 4.72 | 6.82 | (0.134, 0.101) | 48 |
| Example 21 | 109 | 5.40 | 6.16 | (0.134, 0.101) | 37 |
| Example 22 | 161 | 5.42 | 6.65 | (0.134, 0.101) | 33 |
| Example 23 | 282 | 5.19 | 6.41 | (0.134, 0.100) | 35 |
| Example 24 | 350 | 5.17 | 6.30 | (0.134, 0.100) | 38 |
| Comparative | HT1 | 5.19 | 6.11 | (0.134, 0.101) | 33 |
| Example 1 | | | | | |
| Comparative | HT2 | 5.20 | 6.19 | (0.134, 0.101) | 31 |
| Example | | | | | |
| Comparative | HT3 | 5.20 | 6.22 | (0.134, 0.101) | 35 |
| Example 3 | | | | | |
| Comparative | HT4 | 5.23 | 6.11 | (0.134, 0.101) | 32 |
| Example 4 | | | | | |
| Comparative | HT4 | 5.19 | 6.19 | (0.134, 0.101) | 35 |
| Example 5 | | | | | |
| Comparative | HT5 | 5.20 | 6.22 | (0.134, 0.101) | 38 |
| Example 6 | | | | | |
| Comparative | HT6 | 5.20 | 6.11 | (0.134, 0.101) | 35 |
| Example 7 | | | | | |

As can be seen from the results in Table 6, the organic light emitting device using a hole transport layer material of the blue organic light emitting device of the present invention has a low driving voltage and remarkably improved light emitting efficiency and life time compared to the Comparative Examples.

In particular, for the compound of the present invention, it could be confirmed that when an amine derivative was used as a hole transport layer, the unshared electron pair of amine could enhance the flow of holes and improve the hole transfer capacity of the hole transport layer, and the binding of the amine moiety to the substituent with enhanced hole characteristics also enhanced the planarity and glass transition temperature of the amine derivative to enhance the thermal stability of the compound.

Further, since the bandgap and the energy level value of the triple state (T1 value) are adjusted to improve the hole transfer capacity and improve the stability of the molecule, it could be confirmed that the driving voltage of the device is lowered, the light efficiency of the device is improved, and life time characteristics of the device are improved by the thermal stability of the compound. Specifically, it could be confirmed that when a compound having two substituents at one phenyl of a naphthobenzofuran core, that is, an amine group in which one or more aryl groups are substituted and an aryl group, or an amine group in which one or more aryl groups are substituted and a heteroaryl group including 0 or S as in the compound represented by Chemical Formula 1 of the present application is used for a hole transport layer, the light emitting efficiency and life time are excellent because the substituted aryl group in the amine group delocalizes the highest occupied molecular orbital (HOMO) energy level to stabilize the HOMO energy of the compound.

### Experimental Example 2

### 1) Manufacture of Organic Light Emitting Device

### Comparative Examples 8 to 12 and Examples 25 to 48

A glass substrate thinly coated with ITO to have a thickness of 1,500 Å was ultrasonically washed with distilled water. When the washing with distilled water was finished, the glass substrate was ultrasonically washed with a solvent such as acetone, methanol, and isopropyl alcohol, dried and then was subjected to UVO treatment for 5 minutes using UV in a UV cleaning machine. Thereafter, the substrate was transferred to a plasma washing machine (PT), and then was subjected to plasma treatment in a vacuum state for an ITO work function and in order to remove a residual film, and was transferred to a thermal deposition apparatus for organic deposition.

Subsequently, air in the chamber was evacuated until the degree of vacuum in the chamber reached 10-6 torr, and then a hole injection layer having a thickness of 600 Å was deposited on the ITO substrate by applying current to the cell to evaporate 2-TNATA.

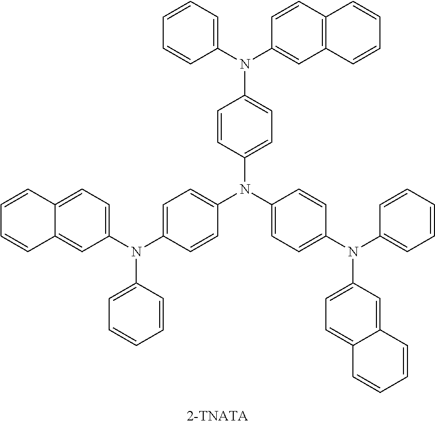 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

A hole transport layer having a thickness of 300 Å was deposited on the hole injection layer by placing the following N,N′-bis(α-naphthyl)-N,N′-diphenyl-4,4′-diamine (NPB) in another cell in the vacuum deposition apparatus and applying current to the cell to evaporate NPB.

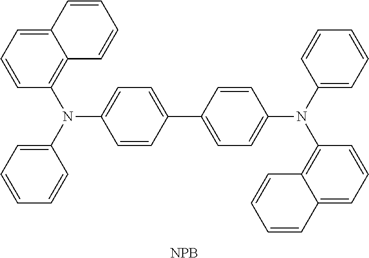 [SMILES: c1ccc(N(c2ccc(-c3ccc(N(c4ccccc4)c4cccc5ccccc45)cc3)cc2)c2cccc3ccccc23)cc1 ; "NPB"]

Thereafter, a prime layer was formed by depositing the compounds shown in the following Table 7 to 100 Å.

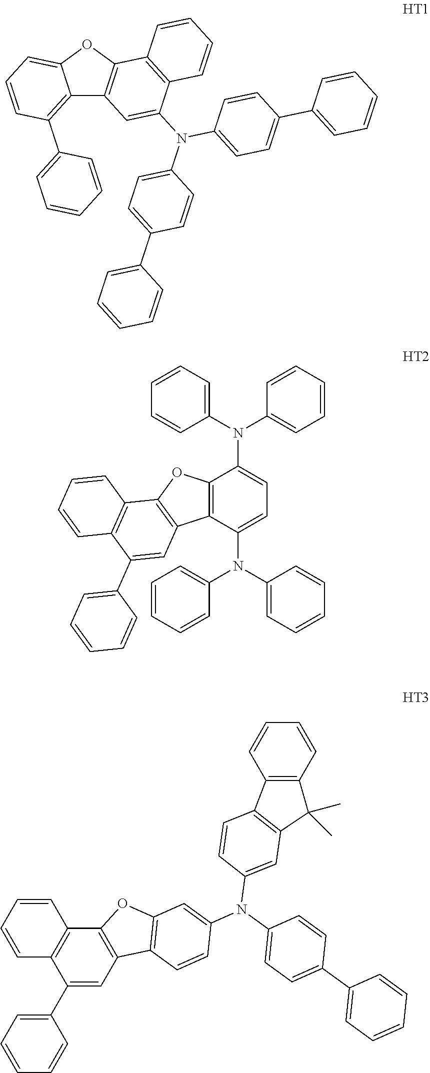 [SMILES: c1ccc(-c2ccc(N(c3ccc(-c4ccccc4)cc3)c3cc4c(oc5cccc(-c6ccccc6)c54)c4ccccc34)cc2)cc1 ; c1ccc(-c2cc3c(oc4c(N(c5ccccc5)c5ccccc5)ccc(N(c5ccccc5)c5ccccc5)c43)c3ccccc23)cc1 ; CC1(C)c2ccccc2-c2ccc(N(c3ccc(-c4ccccc4)cc3)c3ccc4c(c3)oc3c5ccccc5c(-c5ccccc5)cc43)cc21]

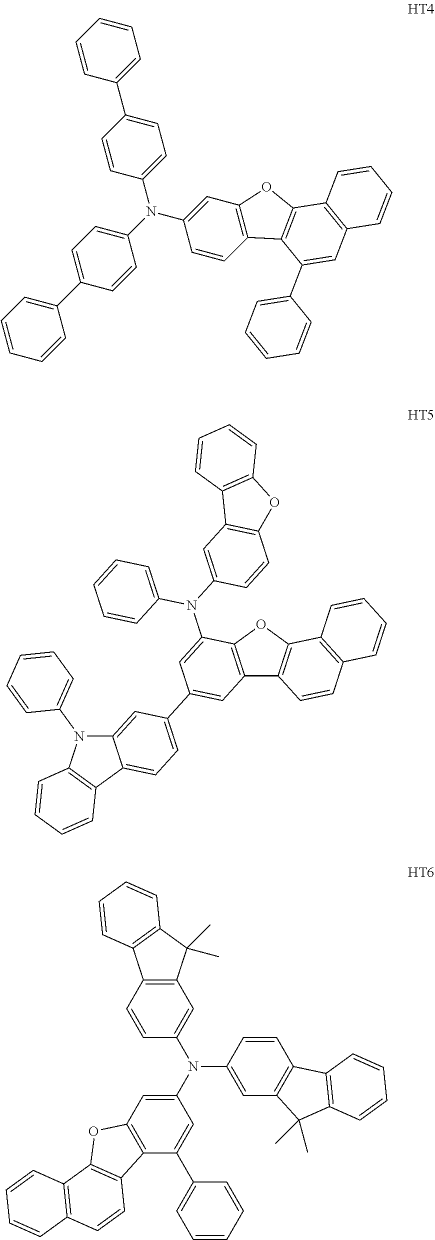 [SMILES: c1ccc(-c2ccc(N(c3ccc(-c4ccccc4)cc3)c3ccc4c(c3)oc3c5ccccc5cc(-c5ccccc5)c43)cc2)cc1 ; c1ccc(N(c2ccc3oc4ccccc4c3c2)c2cc(-c3ccc4c5ccccc5n(-c5ccccc5)c4c3)cc3c2oc2c4ccccc4ccc32)cc1 ; CC1(C)c2ccccc2-c2ccc(N(c3ccc4c(c3)C(C)(C)c3ccccc3-4)c3cc(-c4ccccc4)c4c(c3)oc3c5ccccc5ccc34)cc21]

A light emitting layer was thermally vacuum deposited thereon as follows. The light emitting layer was deposited by depositing a compound of 9-[4-(4,6-diphenyl-1,3,5-triazin-2-yl)phenyl]-9′-phenyl-3,3′-Bi-9H-carbazole as a host to 400 Å and doping the deposited layer with Ir(ppy)₃ as a green phosphorescent dopant by 7% of the deposited thickness of the light emitting layer. Thereafter, BCP as a hole blocking layer was deposited to have a thickness of 60 Å, and Alq₃ as an electron transport layer was deposited to have a thickness of 200 Å thereon.

Finally, an organic light emitting device was manufactured by depositing lithium fluoride (LiF) to have a thickness of 10 Å on the electron transport layer to form an electron injection layer, and then depositing an aluminum (Al) negative electrode to have a thickness of 1,200 Å on the electron injection layer to form a negative electrode.

Meanwhile, all the organic compounds required for manufacturing an organic light emitting device were subjected to vacuum sublimed purification under 10-8 to 10-torr for each material, and used for the manufacture of the organic light emitting device.

### 2) Evaluation of Organic Light Emitting Device

For the organic light emitting devices of Comparative Examples 8 to 12 and Examples 25 to 49 manufactured as above, electroluminescent light emission (EL) characteristics were measured using M7000 manufactured by McScience Inc., and with the measurement results, a life time T₉₅ (unit: h, hour), which was the time when the luminance became 90% compared to the initial luminance when the reference luminance was 6,000 cd/m², was measured using a life time measurement device (M6000) manufactured by McScience Inc, and the measurement results are shown in the following Table 7.

For reference, T₉₀ means the life time of the device measured at the time when the luminance reaches 90 relative to the initial luminance.

TABLE 7

| | | Driving | | |
|---|---|---|---|---|
| | | voltage | Efficiency | Life time |
| | Compound | (V) | (cd/A) | (T₉₅) |
| Example 25 | 1 | 4.73 | 6.68 | 208 |
| Example 26 | 2 | 4.75 | 6.70 | 250 |
| Example 27 | 21 | 4.80 | 6.74 | 220 |
| Example 28 | 36 | 4.77 | 6.66 | 253 |
| Example 29 | 78 | 4.76 | 6.63 | 213 |
| Example 30 | 96 | 4.76 | 6.72 | 213 |
| Example 31 | 97 | 4.73 | 6.80 | 197 |
| Example 32 | 116 | 4.79 | 6.73 | 220 |
| Example 33 | 136 | 4.70 | 6.70 | 210 |
| Example 34 | 175 | 4.76 | 6.69 | 240 |
| Example 35 | 181 | 4.73 | 6.71 | 256 |
| Example 36 | 267 | 4.80 | 6.73 | 224 |
| Example 37 | 279 | 4.73 | 6.69 | 213 |
| Example 38 | 319 | 4.81 | 6.75 | 242 |
| Example 39 | 336 | 4.79 | 6.73 | 222 |
| Example 40 | 361 | 4.70 | 6.70 | 253 |
| Example 41 | 372 | 4.76 | 6.69 | 230 |
| Example 42 | 380 | 4.73 | 6.71 | 200 |
| Example 43 | 41 | 4.80 | 6.73 | 198 |
| Example 44 | 59 | 4.76 | 6.82 | 220 |
| Example 45 | 109 | 4.76 | 6.82 | 246 |
| Example 46 | 161 | 4.76 | 6.82 | 240 |
| Example 47 | 282 | 4.76 | 6.82 | 260 |
| Example 48 | 350 | 4.76 | 6.82 | 224 |
| Comparative | HT1 | 5.36 | 6.14 | 171 |
| Example 8 | | | | |
| Comparative | HT2 | 5.30 | 6.18 | 182 |
| Example 9 | | | | |
| Comparative | HT3 | 5.33 | 6.16 | 167 |
| Example 10 | | | | |
| Comparative | HT4 | 5.30 | 6.10 | 178 |
| Example 11 | | | | |
| Comparative | HT5 | 5.20 | 6.22 | 168 |
| Example 12 | | | | |

From the results of Table 7, it could be confirmed that when the prime layer was formed, in the case of the organic light emitting devices of Examples 25 to 49, in which the compound according to the present application was used, two specific substituents including an amine group in naphthobenzofuran had a substituted structure to delocalize the highest occupied molecular orbital (HOMO) energy level, and effectively prevented electrons from crossing from the opposite side of the electron transport layer by stabilizing the HOMO energy, and as a result, during the formation of the prime layer, the light emitting efficiencies and service lives thereof were better than those of the organic light emitting devices of Comparative Examples 9 to 12 in which the compound according to the present application was not used.

### EXPLANATION OF REFERENCE NUMERALS AND SYMBOLS

100: Substrate 200: Positive electrode
300: Organic material layer 301: Hole injection layer
302: Hole transport layer 303: Light emitting layer
304: Hole blocking layer 305: Electron transport layer
306: Electron injection layer 400: Negative electrode

1. A heterocyclic compound represented by the following Chemical Formula 1: 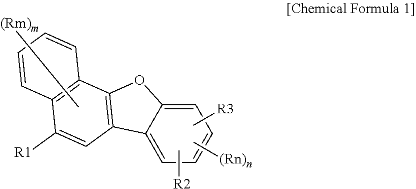
in Chemical Formula 1,
R1 to R3 are the same as or different from each other, and are each independently a substituted or unsubstituted aryl group having 6 to 60 carbon atoms; a substituted or unsubstituted heteroaryl group having 2 to 60 carbon atoms; or -(L1)ₐNR4R5, and one of R1 to R3 is -(L1)ₐNR4R5,
L1 is a direct bond; a substituted or unsubstituted arylene group having 6 to 60 carbon atoms; or a substituted or unsubstituted heteroarylene group having 2 to 60 carbon atoms, and a is an integer from 0 to 3, and when a is 2 or higher, L1's in the parenthesis are the same as or different from each other,
R4 and R5 are the same as or different from each other, and are each independently a substituted or unsubstituted aryl group having 6 to 60 carbon atoms; or a substituted or unsubstituted heteroaryl group having 2 to 60 carbon atoms,
Rm and Rn are the same as or different from each other, and are each independently hydrogen; deuterium; a cyano group; a substituted or unsubstituted aryl group having 6 to 60 carbon atoms; or a substituted or unsubstituted alkyl group having 1 to 60 carbon atoms,
m is an integer from 0 to 5, and when m is 2 or more, Rm's in the parenthesis are the same as or different from each other, and
n is an integer from 0 to 2, and when n is 2, Rn's in the parenthesis are the same as or different from each other.

2. The heterocyclic compound of claim 1, wherein a deuterium content of the heterocyclic compound represented by Chemical Formula 1 is 10% to 100%.

3. The heterocyclic compound of claim 1, wherein Chemical Formula 1 is represented by any one of the following Chemical Formulae 1-1 to 1-3: 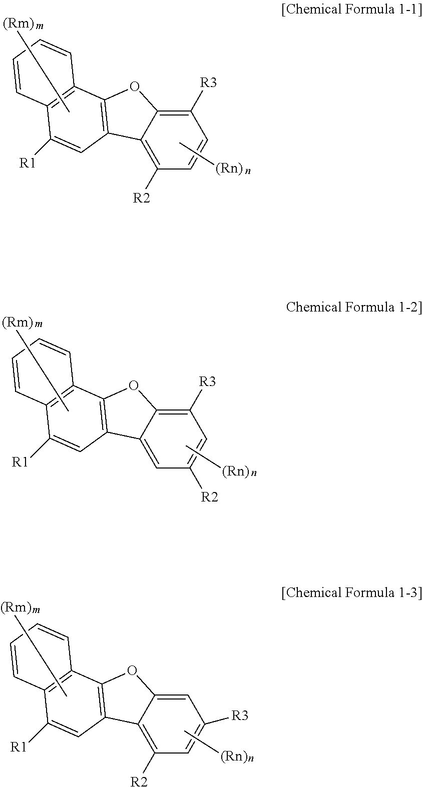
in Chemical Formulae 1-1 to 1-3, the definitions of R1 to R3, Rm, Rn, m and n are the same as those in Chemical Formula 1.

4. The heterocyclic compound of claim 1, wherein Chemical Formula 1 is represented by any one of the following Chemical Formulae 1-4 to 1-6: 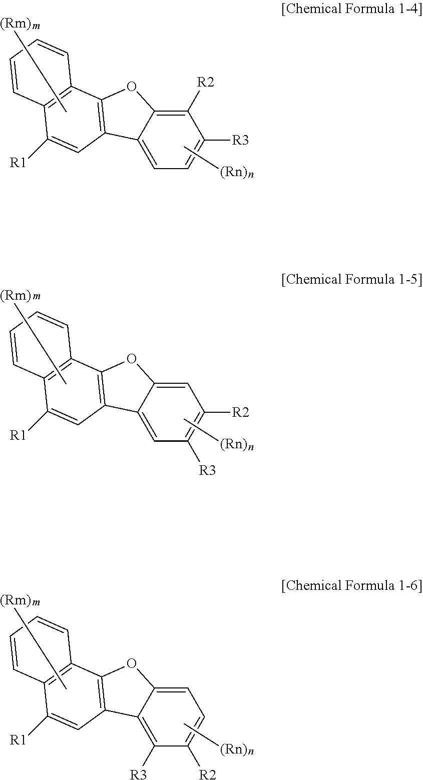
in Chemical Formulae 1-4 to 1-6, the definitions of R1 to R3, Rm, Rn, m and n are the same as those in Chemical Formula 1.

5. The heterocyclic compound of claim 1, wherein when R1 to R3 optionally each independently have a substituent substituted with deuterium and the contents of substituted deuterium of R1 to R3 are each independently 10% to 100%.

6. The heterocyclic compound of claim 1, wherein L 1 is a direct bond; or a phenylene group.

7. The heterocyclic compound of claim 1, wherein Rm and Rn are the same as or different from each other, and each independently comprise hydrogen; or deuterium.

8. The heterocyclic compound of claim 1, wherein Chemical Formula 1 is represented by any one of the following compounds:

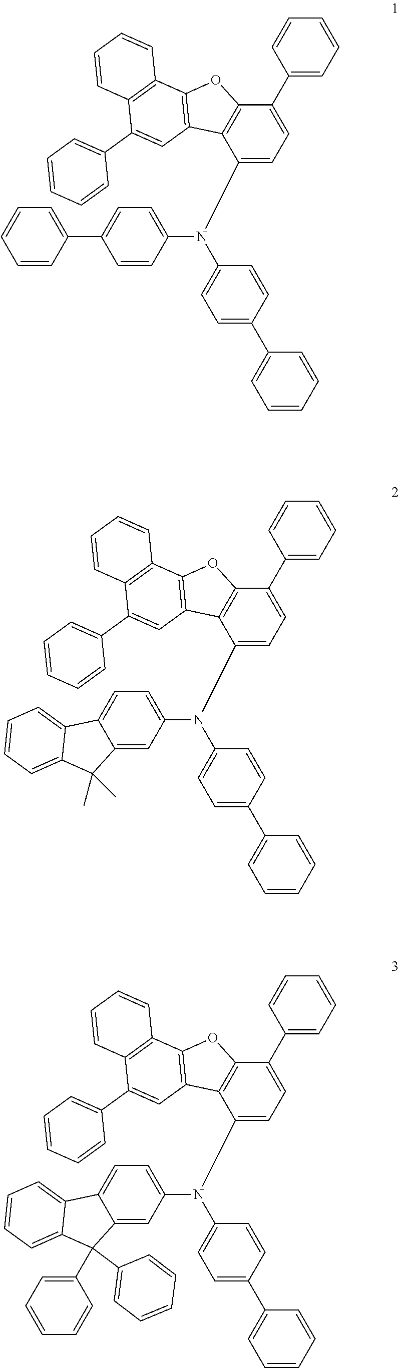 [SMILES: c1ccc(-c2ccc(N(c3ccc(-c4ccccc4)cc3)c3ccc(-c4ccccc4)c4oc5c6ccccc6c(-c6ccccc6)cc5c34)cc2)cc1 ; CC1(C)c2ccccc2-c2ccc(N(c3ccc(-c4ccccc4)cc3)c3ccc(-c4ccccc4)c4oc5c6ccccc6c(-c6ccccc6)cc5c34)cc21 ; c1ccc(-c2ccc(N(c3ccc4c(c3)C(c3ccccc3)(c3ccccc3)c3ccccc3-4)c3ccc(-c4ccccc4)c4oc5c6ccccc6c(-c6ccccc6)cc5c34)cc2)cc1]

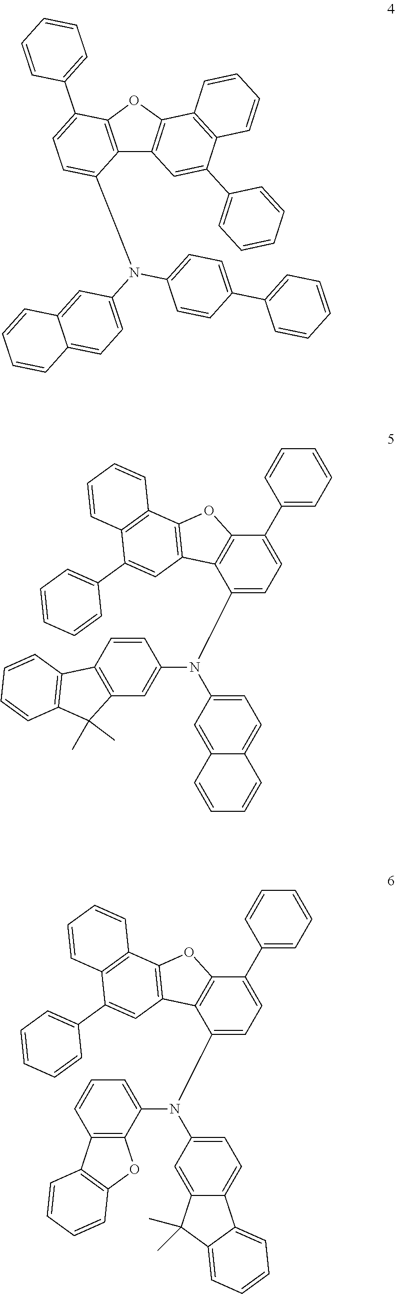 [SMILES: c1ccc(-c2ccc(N(c3ccc4ccccc4c3)c3ccc(-c4ccccc4)c4oc5c6ccccc6c(-c6ccccc6)cc5c34)cc2)cc1 ; CC1(C)c2ccccc2-c2ccc(N(c3ccc4ccccc4c3)c3ccc(-c4ccccc4)c4oc5c6ccccc6c(-c6ccccc6)cc5c34)cc21 ; CC1(C)c2ccccc2-c2ccc(N(c3cccc4c3oc3ccccc34)c3ccc(-c4ccccc4)c4oc5c6ccccc6c(-c6ccccc6)cc5c34)cc21]

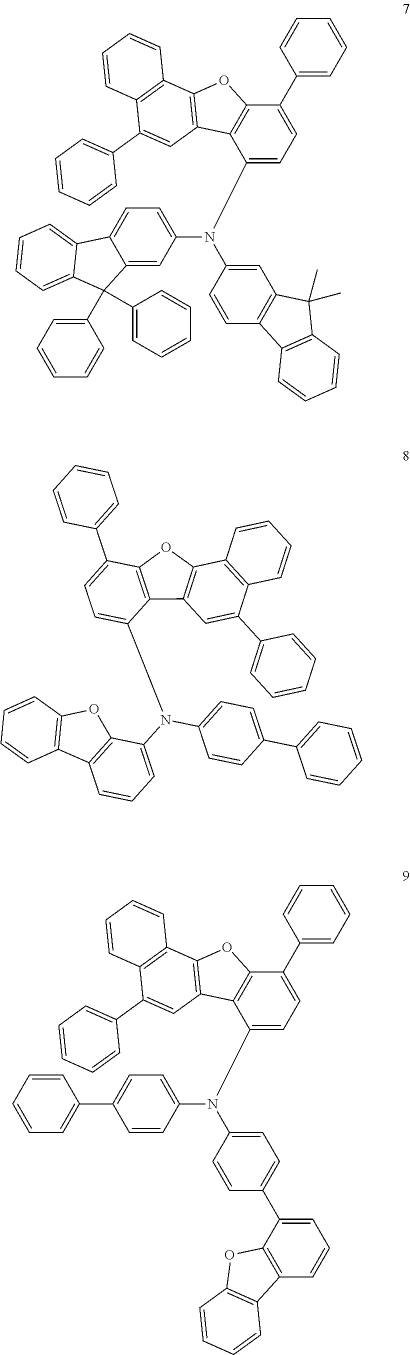 [SMILES: CC1(C)c2ccccc2-c2ccc(N(c3ccc4c(c3)C(c3ccccc3)(c3ccccc3)c3ccccc3-4)c3ccc(-c4ccccc4)c4oc5c6ccccc6c(-c6ccccc6)cc5c34)cc21 ; c1ccc(-c2ccc(N(c3cccc4c3oc3ccccc34)c3ccc(-c4ccccc4)c4oc5c6ccccc6c(-c6ccccc6)cc5c34)cc2)cc1 ; c1ccc(-c2ccc(N(c3ccc(-c4cccc5c4oc4ccccc45)cc3)c3ccc(-c4ccccc4)c4oc5c6ccccc6c(-c6ccccc6)cc5c34)cc2)cc1]

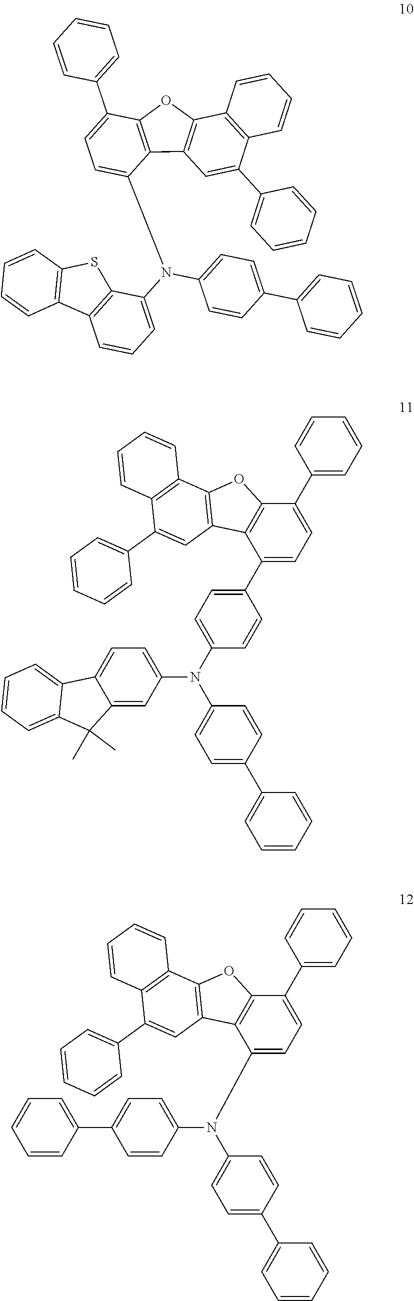 [SMILES: c1ccc(-c2ccc(N(c3cccc4c3sc3ccccc34)c3ccc(-c4ccccc4)c4oc5c6ccccc6c(-c6ccccc6)cc5c34)cc2)cc1 ; CC1(C)c2ccccc2-c2ccc(N(c3ccc(-c4ccccc4)cc3)c3ccc(-c4ccc(-c5ccccc5)c5oc6c7ccccc7c(-c7ccccc7)cc6c45)cc3)cc21 ; c1ccc(-c2ccc(N(c3ccc(-c4ccccc4)cc3)c3ccc(-c4ccccc4)c4oc5c6ccccc6c(-c6ccccc6)cc5c34)cc2)cc1]

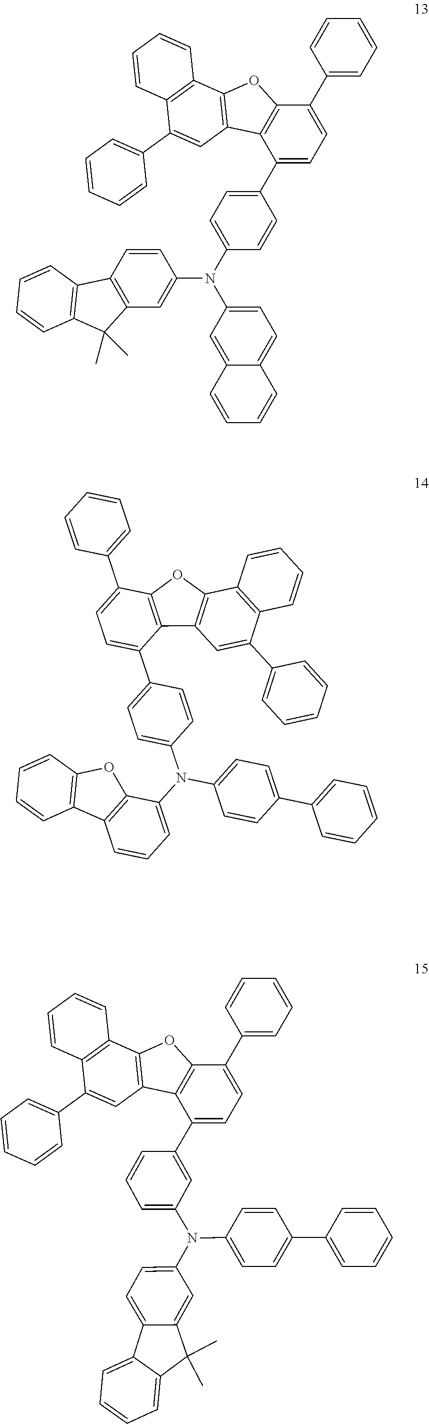 [SMILES: CC1(C)c2ccccc2-c2ccc(N(c3ccc(-c4ccc(-c5ccccc5)c5oc6c7ccccc7c(-c7ccccc7)cc6c45)cc3)c3ccc4ccccc4c3)cc21 ; c1ccc(-c2ccc(N(c3ccc(-c4ccc(-c5ccccc5)c5oc6c7ccccc7c(-c7ccccc7)cc6c45)cc3)c3cccc4c3oc3ccccc34)cc2)cc1 ; CC1(C)c2ccccc2-c2ccc(N(c3ccc(-c4ccccc4)cc3)c3cccc(-c4ccc(-c5ccccc5)c5oc6c7ccccc7c(-c7ccccc7)cc6c45)c3)cc21]

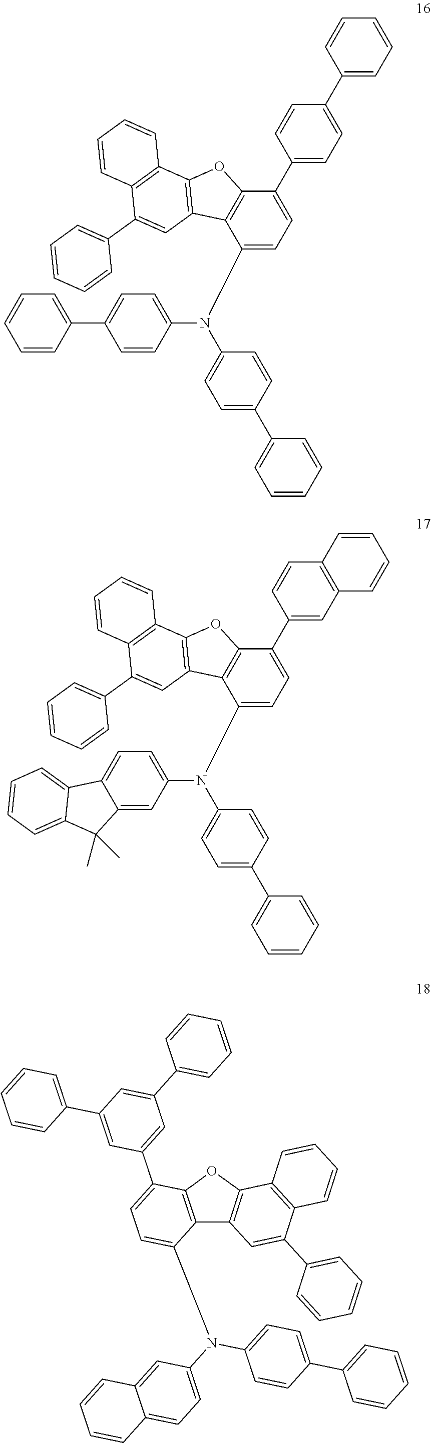 [SMILES: c1ccc(-c2ccc(-c3ccc(N(c4ccc(-c5ccccc5)cc4)c4ccc(-c5ccccc5)cc4)c4c3oc3c5ccccc5c(-c5ccccc5)cc34)cc2)cc1 ; CC1(C)c2ccccc2-c2ccc(N(c3ccc(-c4ccccc4)cc3)c3ccc(-c4ccc5ccccc5c4)c4oc5c6ccccc6c(-c6ccccc6)cc5c34)cc21 ; c1ccc(-c2ccc(N(c3ccc4ccccc4c3)c3ccc(-c4cc(-c5ccccc5)cc(-c5ccccc5)c4)c4oc5c6ccccc6c(-c6ccccc6)cc5c34)cc2)cc1]

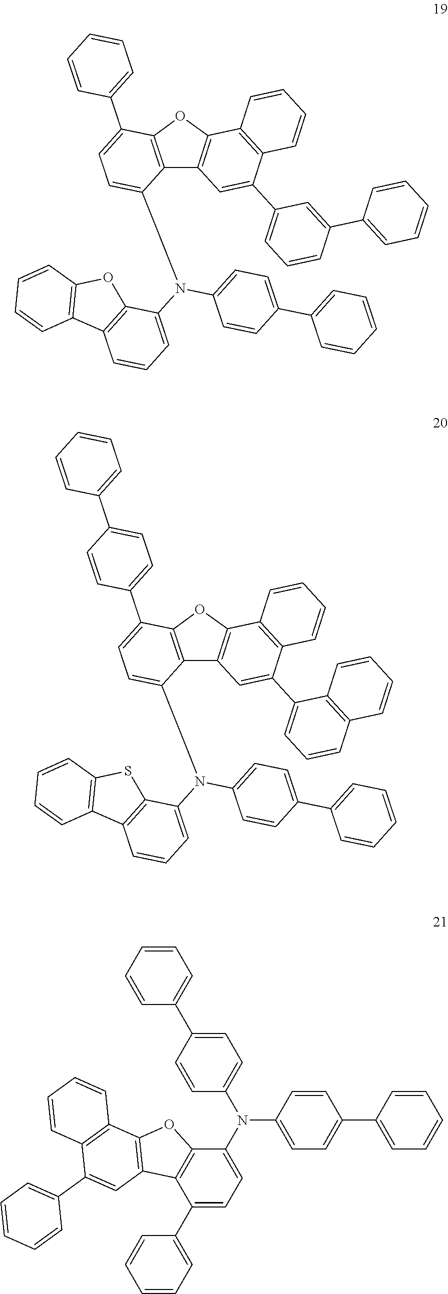 [SMILES: c1ccc(-c2ccc(N(c3cccc4c3oc3ccccc34)c3ccc(-c4ccccc4)c4oc5c6ccccc6c(-c6cccc(-c7ccccc7)c6)cc5c34)cc2)cc1 ; c1ccc(-c2ccc(-c3ccc(N(c4ccc(-c5ccccc5)cc4)c4cccc5c4sc4ccccc45)c4c3oc3c5ccccc5c(-c5cccc6ccccc56)cc34)cc2)cc1 ; c1ccc(-c2ccc(N(c3ccc(-c4ccccc4)cc3)c3ccc(-c4ccccc4)c4c3oc3c5ccccc5c(-c5ccccc5)cc34)cc2)cc1]

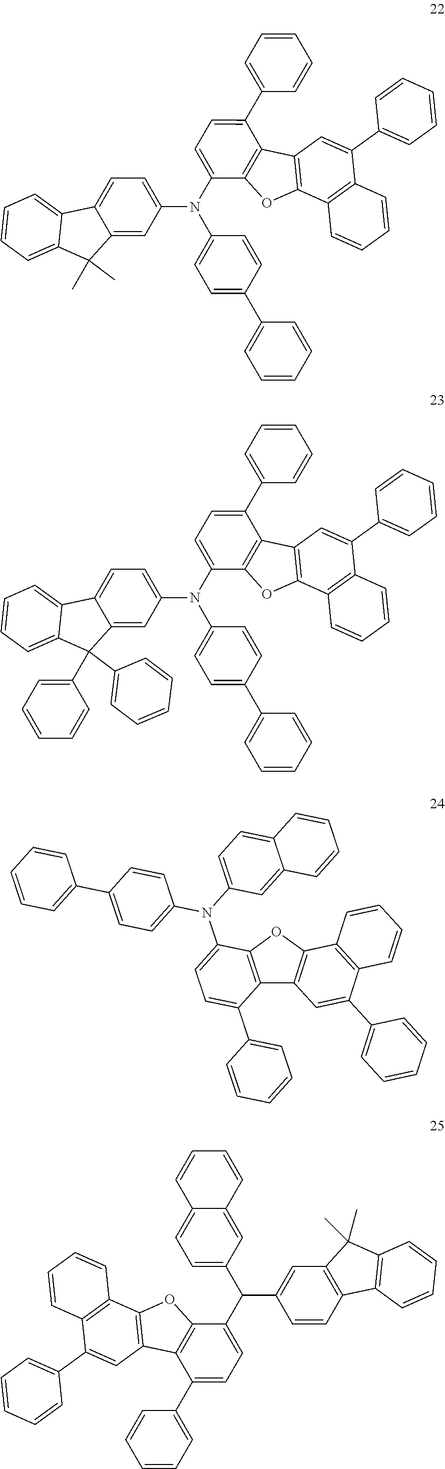

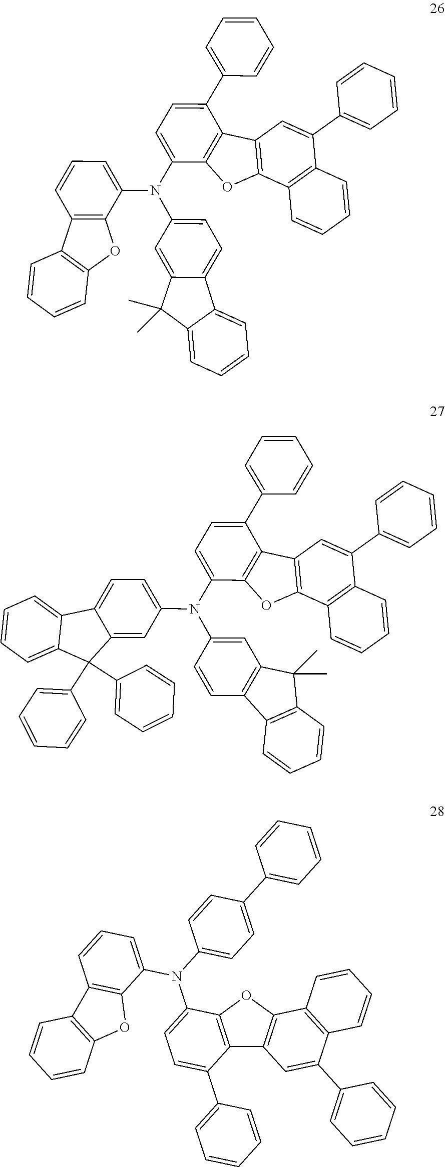 [SMILES: CC1(C)c2ccccc2-c2ccc(N(c3cccc4c3oc3ccccc34)c3ccc(-c4ccccc4)c4c3oc3c5ccccc5c(-c5ccccc5)cc34)cc21 ; CC1(C)c2ccccc2-c2ccc(N(c3ccc4c(c3)C(c3ccccc3)(c3ccccc3)c3ccccc3-4)c3ccc(-c4ccccc4)c4c3oc3c5ccccc5c(-c5ccccc5)cc34)cc21 ; c1ccc(-c2ccc(N(c3cccc4c3oc3ccccc34)c3ccc(-c4ccccc4)c4c3oc3c5ccccc5c(-c5ccccc5)cc34)cc2)cc1]

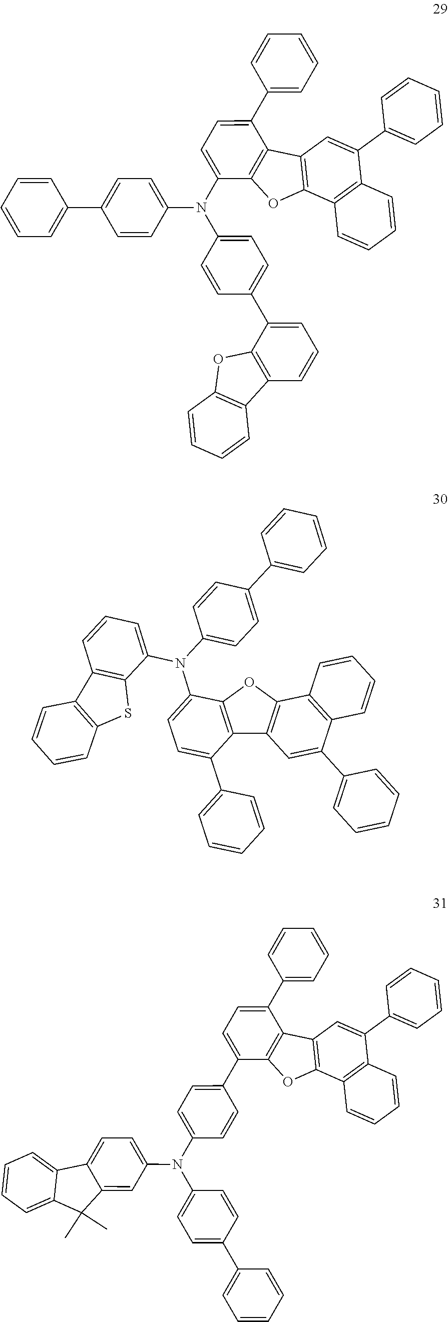 [SMILES: c1ccc(-c2ccc(N(c3ccc(-c4cccc5c4oc4ccccc45)cc3)c3ccc(-c4ccccc4)c4c3oc3c5ccccc5c(-c5ccccc5)cc34)cc2)cc1 ; c1ccc(-c2ccc(N(c3ccc(-c4ccccc4)c4c3oc3c5ccccc5c(-c5ccccc5)cc34)c3cccc4c3sc3ccccc34)cc2)cc1 ; CC1(C)c2ccccc2-c2ccc(N(c3ccc(-c4ccccc4)cc3)c3ccc(-c4ccc(-c5ccccc5)c5c4oc4c6ccccc6c(-c6ccccc6)cc45)cc3)cc21]

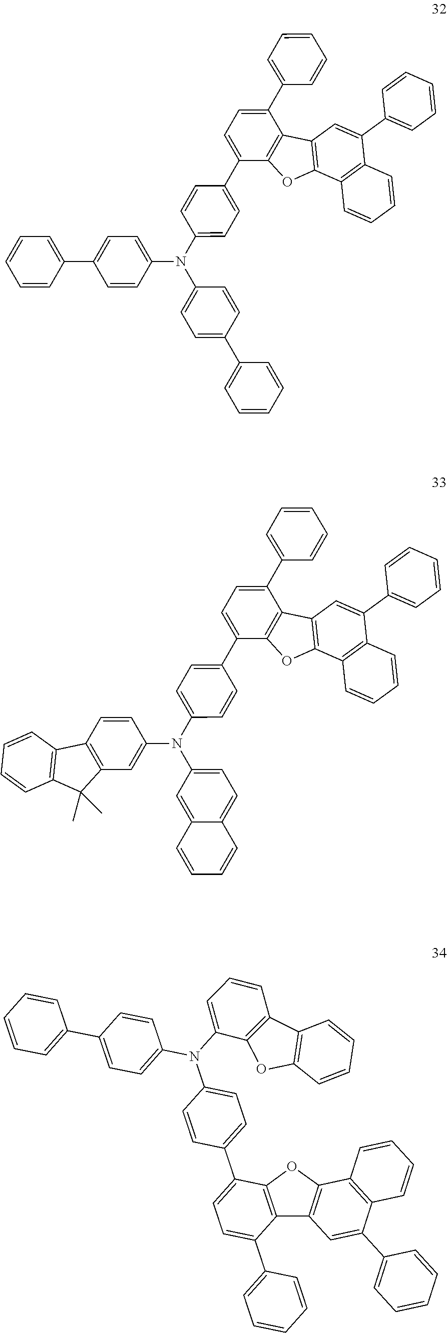 [SMILES: c1ccc(-c2ccc(N(c3ccc(-c4ccccc4)cc3)c3ccc(-c4ccc(-c5ccccc5)c5c4oc4c6ccccc6c(-c6ccccc6)cc45)cc3)cc2)cc1 ; CC1(C)c2ccccc2-c2ccc(N(c3ccc(-c4ccc(-c5ccccc5)c5c4oc4c6ccccc6c(-c6ccccc6)cc45)cc3)c3ccc4ccccc4c3)cc21 ; c1ccc(-c2ccc(N(c3ccc(-c4ccc(-c5ccccc5)c5c4oc4c6ccccc6c(-c6ccccc6)cc45)cc3)c3cccc4c3oc3ccccc34)cc2)cc1]

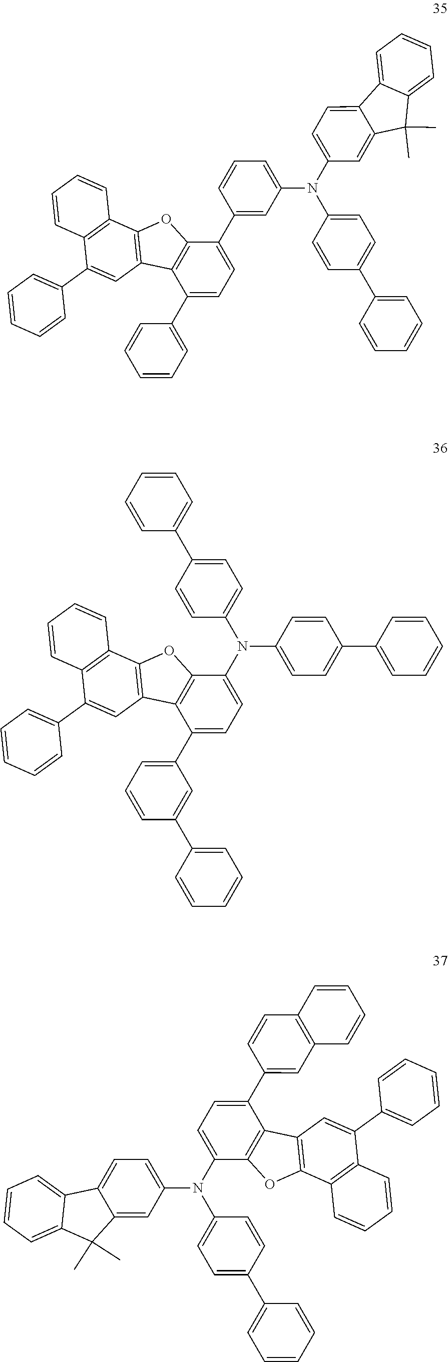 [SMILES: CC1(C)c2ccccc2-c2ccc(N(c3ccc(-c4ccccc4)cc3)c3cccc(-c4ccc(-c5ccccc5)c5c4oc4c6ccccc6c(-c6ccccc6)cc45)c3)cc21 ; c1ccc(-c2ccc(N(c3ccc(-c4ccccc4)cc3)c3ccc(-c4cccc(-c5ccccc5)c4)c4c3oc3c5ccccc5c(-c5ccccc5)cc34)cc2)cc1 ; CC1(C)c2ccccc2-c2ccc(N(c3ccc(-c4ccccc4)cc3)c3ccc(-c4ccc5ccccc5c4)c4c3oc3c5ccccc5c(-c5ccccc5)cc34)cc21]

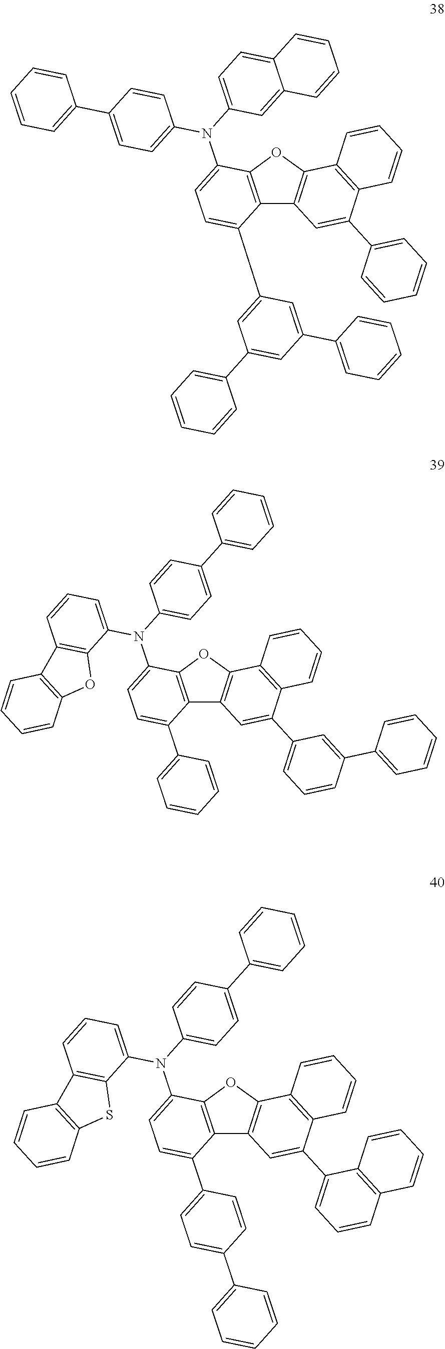 [SMILES: c1ccc(-c2ccc(N(c3ccc4ccccc4c3)c3ccc(-c4cc(-c5ccccc5)cc(-c5ccccc5)c4)c4c3oc3c5ccccc5c(-c5ccccc5)cc34)cc2)cc1 ; c1ccc(-c2ccc(N(c3cccc4c3oc3ccccc34)c3ccc(-c4ccccc4)c4c3oc3c5ccccc5c(-c5cccc(-c6ccccc6)c5)cc34)cc2)cc1 ; c1ccc(-c2ccc(-c3ccc(N(c4ccc(-c5ccccc5)cc4)c4cccc5c4sc4ccccc45)c4oc5c6ccccc6c(-c6cccc7ccccc67)cc5c34)cc2)cc1]

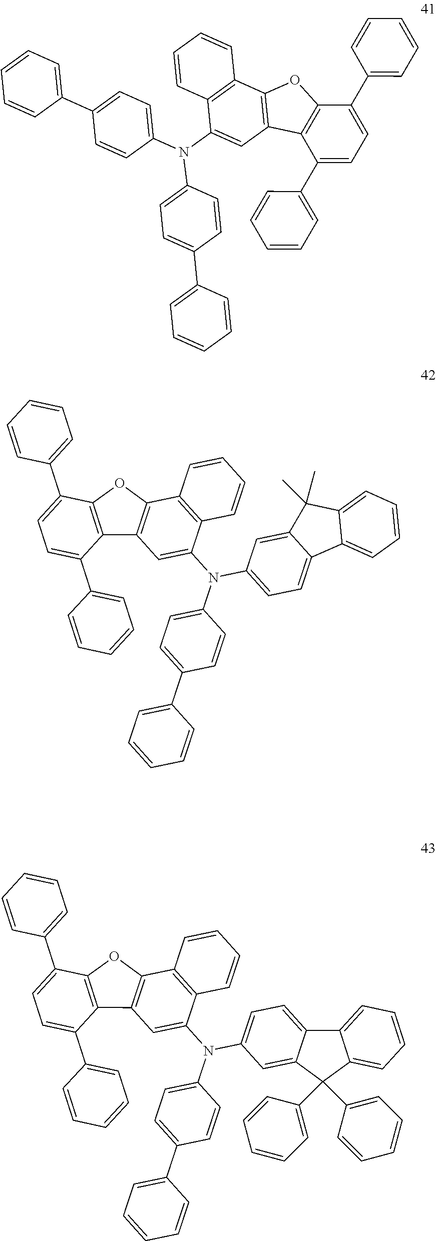 [SMILES: c1ccc(-c2ccc(N(c3ccc(-c4ccccc4)cc3)c3cc4c(oc5c(-c6ccccc6)ccc(-c6ccccc6)c54)c4ccccc34)cc2)cc1 ; CC1(C)c2ccccc2-c2ccc(N(c3ccc(-c4ccccc4)cc3)c3cc4c(oc5c(-c6ccccc6)ccc(-c6ccccc6)c54)c4ccccc34)cc21 ; c1ccc(-c2ccc(N(c3ccc4c(c3)C(c3ccccc3)(c3ccccc3)c3ccccc3-4)c3cc4c(oc5c(-c6ccccc6)ccc(-c6ccccc6)c54)c4ccccc34)cc2)cc1]

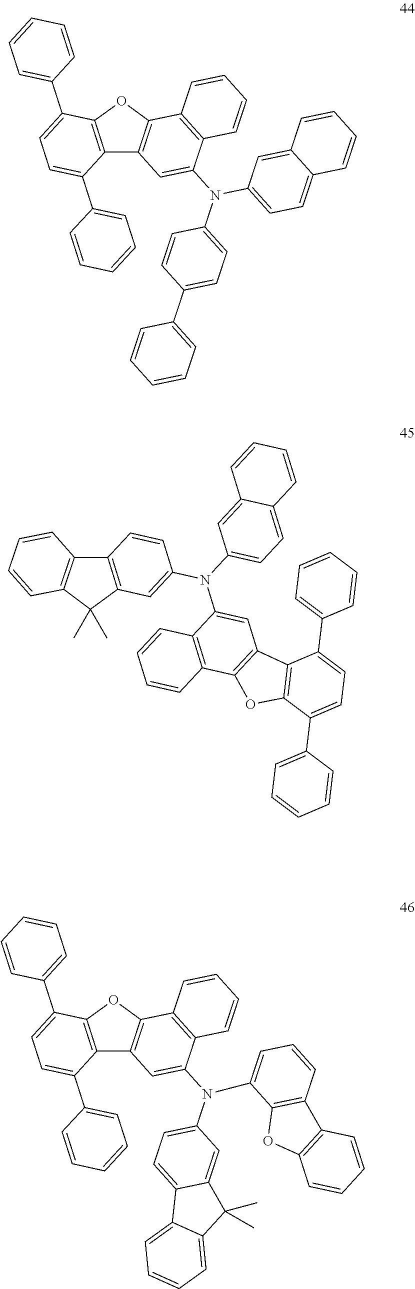 [SMILES: c1ccc(-c2ccc(N(c3ccc4ccccc4c3)c3cc4c(oc5c(-c6ccccc6)ccc(-c6ccccc6)c54)c4ccccc34)cc2)cc1 ; CC1(C)c2ccccc2-c2ccc(N(c3ccc4ccccc4c3)c3cc4c(oc5c(-c6ccccc6)ccc(-c6ccccc6)c54)c4ccccc34)cc21 ; CC1(C)c2ccccc2-c2ccc(N(c3cc4c(oc5c(-c6ccccc6)ccc(-c6ccccc6)c54)c4ccccc34)c3cccc4c3oc3ccccc34)cc21]

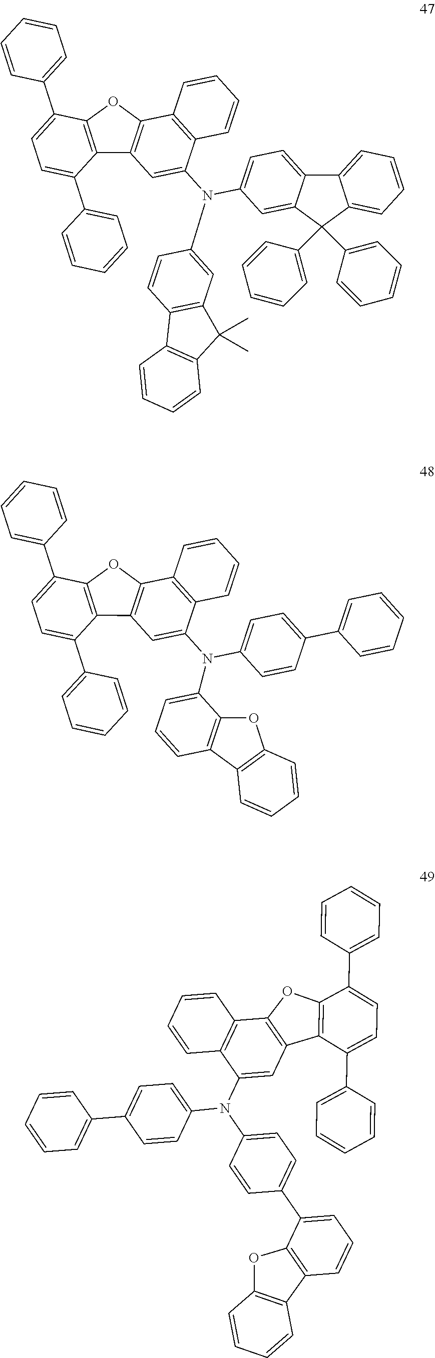 [SMILES: CC1(C)c2ccccc2-c2ccc(N(c3ccc4c(c3)C(c3ccccc3)(c3ccccc3)c3ccccc3-4)c3cc4c(oc5c(-c6ccccc6)ccc(-c6ccccc6)c54)c4ccccc34)cc21 ; c1ccc(-c2ccc(N(c3cc4c(oc5c(-c6ccccc6)ccc(-c6ccccc6)c54)c4ccccc34)c3cccc4c3oc3ccccc34)cc2)cc1 ; c1ccc(-c2ccc(N(c3ccc(-c4cccc5c4oc4ccccc45)cc3)c3cc4c(oc5c(-c6ccccc6)ccc(-c6ccccc6)c54)c4ccccc34)cc2)cc1]

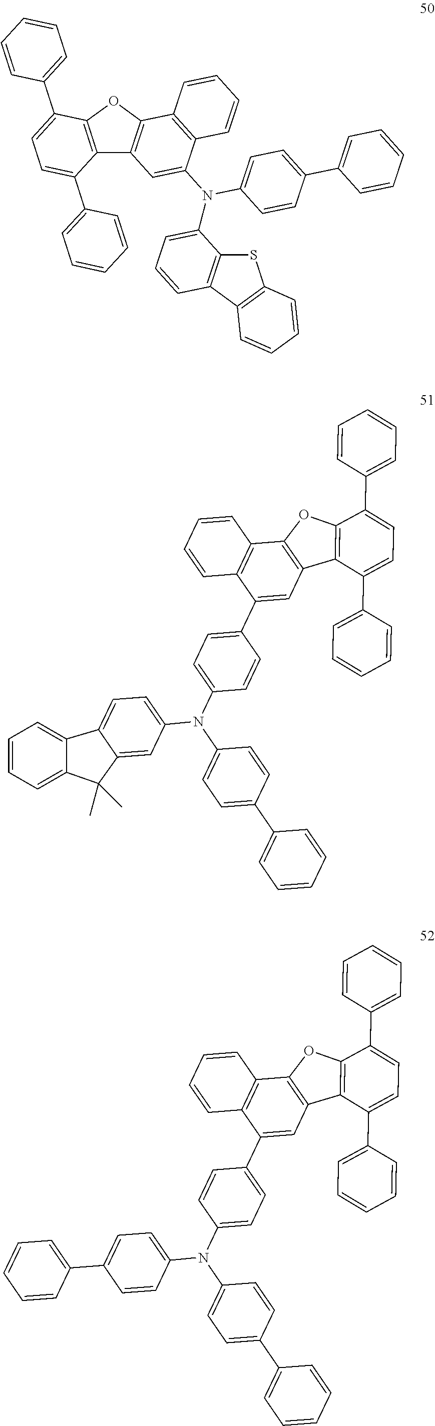 [SMILES: c1ccc(-c2ccc(N(c3cc4c(oc5c(-c6ccccc6)ccc(-c6ccccc6)c54)c4ccccc34)c3cccc4c3sc3ccccc34)cc2)cc1 ; CC1(C)c2ccccc2-c2ccc(N(c3ccc(-c4ccccc4)cc3)c3ccc(-c4cc5c(oc6c(-c7ccccc7)ccc(-c7ccccc7)c65)c5ccccc45)cc3)cc21 ; c1ccc(-c2ccc(N(c3ccc(-c4ccccc4)cc3)c3ccc(-c4cc5c(oc6c(-c7ccccc7)ccc(-c7ccccc7)c65)c5ccccc45)cc3)cc2)cc1]

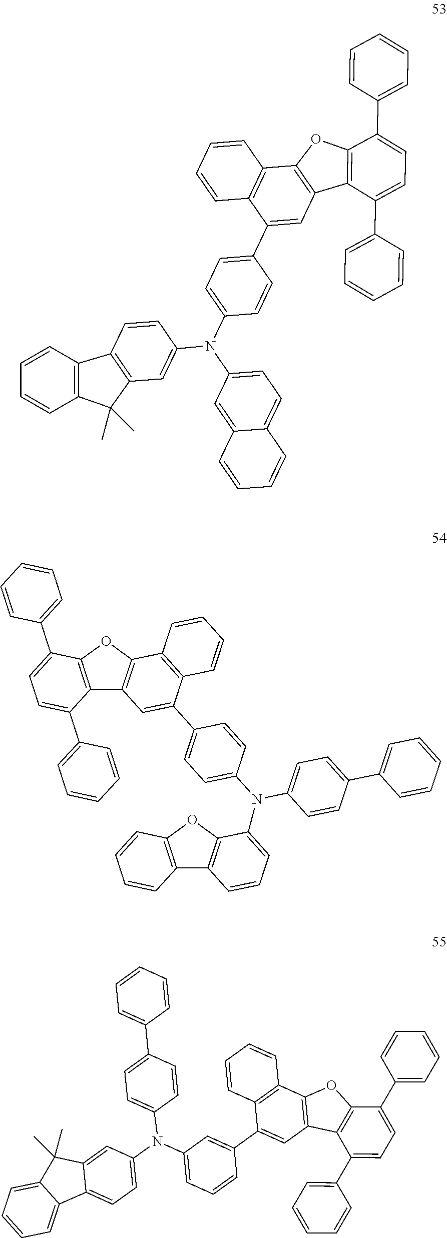 [SMILES: CC1(C)c2ccccc2-c2ccc(N(c3ccc(-c4cc5c(oc6c(-c7ccccc7)ccc(-c7ccccc7)c65)c5ccccc45)cc3)c3ccc4ccccc4c3)cc21 ; c1ccc(-c2ccc(N(c3ccc(-c4cc5c(oc6c(-c7ccccc7)ccc(-c7ccccc7)c65)c5ccccc45)cc3)c3cccc4c3oc3ccccc34)cc2)cc1 ; CC1(C)c2ccccc2-c2ccc(N(c3ccc(-c4ccccc4)cc3)c3cccc(-c4cc5c(oc6c(-c7ccccc7)ccc(-c7ccccc7)c65)c5ccccc45)c3)cc21]

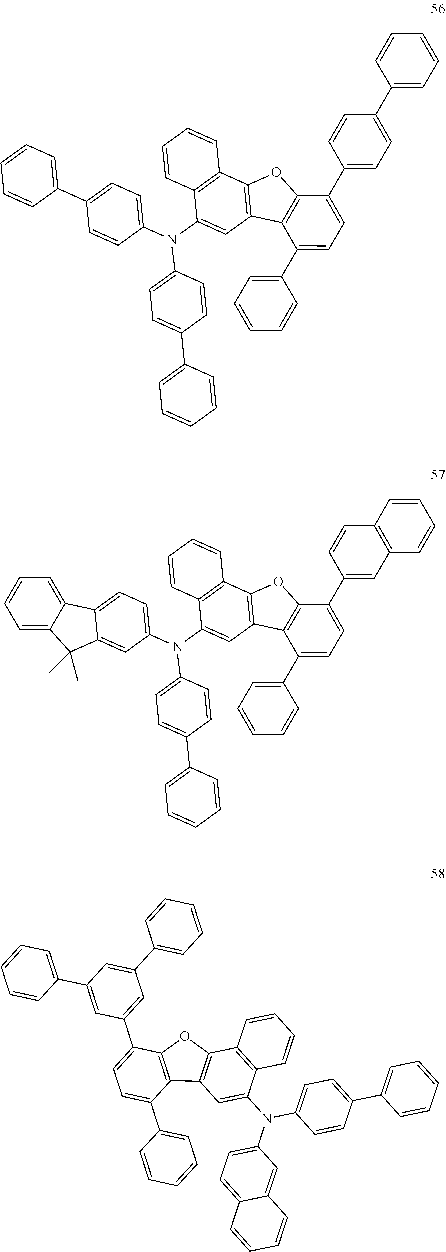 [SMILES: c1ccc(-c2ccc(-c3ccc(-c4ccccc4)c4c3oc3c5ccccc5c(N(c5ccc(-c6ccccc6)cc5)c5ccc(-c6ccccc6)cc5)cc34)cc2)cc1 ; CC1(C)c2ccccc2-c2ccc(N(c3ccc(-c4ccccc4)cc3)c3cc4c(oc5c(-c6ccc7ccccc7c6)ccc(-c6ccccc6)c54)c4ccccc34)cc21 ; c1ccc(-c2ccc(N(c3ccc4ccccc4c3)c3cc4c(oc5c(-c6cc(-c7ccccc7)cc(-c7ccccc7)c6)ccc(-c6ccccc6)c54)c4ccccc34)cc2)cc1]

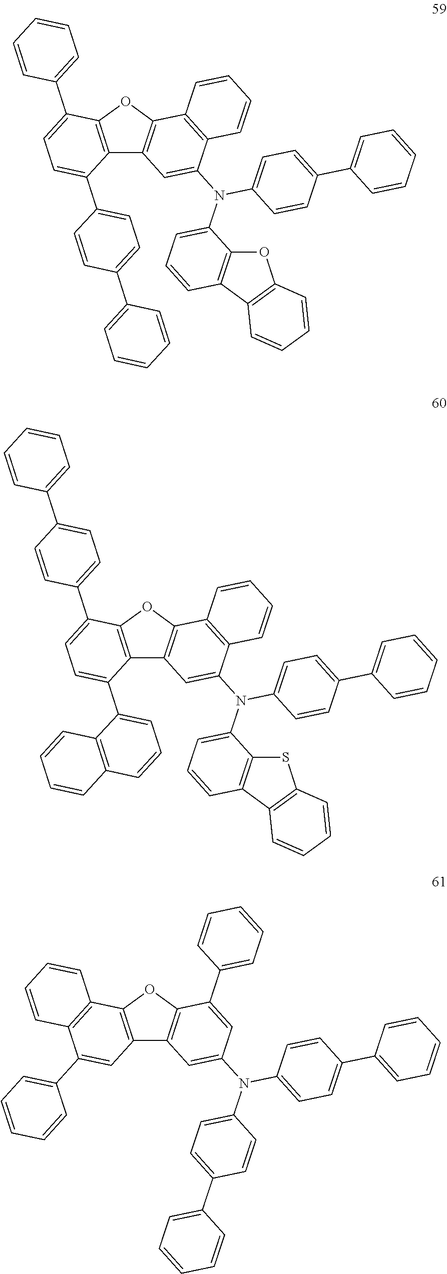 [SMILES: c1ccc(-c2ccc(-c3ccc(-c4ccccc4)c4oc5c6ccccc6c(N(c6ccc(-c7ccccc7)cc6)c6cccc7c6oc6ccccc67)cc5c34)cc2)cc1 ; c1ccc(-c2ccc(-c3ccc(-c4cccc5ccccc45)c4c3oc3c5ccccc5c(N(c5ccc(-c6ccccc6)cc5)c5cccc6c5sc5ccccc56)cc34)cc2)cc1 ; c1ccc(-c2ccc(N(c3ccc(-c4ccccc4)cc3)c3cc(-c4ccccc4)c4oc5c6ccccc6c(-c6ccccc6)cc5c4c3)cc2)cc1]

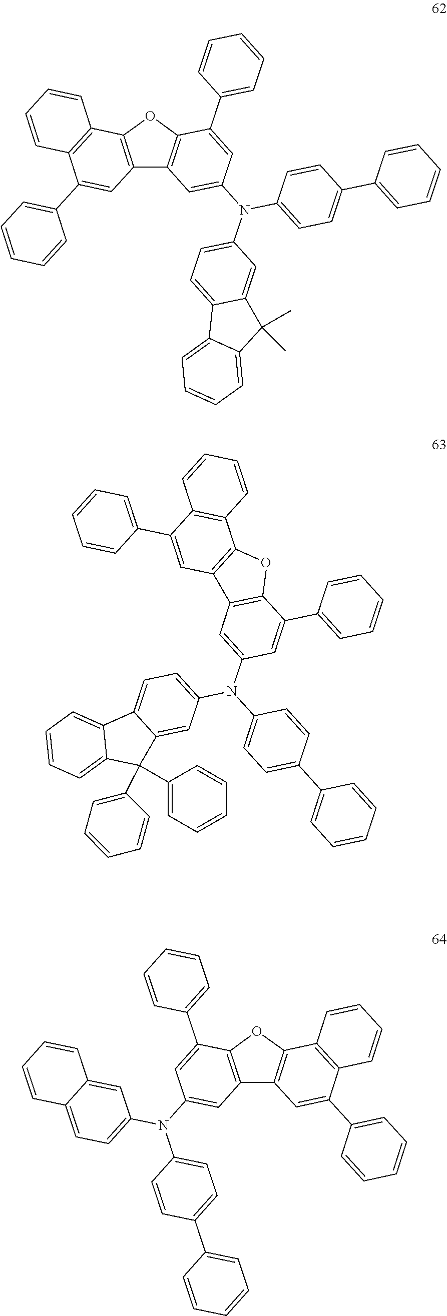 [SMILES: CC1(C)c2ccccc2-c2ccc(N(c3ccc(-c4ccccc4)cc3)c3cc(-c4ccccc4)c4oc5c6ccccc6c(-c6ccccc6)cc5c4c3)cc21 ; c1ccc(-c2ccc(N(c3ccc4c(c3)C(c3ccccc3)(c3ccccc3)c3ccccc3-4)c3cc(-c4ccccc4)c4oc5c6ccccc6c(-c6ccccc6)cc5c4c3)cc2)cc1 ; c1ccc(-c2ccc(N(c3ccc4ccccc4c3)c3cc(-c4ccccc4)c4oc5c6ccccc6c(-c6ccccc6)cc5c4c3)cc2)cc1]

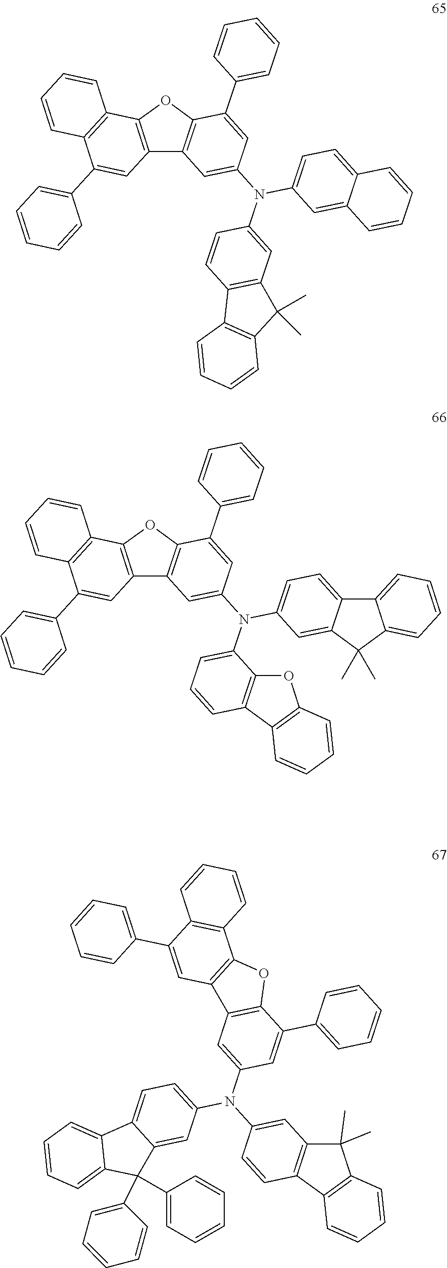 [SMILES: CC1(C)c2ccccc2-c2ccc(N(c3ccc4ccccc4c3)c3cc(-c4ccccc4)c4oc5c6ccccc6c(-c6ccccc6)cc5c4c3)cc21 ; CC1(C)c2ccccc2-c2ccc(N(c3cc(-c4ccccc4)c4oc5c6ccccc6c(-c6ccccc6)cc5c4c3)c3cccc4c3oc3ccccc34)cc21 ; CC1(C)c2ccccc2-c2ccc(N(c3ccc4c(c3)C(c3ccccc3)(c3ccccc3)c3ccccc3-4)c3cc(-c4ccccc4)c4oc5c6ccccc6c(-c6ccccc6)cc5c4c3)cc21]

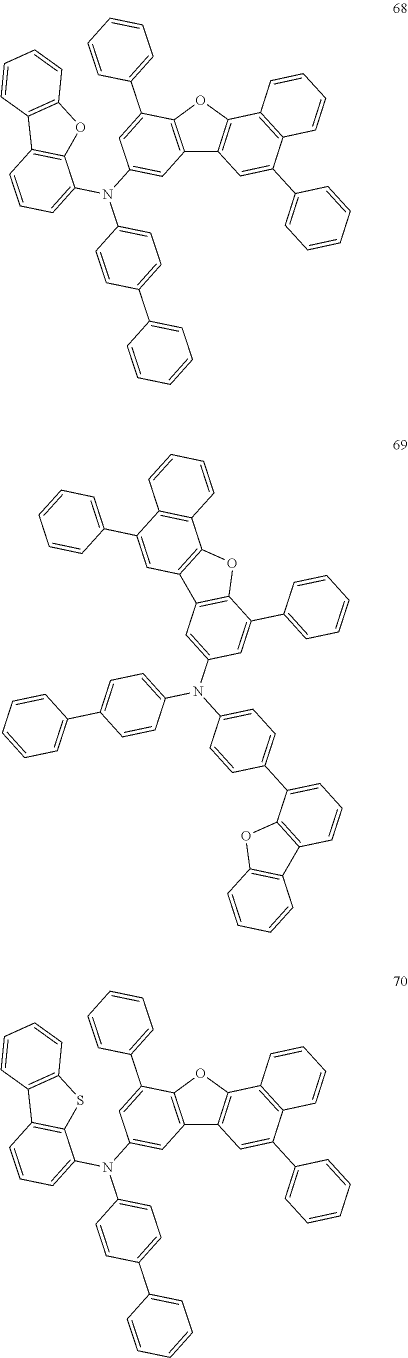 [SMILES: c1ccc(-c2ccc(N(c3cc(-c4ccccc4)c4oc5c6ccccc6c(-c6ccccc6)cc5c4c3)c3cccc4c3oc3ccccc34)cc2)cc1 ; c1ccc(-c2ccc(N(c3ccc(-c4cccc5c4oc4ccccc45)cc3)c3cc(-c4ccccc4)c4oc5c6ccccc6c(-c6ccccc6)cc5c4c3)cc2)cc1 ; c1ccc(-c2ccc(N(c3cc(-c4ccccc4)c4oc5c6ccccc6c(-c6ccccc6)cc5c4c3)c3cccc4c3sc3ccccc34)cc2)cc1]

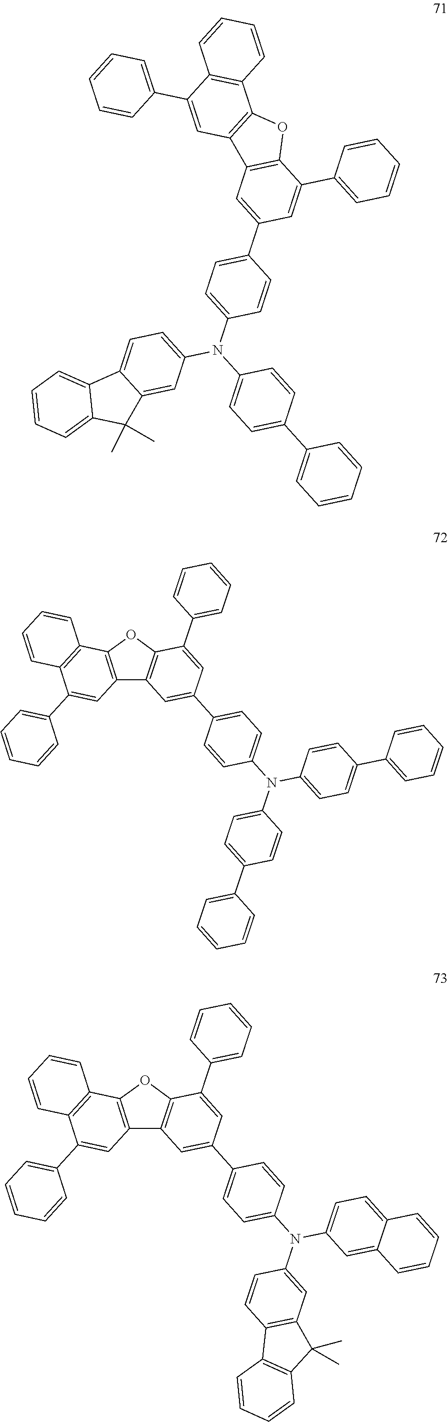 [SMILES: CC1(C)c2ccccc2-c2ccc(N(c3ccc(-c4ccccc4)cc3)c3ccc(-c4cc(-c5ccccc5)c5oc6c7ccccc7c(-c7ccccc7)cc6c5c4)cc3)cc21 ; c1ccc(-c2ccc(N(c3ccc(-c4ccccc4)cc3)c3ccc(-c4cc(-c5ccccc5)c5oc6c7ccccc7c(-c7ccccc7)cc6c5c4)cc3)cc2)cc1 ; CC1(C)c2ccccc2-c2ccc(N(c3ccc(-c4cc(-c5ccccc5)c5oc6c7ccccc7c(-c7ccccc7)cc6c5c4)cc3)c3ccc4ccccc4c3)cc21]

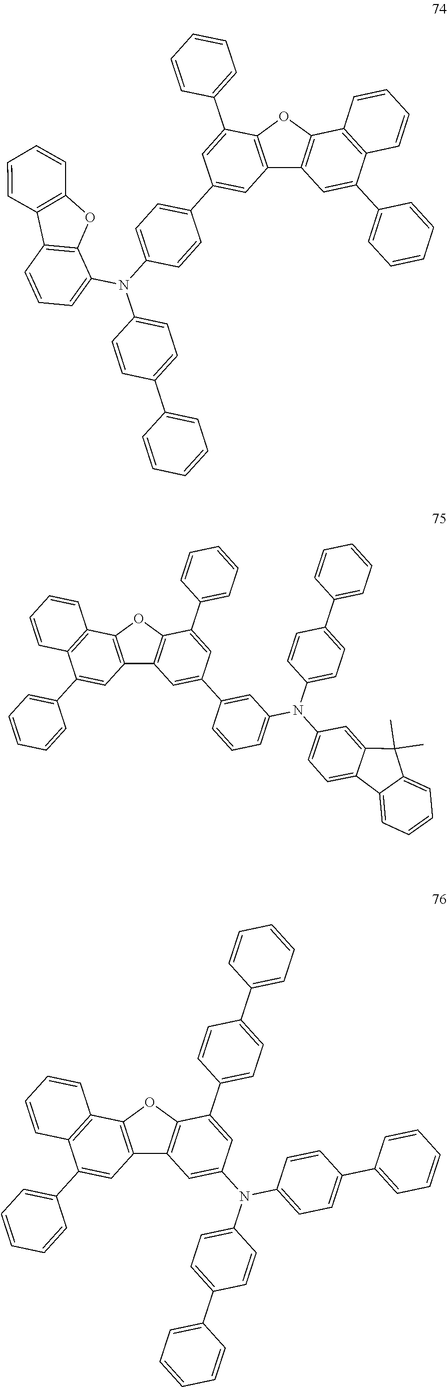 [SMILES: c1ccc(-c2ccc(N(c3ccc(-c4cc(-c5ccccc5)c5oc6c7ccccc7c(-c7ccccc7)cc6c5c4)cc3)c3cccc4c3oc3ccccc34)cc2)cc1 ; CC1(C)c2ccccc2-c2ccc(N(c3ccc(-c4ccccc4)cc3)c3cccc(-c4cc(-c5ccccc5)c5oc6c7ccccc7c(-c7ccccc7)cc6c5c4)c3)cc21 ; c1ccc(-c2ccc(-c3cc(N(c4ccc(-c5ccccc5)cc4)c4ccc(-c5ccccc5)cc4)cc4c3oc3c5ccccc5c(-c5ccccc5)cc43)cc2)cc1]

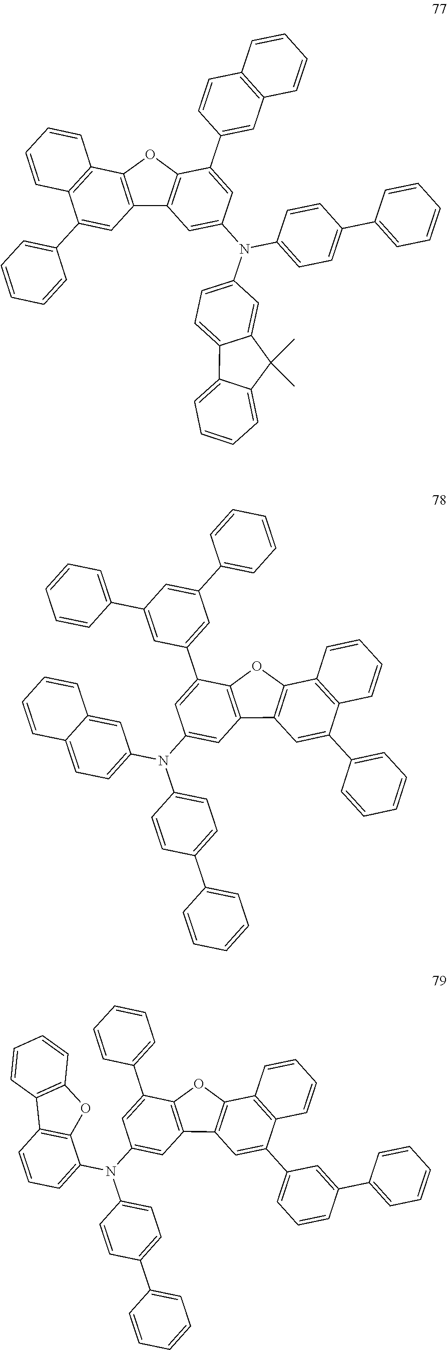 [SMILES: CC1(C)c2ccccc2-c2ccc(N(c3ccc(-c4ccccc4)cc3)c3cc(-c4ccc5ccccc5c4)c4oc5c6ccccc6c(-c6ccccc6)cc5c4c3)cc21 ; c1ccc(-c2ccc(N(c3ccc4ccccc4c3)c3cc(-c4cc(-c5ccccc5)cc(-c5ccccc5)c4)c4oc5c6ccccc6c(-c6ccccc6)cc5c4c3)cc2)cc1 ; c1ccc(-c2ccc(N(c3cc(-c4ccccc4)c4oc5c6ccccc6c(-c6cccc(-c7ccccc7)c6)cc5c4c3)c3cccc4c3oc3ccccc34)cc2)cc1]

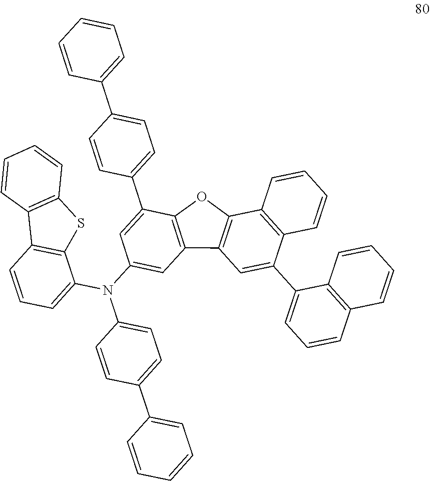 [SMILES: c1ccc(-c2ccc(-c3cc(N(c4ccc(-c5ccccc5)cc4)c4cccc5c4sc4ccccc45)cc4c3oc3c5ccccc5c(-c5cccc6ccccc56)cc43)cc2)cc1]

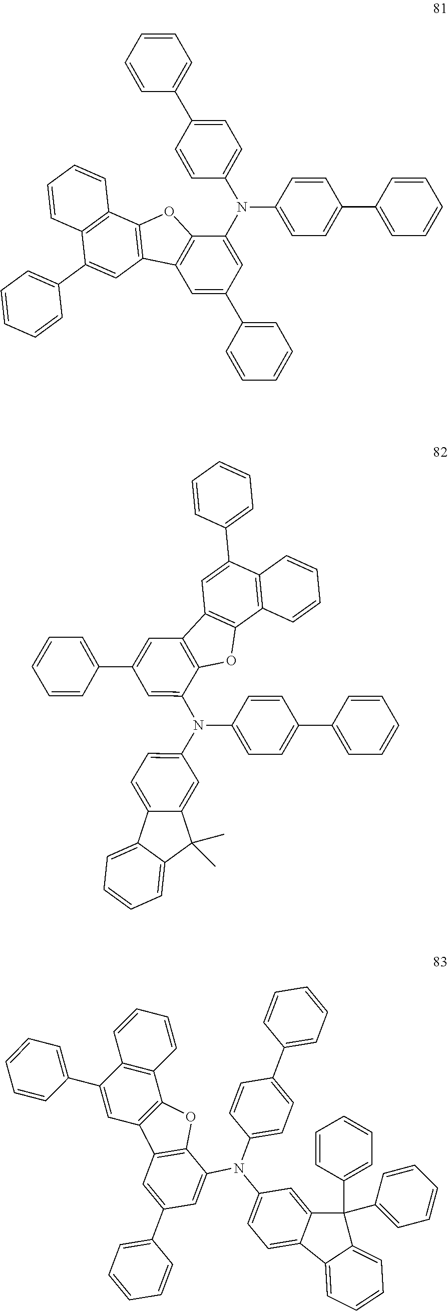 [SMILES: c1ccc(-c2ccc(N(c3ccc(-c4ccccc4)cc3)c3cc(-c4ccccc4)cc4c3oc3c5ccccc5c(-c5ccccc5)cc43)cc2)cc1 ; CC1(C)c2ccccc2-c2ccc(N(c3ccc(-c4ccccc4)cc3)c3cc(-c4ccccc4)cc4c3oc3c5ccccc5c(-c5ccccc5)cc43)cc21 ; c1ccc(-c2ccc(N(c3ccc4c(c3)C(c3ccccc3)(c3ccccc3)c3ccccc3-4)c3cc(-c4ccccc4)cc4c3oc3c5ccccc5c(-c5ccccc5)cc43)cc2)cc1]

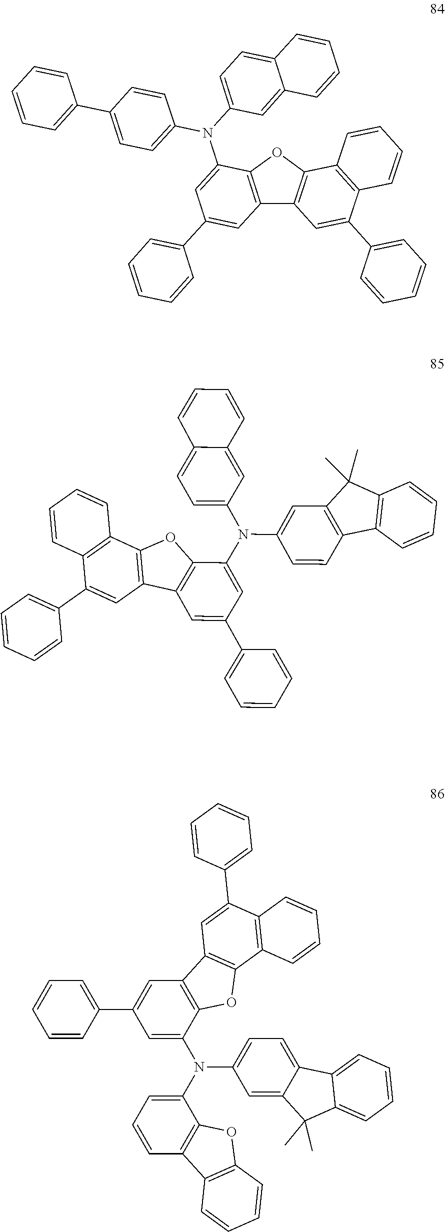 [SMILES: c1ccc(-c2ccc(N(c3ccc4ccccc4c3)c3cc(-c4ccccc4)cc4c3oc3c5ccccc5c(-c5ccccc5)cc43)cc2)cc1 ; CC1(C)c2ccccc2-c2ccc(N(c3ccc4ccccc4c3)c3cc(-c4ccccc4)cc4c3oc3c5ccccc5c(-c5ccccc5)cc43)cc21 ; CC1(C)c2ccccc2-c2ccc(N(c3cccc4c3oc3ccccc34)c3cc(-c4ccccc4)cc4c3oc3c5ccccc5c(-c5ccccc5)cc43)cc21]

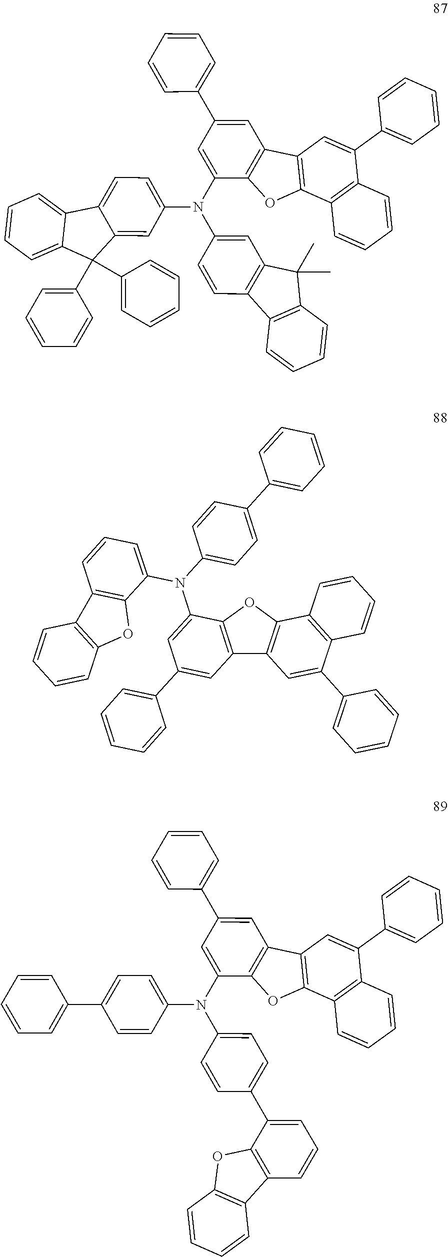 [SMILES: CC1(C)c2ccccc2-c2ccc(N(c3ccc4c(c3)C(c3ccccc3)(c3ccccc3)c3ccccc3-4)c3cc(-c4ccccc4)cc4c3oc3c5ccccc5c(-c5ccccc5)cc43)cc21 ; c1ccc(-c2ccc(N(c3cccc4c3oc3ccccc34)c3cc(-c4ccccc4)cc4c3oc3c5ccccc5c(-c5ccccc5)cc43)cc2)cc1 ; c1ccc(-c2ccc(N(c3ccc(-c4cccc5c4oc4ccccc45)cc3)c3cc(-c4ccccc4)cc4c3oc3c5ccccc5c(-c5ccccc5)cc43)cc2)cc1]

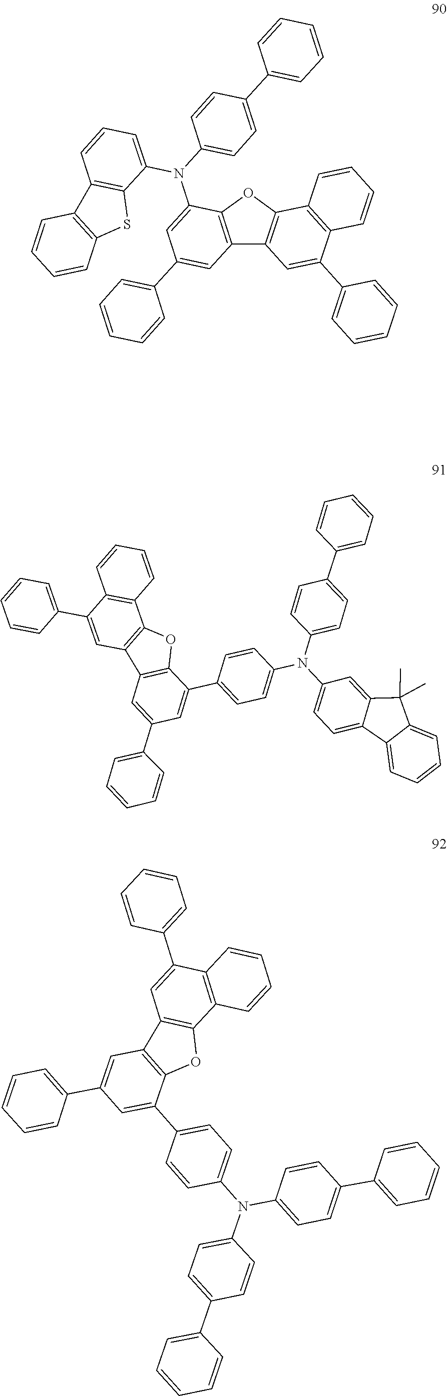 [SMILES: c1ccc(-c2ccc(N(c3cc(-c4ccccc4)cc4c3oc3c5ccccc5c(-c5ccccc5)cc43)c3cccc4c3sc3ccccc34)cc2)cc1 ; CC1(C)c2ccccc2-c2ccc(N(c3ccc(-c4ccccc4)cc3)c3ccc(-c4cc(-c5ccccc5)cc5c4oc4c6ccccc6c(-c6ccccc6)cc54)cc3)cc21 ; c1ccc(-c2ccc(N(c3ccc(-c4ccccc4)cc3)c3ccc(-c4cc(-c5ccccc5)cc5c4oc4c6ccccc6c(-c6ccccc6)cc54)cc3)cc2)cc1]

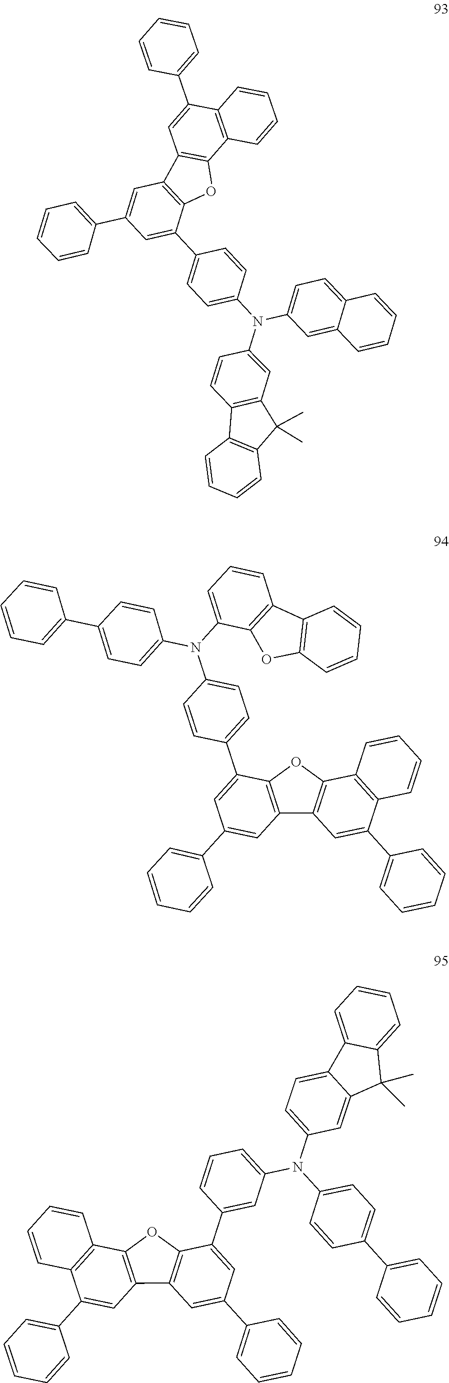 [SMILES: CC1(C)c2ccccc2-c2ccc(N(c3ccc(-c4cc(-c5ccccc5)cc5c4oc4c6ccccc6c(-c6ccccc6)cc54)cc3)c3ccc4ccccc4c3)cc21 ; c1ccc(-c2ccc(N(c3ccc(-c4cc(-c5ccccc5)cc5c4oc4c6ccccc6c(-c6ccccc6)cc54)cc3)c3cccc4c3oc3ccccc34)cc2)cc1 ; CC1(C)c2ccccc2-c2ccc(N(c3ccc(-c4ccccc4)cc3)c3cccc(-c4cc(-c5ccccc5)cc5c4oc4c6ccccc6c(-c6ccccc6)cc54)c3)cc21]

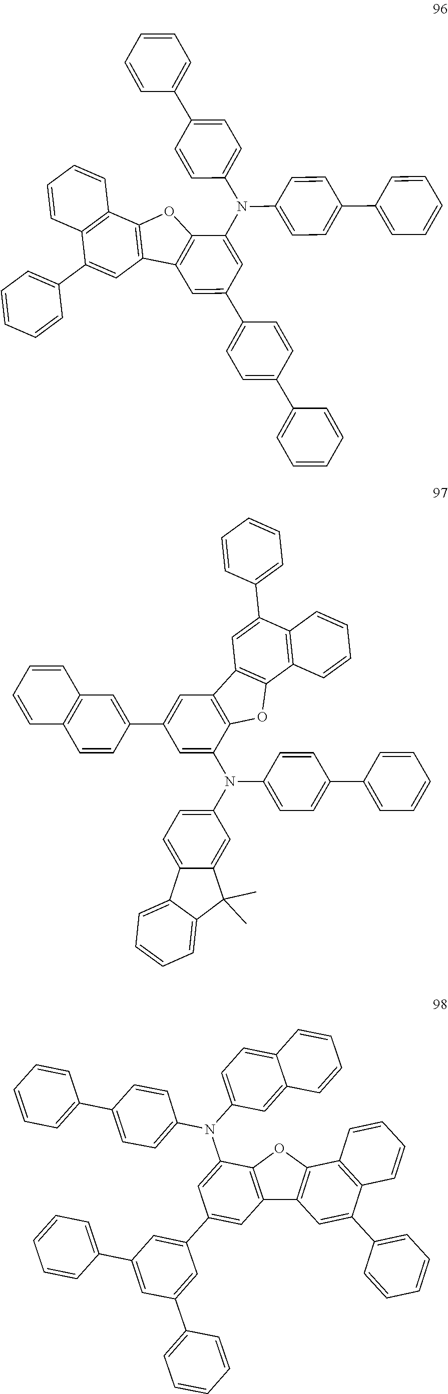 [SMILES: c1ccc(-c2ccc(-c3cc(N(c4ccc(-c5ccccc5)cc4)c4ccc(-c5ccccc5)cc4)c4oc5c6ccccc6c(-c6ccccc6)cc5c4c3)cc2)cc1 ; CC1(C)c2ccccc2-c2ccc(N(c3ccc(-c4ccccc4)cc3)c3cc(-c4ccc5ccccc5c4)cc4c3oc3c5ccccc5c(-c5ccccc5)cc43)cc21 ; c1ccc(-c2ccc(N(c3ccc4ccccc4c3)c3cc(-c4cc(-c5ccccc5)cc(-c5ccccc5)c4)cc4c3oc3c5ccccc5c(-c5ccccc5)cc43)cc2)cc1]

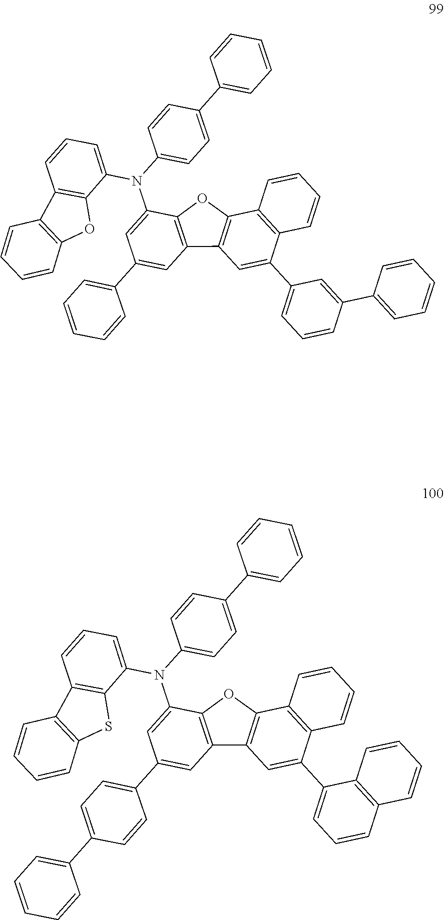 [SMILES: c1ccc(-c2ccc(N(c3cccc4c3oc3ccccc34)c3cc(-c4ccccc4)cc4c3oc3c5ccccc5c(-c5cccc(-c6ccccc6)c5)cc43)cc2)cc1 ; c1ccc(-c2ccc(-c3cc(N(c4ccc(-c5ccccc5)cc4)c4cccc5c4sc4ccccc45)c4oc5c6ccccc6c(-c6cccc7ccccc67)cc5c4c3)cc2)cc1]

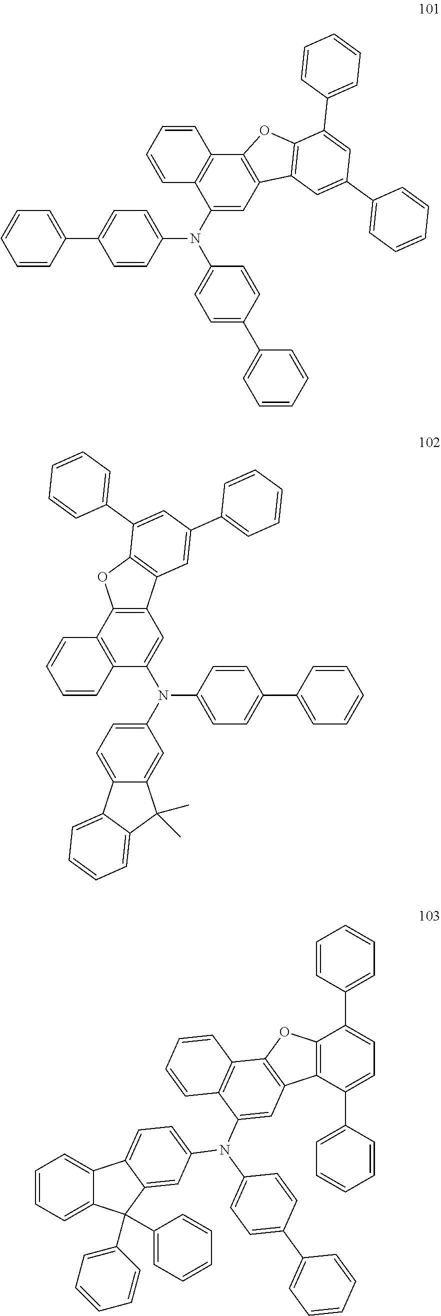 [SMILES: c1ccc(-c2ccc(N(c3ccc(-c4ccccc4)cc3)c3cc4c5cc(-c6ccccc6)cc(-c6ccccc6)c5oc4c4ccccc34)cc2)cc1 ; CC1(C)c2ccccc2-c2ccc(N(c3ccc(-c4ccccc4)cc3)c3cc4c5cc(-c6ccccc6)cc(-c6ccccc6)c5oc4c4ccccc34)cc21 ; c1ccc(-c2ccc(N(c3ccc4c(c3)C(c3ccccc3)(c3ccccc3)c3ccccc3-4)c3cc4c(oc5c(-c6ccccc6)ccc(-c6ccccc6)c54)c4ccccc34)cc2)cc1]

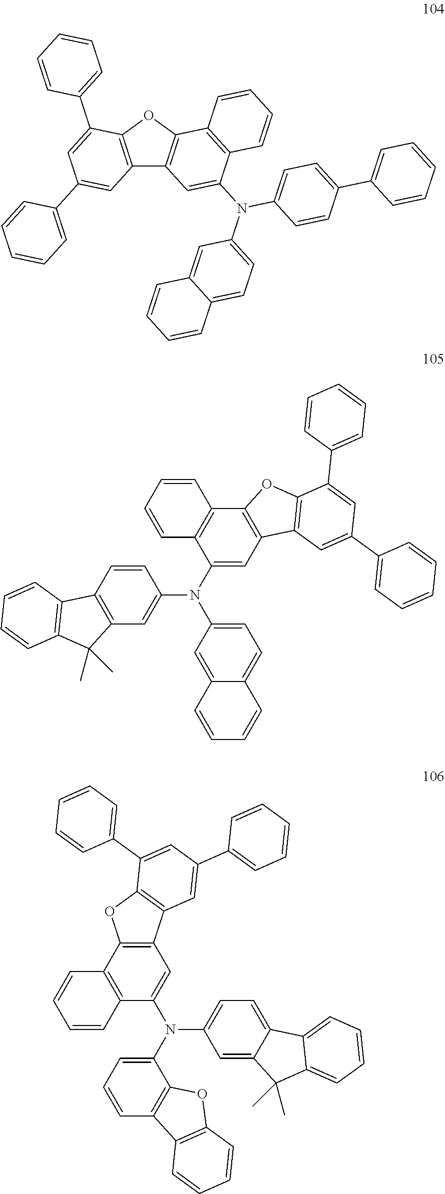 [SMILES: c1ccc(-c2ccc(N(c3ccc4ccccc4c3)c3cc4c5cc(-c6ccccc6)cc(-c6ccccc6)c5oc4c4ccccc34)cc2)cc1 ; CC1(C)c2ccccc2-c2ccc(N(c3ccc4ccccc4c3)c3cc4c5cc(-c6ccccc6)cc(-c6ccccc6)c5oc4c4ccccc34)cc21 ; CC1(C)c2ccccc2-c2ccc(N(c3cc4c5cc(-c6ccccc6)cc(-c6ccccc6)c5oc4c4ccccc34)c3cccc4c3oc3ccccc34)cc21]

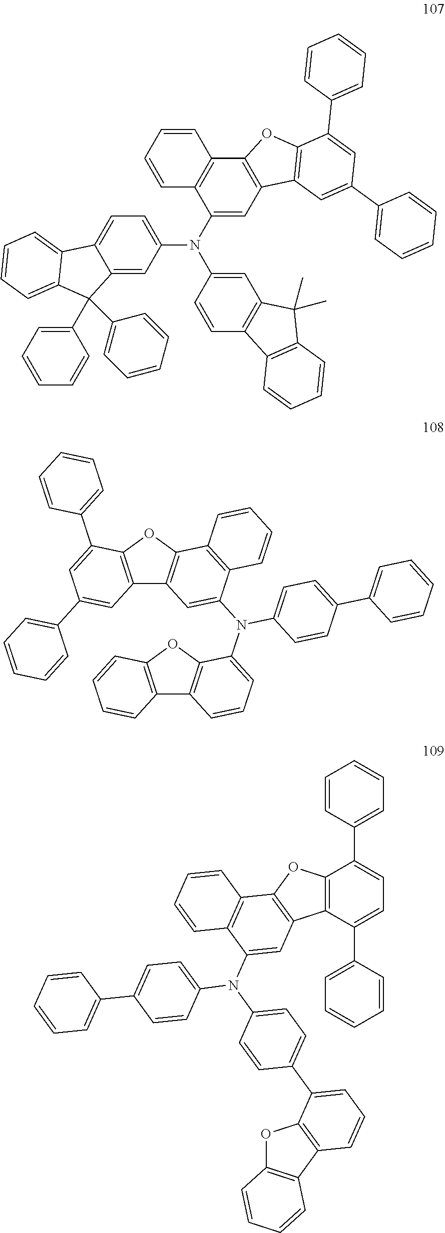 [SMILES: CC1(C)c2ccccc2-c2ccc(N(c3ccc4c(c3)C(c3ccccc3)(c3ccccc3)c3ccccc3-4)c3cc4c5cc(-c6ccccc6)cc(-c6ccccc6)c5oc4c4ccccc34)cc21 ; c1ccc(-c2ccc(N(c3cc4c5cc(-c6ccccc6)cc(-c6ccccc6)c5oc4c4ccccc34)c3cccc4c3oc3ccccc34)cc2)cc1 ; c1ccc(-c2ccc(N(c3ccc(-c4cccc5c4oc4ccccc45)cc3)c3cc4c(oc5c(-c6ccccc6)ccc(-c6ccccc6)c54)c4ccccc34)cc2)cc1]

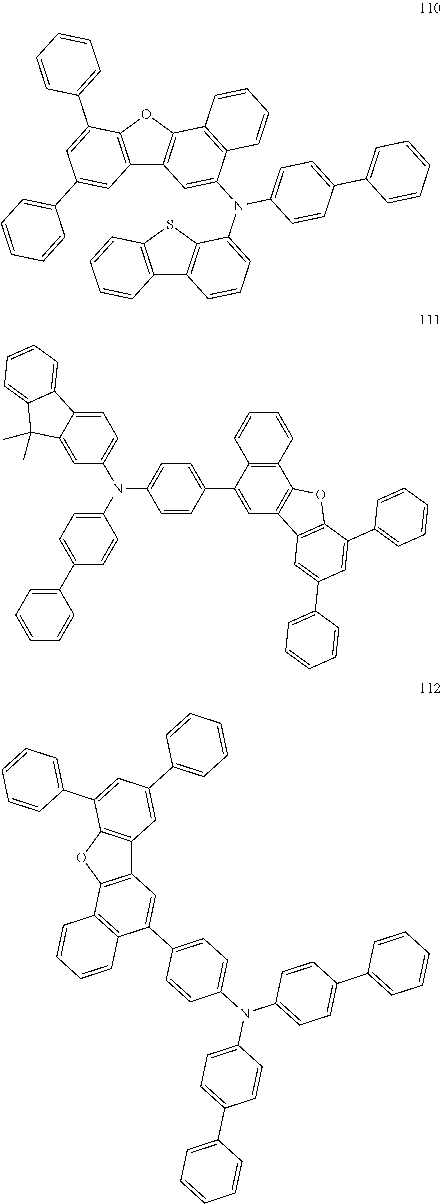 [SMILES: c1ccc(-c2ccc(N(c3cc4c5cc(-c6ccccc6)cc(-c6ccccc6)c5oc4c4ccccc34)c3cccc4c3sc3ccccc34)cc2)cc1 ; CC1(C)c2ccccc2-c2ccc(N(c3ccc(-c4ccccc4)cc3)c3ccc(-c4cc5c6cc(-c7ccccc7)cc(-c7ccccc7)c6oc5c5ccccc45)cc3)cc21 ; c1ccc(-c2ccc(N(c3ccc(-c4ccccc4)cc3)c3ccc(-c4cc5c6cc(-c7ccccc7)cc(-c7ccccc7)c6oc5c5ccccc45)cc3)cc2)cc1]

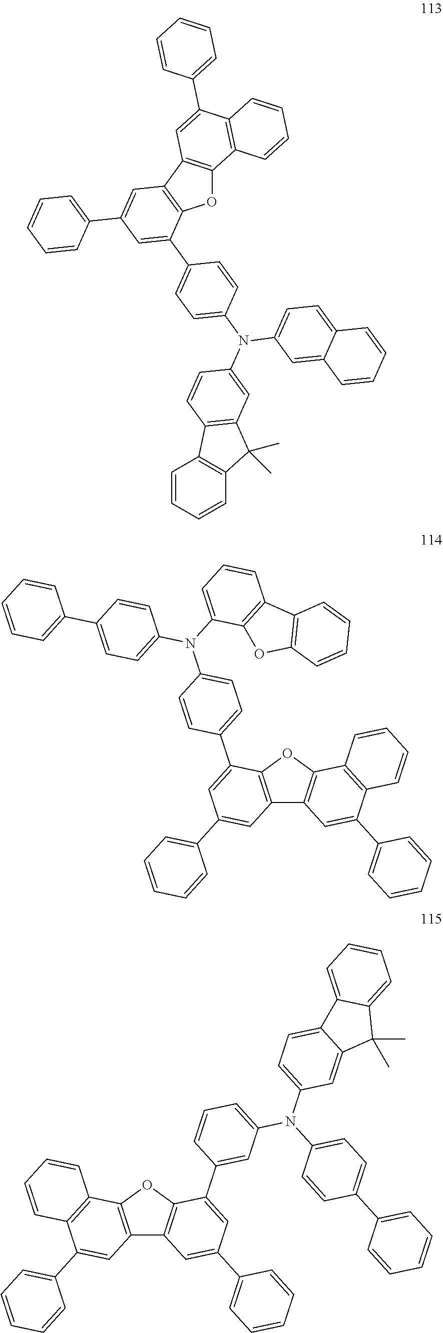 [SMILES: CC1(C)c2ccccc2-c2ccc(N(c3ccc(-c4cc(-c5ccccc5)cc5c4oc4c6ccccc6c(-c6ccccc6)cc54)cc3)c3ccc4ccccc4c3)cc21 ; c1ccc(-c2ccc(N(c3ccc(-c4cc(-c5ccccc5)cc5c4oc4c6ccccc6c(-c6ccccc6)cc54)cc3)c3cccc4c3oc3ccccc34)cc2)cc1 ; CC1(C)c2ccccc2-c2ccc(N(c3ccc(-c4ccccc4)cc3)c3cccc(-c4cc(-c5ccccc5)cc5c4oc4c6ccccc6c(-c6ccccc6)cc54)c3)cc21]

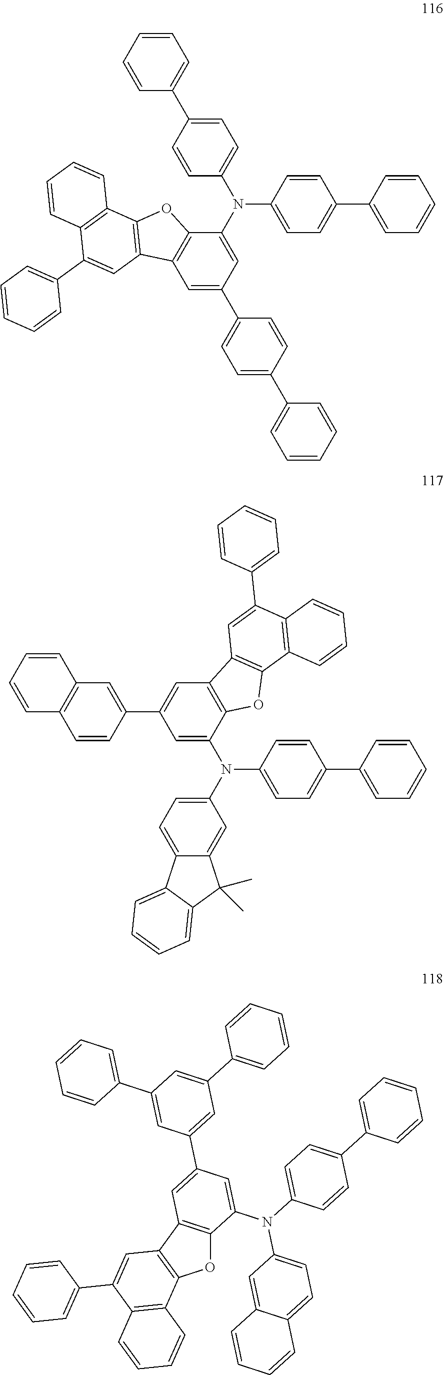 [SMILES: c1ccc(-c2ccc(-c3cc(N(c4ccc(-c5ccccc5)cc4)c4ccc(-c5ccccc5)cc4)c4oc5c6ccccc6c(-c6ccccc6)cc5c4c3)cc2)cc1 ; CC1(C)c2ccccc2-c2ccc(N(c3ccc(-c4ccccc4)cc3)c3cc(-c4ccc5ccccc5c4)cc4c3oc3c5ccccc5c(-c5ccccc5)cc43)cc21 ; c1ccc(-c2ccc(N(c3ccc4ccccc4c3)c3cc(-c4cc(-c5ccccc5)cc(-c5ccccc5)c4)cc4c3oc3c5ccccc5c(-c5ccccc5)cc43)cc2)cc1]

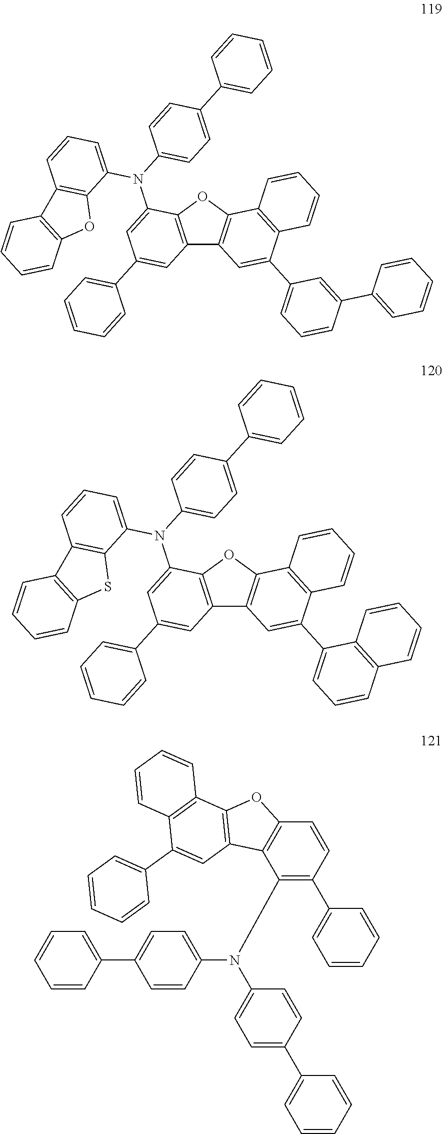 [SMILES: c1ccc(-c2ccc(N(c3cccc4c3oc3ccccc34)c3cc(-c4ccccc4)cc4c3oc3c5ccccc5c(-c5cccc(-c6ccccc6)c5)cc43)cc2)cc1 ; c1ccc(-c2ccc(N(c3cc(-c4ccccc4)cc4c3oc3c5ccccc5c(-c5cccc6ccccc56)cc43)c3cccc4c3sc3ccccc34)cc2)cc1 ; c1ccc(-c2ccc(N(c3ccc(-c4ccccc4)cc3)c3c(-c4ccccc4)ccc4oc5c6ccccc6c(-c6ccccc6)cc5c34)cc2)cc1]

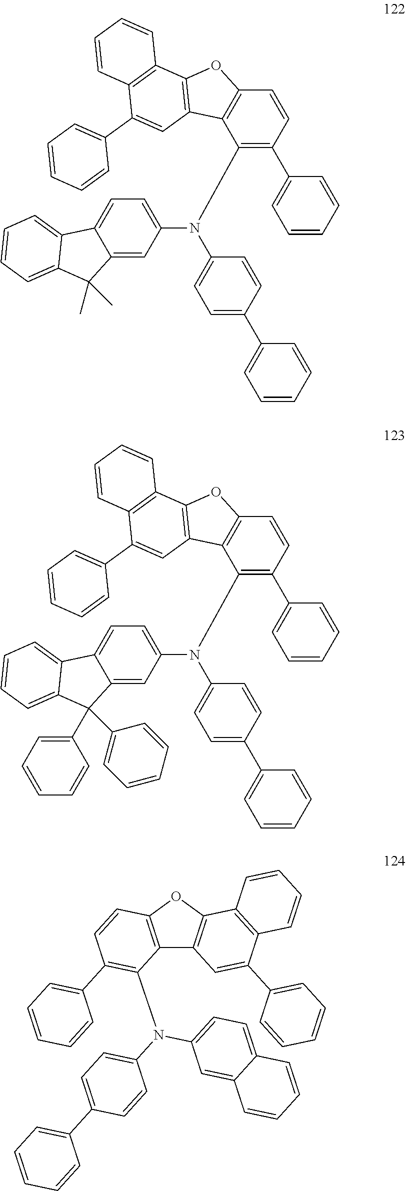

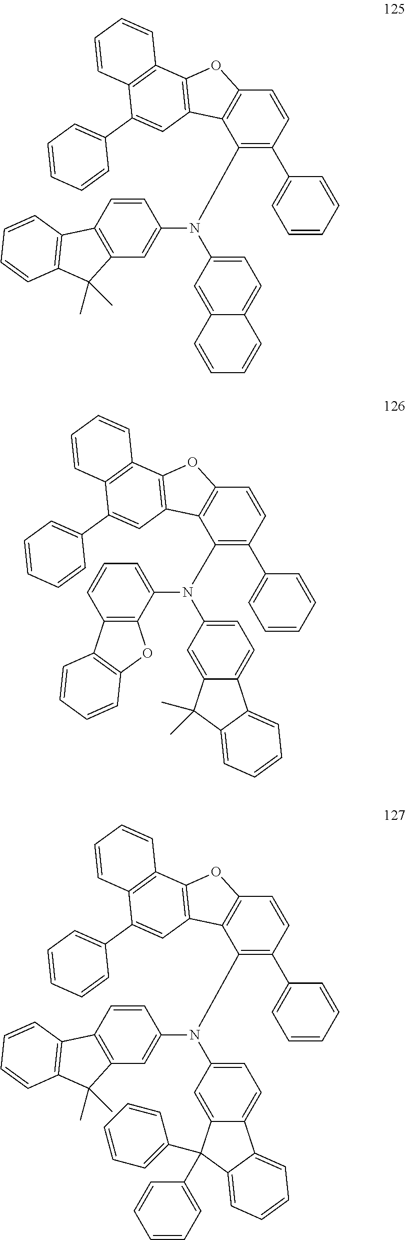 [SMILES: CC1(C)c2ccccc2-c2ccc(N(c3ccc4ccccc4c3)c3c(-c4ccccc4)ccc4oc5c6ccccc6c(-c6ccccc6)cc5c34)cc21 ; CC1(C)c2ccccc2-c2ccc(N(c3cccc4c3oc3ccccc34)c3c(-c4ccccc4)ccc4oc5c6ccccc6c(-c6ccccc6)cc5c34)cc21 ; CC1(C)c2ccccc2-c2ccc(N(c3ccc4c(c3)C(c3ccccc3)(c3ccccc3)c3ccccc3-4)c3c(-c4ccccc4)ccc4oc5c6ccccc6c(-c6ccccc6)cc5c34)cc21]

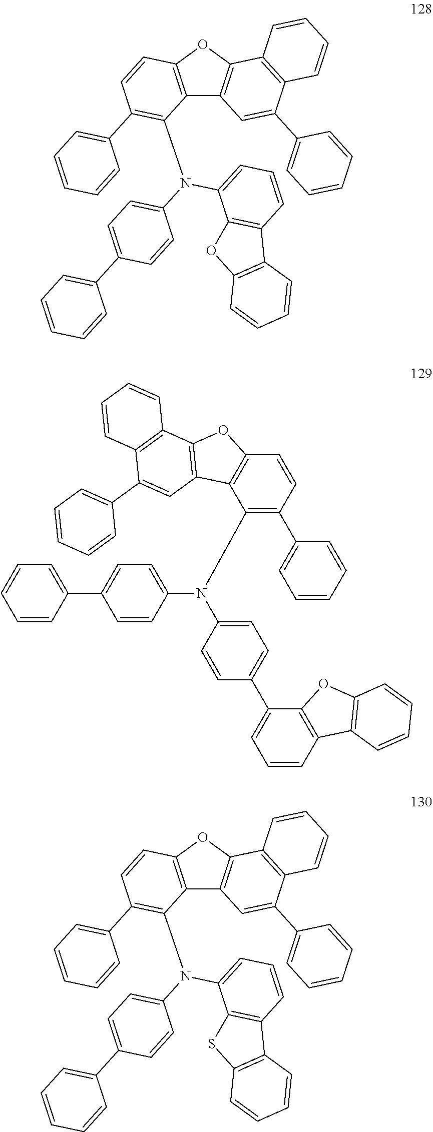

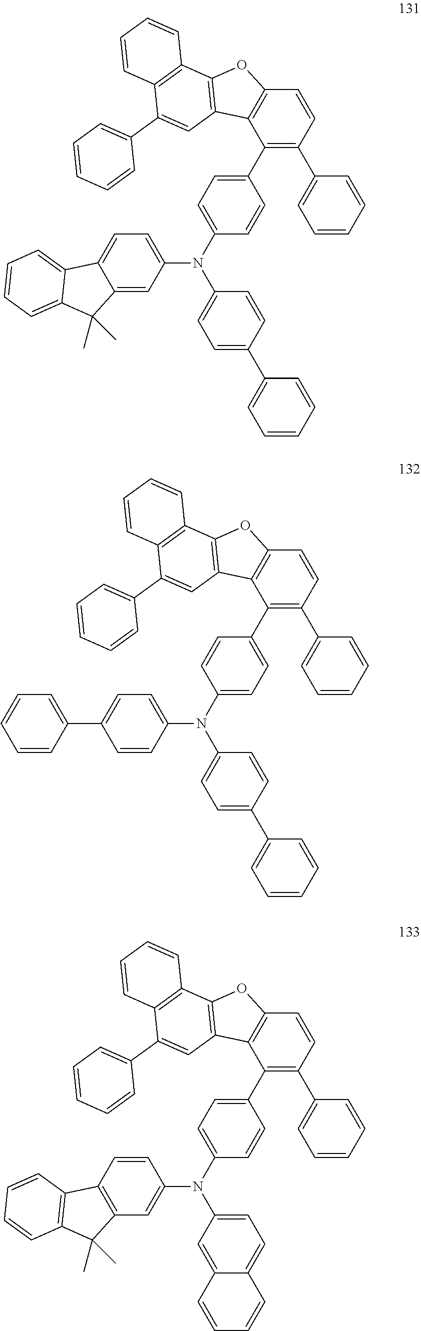 [SMILES: CC1(C)c2ccccc2-c2ccc(N(c3ccc(-c4ccccc4)cc3)c3ccc(-c4c(-c5ccccc5)ccc5oc6c7ccccc7c(-c7ccccc7)cc6c45)cc3)cc21 ; c1ccc(-c2ccc(N(c3ccc(-c4ccccc4)cc3)c3ccc(-c4c(-c5ccccc5)ccc5oc6c7ccccc7c(-c7ccccc7)cc6c45)cc3)cc2)cc1 ; CC1(C)c2ccccc2-c2ccc(N(c3ccc(-c4c(-c5ccccc5)ccc5oc6c7ccccc7c(-c7ccccc7)cc6c45)cc3)c3ccc4ccccc4c3)cc21]

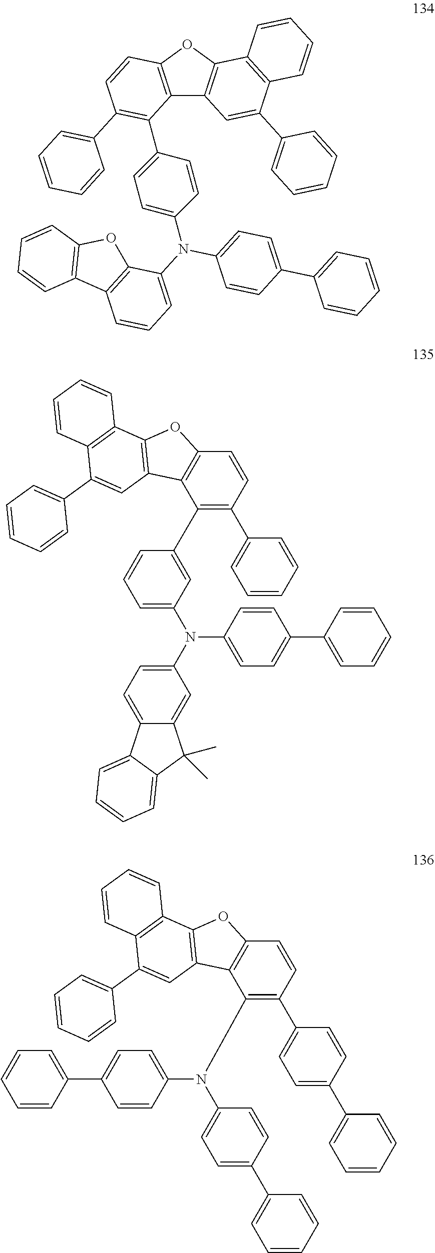 [SMILES: c1ccc(-c2ccc(N(c3ccc(-c4c(-c5ccccc5)ccc5oc6c7ccccc7c(-c7ccccc7)cc6c45)cc3)c3cccc4c3oc3ccccc34)cc2)cc1 ; CC1(C)c2ccccc2-c2ccc(N(c3ccc(-c4ccccc4)cc3)c3cccc(-c4c(-c5ccccc5)ccc5oc6c7ccccc7c(-c7ccccc7)cc6c45)c3)cc21 ; c1ccc(-c2ccc(-c3ccc4oc5c6ccccc6c(-c6ccccc6)cc5c4c3N(c3ccc(-c4ccccc4)cc3)c3ccc(-c4ccccc4)cc3)cc2)cc1]

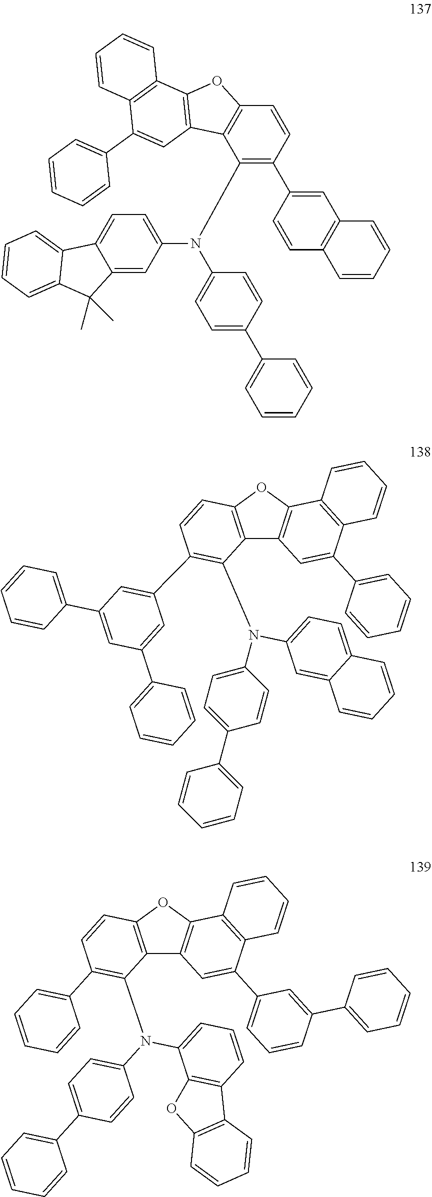 [SMILES: CC1(C)c2ccccc2-c2ccc(N(c3ccc(-c4ccccc4)cc3)c3c(-c4ccc5ccccc5c4)ccc4oc5c6ccccc6c(-c6ccccc6)cc5c34)cc21 ; c1ccc(-c2ccc(N(c3ccc4ccccc4c3)c3c(-c4cc(-c5ccccc5)cc(-c5ccccc5)c4)ccc4oc5c6ccccc6c(-c6ccccc6)cc5c34)cc2)cc1 ; c1ccc(-c2ccc(N(c3cccc4c3oc3ccccc34)c3c(-c4ccccc4)ccc4oc5c6ccccc6c(-c6cccc(-c7ccccc7)c6)cc5c34)cc2)cc1]

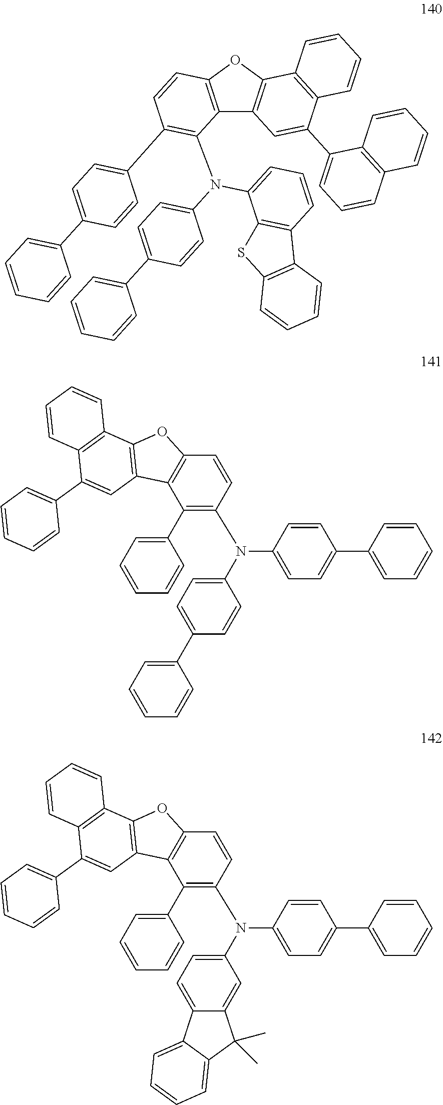 [SMILES: c1ccc(-c2ccc(-c3ccc4oc5c6ccccc6c(-c6cccc7ccccc67)cc5c4c3N(c3ccc(-c4ccccc4)cc3)c3cccc4c3sc3ccccc34)cc2)cc1 ; c1ccc(-c2ccc(N(c3ccc(-c4ccccc4)cc3)c3ccc4oc5c6ccccc6c(-c6ccccc6)cc5c4c3-c3ccccc3)cc2)cc1 ; CC1(C)c2ccccc2-c2ccc(N(c3ccc(-c4ccccc4)cc3)c3ccc4oc5c6ccccc6c(-c6ccccc6)cc5c4c3-c3ccccc3)cc21]

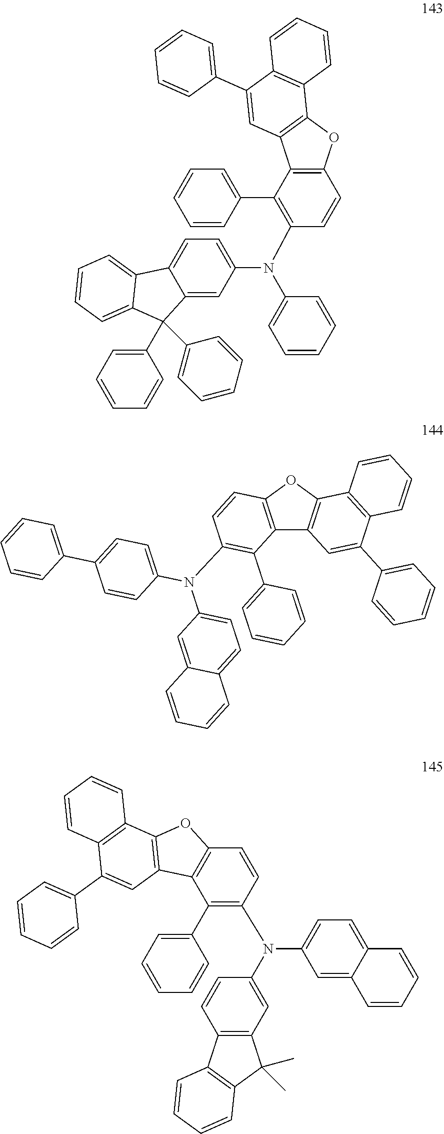 [SMILES: c1ccc(-c2cc3c(oc4ccc(N(c5ccccc5)c5ccc6c(c5)C(c5ccccc5)(c5ccccc5)c5ccccc5-6)c(-c5ccccc5)c43)c3ccccc23)cc1 ; c1ccc(-c2ccc(N(c3ccc4ccccc4c3)c3ccc4oc5c6ccccc6c(-c6ccccc6)cc5c4c3-c3ccccc3)cc2)cc1 ; CC1(C)c2ccccc2-c2ccc(N(c3ccc4ccccc4c3)c3ccc4oc5c6ccccc6c(-c6ccccc6)cc5c4c3-c3ccccc3)cc21]

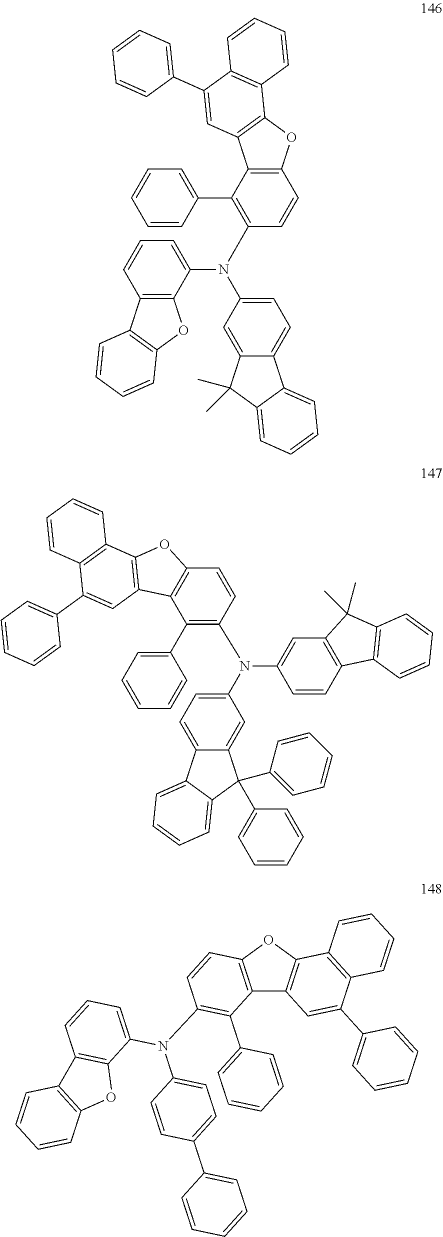 [SMILES: CC1(C)c2ccccc2-c2ccc(N(c3ccc4oc5c6ccccc6c(-c6ccccc6)cc5c4c3-c3ccccc3)c3cccc4c3oc3ccccc34)cc21 ; CC1(C)c2ccccc2-c2ccc(N(c3ccc4c(c3)C(c3ccccc3)(c3ccccc3)c3ccccc3-4)c3ccc4oc5c6ccccc6c(-c6ccccc6)cc5c4c3-c3ccccc3)cc21 ; c1ccc(-c2ccc(N(c3ccc4oc5c6ccccc6c(-c6ccccc6)cc5c4c3-c3ccccc3)c3cccc4c3oc3ccccc34)cc2)cc1]

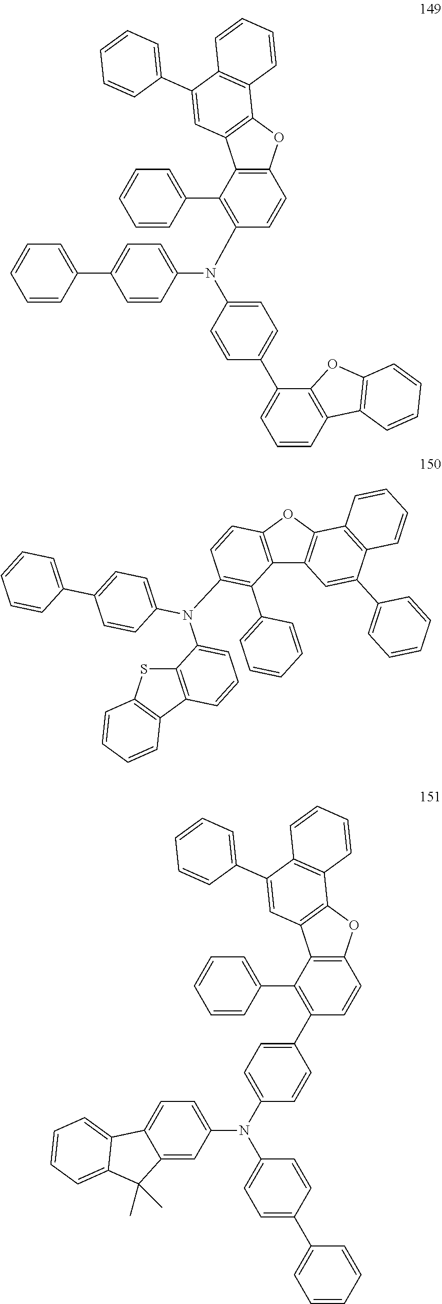 [SMILES: c1ccc(-c2ccc(N(c3ccc(-c4cccc5c4oc4ccccc45)cc3)c3ccc4oc5c6ccccc6c(-c6ccccc6)cc5c4c3-c3ccccc3)cc2)cc1 ; c1ccc(-c2ccc(N(c3ccc4oc5c6ccccc6c(-c6ccccc6)cc5c4c3-c3ccccc3)c3cccc4c3sc3ccccc34)cc2)cc1 ; CC1(C)c2ccccc2-c2ccc(N(c3ccc(-c4ccccc4)cc3)c3ccc(-c4ccc5oc6c7ccccc7c(-c7ccccc7)cc6c5c4-c4ccccc4)cc3)cc21]

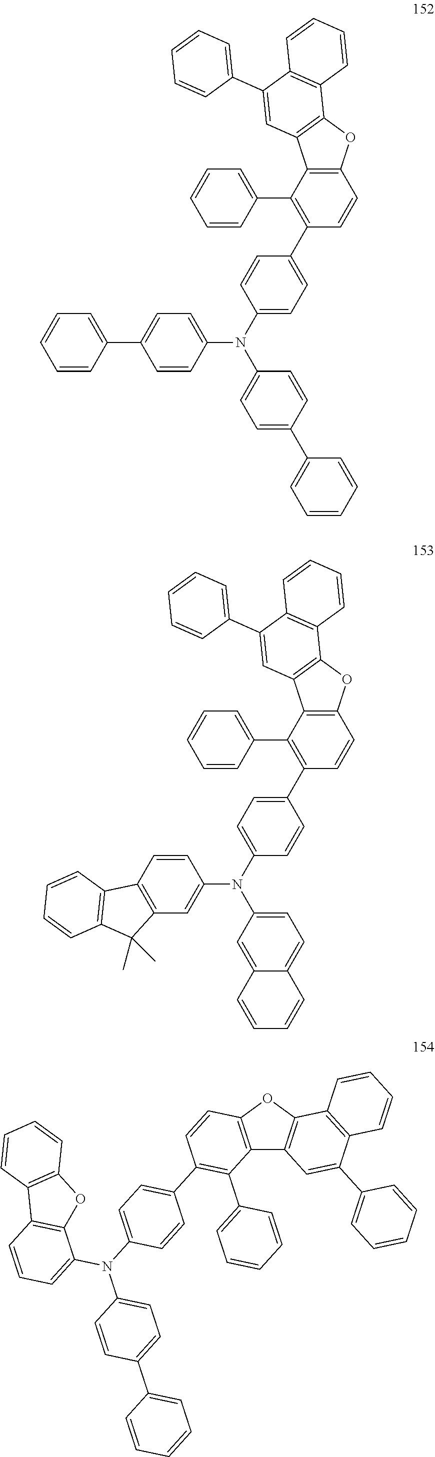

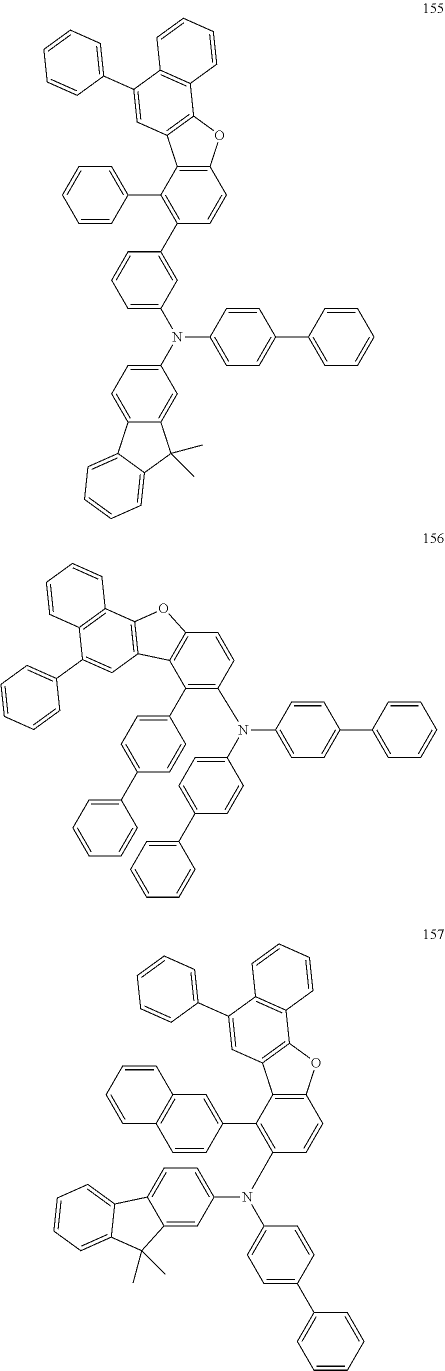 [SMILES: CC1(C)c2ccccc2-c2ccc(N(c3ccc(-c4ccccc4)cc3)c3cccc(-c4ccc5oc6c7ccccc7c(-c7ccccc7)cc6c5c4-c4ccccc4)c3)cc21 ; c1ccc(-c2ccc(-c3c(N(c4ccc(-c5ccccc5)cc4)c4ccc(-c5ccccc5)cc4)ccc4oc5c6ccccc6c(-c6ccccc6)cc5c34)cc2)cc1 ; CC1(C)c2ccccc2-c2ccc(N(c3ccc(-c4ccccc4)cc3)c3ccc4oc5c6ccccc6c(-c6ccccc6)cc5c4c3-c3ccc4ccccc4c3)cc21]

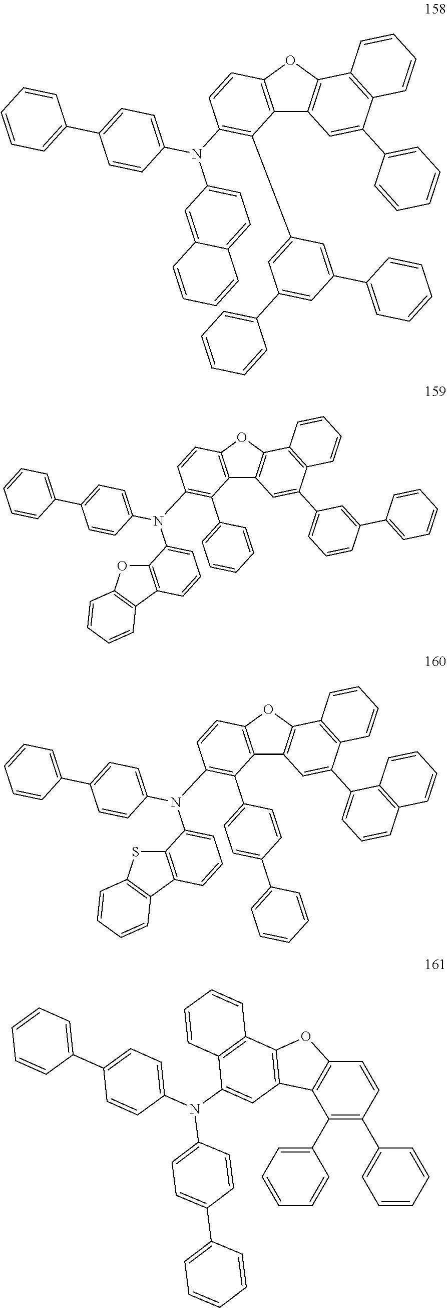 [SMILES: c1ccc(-c2ccc(N(c3ccc4ccccc4c3)c3ccc4oc5c6ccccc6c(-c6ccccc6)cc5c4c3-c3cc(-c4ccccc4)cc(-c4ccccc4)c3)cc2)cc1 ; c1ccc(-c2ccc(N(c3ccc4oc5c6ccccc6c(-c6cccc(-c7ccccc7)c6)cc5c4c3-c3ccccc3)c3cccc4c3oc3ccccc34)cc2)cc1 ; c1ccc(-c2ccc(-c3c(N(c4ccc(-c5ccccc5)cc4)c4cccc5c4sc4ccccc45)ccc4oc5c6ccccc6c(-c6cccc7ccccc67)cc5c34)cc2)cc1 ; c1ccc(-c2ccc(N(c3ccc(-c4ccccc4)cc3)c3cc4c(oc5ccc(-c6ccccc6)c(-c6ccccc6)c54)c4ccccc34)cc2)cc1]

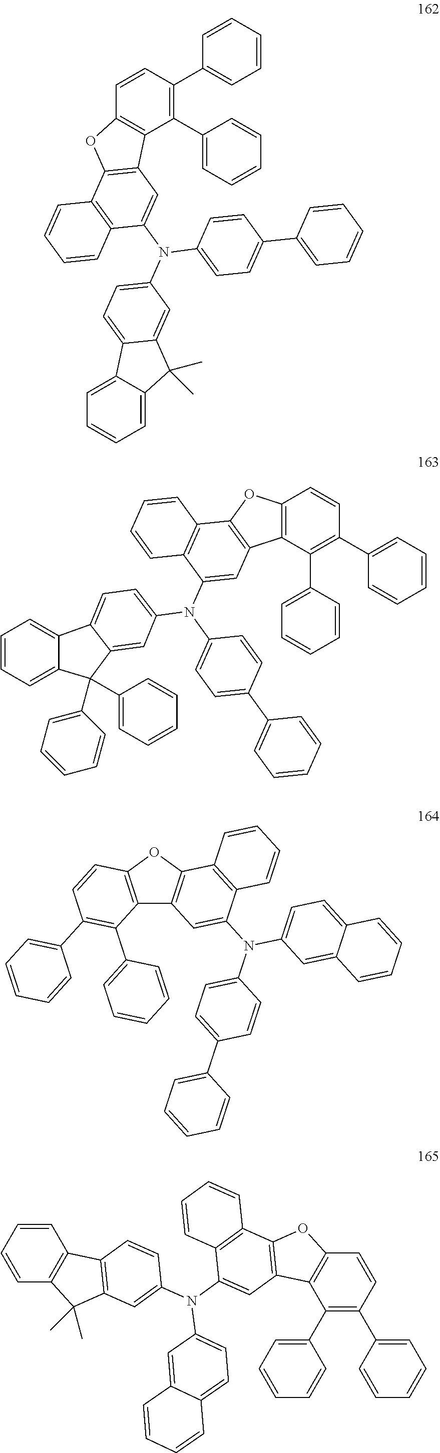 [SMILES: CC1(C)c2ccccc2-c2ccc(N(c3ccc(-c4ccccc4)cc3)c3cc4c(oc5ccc(-c6ccccc6)c(-c6ccccc6)c54)c4ccccc34)cc21 ; c1ccc(-c2ccc(N(c3ccc4c(c3)C(c3ccccc3)(c3ccccc3)c3ccccc3-4)c3cc4c(oc5ccc(-c6ccccc6)c(-c6ccccc6)c54)c4ccccc34)cc2)cc1 ; c1ccc(-c2ccc(N(c3ccc4ccccc4c3)c3cc4c(oc5ccc(-c6ccccc6)c(-c6ccccc6)c54)c4ccccc34)cc2)cc1 ; CC1(C)c2ccccc2-c2ccc(N(c3ccc4ccccc4c3)c3cc4c(oc5ccc(-c6ccccc6)c(-c6ccccc6)c54)c4ccccc34)cc21]

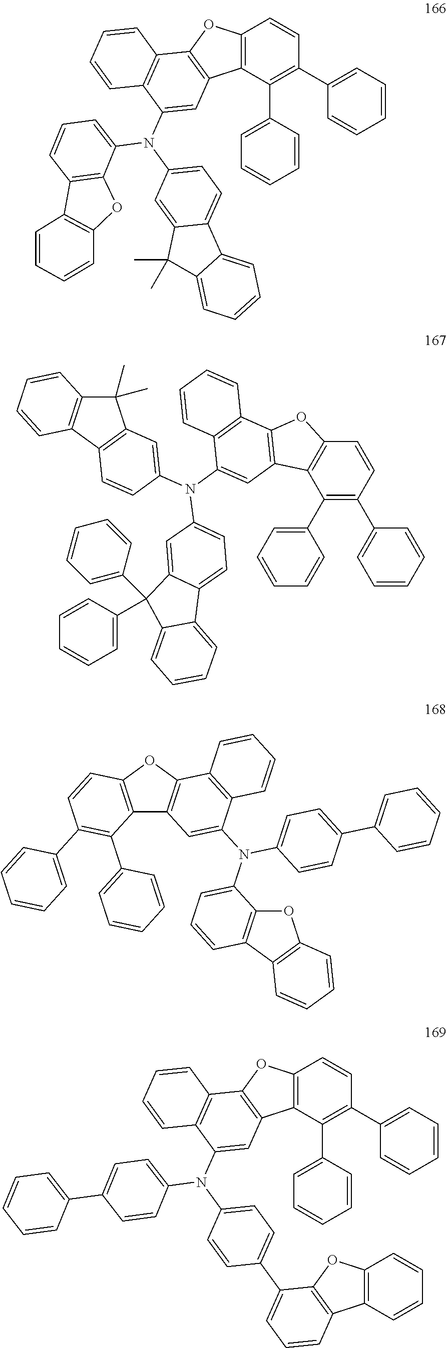 [SMILES: CC1(C)c2ccccc2-c2ccc(N(c3cc4c(oc5ccc(-c6ccccc6)c(-c6ccccc6)c54)c4ccccc34)c3cccc4c3oc3ccccc34)cc21 ; CC1(C)c2ccccc2-c2ccc(N(c3ccc4c(c3)C(c3ccccc3)(c3ccccc3)c3ccccc3-4)c3cc4c(oc5ccc(-c6ccccc6)c(-c6ccccc6)c54)c4ccccc34)cc21 ; c1ccc(-c2ccc(N(c3cc4c(oc5ccc(-c6ccccc6)c(-c6ccccc6)c54)c4ccccc34)c3cccc4c3oc3ccccc34)cc2)cc1 ; c1ccc(-c2ccc(N(c3ccc(-c4cccc5c4oc4ccccc45)cc3)c3cc4c(oc5ccc(-c6ccccc6)c(-c6ccccc6)c54)c4ccccc34)cc2)cc1]

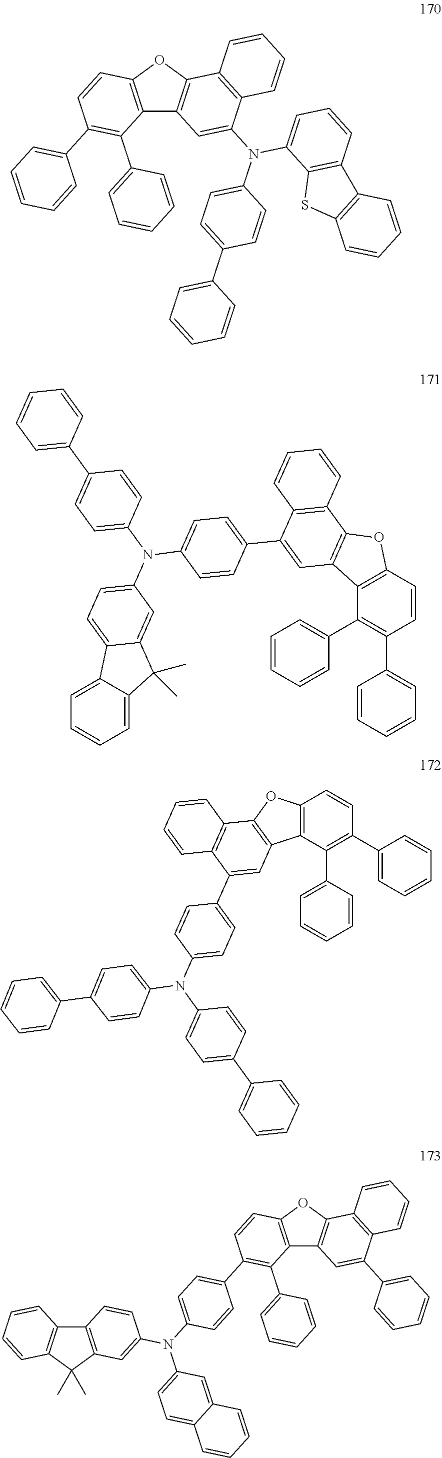 [SMILES: c1ccc(-c2ccc(N(c3cc4c(oc5ccc(-c6ccccc6)c(-c6ccccc6)c54)c4ccccc34)c3cccc4c3sc3ccccc34)cc2)cc1 ; CC1(C)c2ccccc2-c2ccc(N(c3ccc(-c4ccccc4)cc3)c3ccc(-c4cc5c(oc6ccc(-c7ccccc7)c(-c7ccccc7)c65)c5ccccc45)cc3)cc21 ; c1ccc(-c2ccc(N(c3ccc(-c4ccccc4)cc3)c3ccc(-c4cc5c(oc6ccc(-c7ccccc7)c(-c7ccccc7)c65)c5ccccc45)cc3)cc2)cc1 ; CC1(C)c2ccccc2-c2ccc(N(c3ccc(-c4ccc5oc6c7ccccc7c(-c7ccccc7)cc6c5c4-c4ccccc4)cc3)c3ccc4ccccc4c3)cc21]

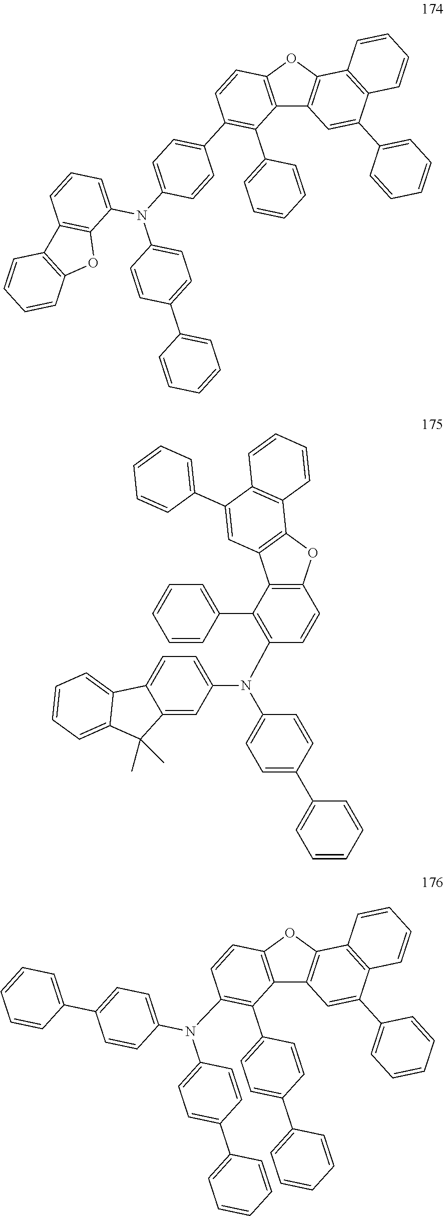 [SMILES: c1ccc(-c2ccc(N(c3ccc(-c4ccc5oc6c7ccccc7c(-c7ccccc7)cc6c5c4-c4ccccc4)cc3)c3cccc4c3oc3ccccc34)cc2)cc1 ; CC1(C)c2ccccc2-c2ccc(N(c3ccc(-c4ccccc4)cc3)c3ccc4oc5c6ccccc6c(-c6ccccc6)cc5c4c3-c3ccccc3)cc21 ; c1ccc(-c2ccc(-c3c(N(c4ccc(-c5ccccc5)cc4)c4ccc(-c5ccccc5)cc4)ccc4oc5c6ccccc6c(-c6ccccc6)cc5c34)cc2)cc1]

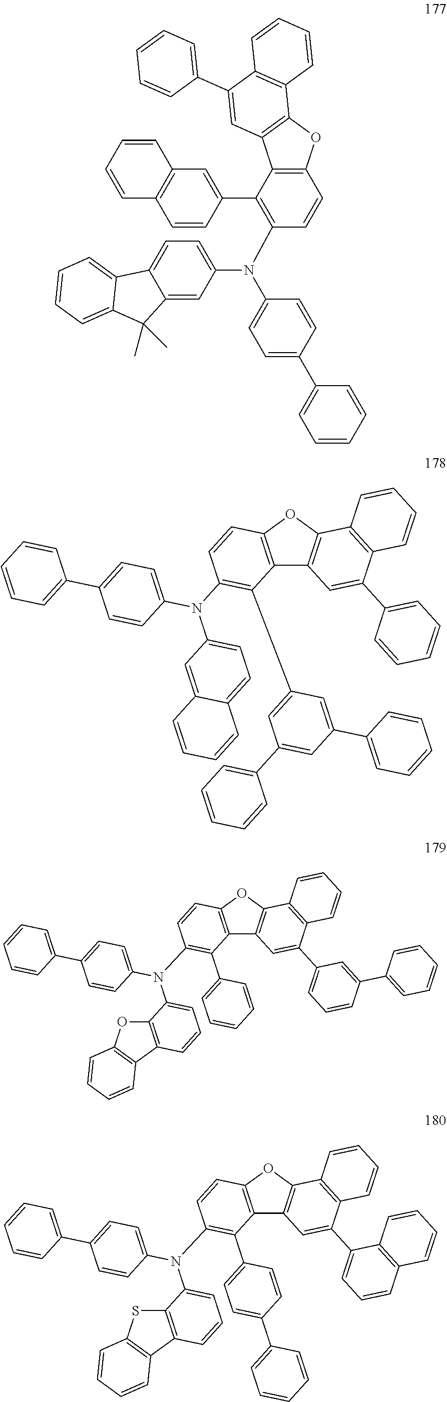 [SMILES: CC1(C)c2ccccc2-c2ccc(N(c3ccc(-c4ccccc4)cc3)c3ccc4oc5c6ccccc6c(-c6ccccc6)cc5c4c3-c3ccc4ccccc4c3)cc21 ; c1ccc(-c2ccc(N(c3ccc4ccccc4c3)c3ccc4oc5c6ccccc6c(-c6ccccc6)cc5c4c3-c3cc(-c4ccccc4)cc(-c4ccccc4)c3)cc2)cc1 ; c1ccc(-c2ccc(N(c3ccc4oc5c6ccccc6c(-c6cccc(-c7ccccc7)c6)cc5c4c3-c3ccccc3)c3cccc4c3oc3ccccc34)cc2)cc1 ; c1ccc(-c2ccc(-c3c(N(c4ccc(-c5ccccc5)cc4)c4cccc5c4sc4ccccc45)ccc4oc5c6ccccc6c(-c6cccc7ccccc67)cc5c34)cc2)cc1]

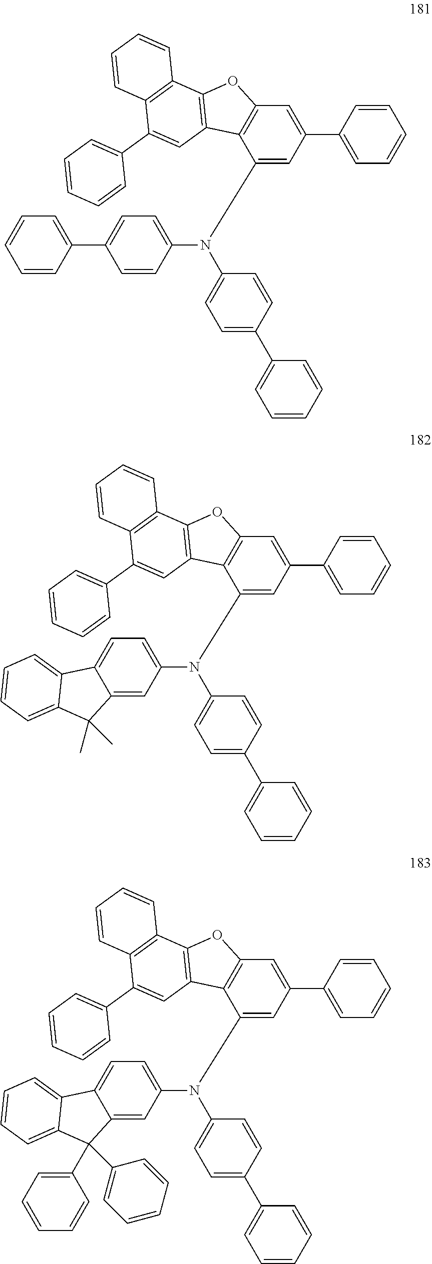 [SMILES: c1ccc(-c2ccc(N(c3ccc(-c4ccccc4)cc3)c3cc(-c4ccccc4)cc4oc5c6ccccc6c(-c6ccccc6)cc5c34)cc2)cc1 ; CC1(C)c2ccccc2-c2ccc(N(c3ccc(-c4ccccc4)cc3)c3cc(-c4ccccc4)cc4oc5c6ccccc6c(-c6ccccc6)cc5c34)cc21 ; c1ccc(-c2ccc(N(c3ccc4c(c3)C(c3ccccc3)(c3ccccc3)c3ccccc3-4)c3cc(-c4ccccc4)cc4oc5c6ccccc6c(-c6ccccc6)cc5c34)cc2)cc1]

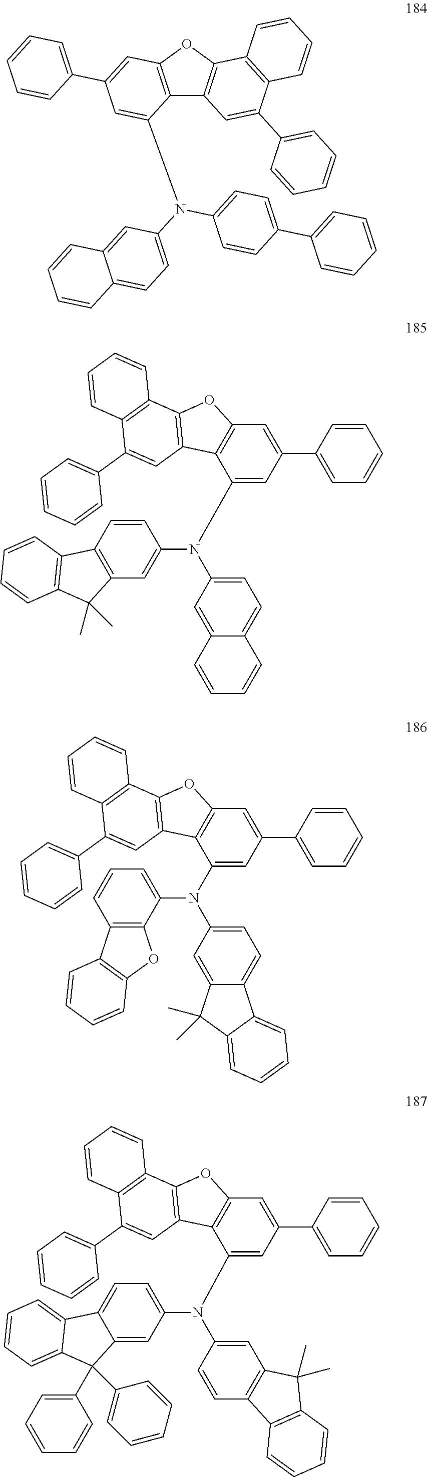 [SMILES: c1ccc(-c2ccc(N(c3ccc4ccccc4c3)c3cc(-c4ccccc4)cc4oc5c6ccccc6c(-c6ccccc6)cc5c34)cc2)cc1 ; CC1(C)c2ccccc2-c2ccc(N(c3ccc4ccccc4c3)c3cc(-c4ccccc4)cc4oc5c6ccccc6c(-c6ccccc6)cc5c34)cc21 ; CC1(C)c2ccccc2-c2ccc(N(c3cccc4c3oc3ccccc34)c3cc(-c4ccccc4)cc4oc5c6ccccc6c(-c6ccccc6)cc5c34)cc21 ; CC1(C)c2ccccc2-c2ccc(N(c3ccc4c(c3)C(c3ccccc3)(c3ccccc3)c3ccccc3-4)c3cc(-c4ccccc4)cc4oc5c6ccccc6c(-c6ccccc6)cc5c34)cc21]

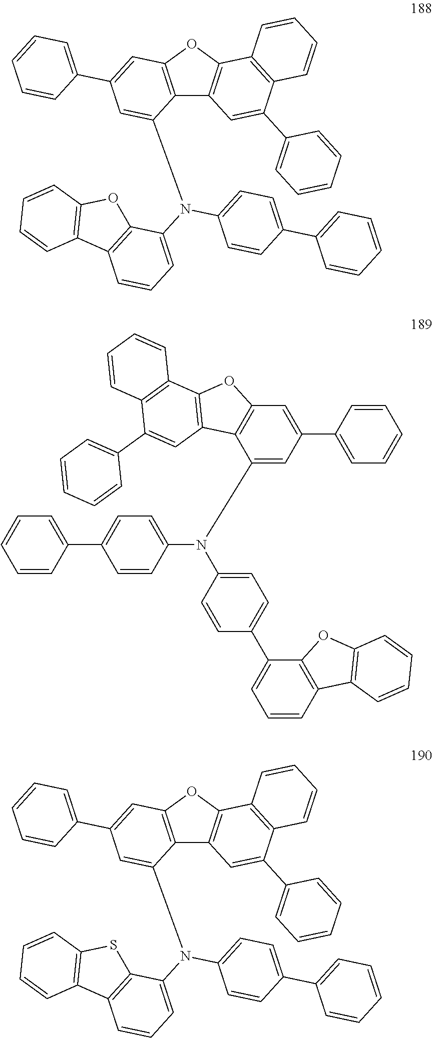 [SMILES: c1ccc(-c2ccc(N(c3cccc4c3oc3ccccc34)c3cc(-c4ccccc4)cc4oc5c6ccccc6c(-c6ccccc6)cc5c34)cc2)cc1 ; c1ccc(-c2ccc(N(c3ccc(-c4cccc5c4oc4ccccc45)cc3)c3cc(-c4ccccc4)cc4oc5c6ccccc6c(-c6ccccc6)cc5c34)cc2)cc1 ; c1ccc(-c2ccc(N(c3cccc4c3sc3ccccc34)c3cc(-c4ccccc4)cc4oc5c6ccccc6c(-c6ccccc6)cc5c34)cc2)cc1]

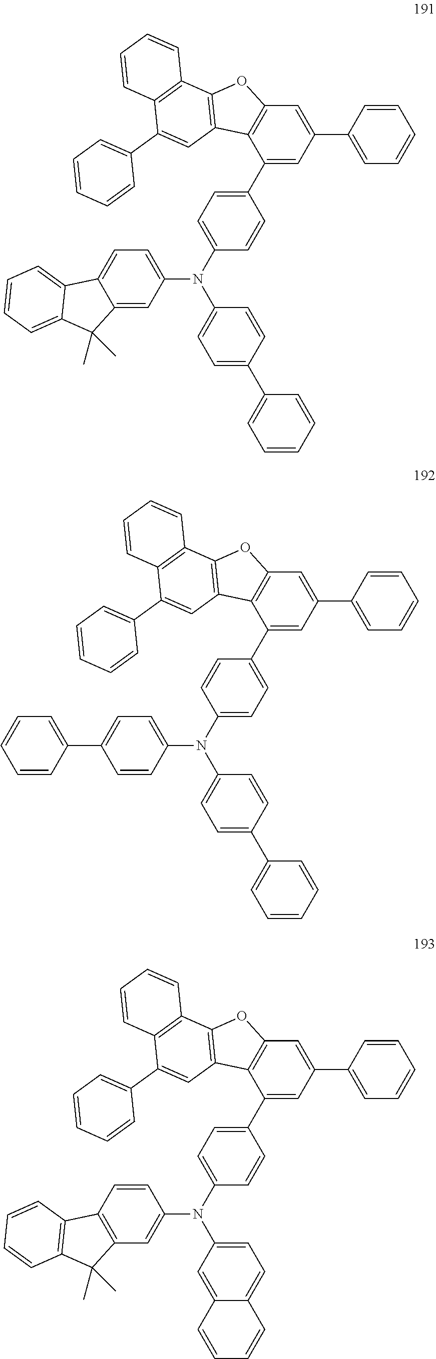 [SMILES: CC1(C)c2ccccc2-c2ccc(N(c3ccc(-c4ccccc4)cc3)c3ccc(-c4cc(-c5ccccc5)cc5oc6c7ccccc7c(-c7ccccc7)cc6c45)cc3)cc21 ; c1ccc(-c2ccc(N(c3ccc(-c4ccccc4)cc3)c3ccc(-c4cc(-c5ccccc5)cc5oc6c7ccccc7c(-c7ccccc7)cc6c45)cc3)cc2)cc1 ; CC1(C)c2ccccc2-c2ccc(N(c3ccc(-c4cc(-c5ccccc5)cc5oc6c7ccccc7c(-c7ccccc7)cc6c45)cc3)c3ccc4ccccc4c3)cc21]

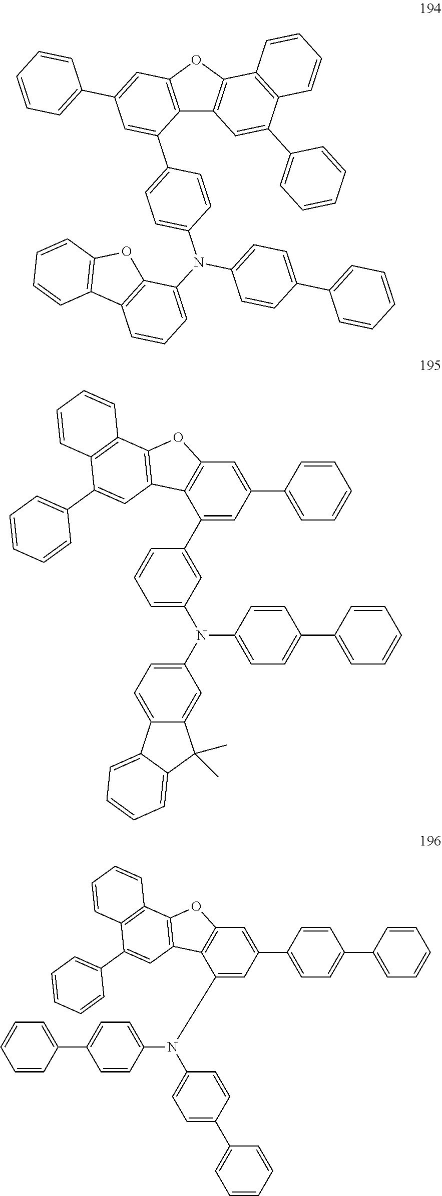 [SMILES: c1ccc(-c2ccc(N(c3ccc(-c4cc(-c5ccccc5)cc5oc6c7ccccc7c(-c7ccccc7)cc6c45)cc3)c3cccc4c3oc3ccccc34)cc2)cc1 ; CC1(C)c2ccccc2-c2ccc(N(c3ccc(-c4ccccc4)cc3)c3cccc(-c4cc(-c5ccccc5)cc5oc6c7ccccc7c(-c7ccccc7)cc6c45)c3)cc21 ; c1ccc(-c2ccc(-c3cc(N(c4ccc(-c5ccccc5)cc4)c4ccc(-c5ccccc5)cc4)c4c(c3)oc3c5ccccc5c(-c5ccccc5)cc34)cc2)cc1]

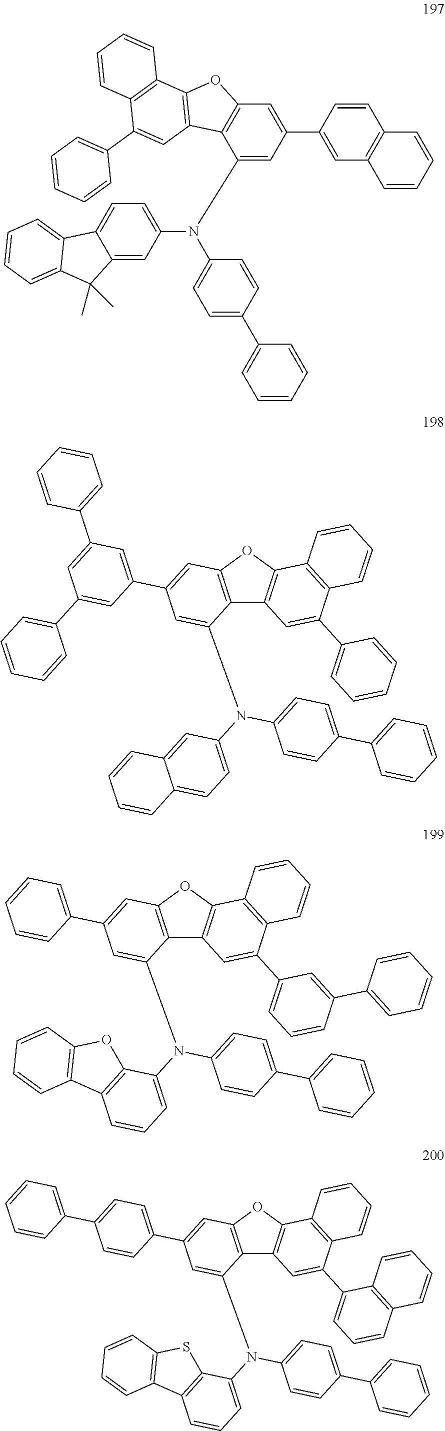 [SMILES: CC1(C)c2ccccc2-c2ccc(N(c3ccc(-c4ccccc4)cc3)c3cc(-c4ccc5ccccc5c4)cc4oc5c6ccccc6c(-c6ccccc6)cc5c34)cc21 ; c1ccc(-c2ccc(N(c3ccc4ccccc4c3)c3cc(-c4cc(-c5ccccc5)cc(-c5ccccc5)c4)cc4oc5c6ccccc6c(-c6ccccc6)cc5c34)cc2)cc1 ; c1ccc(-c2ccc(N(c3cccc4c3oc3ccccc34)c3cc(-c4ccccc4)cc4oc5c6ccccc6c(-c6cccc(-c7ccccc7)c6)cc5c34)cc2)cc1 ; c1ccc(-c2ccc(-c3cc(N(c4ccc(-c5ccccc5)cc4)c4cccc5c4sc4ccccc45)c4c(c3)oc3c5ccccc5c(-c5cccc6ccccc56)cc34)cc2)cc1]

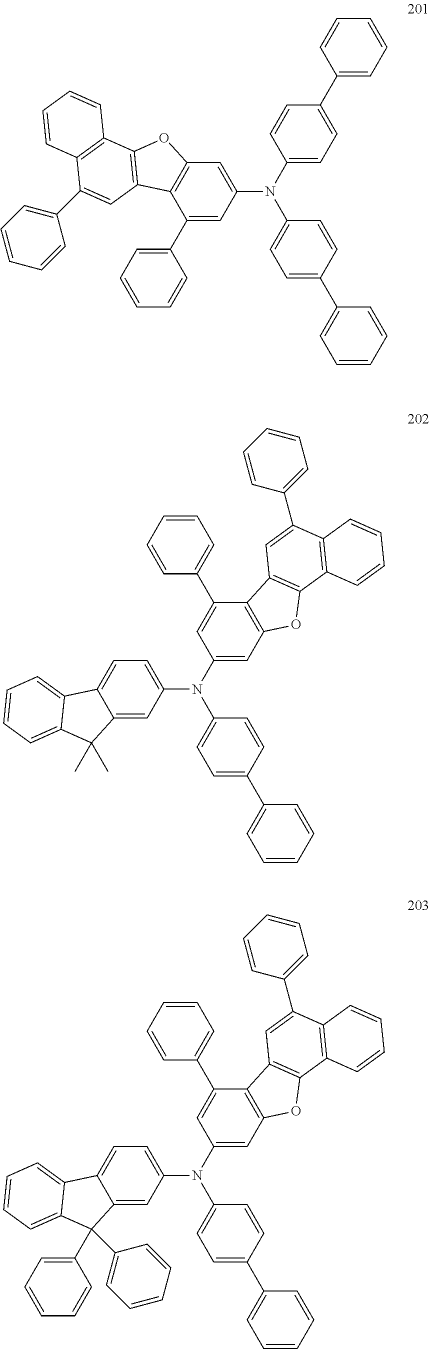 [SMILES: c1ccc(-c2ccc(N(c3ccc(-c4ccccc4)cc3)c3cc(-c4ccccc4)c4c(c3)oc3c5ccccc5c(-c5ccccc5)cc34)cc2)cc1 ; CC1(C)c2ccccc2-c2ccc(N(c3ccc(-c4ccccc4)cc3)c3cc(-c4ccccc4)c4c(c3)oc3c5ccccc5c(-c5ccccc5)cc34)cc21 ; c1ccc(-c2ccc(N(c3ccc4c(c3)C(c3ccccc3)(c3ccccc3)c3ccccc3-4)c3cc(-c4ccccc4)c4c(c3)oc3c5ccccc5c(-c5ccccc5)cc34)cc2)cc1]

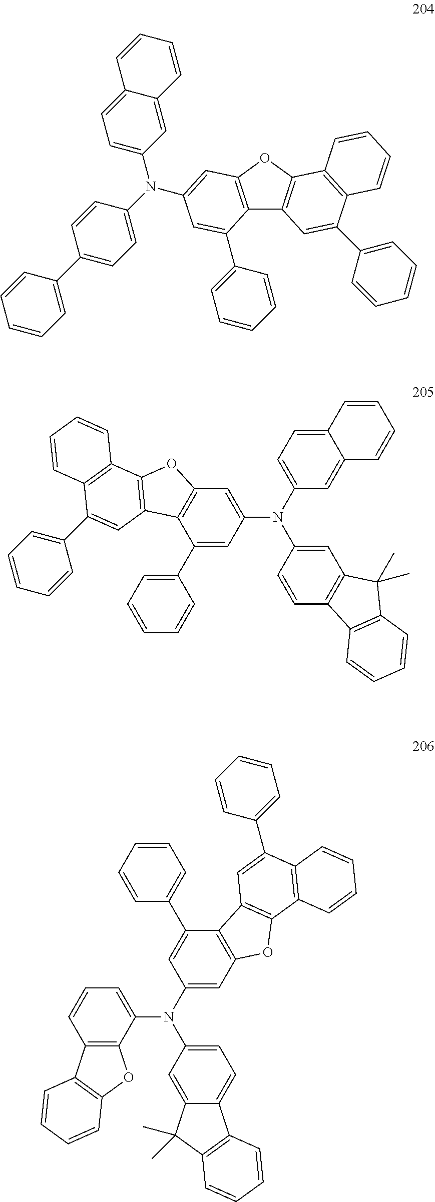 [SMILES: c1ccc(-c2ccc(N(c3ccc4ccccc4c3)c3cc(-c4ccccc4)c4c(c3)oc3c5ccccc5c(-c5ccccc5)cc34)cc2)cc1 ; CC1(C)c2ccccc2-c2ccc(N(c3ccc4ccccc4c3)c3cc(-c4ccccc4)c4c(c3)oc3c5ccccc5c(-c5ccccc5)cc34)cc21 ; CC1(C)c2ccccc2-c2ccc(N(c3cc(-c4ccccc4)c4c(c3)oc3c5ccccc5c(-c5ccccc5)cc34)c3cccc4c3oc3ccccc34)cc21]

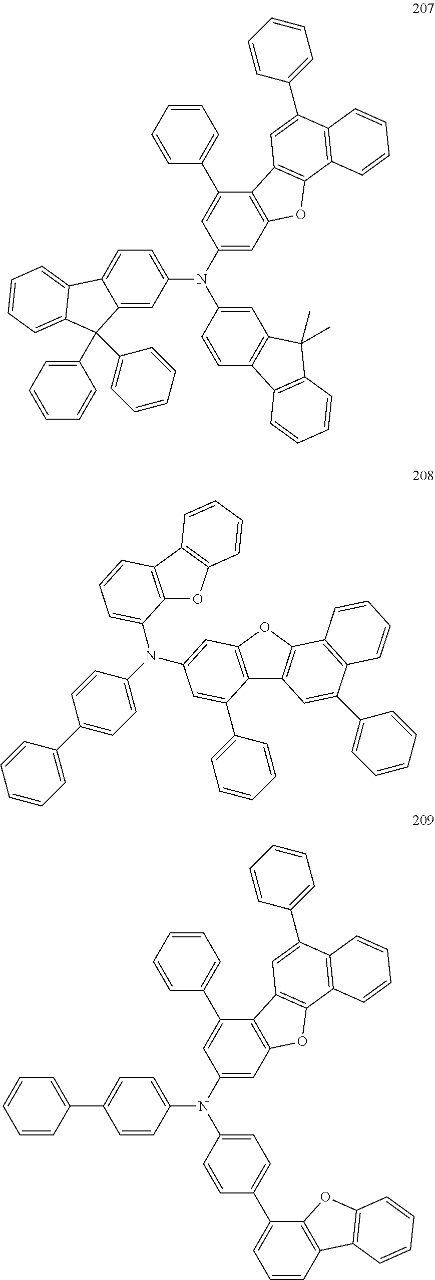 [SMILES: CC1(C)c2ccccc2-c2ccc(N(c3ccc4c(c3)C(c3ccccc3)(c3ccccc3)c3ccccc3-4)c3cc(-c4ccccc4)c4c(c3)oc3c5ccccc5c(-c5ccccc5)cc34)cc21 ; c1ccc(-c2ccc(N(c3cc(-c4ccccc4)c4c(c3)oc3c5ccccc5c(-c5ccccc5)cc34)c3cccc4c3oc3ccccc34)cc2)cc1 ; c1ccc(-c2ccc(N(c3ccc(-c4cccc5c4oc4ccccc45)cc3)c3cc(-c4ccccc4)c4c(c3)oc3c5ccccc5c(-c5ccccc5)cc34)cc2)cc1]

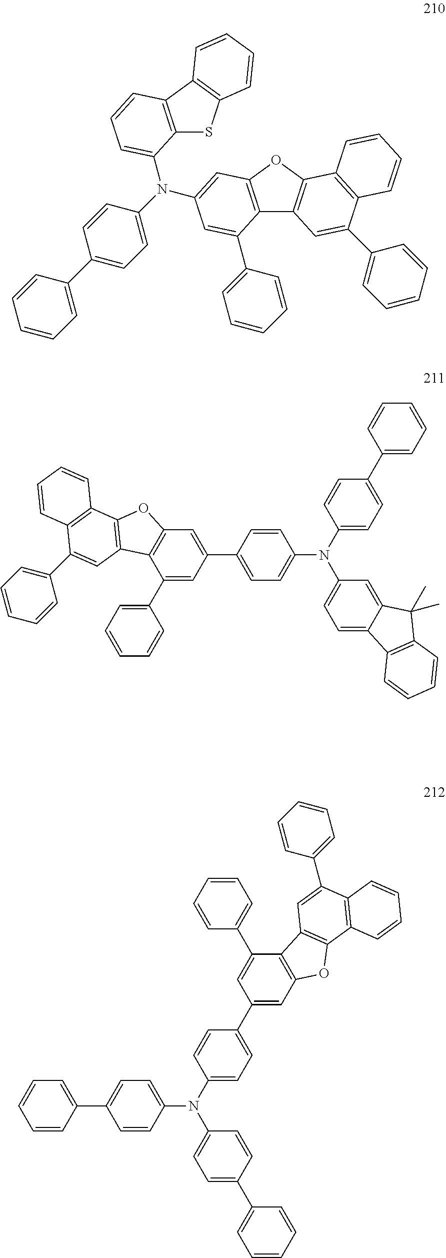 [SMILES: c1ccc(-c2ccc(N(c3cc(-c4ccccc4)c4c(c3)oc3c5ccccc5c(-c5ccccc5)cc34)c3cccc4c3sc3ccccc34)cc2)cc1 ; CC1(C)c2ccccc2-c2ccc(N(c3ccc(-c4ccccc4)cc3)c3ccc(-c4cc(-c5ccccc5)c5c(c4)oc4c6ccccc6c(-c6ccccc6)cc45)cc3)cc21 ; c1ccc(-c2ccc(N(c3ccc(-c4ccccc4)cc3)c3ccc(-c4cc(-c5ccccc5)c5c(c4)oc4c6ccccc6c(-c6ccccc6)cc45)cc3)cc2)cc1]

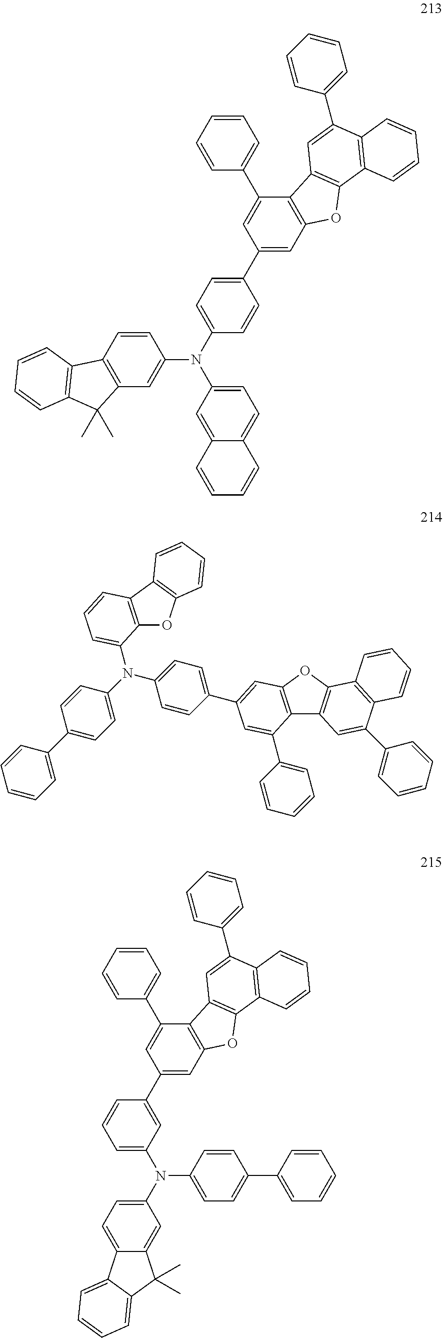 [SMILES: CC1(C)c2ccccc2-c2ccc(N(c3ccc(-c4cc(-c5ccccc5)c5c(c4)oc4c6ccccc6c(-c6ccccc6)cc45)cc3)c3ccc4ccccc4c3)cc21 ; c1ccc(-c2ccc(N(c3ccc(-c4cc(-c5ccccc5)c5c(c4)oc4c6ccccc6c(-c6ccccc6)cc45)cc3)c3cccc4c3oc3ccccc34)cc2)cc1 ; CC1(C)c2ccccc2-c2ccc(N(c3ccc(-c4ccccc4)cc3)c3cccc(-c4cc(-c5ccccc5)c5c(c4)oc4c6ccccc6c(-c6ccccc6)cc45)c3)cc21]

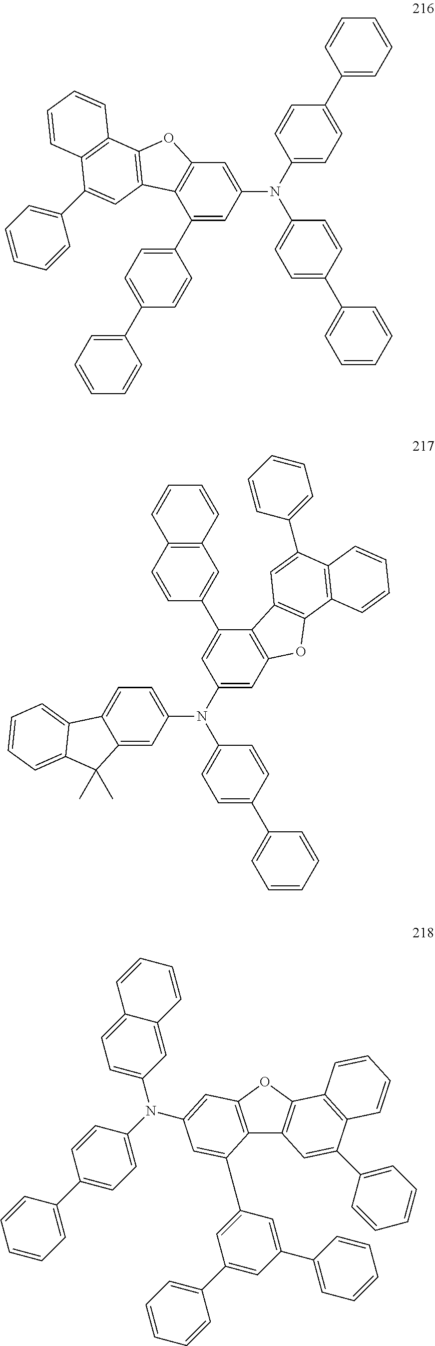 [SMILES: c1ccc(-c2ccc(-c3cc(N(c4ccc(-c5ccccc5)cc4)c4ccc(-c5ccccc5)cc4)cc4oc5c6ccccc6c(-c6ccccc6)cc5c34)cc2)cc1 ; CC1(C)c2ccccc2-c2ccc(N(c3ccc(-c4ccccc4)cc3)c3cc(-c4ccc5ccccc5c4)c4c(c3)oc3c5ccccc5c(-c5ccccc5)cc34)cc21 ; c1ccc(-c2ccc(N(c3ccc4ccccc4c3)c3cc(-c4cc(-c5ccccc5)cc(-c5ccccc5)c4)c4c(c3)oc3c5ccccc5c(-c5ccccc5)cc34)cc2)cc1]

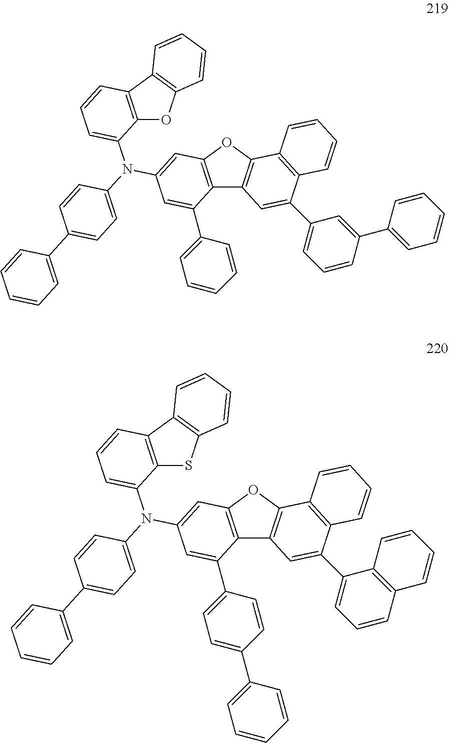 [SMILES: c1ccc(-c2ccc(N(c3cc(-c4ccccc4)c4c(c3)oc3c5ccccc5c(-c5cccc(-c6ccccc6)c5)cc34)c3cccc4c3oc3ccccc34)cc2)cc1 ; c1ccc(-c2ccc(-c3cc(N(c4ccc(-c5ccccc5)cc4)c4cccc5c4sc4ccccc45)cc4oc5c6ccccc6c(-c6cccc7ccccc67)cc5c34)cc2)cc1]

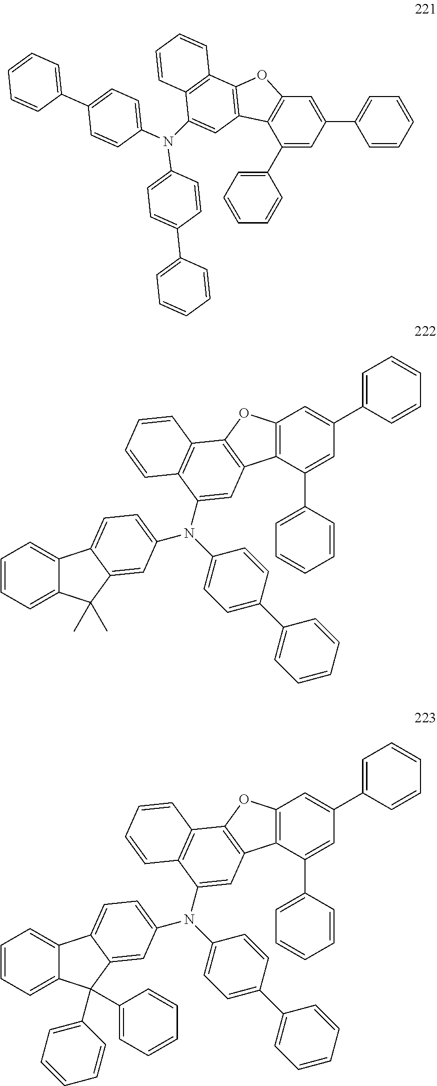 [SMILES: c1ccc(-c2ccc(N(c3ccc(-c4ccccc4)cc3)c3cc4c(oc5cc(-c6ccccc6)cc(-c6ccccc6)c54)c4ccccc34)cc2)cc1 ; CC1(C)c2ccccc2-c2ccc(N(c3ccc(-c4ccccc4)cc3)c3cc4c(oc5cc(-c6ccccc6)cc(-c6ccccc6)c54)c4ccccc34)cc21 ; c1ccc(-c2ccc(N(c3ccc4c(c3)C(c3ccccc3)(c3ccccc3)c3ccccc3-4)c3cc4c(oc5cc(-c6ccccc6)cc(-c6ccccc6)c54)c4ccccc34)cc2)cc1]

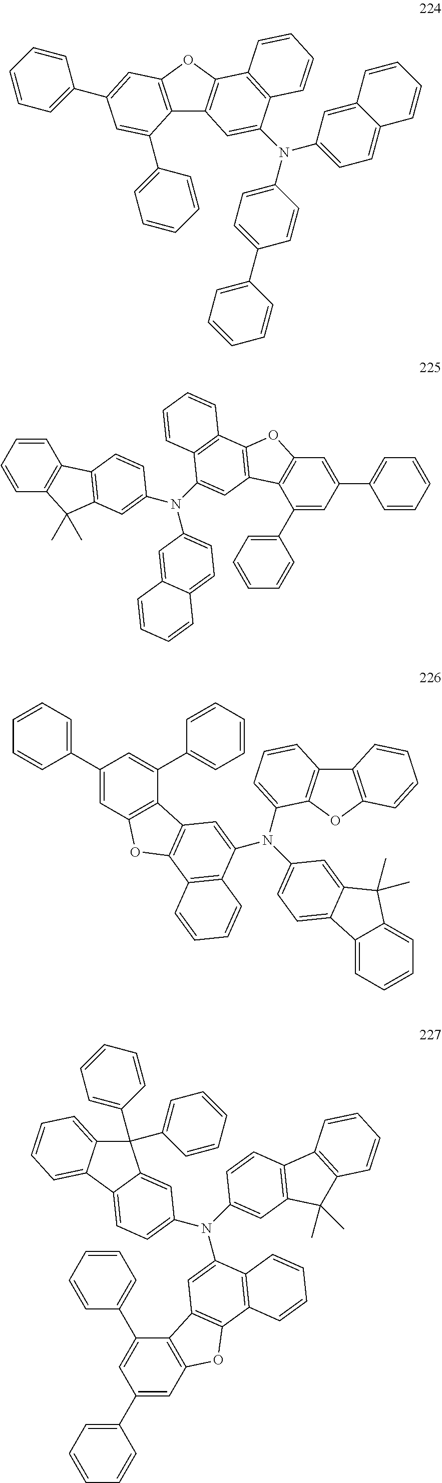 [SMILES: c1ccc(-c2ccc(N(c3ccc4ccccc4c3)c3cc4c(oc5cc(-c6ccccc6)cc(-c6ccccc6)c54)c4ccccc34)cc2)cc1 ; CC1(C)c2ccccc2-c2ccc(N(c3ccc4ccccc4c3)c3cc4c(oc5cc(-c6ccccc6)cc(-c6ccccc6)c54)c4ccccc34)cc21 ; CC1(C)c2ccccc2-c2ccc(N(c3cc4c(oc5cc(-c6ccccc6)cc(-c6ccccc6)c54)c4ccccc34)c3cccc4c3oc3ccccc34)cc21 ; CC1(C)c2ccccc2-c2ccc(N(c3ccc4c(c3)C(c3ccccc3)(c3ccccc3)c3ccccc3-4)c3cc4c(oc5cc(-c6ccccc6)cc(-c6ccccc6)c54)c4ccccc34)cc21]

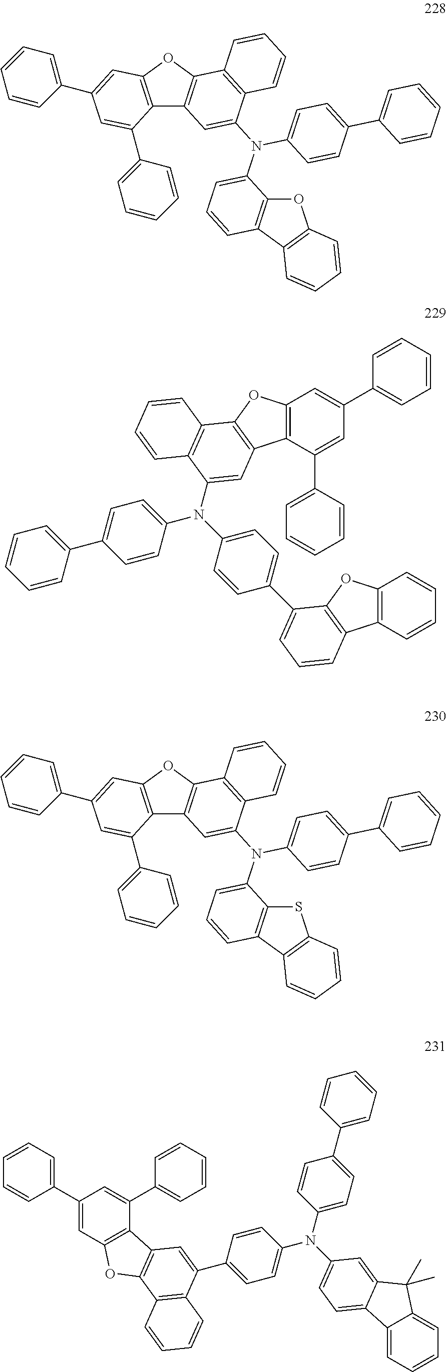 [SMILES: c1ccc(-c2ccc(N(c3cc4c(oc5cc(-c6ccccc6)cc(-c6ccccc6)c54)c4ccccc34)c3cccc4c3oc3ccccc34)cc2)cc1 ; c1ccc(-c2ccc(N(c3ccc(-c4cccc5c4oc4ccccc45)cc3)c3cc4c(oc5cc(-c6ccccc6)cc(-c6ccccc6)c54)c4ccccc34)cc2)cc1 ; c1ccc(-c2ccc(N(c3cc4c(oc5cc(-c6ccccc6)cc(-c6ccccc6)c54)c4ccccc34)c3cccc4c3sc3ccccc34)cc2)cc1 ; CC1(C)c2ccccc2-c2ccc(N(c3ccc(-c4ccccc4)cc3)c3ccc(-c4cc5c(oc6cc(-c7ccccc7)cc(-c7ccccc7)c65)c5ccccc45)cc3)cc21]

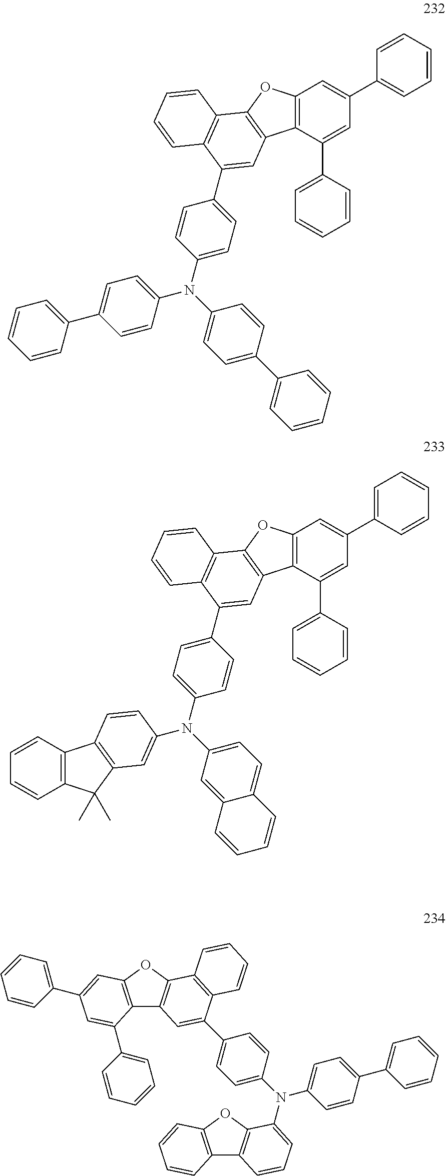 [SMILES: c1ccc(-c2ccc(N(c3ccc(-c4ccccc4)cc3)c3ccc(-c4cc5c(oc6cc(-c7ccccc7)cc(-c7ccccc7)c65)c5ccccc45)cc3)cc2)cc1 ; CC1(C)c2ccccc2-c2ccc(N(c3ccc(-c4cc5c(oc6cc(-c7ccccc7)cc(-c7ccccc7)c65)c5ccccc45)cc3)c3ccc4ccccc4c3)cc21 ; c1ccc(-c2ccc(N(c3ccc(-c4cc5c(oc6cc(-c7ccccc7)cc(-c7ccccc7)c65)c5ccccc45)cc3)c3cccc4c3oc3ccccc34)cc2)cc1]

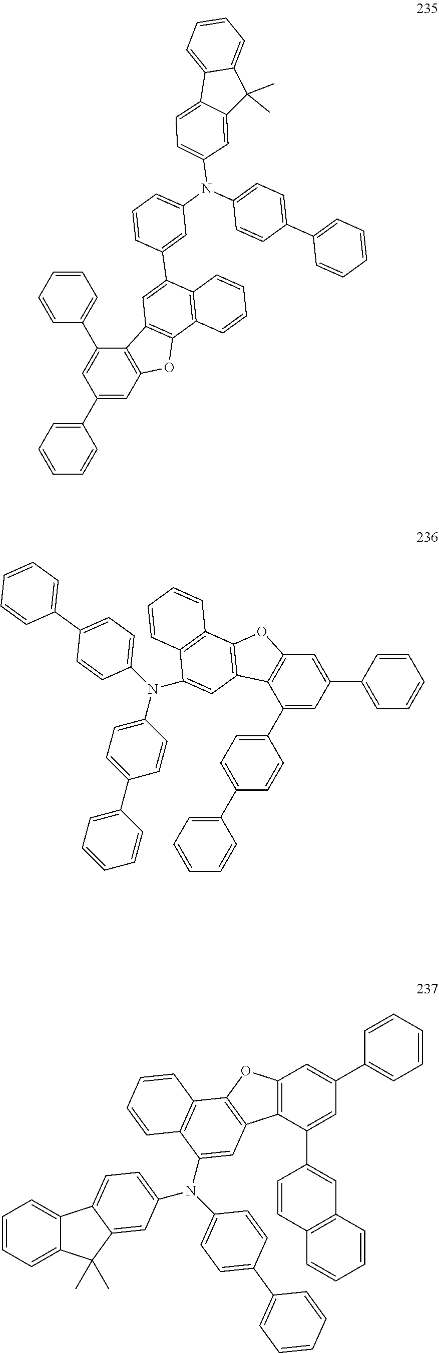 [SMILES: CC1(C)c2ccccc2-c2ccc(N(c3ccc(-c4ccccc4)cc3)c3cccc(-c4cc5c(oc6cc(-c7ccccc7)cc(-c7ccccc7)c65)c5ccccc45)c3)cc21 ; c1ccc(-c2ccc(-c3cc(-c4ccccc4)cc4oc5c6ccccc6c(N(c6ccc(-c7ccccc7)cc6)c6ccc(-c7ccccc7)cc6)cc5c34)cc2)cc1 ; CC1(C)c2ccccc2-c2ccc(N(c3ccc(-c4ccccc4)cc3)c3cc4c(oc5cc(-c6ccccc6)cc(-c6ccc7ccccc7c6)c54)c4ccccc34)cc21]

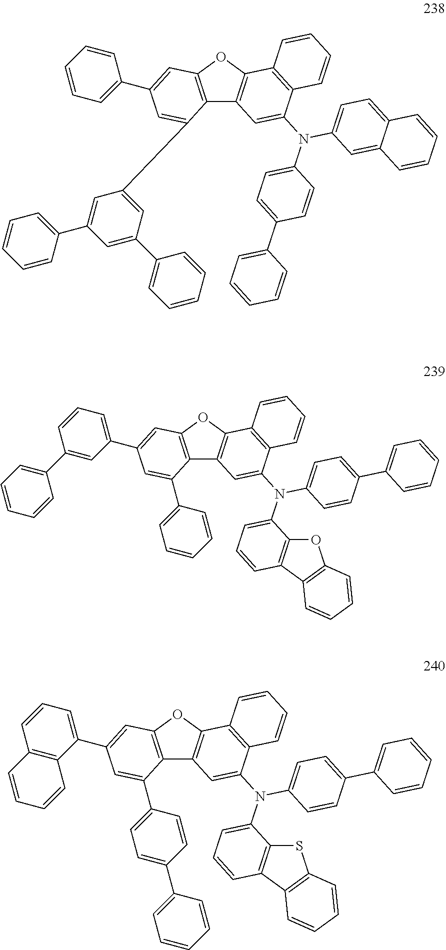 [SMILES: c1ccc(-c2ccc(N(c3ccc4ccccc4c3)c3cc4c(oc5cc(-c6ccccc6)cc(-c6cc(-c7ccccc7)cc(-c7ccccc7)c6)c54)c4ccccc34)cc2)cc1 ; c1ccc(-c2ccc(N(c3cc4c(oc5cc(-c6cccc(-c7ccccc7)c6)cc(-c6ccccc6)c54)c4ccccc34)c3cccc4c3oc3ccccc34)cc2)cc1 ; c1ccc(-c2ccc(-c3cc(-c4cccc5ccccc45)cc4oc5c6ccccc6c(N(c6ccc(-c7ccccc7)cc6)c6cccc7c6sc6ccccc67)cc5c34)cc2)cc1]

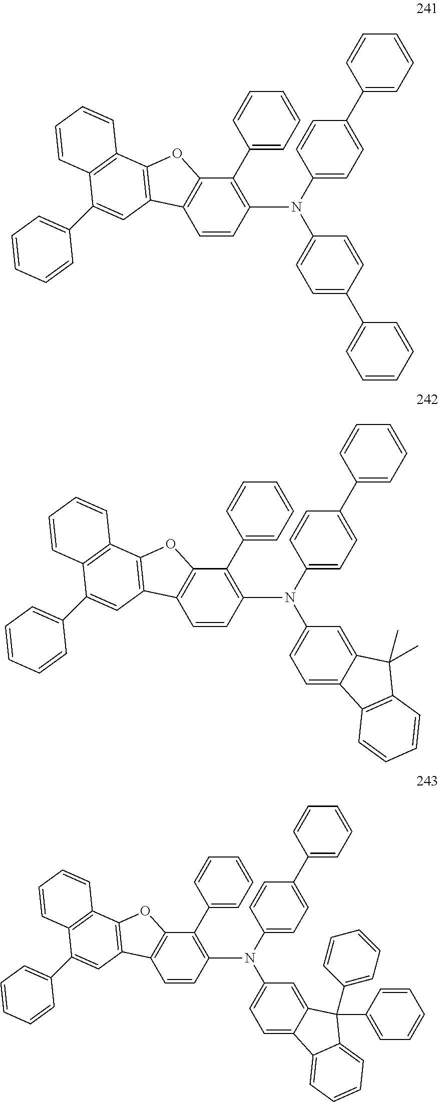

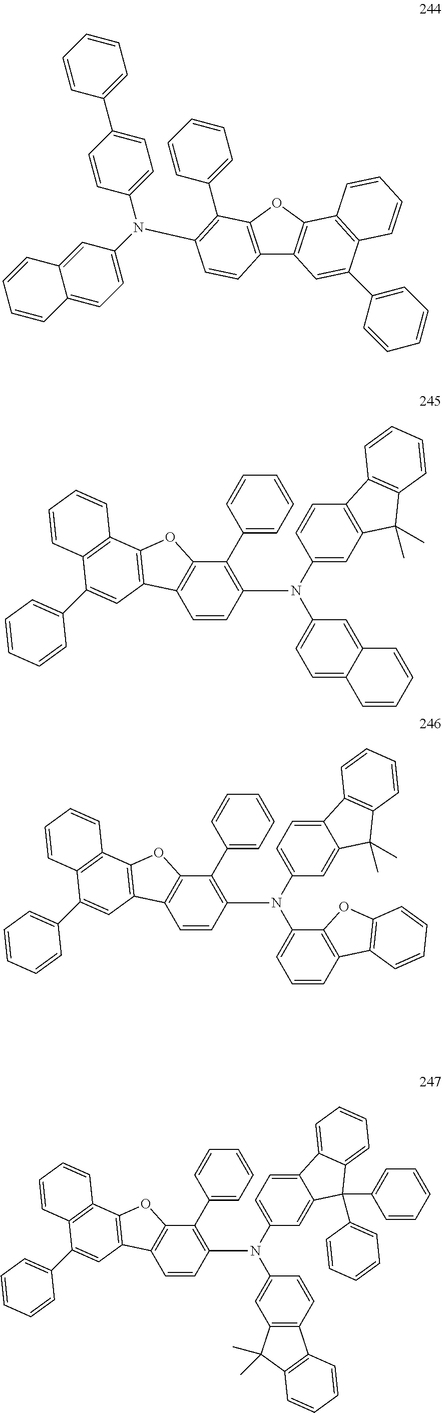

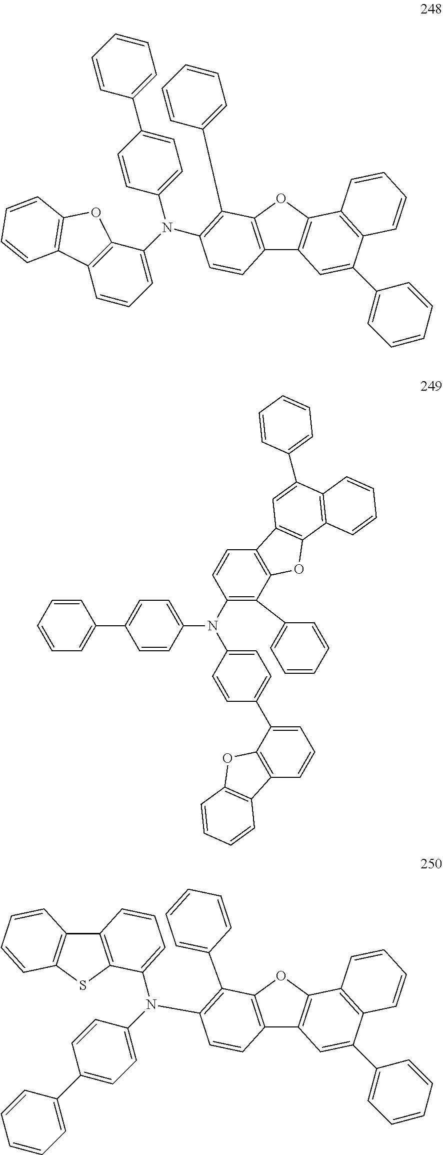 [SMILES: c1ccc(-c2ccc(N(c3ccc4c(oc5c6ccccc6c(-c6ccccc6)cc45)c3-c3ccccc3)c3cccc4c3oc3ccccc34)cc2)cc1 ; c1ccc(-c2ccc(N(c3ccc(-c4cccc5c4oc4ccccc45)cc3)c3ccc4c(oc5c6ccccc6c(-c6ccccc6)cc45)c3-c3ccccc3)cc2)cc1 ; c1ccc(-c2ccc(N(c3ccc4c(oc5c6ccccc6c(-c6ccccc6)cc45)c3-c3ccccc3)c3cccc4c3sc3ccccc34)cc2)cc1]

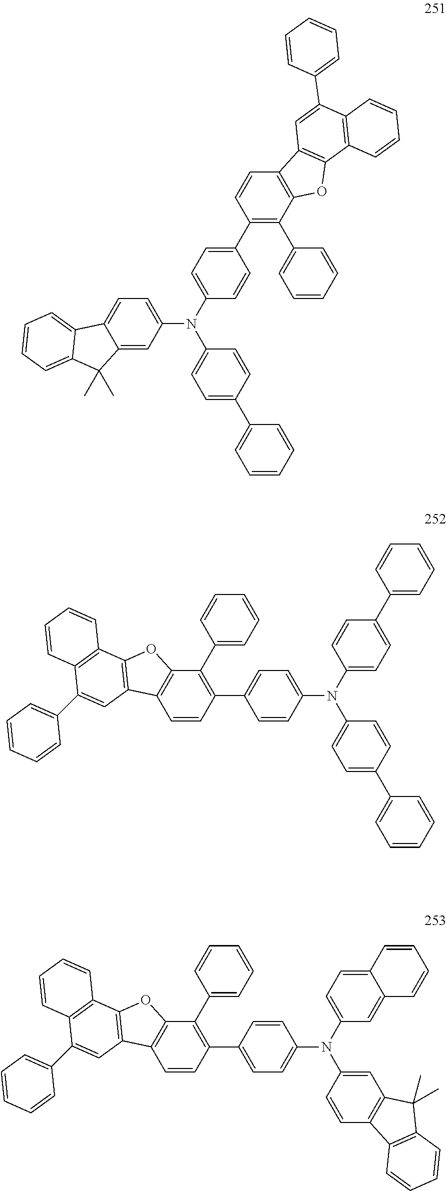 [SMILES: CC1(C)c2ccccc2-c2ccc(N(c3ccc(-c4ccccc4)cc3)c3ccc(-c4ccc5c(oc6c7ccccc7c(-c7ccccc7)cc56)c4-c4ccccc4)cc3)cc21 ; c1ccc(-c2ccc(N(c3ccc(-c4ccccc4)cc3)c3ccc(-c4ccc5c(oc6c7ccccc7c(-c7ccccc7)cc56)c4-c4ccccc4)cc3)cc2)cc1 ; CC1(C)c2ccccc2-c2ccc(N(c3ccc(-c4ccc5c(oc6c7ccccc7c(-c7ccccc7)cc56)c4-c4ccccc4)cc3)c3ccc4ccccc4c3)cc21]

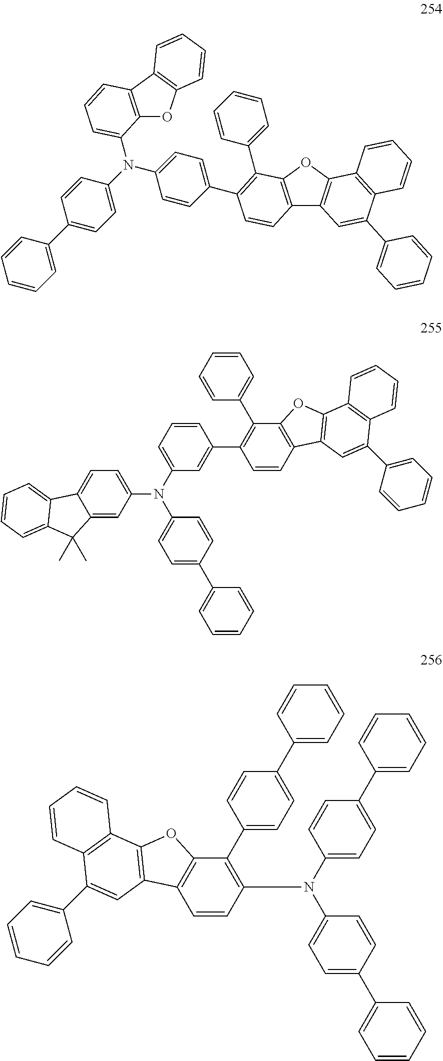 [SMILES: c1ccc(-c2ccc(N(c3ccc(-c4ccc5c(oc6c7ccccc7c(-c7ccccc7)cc56)c4-c4ccccc4)cc3)c3cccc4c3oc3ccccc34)cc2)cc1 ; CC1(C)c2ccccc2-c2ccc(N(c3ccc(-c4ccccc4)cc3)c3cccc(-c4ccc5c(oc6c7ccccc7c(-c7ccccc7)cc56)c4-c4ccccc4)c3)cc21 ; c1ccc(-c2ccc(-c3c(N(c4ccc(-c5ccccc5)cc4)c4ccc(-c5ccccc5)cc4)ccc4c3oc3c5ccccc5c(-c5ccccc5)cc43)cc2)cc1]

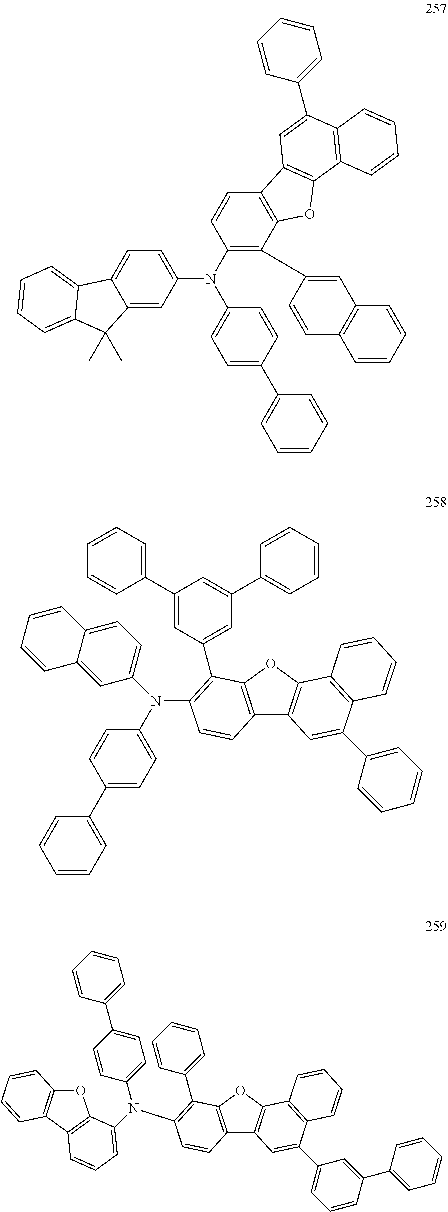 [SMILES: CC1(C)c2ccccc2-c2ccc(N(c3ccc(-c4ccccc4)cc3)c3ccc4c(oc5c6ccccc6c(-c6ccccc6)cc45)c3-c3ccc4ccccc4c3)cc21 ; c1ccc(-c2ccc(N(c3ccc4ccccc4c3)c3ccc4c(oc5c6ccccc6c(-c6ccccc6)cc45)c3-c3cc(-c4ccccc4)cc(-c4ccccc4)c3)cc2)cc1 ; c1ccc(-c2ccc(N(c3ccc4c(oc5c6ccccc6c(-c6cccc(-c7ccccc7)c6)cc45)c3-c3ccccc3)c3cccc4c3oc3ccccc34)cc2)cc1]

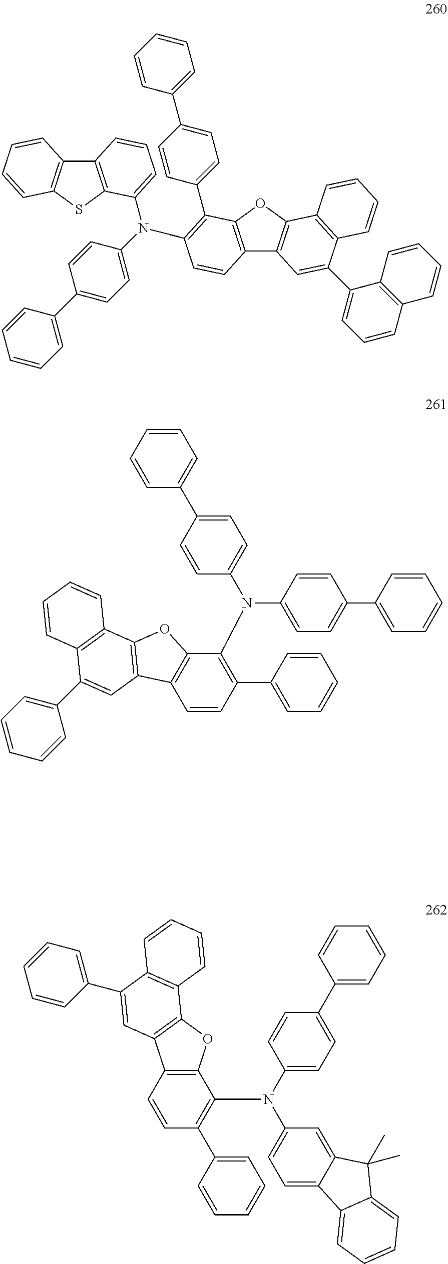 [SMILES: c1ccc(-c2ccc(-c3c(N(c4ccc(-c5ccccc5)cc4)c4cccc5c4sc4ccccc45)ccc4c3oc3c5ccccc5c(-c5cccc6ccccc56)cc43)cc2)cc1 ; c1ccc(-c2ccc(N(c3ccc(-c4ccccc4)cc3)c3c(-c4ccccc4)ccc4c3oc3c5ccccc5c(-c5ccccc5)cc43)cc2)cc1 ; CC1(C)c2ccccc2-c2ccc(N(c3ccc(-c4ccccc4)cc3)c3c(-c4ccccc4)ccc4c3oc3c5ccccc5c(-c5ccccc5)cc43)cc21]

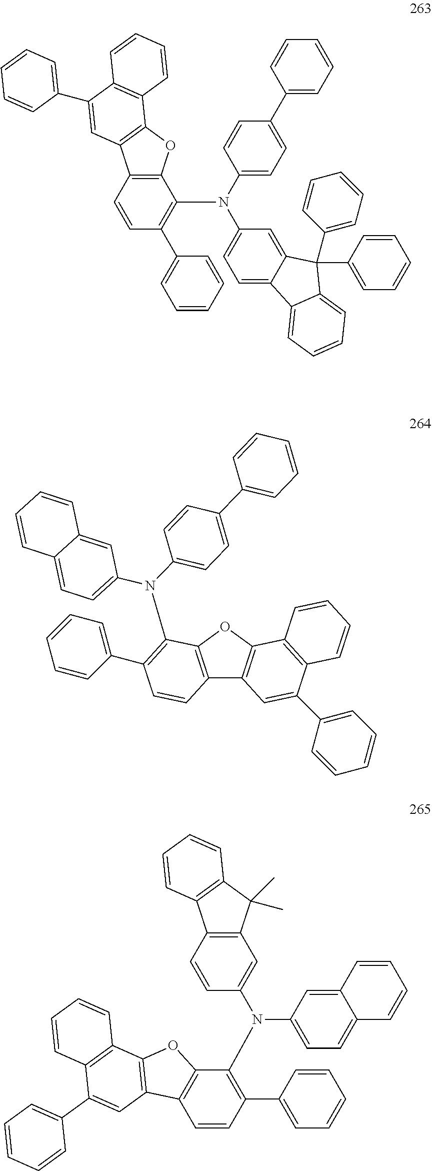 [SMILES: c1ccc(-c2ccc(N(c3ccc4c(c3)C(c3ccccc3)(c3ccccc3)c3ccccc3-4)c3c(-c4ccccc4)ccc4c3oc3c5ccccc5c(-c5ccccc5)cc43)cc2)cc1 ; c1ccc(-c2ccc(N(c3ccc4ccccc4c3)c3c(-c4ccccc4)ccc4c3oc3c5ccccc5c(-c5ccccc5)cc43)cc2)cc1 ; CC1(C)c2ccccc2-c2ccc(N(c3ccc4ccccc4c3)c3c(-c4ccccc4)ccc4c3oc3c5ccccc5c(-c5ccccc5)cc43)cc21]

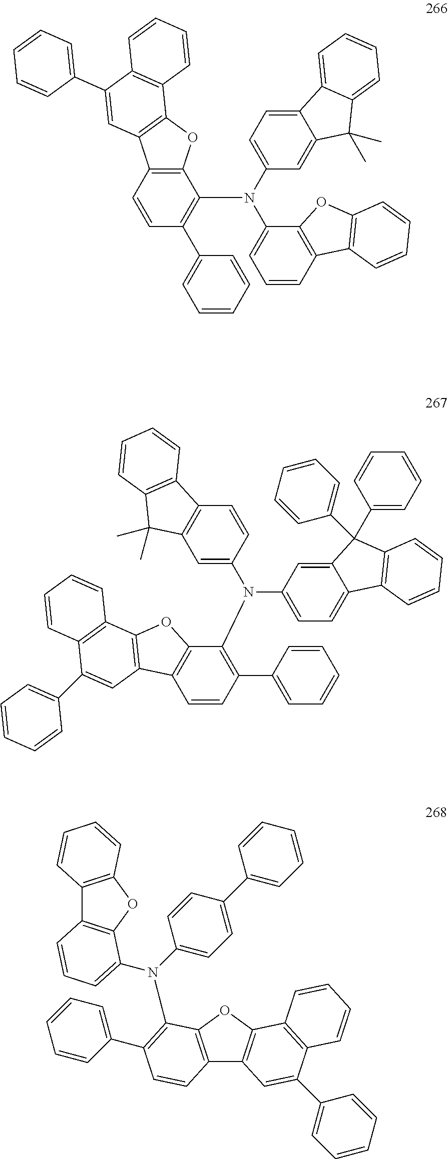 [SMILES: CC1(C)c2ccccc2-c2ccc(N(c3cccc4c3oc3ccccc34)c3c(-c4ccccc4)ccc4c3oc3c5ccccc5c(-c5ccccc5)cc43)cc21 ; CC1(C)c2ccccc2-c2ccc(N(c3ccc4c(c3)C(c3ccccc3)(c3ccccc3)c3ccccc3-4)c3c(-c4ccccc4)ccc4c3oc3c5ccccc5c(-c5ccccc5)cc43)cc21 ; c1ccc(-c2ccc(N(c3cccc4c3oc3ccccc34)c3c(-c4ccccc4)ccc4c3oc3c5ccccc5c(-c5ccccc5)cc43)cc2)cc1]

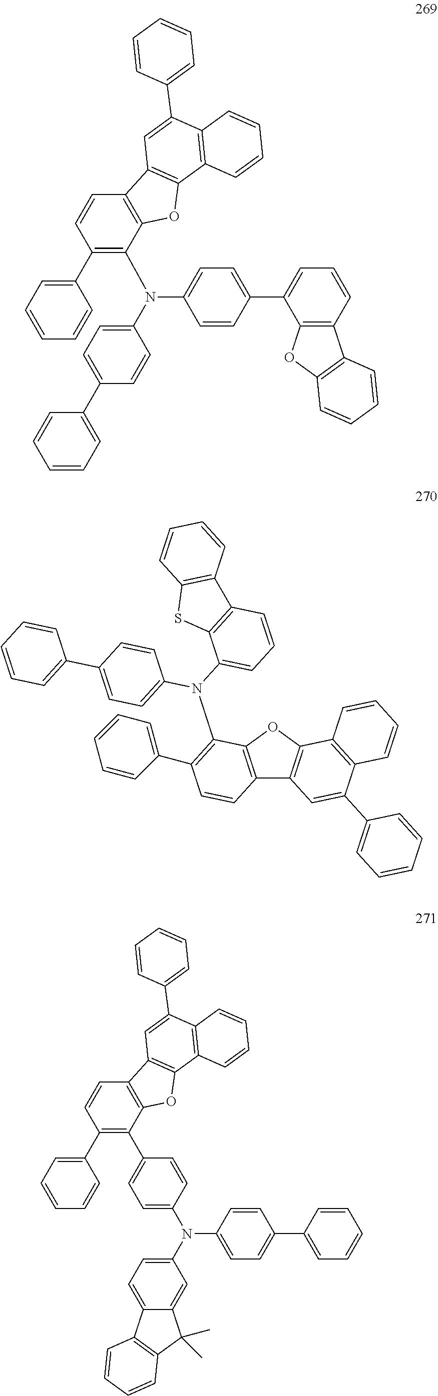 [SMILES: c1ccc(-c2ccc(N(c3ccc(-c4cccc5c4oc4ccccc45)cc3)c3c(-c4ccccc4)ccc4c3oc3c5ccccc5c(-c5ccccc5)cc43)cc2)cc1 ; c1ccc(-c2ccc(N(c3c(-c4ccccc4)ccc4c3oc3c5ccccc5c(-c5ccccc5)cc43)c3cccc4c3sc3ccccc34)cc2)cc1 ; CC1(C)c2ccccc2-c2ccc(N(c3ccc(-c4ccccc4)cc3)c3ccc(-c4c(-c5ccccc5)ccc5c4oc4c6ccccc6c(-c6ccccc6)cc54)cc3)cc21]

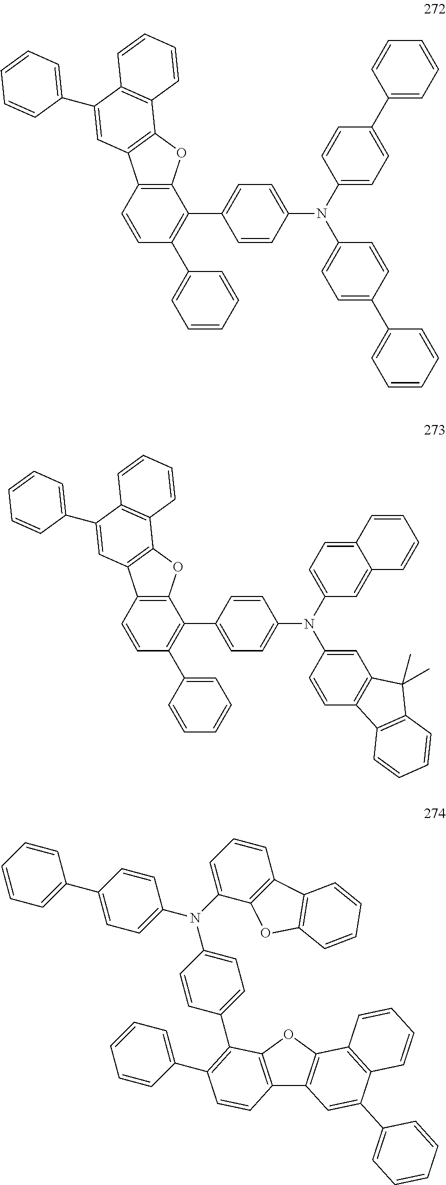 [SMILES: c1ccc(-c2ccc(N(c3ccc(-c4ccccc4)cc3)c3ccc(-c4c(-c5ccccc5)ccc5c4oc4c6ccccc6c(-c6ccccc6)cc54)cc3)cc2)cc1 ; CC1(C)c2ccccc2-c2ccc(N(c3ccc(-c4c(-c5ccccc5)ccc5c4oc4c6ccccc6c(-c6ccccc6)cc54)cc3)c3ccc4ccccc4c3)cc21 ; c1ccc(-c2ccc(N(c3ccc(-c4c(-c5ccccc5)ccc5c4oc4c6ccccc6c(-c6ccccc6)cc54)cc3)c3cccc4c3oc3ccccc34)cc2)cc1]

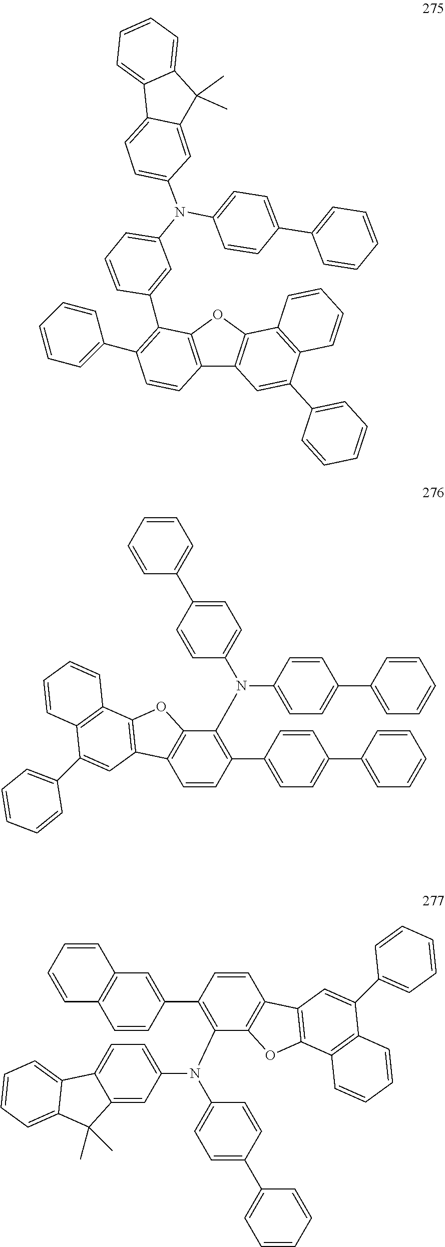 [SMILES: CC1(C)c2ccccc2-c2ccc(N(c3ccc(-c4ccccc4)cc3)c3cccc(-c4c(-c5ccccc5)ccc5c4oc4c6ccccc6c(-c6ccccc6)cc54)c3)cc21 ; c1ccc(-c2ccc(-c3ccc4c(oc5c6ccccc6c(-c6ccccc6)cc45)c3N(c3ccc(-c4ccccc4)cc3)c3ccc(-c4ccccc4)cc3)cc2)cc1 ; CC1(C)c2ccccc2-c2ccc(N(c3ccc(-c4ccccc4)cc3)c3c(-c4ccc5ccccc5c4)ccc4c3oc3c5ccccc5c(-c5ccccc5)cc43)cc21]

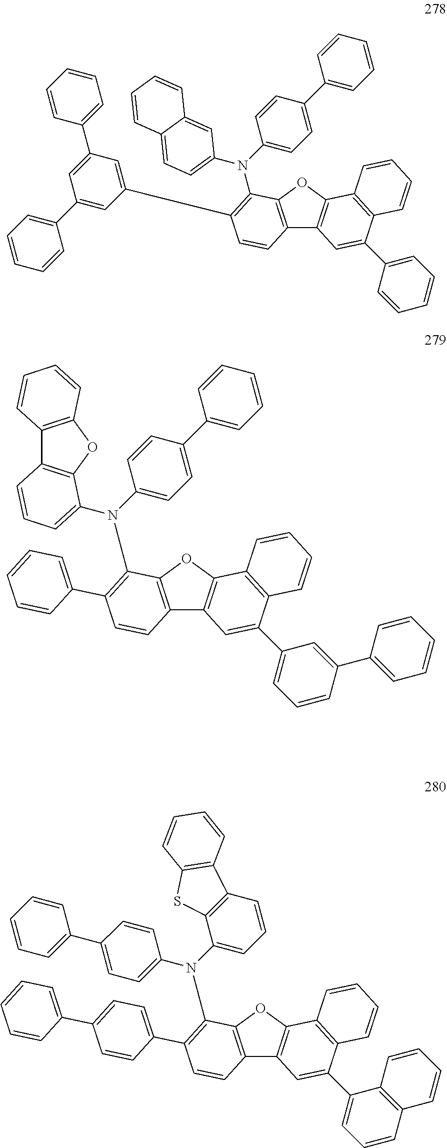 [SMILES: c1ccc(-c2ccc(N(c3ccc4ccccc4c3)c3c(-c4cc(-c5ccccc5)cc(-c5ccccc5)c4)ccc4c3oc3c5ccccc5c(-c5ccccc5)cc43)cc2)cc1 ; c1ccc(-c2ccc(N(c3cccc4c3oc3ccccc34)c3c(-c4ccccc4)ccc4c3oc3c5ccccc5c(-c5cccc(-c6ccccc6)c5)cc43)cc2)cc1 ; c1ccc(-c2ccc(-c3ccc4c(oc5c6ccccc6c(-c6cccc7ccccc67)cc45)c3N(c3ccc(-c4ccccc4)cc3)c3cccc4c3sc3ccccc34)cc2)cc1]

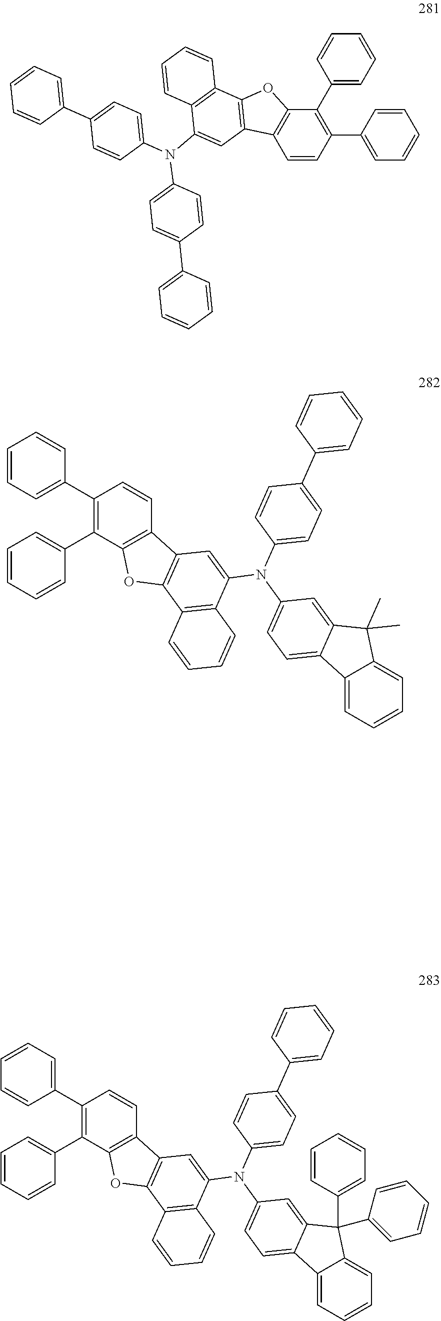 [SMILES: c1ccc(-c2ccc(N(c3ccc(-c4ccccc4)cc3)c3cc4c5ccc(-c6ccccc6)c(-c6ccccc6)c5oc4c4ccccc34)cc2)cc1 ; CC1(C)c2ccccc2-c2ccc(N(c3ccc(-c4ccccc4)cc3)c3cc4c5ccc(-c6ccccc6)c(-c6ccccc6)c5oc4c4ccccc34)cc21 ; c1ccc(-c2ccc(N(c3ccc4c(c3)C(c3ccccc3)(c3ccccc3)c3ccccc3-4)c3cc4c5ccc(-c6ccccc6)c(-c6ccccc6)c5oc4c4ccccc34)cc2)cc1]

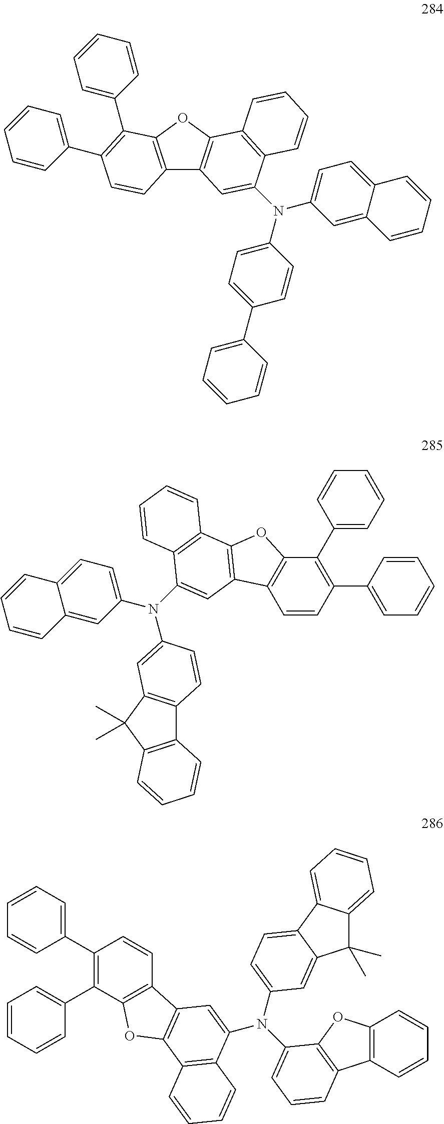 [SMILES: c1ccc(-c2ccc(N(c3ccc4ccccc4c3)c3cc4c5ccc(-c6ccccc6)c(-c6ccccc6)c5oc4c4ccccc34)cc2)cc1 ; CC1(C)c2ccccc2-c2ccc(N(c3ccc4ccccc4c3)c3cc4c5ccc(-c6ccccc6)c(-c6ccccc6)c5oc4c4ccccc34)cc21 ; CC1(C)c2ccccc2-c2ccc(N(c3cc4c5ccc(-c6ccccc6)c(-c6ccccc6)c5oc4c4ccccc34)c3cccc4c3oc3ccccc34)cc21]

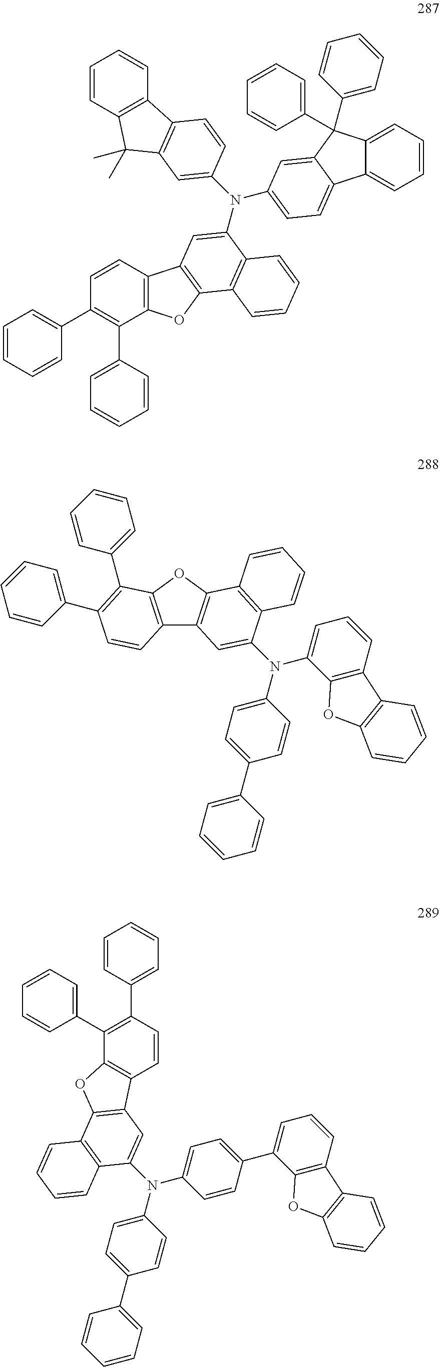 [SMILES: CC1(C)c2ccccc2-c2ccc(N(c3ccc4c(c3)C(c3ccccc3)(c3ccccc3)c3ccccc3-4)c3cc4c5ccc(-c6ccccc6)c(-c6ccccc6)c5oc4c4ccccc34)cc21 ; c1ccc(-c2ccc(N(c3cc4c5ccc(-c6ccccc6)c(-c6ccccc6)c5oc4c4ccccc34)c3cccc4c3oc3ccccc34)cc2)cc1 ; c1ccc(-c2ccc(N(c3ccc(-c4cccc5c4oc4ccccc45)cc3)c3cc4c5ccc(-c6ccccc6)c(-c6ccccc6)c5oc4c4ccccc34)cc2)cc1]

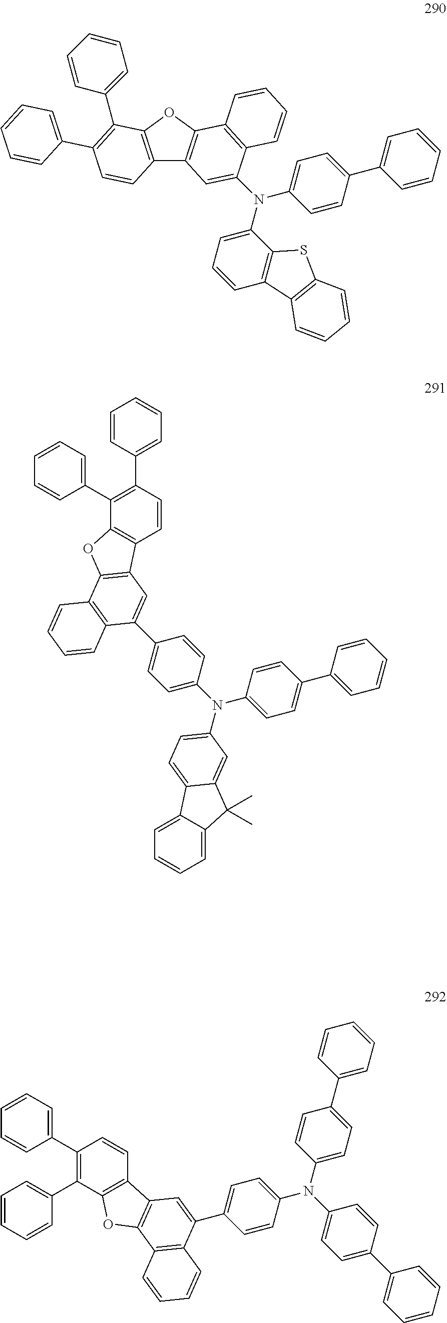 [SMILES: c1ccc(-c2ccc(N(c3cc4c5ccc(-c6ccccc6)c(-c6ccccc6)c5oc4c4ccccc34)c3cccc4c3sc3ccccc34)cc2)cc1 ; CC1(C)c2ccccc2-c2ccc(N(c3ccc(-c4ccccc4)cc3)c3ccc(-c4cc5c6ccc(-c7ccccc7)c(-c7ccccc7)c6oc5c5ccccc45)cc3)cc21 ; c1ccc(-c2ccc(N(c3ccc(-c4ccccc4)cc3)c3ccc(-c4cc5c6ccc(-c7ccccc7)c(-c7ccccc7)c6oc5c5ccccc45)cc3)cc2)cc1]

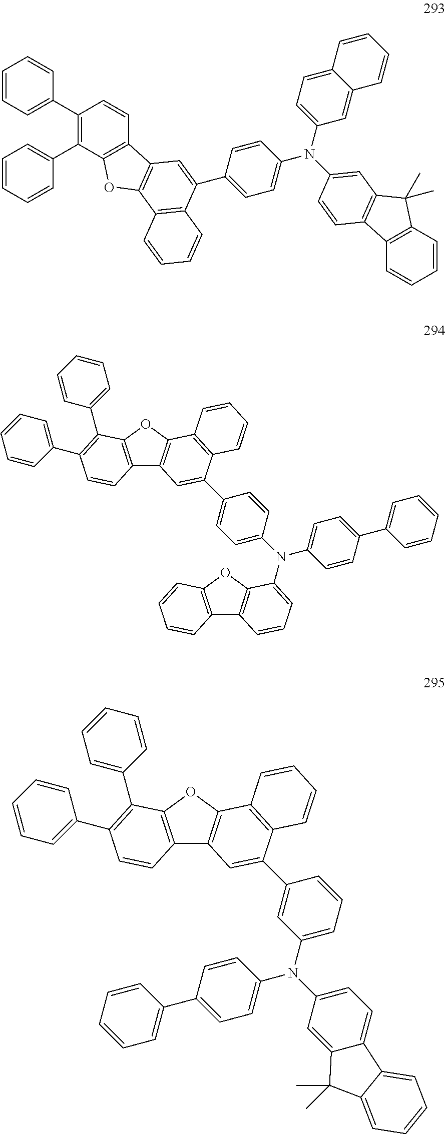 [SMILES: CC1(C)c2ccccc2-c2ccc(N(c3ccc(-c4cc5c6ccc(-c7ccccc7)c(-c7ccccc7)c6oc5c5ccccc45)cc3)c3ccc4ccccc4c3)cc21 ; c1ccc(-c2ccc(N(c3ccc(-c4cc5c6ccc(-c7ccccc7)c(-c7ccccc7)c6oc5c5ccccc45)cc3)c3cccc4c3oc3ccccc34)cc2)cc1 ; CC1(C)c2ccccc2-c2ccc(N(c3ccc(-c4ccccc4)cc3)c3cccc(-c4cc5c6ccc(-c7ccccc7)c(-c7ccccc7)c6oc5c5ccccc45)c3)cc21]

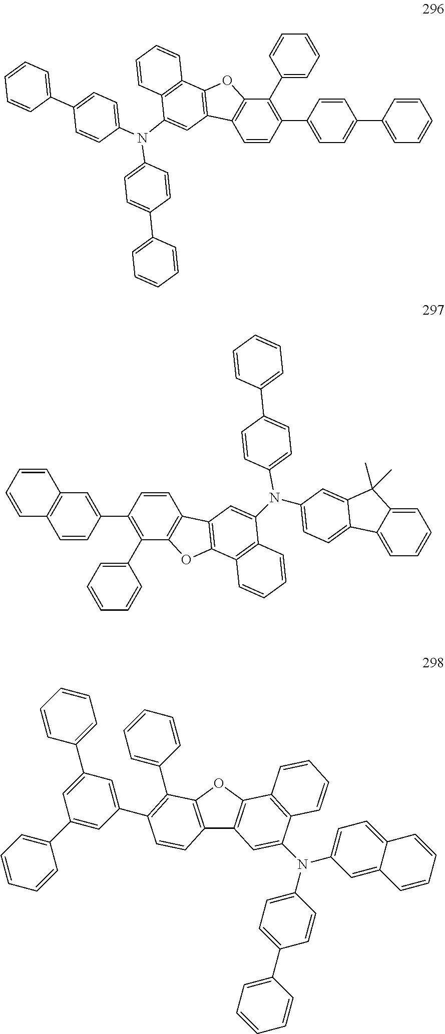 [SMILES: c1ccc(-c2ccc(-c3ccc4c(oc5c6ccccc6c(N(c6ccc(-c7ccccc7)cc6)c6ccc(-c7ccccc7)cc6)cc45)c3-c3ccccc3)cc2)cc1 ; CC1(C)c2ccccc2-c2ccc(N(c3ccc(-c4ccccc4)cc3)c3cc4c5ccc(-c6ccc7ccccc7c6)c(-c6ccccc6)c5oc4c4ccccc34)cc21 ; c1ccc(-c2ccc(N(c3ccc4ccccc4c3)c3cc4c5ccc(-c6cc(-c7ccccc7)cc(-c7ccccc7)c6)c(-c6ccccc6)c5oc4c4ccccc34)cc2)cc1]

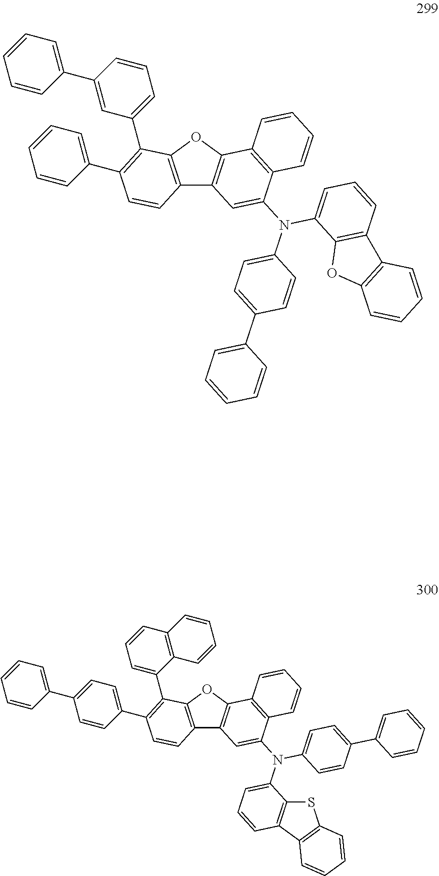 [SMILES: c1ccc(-c2ccc(N(c3cc4c5ccc(-c6ccccc6)c(-c6cccc(-c7ccccc7)c6)c5oc4c4ccccc34)c3cccc4c3oc3ccccc34)cc2)cc1 ; c1ccc(-c2ccc(-c3ccc4c(oc5c6ccccc6c(N(c6ccc(-c7ccccc7)cc6)c6cccc7c6sc6ccccc67)cc45)c3-c3cccc4ccccc34)cc2)cc1]

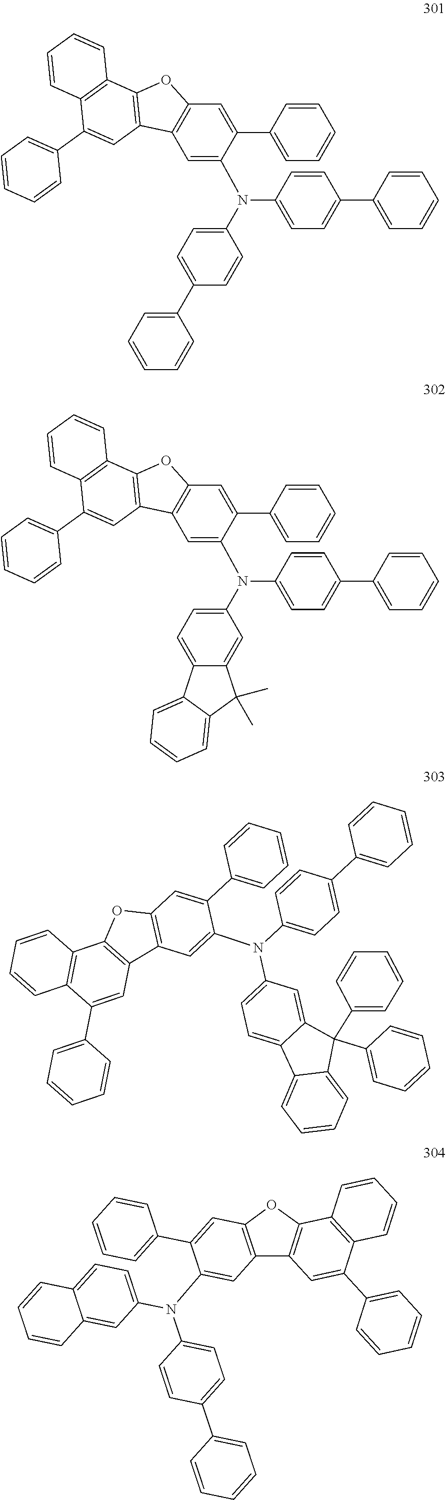 [SMILES: c1ccc(-c2ccc(N(c3ccc(-c4ccccc4)cc3)c3cc4c(cc3-c3ccccc3)oc3c5ccccc5c(-c5ccccc5)cc43)cc2)cc1 ; CC1(C)c2ccccc2-c2ccc(N(c3ccc(-c4ccccc4)cc3)c3cc4c(cc3-c3ccccc3)oc3c5ccccc5c(-c5ccccc5)cc43)cc21 ; c1ccc(-c2ccc(N(c3ccc4c(c3)C(c3ccccc3)(c3ccccc3)c3ccccc3-4)c3cc4c(cc3-c3ccccc3)oc3c5ccccc5c(-c5ccccc5)cc43)cc2)cc1 ; c1ccc(-c2ccc(N(c3ccc4ccccc4c3)c3cc4c(cc3-c3ccccc3)oc3c5ccccc5c(-c5ccccc5)cc43)cc2)cc1]

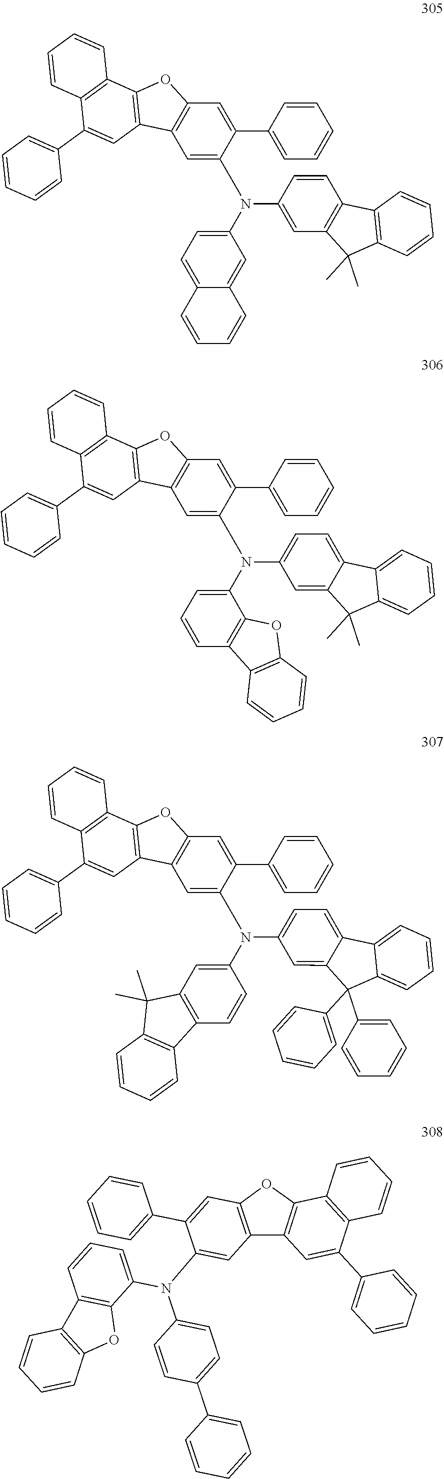 [SMILES: CC1(C)c2ccccc2-c2ccc(N(c3ccc4ccccc4c3)c3cc4c(cc3-c3ccccc3)oc3c5ccccc5c(-c5ccccc5)cc43)cc21 ; CC1(C)c2ccccc2-c2ccc(N(c3cc4c(cc3-c3ccccc3)oc3c5ccccc5c(-c5ccccc5)cc43)c3cccc4c3oc3ccccc34)cc21 ; CC1(C)c2ccccc2-c2ccc(N(c3ccc4c(c3)C(c3ccccc3)(c3ccccc3)c3ccccc3-4)c3cc4c(cc3-c3ccccc3)oc3c5ccccc5c(-c5ccccc5)cc43)cc21 ; c1ccc(-c2ccc(N(c3cc4c(cc3-c3ccccc3)oc3c5ccccc5c(-c5ccccc5)cc43)c3cccc4c3oc3ccccc34)cc2)cc1]

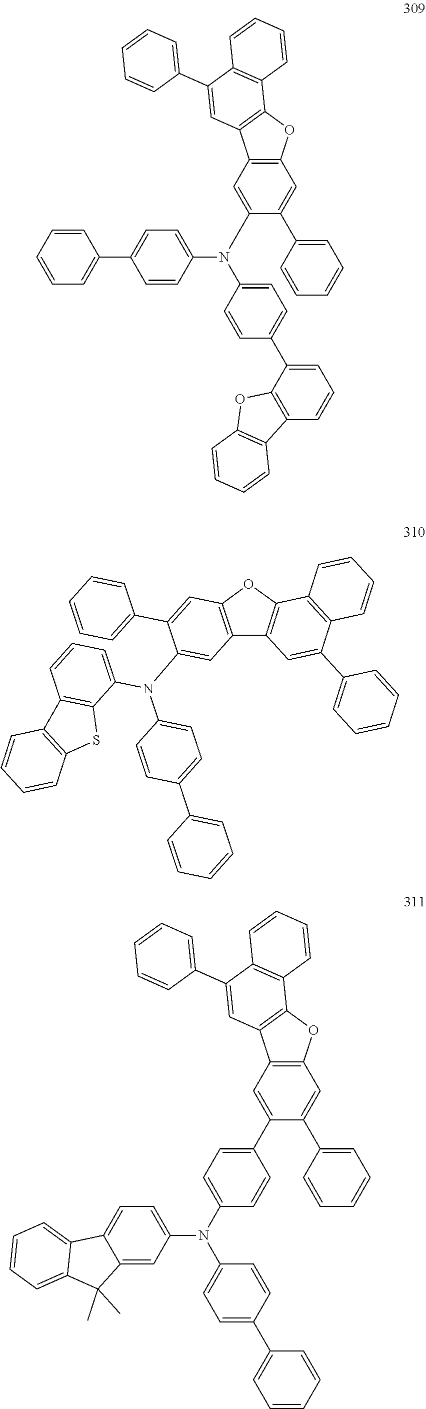 [SMILES: c1ccc(-c2ccc(N(c3ccc(-c4cccc5c4oc4ccccc45)cc3)c3cc4c(cc3-c3ccccc3)oc3c5ccccc5c(-c5ccccc5)cc43)cc2)cc1 ; c1ccc(-c2ccc(N(c3cc4c(cc3-c3ccccc3)oc3c5ccccc5c(-c5ccccc5)cc43)c3cccc4c3sc3ccccc34)cc2)cc1 ; CC1(C)c2ccccc2-c2ccc(N(c3ccc(-c4ccccc4)cc3)c3ccc(-c4cc5c(cc4-c4ccccc4)oc4c6ccccc6c(-c6ccccc6)cc54)cc3)cc21]

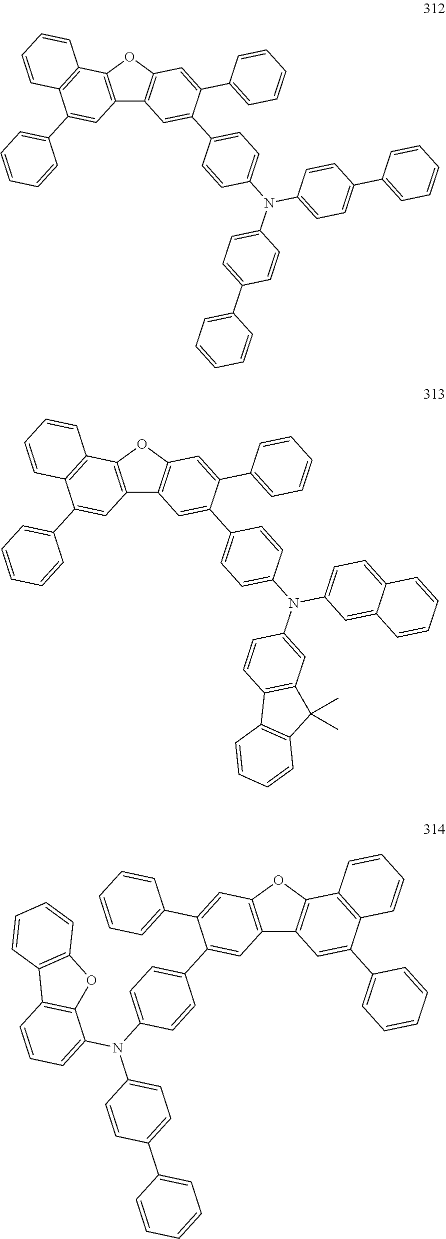 [SMILES: c1ccc(-c2ccc(N(c3ccc(-c4ccccc4)cc3)c3ccc(-c4cc5c(cc4-c4ccccc4)oc4c6ccccc6c(-c6ccccc6)cc54)cc3)cc2)cc1 ; CC1(C)c2ccccc2-c2ccc(N(c3ccc(-c4cc5c(cc4-c4ccccc4)oc4c6ccccc6c(-c6ccccc6)cc54)cc3)c3ccc4ccccc4c3)cc21 ; c1ccc(-c2ccc(N(c3ccc(-c4cc5c(cc4-c4ccccc4)oc4c6ccccc6c(-c6ccccc6)cc54)cc3)c3cccc4c3oc3ccccc34)cc2)cc1]

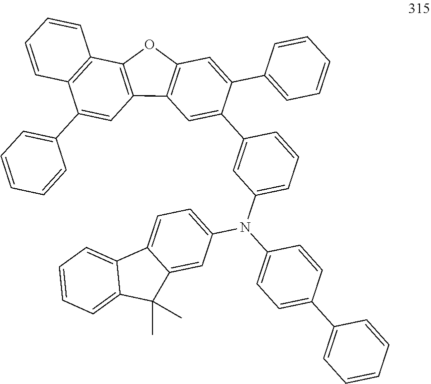 [SMILES: CC1(C)c2ccccc2-c2ccc(N(c3ccc(-c4ccccc4)cc3)c3cccc(-c4cc5c(cc4-c4ccccc4)oc4c6ccccc6c(-c6ccccc6)cc54)c3)cc21]

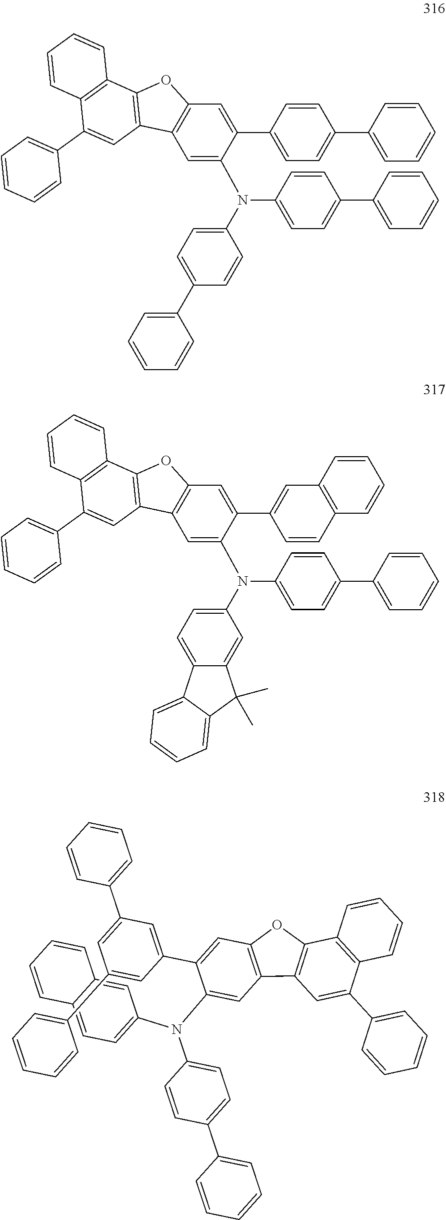 [SMILES: c1ccc(-c2ccc(-c3cc4oc5c6ccccc6c(-c6ccccc6)cc5c4cc3N(c3ccc(-c4ccccc4)cc3)c3ccc(-c4ccccc4)cc3)cc2)cc1 ; CC1(C)c2ccccc2-c2ccc(N(c3ccc(-c4ccccc4)cc3)c3cc4c(cc3-c3ccc5ccccc5c3)oc3c5ccccc5c(-c5ccccc5)cc43)cc21 ; c1ccc(-c2ccc(N(c3ccc4ccccc4c3)c3cc4c(cc3-c3cc(-c5ccccc5)cc(-c5ccccc5)c3)oc3c5ccccc5c(-c5ccccc5)cc43)cc2)cc1]

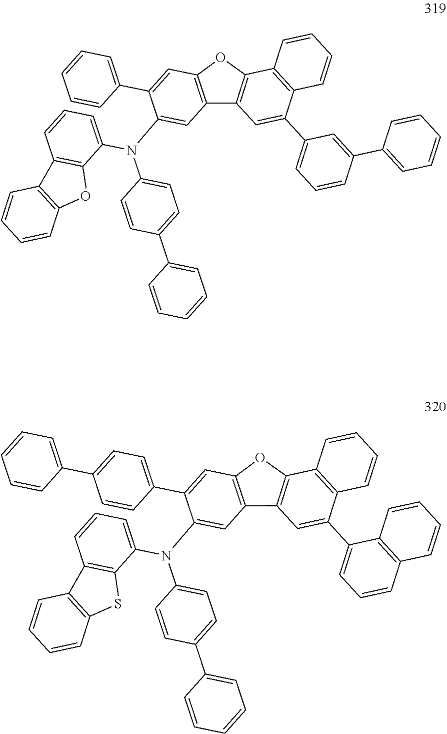 [SMILES: c1ccc(-c2ccc(N(c3cc4c(cc3-c3ccccc3)oc3c5ccccc5c(-c5cccc(-c6ccccc6)c5)cc43)c3cccc4c3oc3ccccc34)cc2)cc1 ; c1ccc(-c2ccc(-c3cc4oc5c6ccccc6c(-c6cccc7ccccc67)cc5c4cc3N(c3ccc(-c4ccccc4)cc3)c3cccc4c3sc3ccccc34)cc2)cc1]

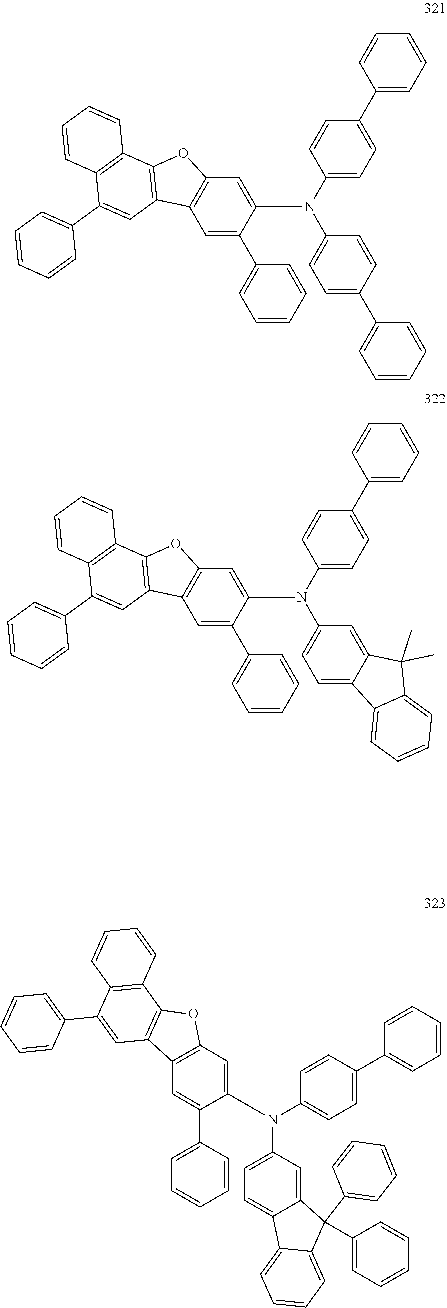

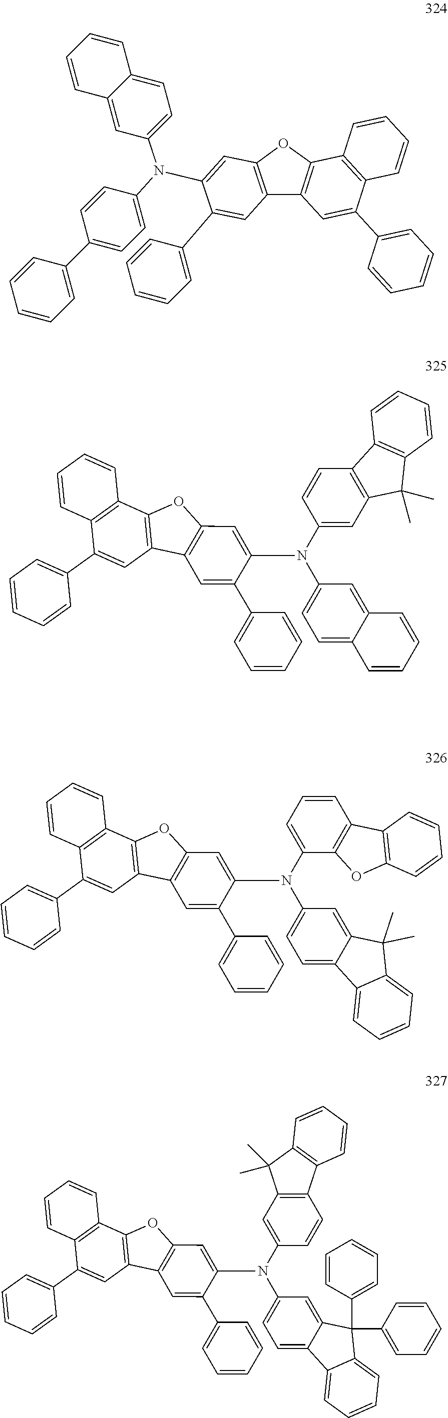 [SMILES: c1ccc(-c2ccc(N(c3ccc4ccccc4c3)c3cc4oc5c6ccccc6c(-c6ccccc6)cc5c4cc3-c3ccccc3)cc2)cc1 ; CC1(C)c2ccccc2-c2ccc(N(c3ccc4ccccc4c3)c3cc4oc5c6ccccc6c(-c6ccccc6)cc5c4cc3-c3ccccc3)cc21 ; CC1(C)c2ccccc2-c2ccc(N(c3cc4oc5c6ccccc6c(-c6ccccc6)cc5c4cc3-c3ccccc3)c3cccc4c3oc3ccccc34)cc21 ; CC1(C)c2ccccc2-c2ccc(N(c3ccc4c(c3)C(c3ccccc3)(c3ccccc3)c3ccccc3-4)c3cc4oc5c6ccccc6c(-c6ccccc6)cc5c4cc3-c3ccccc3)cc21]

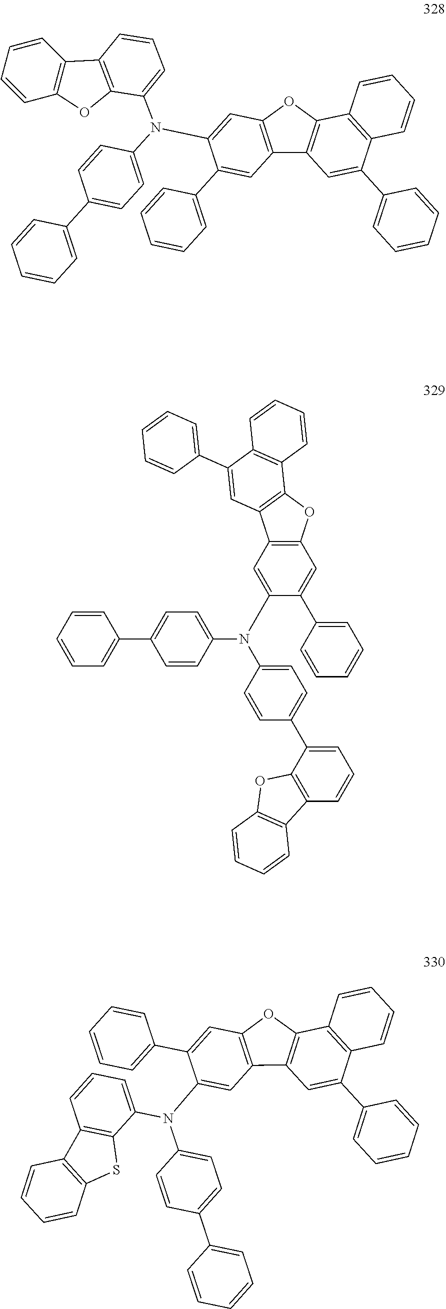 [SMILES: c1ccc(-c2ccc(N(c3cc4oc5c6ccccc6c(-c6ccccc6)cc5c4cc3-c3ccccc3)c3cccc4c3oc3ccccc34)cc2)cc1 ; c1ccc(-c2ccc(N(c3ccc(-c4cccc5c4oc4ccccc45)cc3)c3cc4c(cc3-c3ccccc3)oc3c5ccccc5c(-c5ccccc5)cc43)cc2)cc1 ; c1ccc(-c2ccc(N(c3cc4c(cc3-c3ccccc3)oc3c5ccccc5c(-c5ccccc5)cc43)c3cccc4c3sc3ccccc34)cc2)cc1]

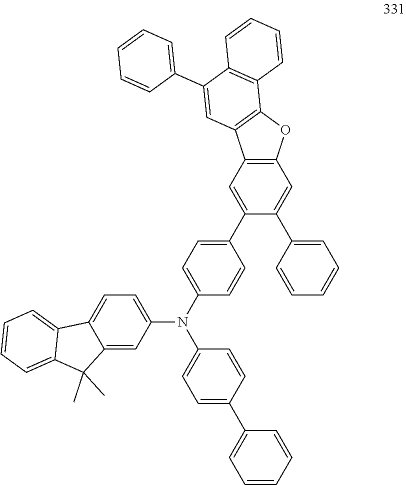 [SMILES: CC1(C)c2ccccc2-c2ccc(N(c3ccc(-c4ccccc4)cc3)c3ccc(-c4cc5c(cc4-c4ccccc4)oc4c6ccccc6c(-c6ccccc6)cc54)cc3)cc21]

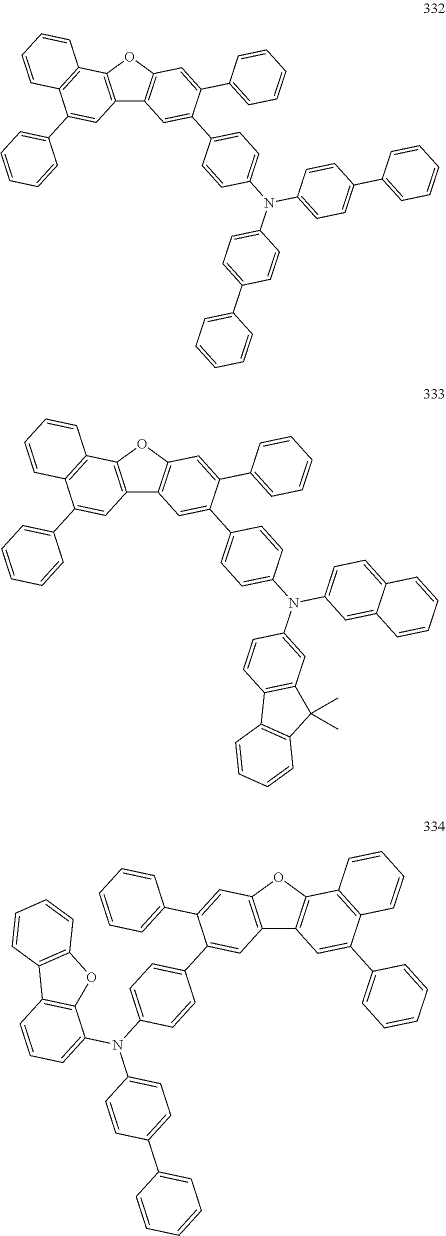 [SMILES: c1ccc(-c2ccc(N(c3ccc(-c4ccccc4)cc3)c3ccc(-c4cc5c(cc4-c4ccccc4)oc4c6ccccc6c(-c6ccccc6)cc54)cc3)cc2)cc1 ; CC1(C)c2ccccc2-c2ccc(N(c3ccc(-c4cc5c(cc4-c4ccccc4)oc4c6ccccc6c(-c6ccccc6)cc54)cc3)c3ccc4ccccc4c3)cc21 ; c1ccc(-c2ccc(N(c3ccc(-c4cc5c(cc4-c4ccccc4)oc4c6ccccc6c(-c6ccccc6)cc54)cc3)c3cccc4c3oc3ccccc34)cc2)cc1]

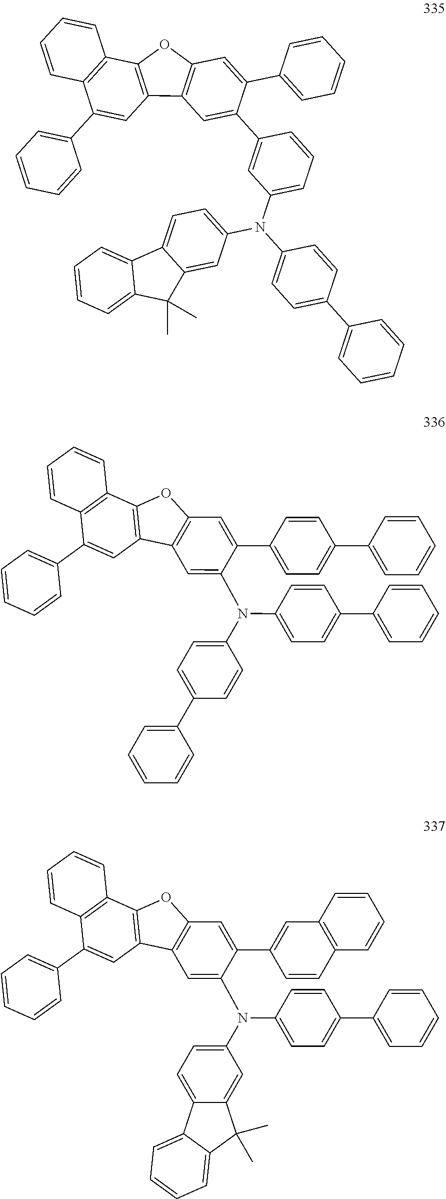 [SMILES: CC1(C)c2ccccc2-c2ccc(N(c3ccc(-c4ccccc4)cc3)c3cccc(-c4cc5c(cc4-c4ccccc4)oc4c6ccccc6c(-c6ccccc6)cc54)c3)cc21 ; c1ccc(-c2ccc(-c3cc4oc5c6ccccc6c(-c6ccccc6)cc5c4cc3N(c3ccc(-c4ccccc4)cc3)c3ccc(-c4ccccc4)cc3)cc2)cc1 ; CC1(C)c2ccccc2-c2ccc(N(c3ccc(-c4ccccc4)cc3)c3cc4c(cc3-c3ccc5ccccc5c3)oc3c5ccccc5c(-c5ccccc5)cc43)cc21]

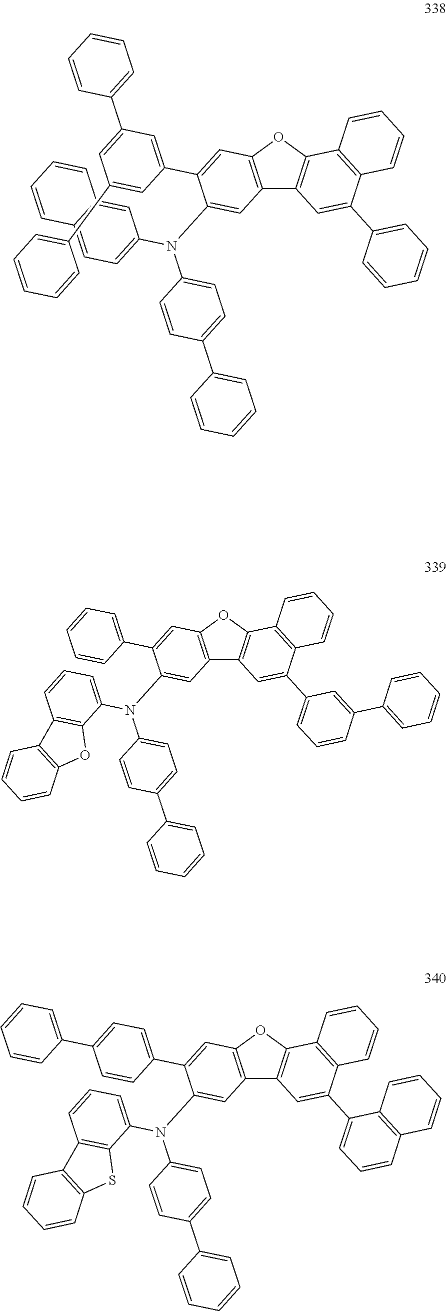 [SMILES: c1ccc(-c2ccc(N(c3ccc4ccccc4c3)c3cc4c(cc3-c3cc(-c5ccccc5)cc(-c5ccccc5)c3)oc3c5ccccc5c(-c5ccccc5)cc43)cc2)cc1 ; c1ccc(-c2ccc(N(c3cc4c(cc3-c3ccccc3)oc3c5ccccc5c(-c5cccc(-c6ccccc6)c5)cc43)c3cccc4c3oc3ccccc34)cc2)cc1 ; c1ccc(-c2ccc(-c3cc4oc5c6ccccc6c(-c6cccc7ccccc67)cc5c4cc3N(c3ccc(-c4ccccc4)cc3)c3cccc4c3sc3ccccc34)cc2)cc1]

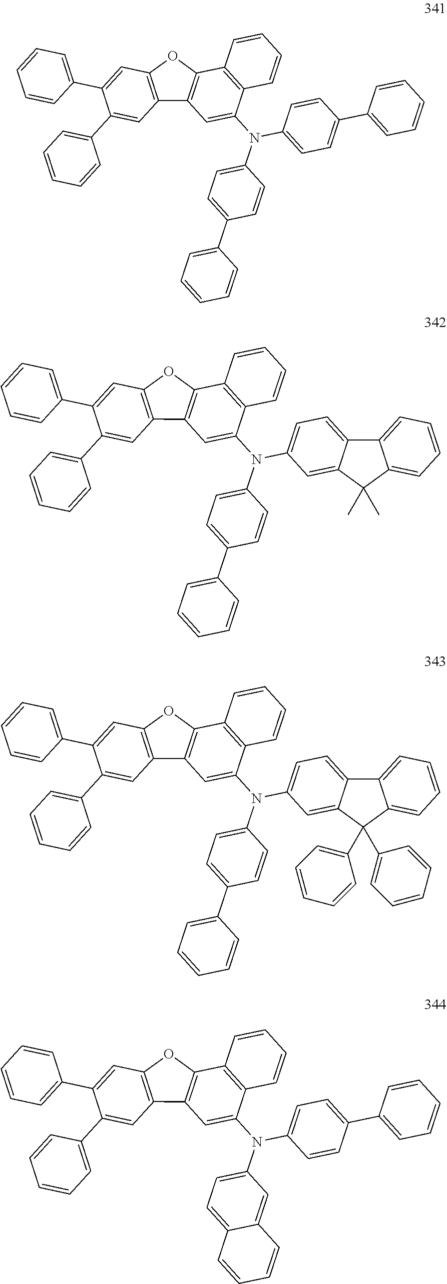 [SMILES: c1ccc(-c2ccc(N(c3ccc(-c4ccccc4)cc3)c3cc4c5cc(-c6ccccc6)c(-c6ccccc6)cc5oc4c4ccccc34)cc2)cc1 ; CC1(C)c2ccccc2-c2ccc(N(c3ccc(-c4ccccc4)cc3)c3cc4c5cc(-c6ccccc6)c(-c6ccccc6)cc5oc4c4ccccc34)cc21 ; c1ccc(-c2ccc(N(c3ccc4c(c3)C(c3ccccc3)(c3ccccc3)c3ccccc3-4)c3cc4c5cc(-c6ccccc6)c(-c6ccccc6)cc5oc4c4ccccc34)cc2)cc1 ; c1ccc(-c2ccc(N(c3ccc4ccccc4c3)c3cc4c5cc(-c6ccccc6)c(-c6ccccc6)cc5oc4c4ccccc34)cc2)cc1]

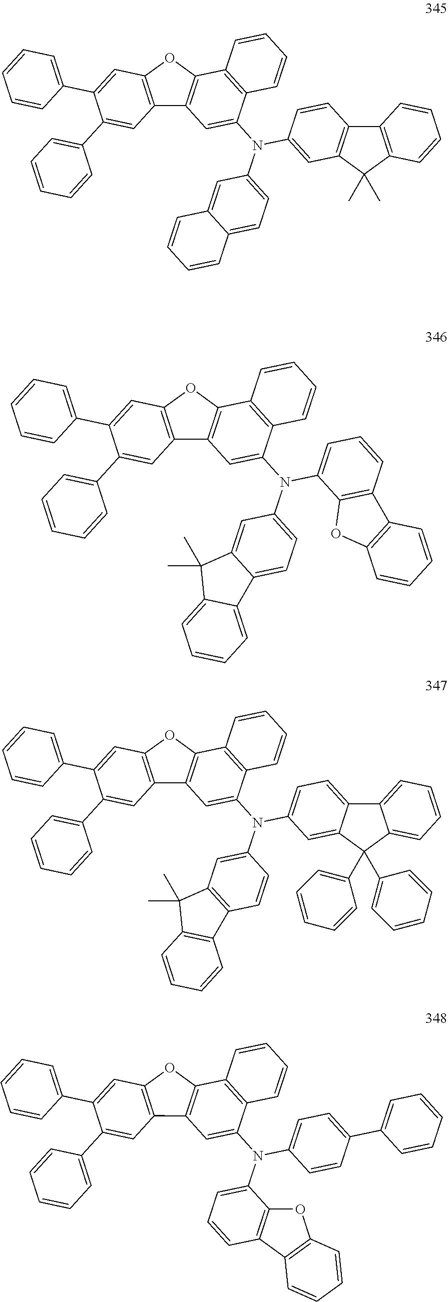 [SMILES: CC1(C)c2ccccc2-c2ccc(N(c3ccc4ccccc4c3)c3cc4c5cc(-c6ccccc6)c(-c6ccccc6)cc5oc4c4ccccc34)cc21 ; CC1(C)c2ccccc2-c2ccc(N(c3cc4c5cc(-c6ccccc6)c(-c6ccccc6)cc5oc4c4ccccc34)c3cccc4c3oc3ccccc34)cc21 ; CC1(C)c2ccccc2-c2ccc(N(c3ccc4c(c3)C(c3ccccc3)(c3ccccc3)c3ccccc3-4)c3cc4c5cc(-c6ccccc6)c(-c6ccccc6)cc5oc4c4ccccc34)cc21 ; c1ccc(-c2ccc(N(c3cc4c5cc(-c6ccccc6)c(-c6ccccc6)cc5oc4c4ccccc34)c3cccc4c3oc3ccccc34)cc2)cc1]

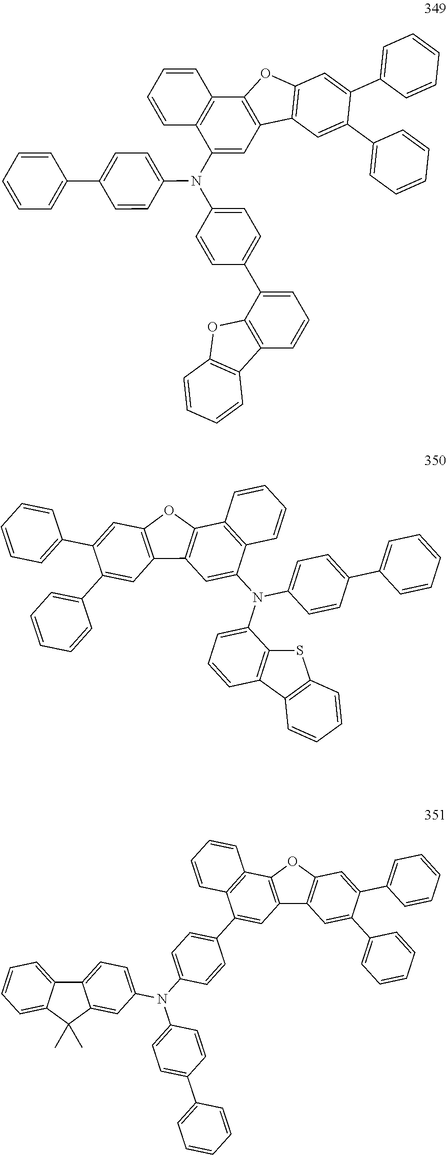 [SMILES: c1ccc(-c2ccc(N(c3ccc(-c4cccc5c4oc4ccccc45)cc3)c3cc4c5cc(-c6ccccc6)c(-c6ccccc6)cc5oc4c4ccccc34)cc2)cc1 ; c1ccc(-c2ccc(N(c3cc4c5cc(-c6ccccc6)c(-c6ccccc6)cc5oc4c4ccccc34)c3cccc4c3sc3ccccc34)cc2)cc1 ; CC1(C)c2ccccc2-c2ccc(N(c3ccc(-c4ccccc4)cc3)c3ccc(-c4cc5c6cc(-c7ccccc7)c(-c7ccccc7)cc6oc5c5ccccc45)cc3)cc21]

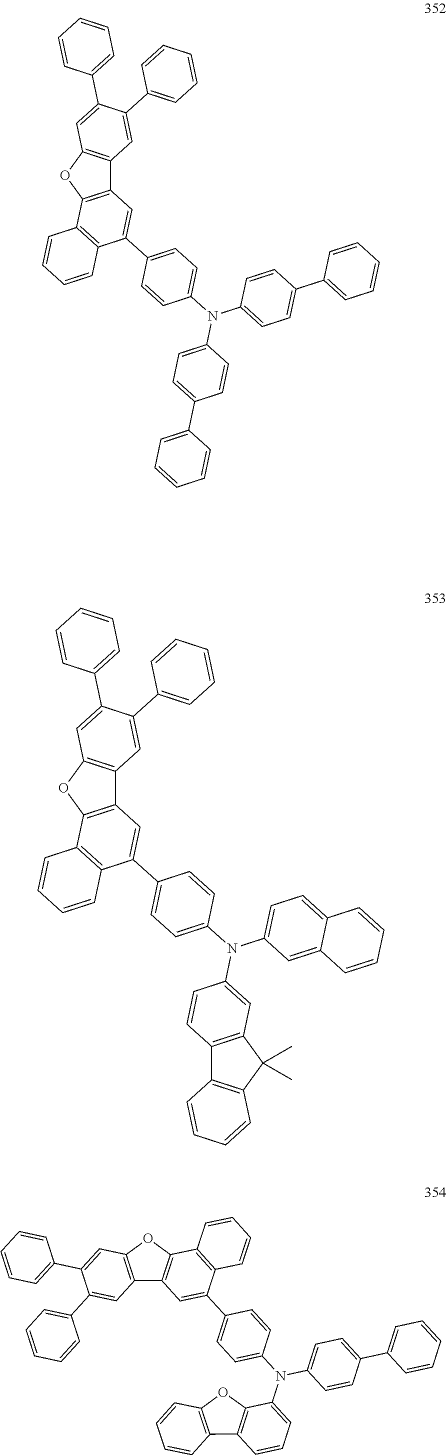 [SMILES: c1ccc(-c2ccc(N(c3ccc(-c4ccccc4)cc3)c3ccc(-c4cc5c6cc(-c7ccccc7)c(-c7ccccc7)cc6oc5c5ccccc45)cc3)cc2)cc1 ; CC1(C)c2ccccc2-c2ccc(N(c3ccc(-c4cc5c6cc(-c7ccccc7)c(-c7ccccc7)cc6oc5c5ccccc45)cc3)c3ccc4ccccc4c3)cc21 ; c1ccc(-c2ccc(N(c3ccc(-c4cc5c6cc(-c7ccccc7)c(-c7ccccc7)cc6oc5c5ccccc45)cc3)c3cccc4c3oc3ccccc34)cc2)cc1]

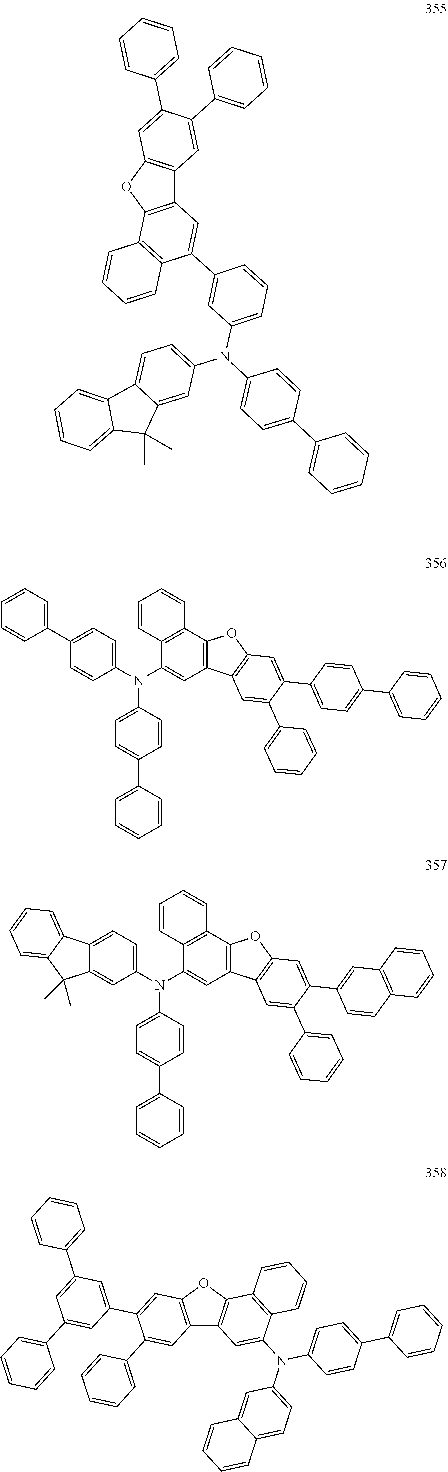 [SMILES: CC1(C)c2ccccc2-c2ccc(N(c3ccc(-c4ccccc4)cc3)c3cccc(-c4cc5c6cc(-c7ccccc7)c(-c7ccccc7)cc6oc5c5ccccc45)c3)cc21 ; c1ccc(-c2ccc(-c3cc4oc5c6ccccc6c(N(c6ccc(-c7ccccc7)cc6)c6ccc(-c7ccccc7)cc6)cc5c4cc3-c3ccccc3)cc2)cc1 ; CC1(C)c2ccccc2-c2ccc(N(c3ccc(-c4ccccc4)cc3)c3cc4c5cc(-c6ccccc6)c(-c6ccc7ccccc7c6)cc5oc4c4ccccc34)cc21 ; c1ccc(-c2ccc(N(c3ccc4ccccc4c3)c3cc4c5cc(-c6ccccc6)c(-c6cc(-c7ccccc7)cc(-c7ccccc7)c6)cc5oc4c4ccccc34)cc2)cc1]

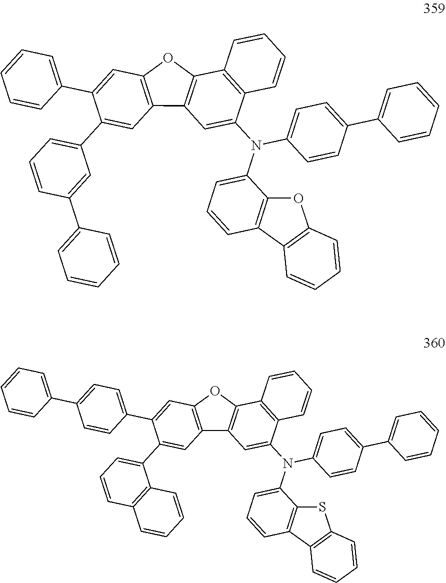 [SMILES: c1ccc(-c2ccc(N(c3cc4c5cc(-c6cccc(-c7ccccc7)c6)c(-c6ccccc6)cc5oc4c4ccccc34)c3cccc4c3oc3ccccc34)cc2)cc1 ; c1ccc(-c2ccc(-c3cc4oc5c6ccccc6c(N(c6ccc(-c7ccccc7)cc6)c6cccc7c6sc6ccccc67)cc5c4cc3-c3cccc4ccccc34)cc2)cc1]

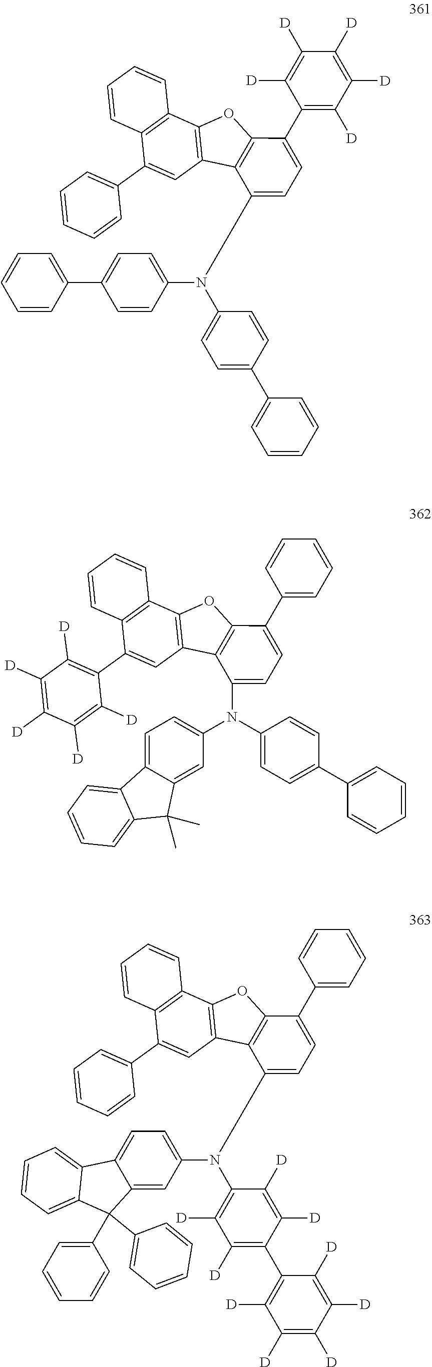 [SMILES: [2H]c1c([2H])c([2H])c(-c2ccc(N(c3ccc(-c4ccccc4)cc3)c3ccc(-c4ccccc4)cc3)c3c2oc2c4ccccc4c(-c4ccccc4)cc23)c([2H])c1[2H] ; [2H]c1c([2H])c([2H])c(-c2cc3c(oc4c(-c5ccccc5)ccc(N(c5ccc(-c6ccccc6)cc5)c5ccc6c(c5)C(C)(C)c5ccccc5-6)c43)c3ccccc23)c([2H])c1[2H] ; [2H]c1c([2H])c([2H])c(-c2c([2H])c([2H])c(N(c3ccc4c(c3)C(c3ccccc3)(c3ccccc3)c3ccccc3-4)c3ccc(-c4ccccc4)c4oc5c6ccccc6c(-c6ccccc6)cc5c34)c([2H])c2[2H])c([2H])c1[2H]]

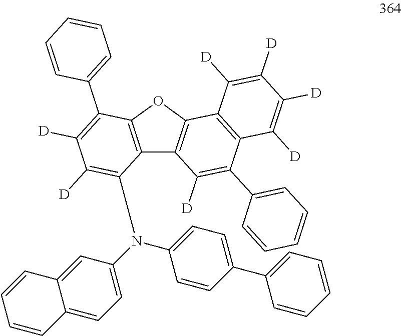 [SMILES: [2H]c1c([2H])c(N(c2ccc(-c3ccccc3)cc2)c2ccc3ccccc3c2)c2c(oc3c4c([2H])c([2H])c([2H])c([2H])c4c(-c4ccccc4)c([2H])c32)c1-c1ccccc1]

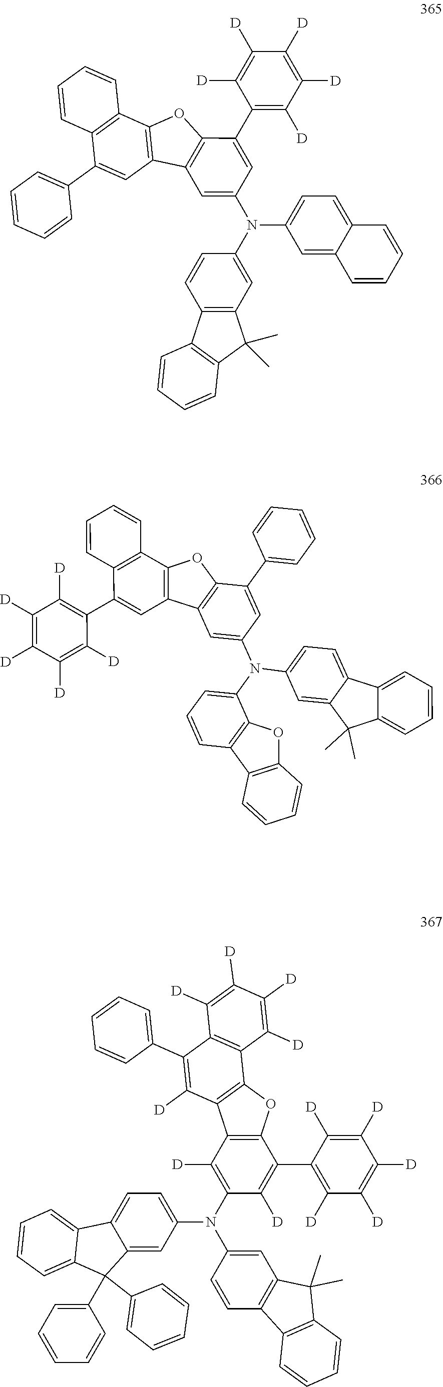 [SMILES: [2H]c1c([2H])c([2H])c(-c2cc(N(c3ccc4c(c3)C(C)(C)c3ccccc3-4)c3ccc4ccccc4c3)cc3c2oc2c4ccccc4c(-c4ccccc4)cc32)c([2H])c1[2H] ; [2H]c1c([2H])c([2H])c(-c2cc3c4cc(N(c5ccc6c(c5)C(C)(C)c5ccccc5-6)c5cccc6c5oc5ccccc56)cc(-c5ccccc5)c4oc3c3ccccc23)c([2H])c1[2H] ; [2H]c1c([2H])c([2H])c(-c2c([2H])c(N(c3ccc4c(c3)C(C)(C)c3ccccc3-4)c3ccc4c(c3)C(c3ccccc3)(c3ccccc3)c3ccccc3-4)c([2H])c3c2oc2c4c([2H])c([2H])c([2H])c([2H])c4c(-c4ccccc4)c([2H])c23)c([2H])c1[2H]]

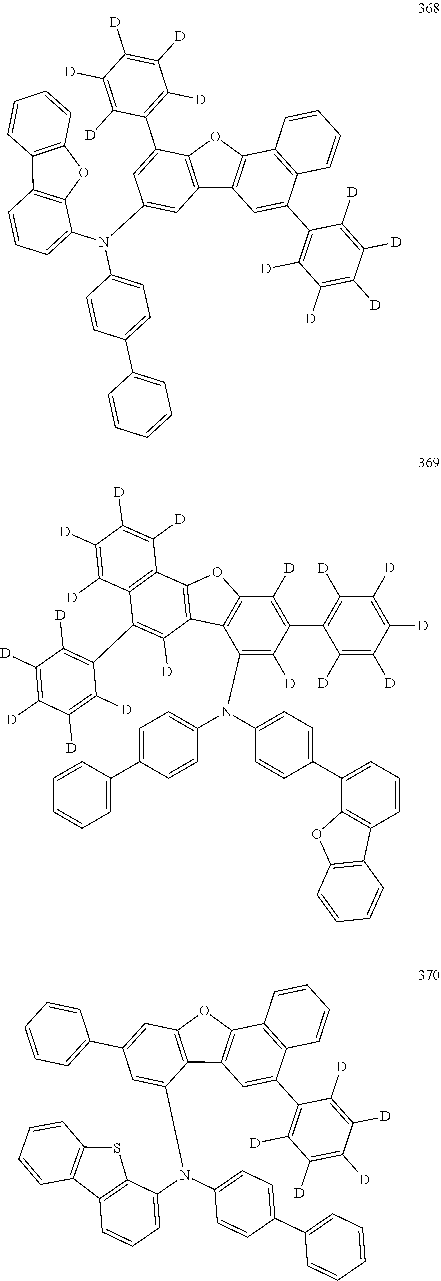 [SMILES: [2H]c1c([2H])c([2H])c(-c2cc3c4cc(N(c5ccc(-c6ccccc6)cc5)c5cccc6c5oc5ccccc56)cc(-c5c([2H])c([2H])c([2H])c([2H])c5[2H])c4oc3c3ccccc23)c([2H])c1[2H] ; [2H]c1c([2H])c([2H])c(-c2c([2H])c(N(c3ccc(-c4ccccc4)cc3)c3ccc(-c4cccc5c4oc4ccccc45)cc3)c3c(oc4c5c([2H])c([2H])c([2H])c([2H])c5c(-c5c([2H])c([2H])c([2H])c([2H])c5[2H])c([2H])c43)c2[2H])c([2H])c1[2H] ; [2H]c1c([2H])c([2H])c(-c2cc3c(oc4cc(-c5ccccc5)cc(N(c5ccc(-c6ccccc6)cc5)c5cccc6c5sc5ccccc56)c43)c3ccccc23)c([2H])c1[2H]]

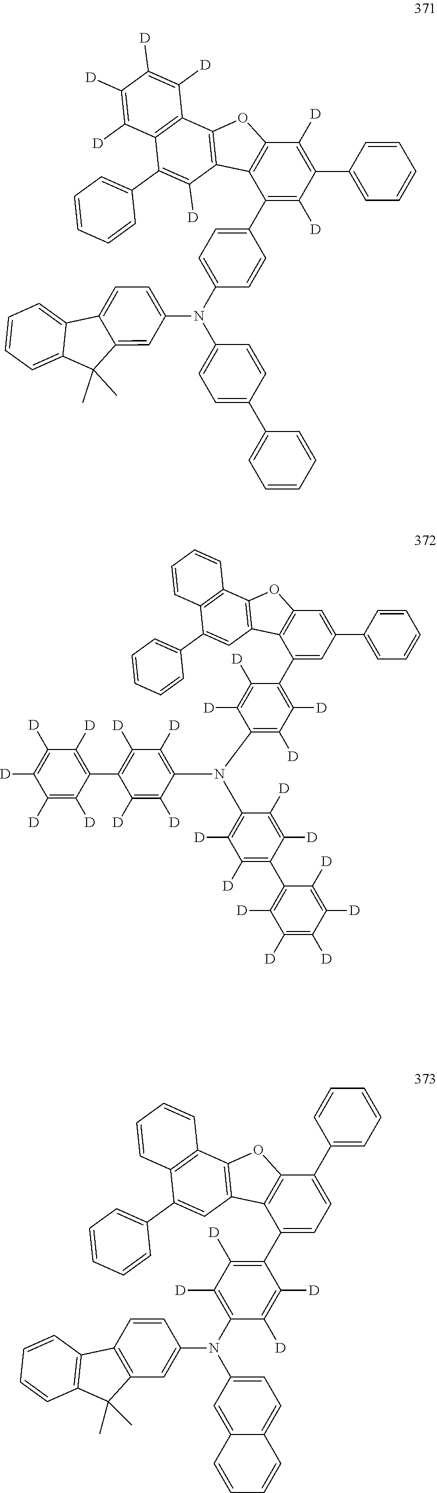

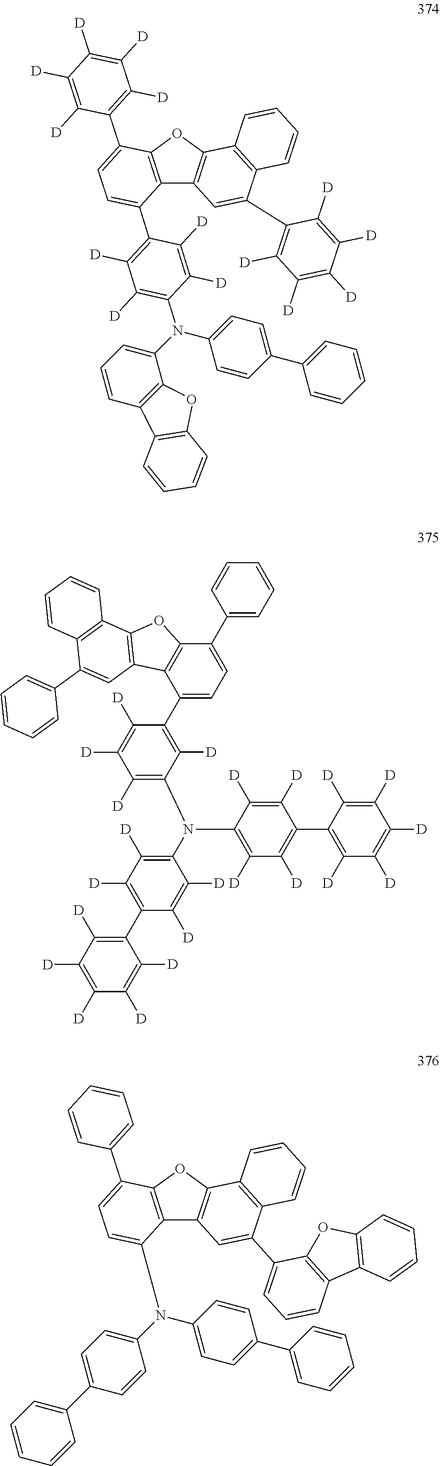

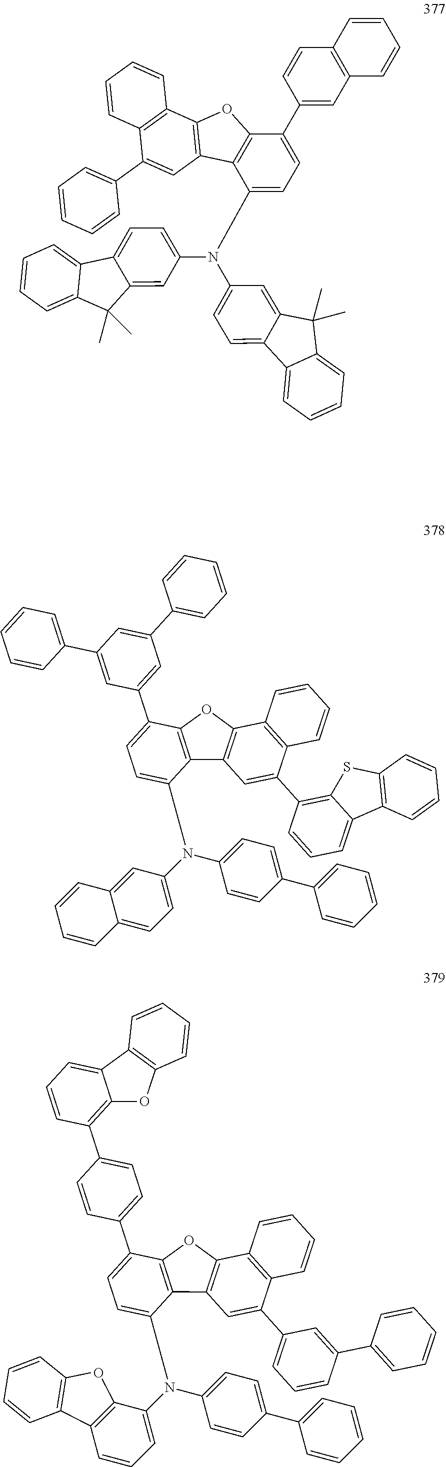 [SMILES: CC1(C)c2ccccc2-c2ccc(N(c3ccc4c(c3)C(C)(C)c3ccccc3-4)c3ccc(-c4ccc5ccccc5c4)c4oc5c6ccccc6c(-c6ccccc6)cc5c34)cc21 ; c1ccc(-c2ccc(N(c3ccc4ccccc4c3)c3ccc(-c4cc(-c5ccccc5)cc(-c5ccccc5)c4)c4oc5c6ccccc6c(-c6cccc7c6sc6ccccc67)cc5c34)cc2)cc1 ; c1ccc(-c2ccc(N(c3cccc4c3oc3ccccc34)c3ccc(-c4ccc(-c5cccc6c5oc5ccccc56)cc4)c4oc5c6ccccc6c(-c6cccc(-c7ccccc7)c6)cc5c34)cc2)cc1]

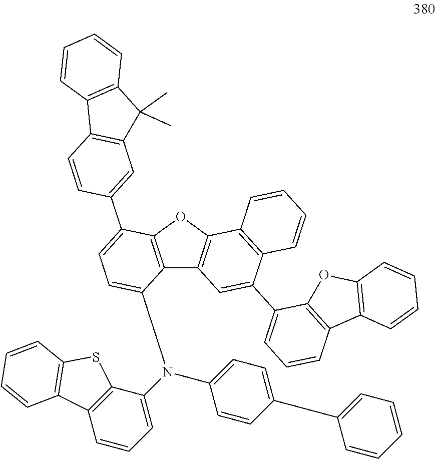 [SMILES: CC1(C)c2ccccc2-c2ccc(-c3ccc(N(c4ccc(-c5ccccc5)cc4)c4cccc5c4sc4ccccc45)c4c3oc3c5ccccc5c(-c5cccc6c5oc5ccccc56)cc34)cc21]

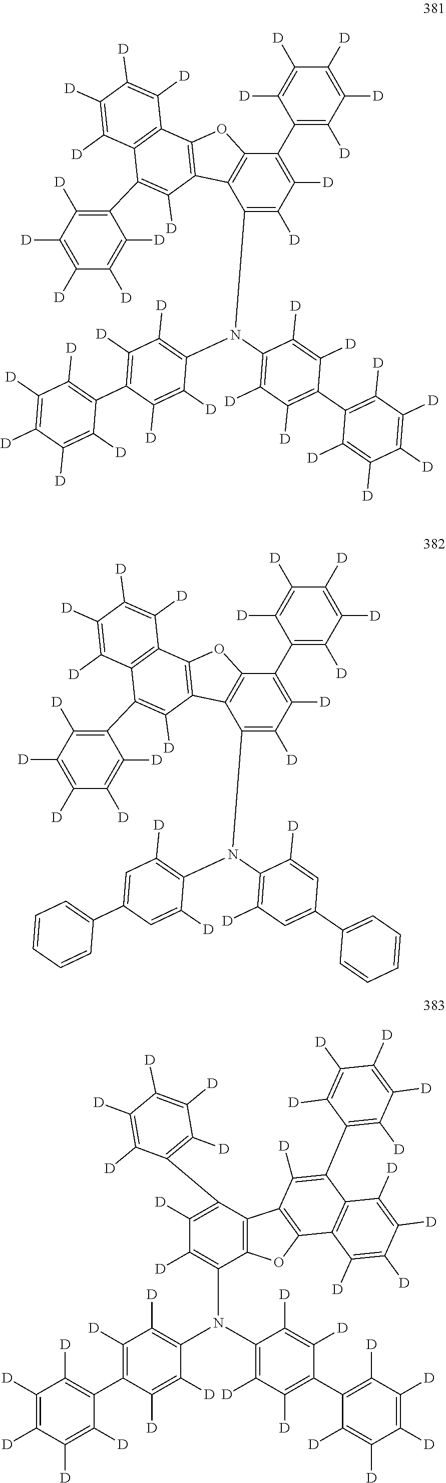

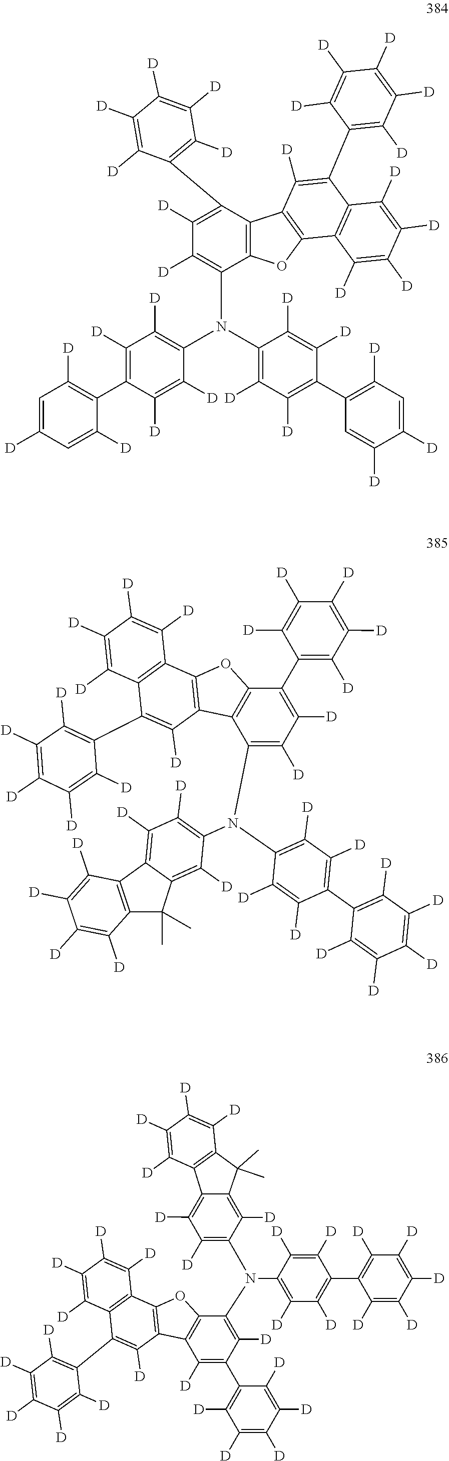

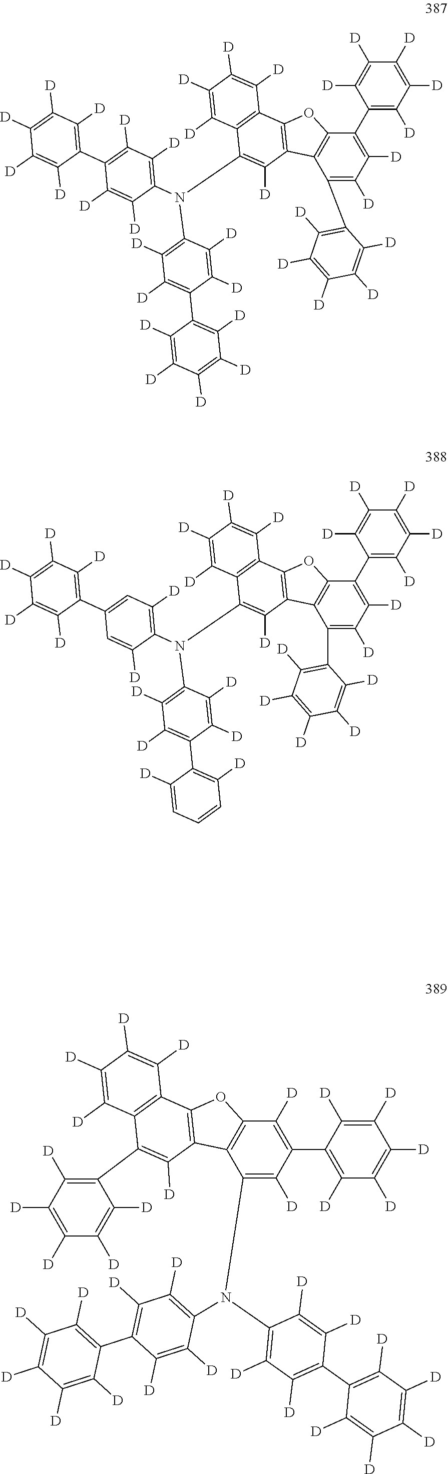

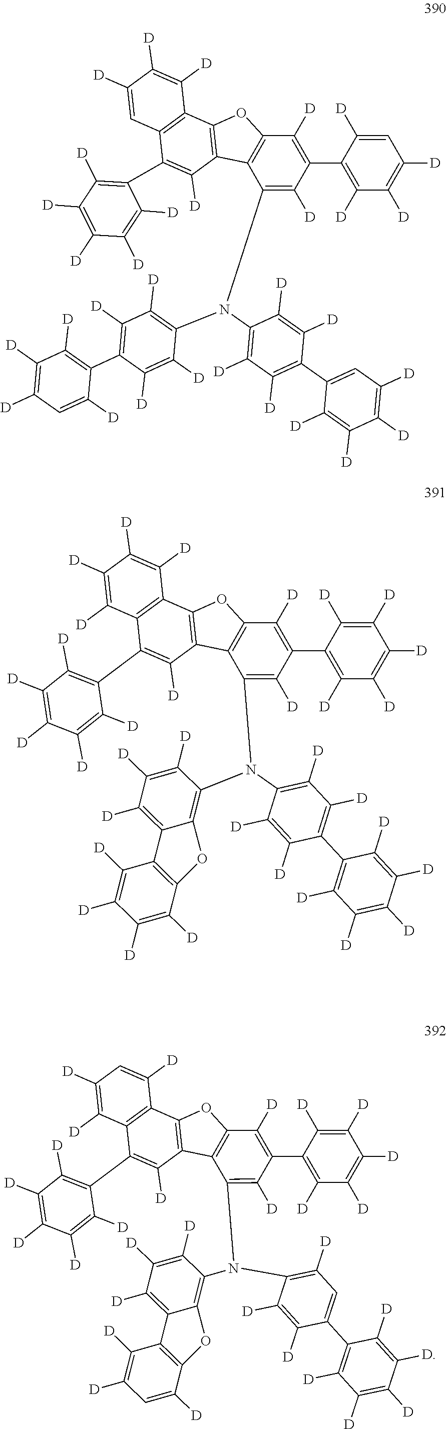

9. An organic light emitting device comprising:
a first electrode;
a second electrode; and
an organic material layer having one or more layers provided between the first electrode and the second electrode,
wherein one or more layers of the organic material layer comprise the heterocyclic compound according to claim 1.

10. The organic light emitting device of claim 9, wherein the organic material layer comprises a hole transport layer, and the hole transport layer comprises the heterocyclic compound.

11. The organic light emitting device of claim 9, wherein the organic material layer comprises an electron blocking layer, and the electron blocking layer comprises the heterocyclic compound.

12. The organic light emitting device of claim 9, further comprising one or two or more layers selected from the group consisting of a light emitting layer, a hole injection layer, a hole transport layer, an electron injection layer, an electron transport layer, a hole auxiliary layer, and a hole blocking layer.
